(19) United States
(12) Patent Application Publication
(10) Pub. No.: US 2026/0327325 A1
(43) Pub. Date: Oct. 22, 2026

(54) MULTISPECIFIC IMMUNE CELL ENGAGER

(71) Applicant: FORTE SUBSIDIARY, INC., Dallas, TX (US)

(72) Inventor: Paul A. WAGNER, San Juan, PR (US)

(21) Appl. No.: 19/545,974

(22) Filed: Feb. 20, 2026

Related U.S. Application Data
(63) Continuation of application No. PCT/US2024/043478, filed on Aug. 22, 2024.
(60) Provisional application No. 63/578,298, filed on Aug. 23, 2023.
(60) Provisional application No. 63/561,447, filed on Mar. 5, 2024.

(51) Int. Cl. C07K 16/28; A61K 39/00; A61P 35/00

(52) U.S. Cl. CPC C07K 16/2878; A61P 35/00; C07K 16/28; C07K 16/2809; C07K 16/283; A61K 2039/505; C07K 2317/31; C07K 2317/73

(57) ABSTRACT

Provided herein, in certain aspects, are multispecific binder molecules able to bind tumor associated antigens and engage immune cells. The multispecific binder molecules comprise a combination of GPRC5D, BCMA, CD38, SLAMF7, FcRH5, and CD3 binding modalities. Also provided here are methods for use of the multispecific binder molecules for inducing an immune response against tumor cells and for use in the treatment of hematological cancers.

### CROSS-REFERENCE TO RELATED APPLICATIONS

This application is a continuation of International Patent Application No. PCT/US2024/043478 filed Aug. 22, 2024, which claims the benefit of U.S. Provisional Patent Application No. 63/578,298, filed on Aug. 23, 2023, and U.S. Provisional Patent Application No. 63/561,447, filed on Mar. 5, 2024, both of which are incorporated by reference herein in their entireties.

### INCORPORATION BY REFERENCE OF SEQUENCE LISTING

The present application is being filed along with a Sequence Listing in electronic format. The Sequence Listing is provided as a file entitled 53654-725.301_SL.XML, created Feb. 20, 2026, which is 430,356 bytes in size. The information in the electronic format of the Sequence Listing is incorporated by reference in its entirety.

### TECHNICAL FIELD

The disclosure provided herein relates to multispecific binder molecules that bind to a combination of the proteins: B-cell maturation antigen (BCMA), G-protein coupled receptor, class C, group 5, member D (GPRC5D), CD38 molecule (CD38), SLAM family member 7 (SLAMF7), Fc receptor like 5 (FCRL5)—also known as FcRH5, and/or cluster determinant 3 (CD3), and methods for use of these multispecific binder molecules.

### BACKGROUND

Hematological tumor cells from some cancers are known to express certain cell surface markers at a higher protein level than in non-malignant cells. These cell surface markers can be termed tumor associated antigens, as an elevated level of expression of one or more of these markers is often indicative of a tumor cell phenotype. Targeted therapeutics aiming to stimulate immune cell recognition of hematological tumor cells and induce a directed cytotoxic response is a promising approach to develop novel treatments for a variety of hematological cancers. Targeted therapeutics may engage immune cells such as T cells, NK cells, and/or NKT cells and bring these cells in proximity to hematological tumor cells expressing an abnormally high level of one or more tumor associated antigens. Stimulation of these immune cells can lead to a targeted cytotoxic response directed specifically against the tumor cells expressing elevated levels of the one or more tumor associated antigens selected to identify malignant cells.

Multiple myeloma accounts for approximately 10% of hematological cancers in the United States and the treatment burden for patients diagnosed with multiple myeloma is extensive. Certain cells surface markers including BCMA are known to be overexpressed in multiple myeloma cells from a majority of patients. Strategies using an immune cell engager to bring T cells in proximity to BCMA+ multiple myeloma cells have met with some clinical success. However, many subjects receiving this type of treatment still do not reach a satisfactory clinical outcome or suffer from a relapse of cancer. Improvements are needed to effectively target multiple myeloma cells and induce immune responses directed against these tumor cells in order to achieve better clinical outcomes in patients.

### SUMMARY OF THE DISCLOSURE

Described herein, in some aspects, are multispecific binder molecules comprising three distinct tumor antigen binding moieties (a-c) comprising: a) a TNFRSF17 (BCMA) binding moiety, b) a GPRC5D binding moiety, and c) a FcRH5 binding moiety; and d) an immune cell engager. In some embodiments, the immune cell engager comprises a T cell binding moiety, an NK cell binding moiety, or an NKT cell binding moiety. In some embodiments, the immune cell engager comprises a T cell binding moiety. In some embodiments, the T cell binding moiety comprises a CD3 binding moiety. In some embodiments of the multispecific binder molecule described herein, at least one of the three distinct tumor antigen binding moieties is an antibody or antigen binding fragment thereof. In some embodiments, the antibody or antigen binding fragment thereof is a human antibody, a humanized antibody, or antigen binding fragment thereof. In some embodiments, the antibody or antigen binding fragment thereof comprises a chimeric antibody, a monoclonal antibody, a monovalent Fab′, a divalent Fab2, a single-chain variable fragment (scFv), a diabody, a minibody, a nanobody, a single-domain antibody (sdAb), a camelid antibody, or antigen binding fragment thereof. In some embodiments, the multispecific binder molecule described herein comprises a first immunoglobulin chain constant region and a second immunoglobulin chain constant region. In some embodiments, the TNFRSF17 (BCMA) binding moiety and the GPRC5D binding moiety are linked to the first immunoglobulin chain constant region, and wherein the CD3 binding moiety and the FcRH5 binding moiety are linked to the second immunoglobulin chain constant region. In some embodiments, the TNFRSF17 (BCMA) binding moiety and the CD3 binding moiety are linked to the first immunoglobulin chain constant region, and wherein the GPRC5D binding moiety and the FcRH5 binding moiety are linked to the second immunoglobulin chain constant region. In some embodiments, the TNFRSF17 (BCMA) binding moiety and the FcRH5 binding moiety are linked to the first immunoglobulin chain constant region, and wherein the GPRC5D binding moiety and the CD3 binding moiety are linked to the second immunoglobulin chain constant region. In some embodiments, the a) TNFRSF17 (BCMA) binding moiety, b) GPRC5D binding moiety, c) FcRH5 binding moiety, and d) CD3 binding moiety, comprise an antibody or antigen binding fragment thereof, wherein the antibody or antigen binding fragment thereof comprises Fab′, scFv, IgG-scFv, nanobody, mini-antibody, minibody, scFv-CH3 KiH, or scFv-KiH. In some embodiments, the first or second immunoglobulin chain constant region comprises a heavy chain constant region of IgG1, IgG2, and IgG4, or a variant thereof. In some embodiments, the first or second immunoglobulin chain constant regions form a IgG1 heavy chain or a variant thereof. In some embodiments, the TNFRSF17 (BCMA) binding moiety is coupled to the N-terminus of the IgG1 heavy chain or a variant thereof. In some embodiments, the TNFRSF17 (BCMA) binding moiety is coupled to the C-terminus of the IgG1 heavy chain or a variant thereof. In some embodiments, the GPRC5D binding moiety is coupled to the N-terminus of the IgG1 heavy chain or a variant thereof. In some embodiments, the GPRC5D binding moiety is coupled to the C-terminus of the IgG1 heavy chain or a variant thereof. In some embodiments, the FcRH5 binding moiety is coupled to the N-terminus of the IgG1 heavy chain or a variant thereof. In some embodiments, the FcRH5 binding moiety is coupled to the C-terminus of the IgG1 heavy chain or a variant thereof. In some embodiments, the CD3 binding moiety is coupled to the N-terminus of the IgG1 heavy chain or a variant thereof. In some embodiments, the CD3 binding moiety is coupled to the C-terminus of the IgG1 heavy chain or a variant thereof. In some embodiments, the IgG1 heavy chain comprises a mutation that reduces FcγR binding. In some embodiments, the mutation comprises N297A mutation. In some embodiments of the multispecific binder molecule described herein, at least one of the three distinct tumor antigen binding moieties is coupled to an immunoglobulin kappa light chain domain. In some embodiments of the multispecific binder molecule described herein, at least one of the three distinct tumor antigen binding moieties is coupled to an immunoglobulin lambda light chain domain. In some embodiments, one of the three distinct tumor antigen binding moieties is coupled to an immunoglobulin lambda light chain domain and another of the three distinct tumor antigen binding moieties is coupled to an immunoglobulin kappa light chain domain. In some embodiments, the TNFRSF17 (BCMA) binding moiety is derived from Teclistamab, AMG 701, Alnuctamab, Belantamab mafodotin, Elranatamab, Linvoseltamab, ABBV-383, HPN217, ISB 2001, GSK287916, MEDI2228, TNB-383B, REGN545, or AMG 420. In some embodiments, the GPRC5D binding moiety is derived from Talquetamab or RG6234. In some embodiments, the FcRH5 binding moiety is derived from Cevostamab or DFRF4539A. In some embodiments, the CD3 binding moiety is derived from AMG 701, Alnuctamab, Elranatamab, Linvoseltamab, Teclistamab, ABBV-383, HPN217, XmAb969, ISB1347, Talquetamab, RG6234, Cevostamab, SAR442257, F182112, or ISB 2001.

Described herein, in some aspects, are methods for inducing an immune response against a tumor cell, the methods comprising contacting the tumor cell with a multispecific binder molecule described herein. In some embodiments, the contacting induces proximity between the tumor cell and one or more immune effector cells. In some embodiments, the contacting supports formation of a cytolytic immunological synapse between the tumor cell and one or more immune effector cells. In some embodiments, the one or more immune effector cells comprise one or more activated T cells. In some embodiments, the one or more activated T cells release primary cytokines that induce the activation of other immune cell types wherein the other immune cell types comprise macrophages, dendritic cells, monocytes, NK cells, or any combination thereof. In some embodiments, the other immune cell types produce elevated levels of secondary cytokines, wherein the secondary cytokines comprise IL6, IL10, IL5, or any combination thereof. In some embodiments, the one or more immune effector cells induce apoptosis in a target tumor cell. In some embodiments, the one or more immune effector cells induce pyroptosis in a target tumor cell. In some embodiments, the one or more immune effector cells release one or more cytotoxic proteases of the granzyme family following the contacting. In some embodiments, the one or more immune effector cells release one or more cytotoxic proteases of the granzyme family following the contacting the one or more cytotoxic proteases of the granzyme family comprise Granzyme B (GrB). In some embodiments, the one or more immune effector cells one or more lytic proteins following the contacting. In some embodiments, the one or more lytic proteins comprise perforin. In some embodiments, the contacting engages a T cell receptor (TCR) on one or more T cells. In some embodiments, the one or more T cells comprise naïve T cells, helper T cells, cytotoxic T cells, or any combination thereof. In some embodiments, the one or more T cells receive a co-stimulatory signal resulting in T cell proliferation, differentiation, and survival. In some embodiments, the contacting induces co-stimulation independent polyclonal T-cell activation. In some embodiments, the tumor cell is a multiple myeloma tumor cell.

Described herein, in some aspects, are methods for treating a cancer in a subject in need thereof, the methods comprising administering a therapeutically effective amount of a multispecific binder molecule described herein. In some embodiments, the cancer is a hematological cancer. In some embodiments, the cancer is myeloma. In some embodiments, the cancer is multiple myeloma. In some embodiments, the therapeutically effective amount reduces a risk of the subject developing cytokine release syndrome (CRS) following the administering.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a diagram of a tetraspecific binder molecule formed from six polypeptide sequences in an IgG-Fab configuration. One modified heavy chain is configured from N-terminal to C-terminal as: i) Binding arm A heavy chain variable region (VH), ii) a first constant Ig domain of a heavy chain (CH1), iii) a second constant Ig domain of a heavy chain (CH2), iv) a mutated third Ig domain of a heavy chain (CH3) comprising KiH knob substitutions, v) Binding arm C VH, and vi) CH1. A second modified heavy chain is configured from N-terminal to C-terminal as: i) Binding arm B VH, ii) CH1, iii) CH2 iv) a mutated CH3 comprising KiH hole substitutions, v) Binding arm D VH, and vi) CH1. A first light chain is configured from N-terminal to C-terminal as: i) Binding arm A light chain variable region (VL). and ii) a constant Ig domain of a light chain (CL-κ set 1 or CL-κ set 2). Paired Binding arm A VH and Binding arm A VL form a first binding moiety. A second light chain is configured from N-terminal to C-terminal as: i) Binding arm B VL and ii) CL-κ set 1 or CL-κ set 2. Paired Binding arm B VH and Binding arm B VL form a second binding moiety. A third light chain is configured from N-terminal to C-terminal as: i) Binding arm C VL and ii) CL-κ set 1 or CL-κ set 2. Paired Binding arm C VH and Binding arm C VL form a third binding moiety. A fourth light chain is configured from N-terminal to C-terminal as: i) Binding arm D VL, and ii) CL-κ set 1 or CL-κ set 2. Paired Binding arm D VH and Binding arm D VL form a fourth binding moiety.

FIG. 2 shows a diagram of a tetraspecific binder molecule formed from six polypeptide sequences in an IgG-Fab configuration. Paired Binding arm A VH and Binding arm A VL form a BCMA binding moiety. Paired Binding arm B VH and Binding arm B VL form a GPRC5D binding moiety. Paired Binding arm C VH and Binding arm C VL form an FcRH5 binding moiety. Paired Binding arm D VH and Binding arm D VL form a CD3 binding moiety.

FIG. 3 shows a diagram of a tetraspecific binder molecule formed from six polypeptide sequences in an IgG-Fab configuration. Two light chains are paired with each of two modified heavy chains with the respective VH and VL regions arranged to form a BCMA binding moiety, a GPRC5D binding moiety, a CD3 binding moiety, and a CD38 binding moiety.

FIG. 4 shows a diagram of a tetraspecific binder molecule formed from six polypeptide sequences in an IgG-Fab configuration. Two light chains are paired with each of two modified heavy chains with the respective VH and VL regions arranged to form a BCMA binding moiety, a GPRC5D binding moiety, a CD3 binding moiety, and a SLAMF7 binding moiety.

FIG. 5 shows a diagram of a tetraspecific binder molecule formed from six polypeptide sequences in an IgG-Fab configuration. Two light chains are paired with each of two modified heavy chains with the respective VH and VL regions arranged to form a SLAMF7 binding moiety, a GPRC5D binding moiety, a CD3 binding moiety, and a CD38 binding moiety.

FIG. 6 shows a diagram of a tetraspecific binder molecule formed from six polypeptide sequences in an IgG-Fab configuration. Two light chains are paired with each of two modified heavy chains with the respective VH and VL regions arranged to form a BCMA binding moiety, a SLAMF7 binding moiety, a CD3 binding moiety, and a CD38 binding moiety.

FIG. 7 shows a diagram of a tetraspecific binder molecule formed from six polypeptide sequences in an IgG-Fab configuration. Two light chains are paired with each of two modified heavy chains with the respective VH and VL regions arranged to form a BCMA binding moiety in binding arm A, a CD3 binding moiety in binding arm B, a FcRH5 binding moiety in binding arm C, and a GPRC5D binding moiety in binding arm D.

FIG. 8 shows a diagram of a tetraspecific binder molecule formed from six polypeptide sequences in an IgG-Fab configuration. Two light chains are paired with each of two modified heavy chains with the respective VH and VL regions arranged to form a BCMA binding moiety in binding arm A, a CD3 binding moiety in binding arm B, a GPRC5D binding moiety in binding arm C, and a FcRH5 binding moiety in binding arm D.

FIG. 9 shows a diagram of a tetraspecific binder molecule formed from six polypeptide sequences in an IgG-Fab configuration. Two light chains are paired with each of two modified heavy chains with the respective VH and VL regions arranged to form a FcRH5 binding moiety in binding arm A, a CD3 binding moiety in binding arm B, a GPRC5D binding moiety in binding arm C, and a BCMA binding moiety in binding arm D.

FIG. 10 shows a diagram of a tetraspecific binder molecule formed from six polypeptide sequences in an IgG-Fab configuration. Two light chains are paired with each of two modified heavy chains with the respective VH and VL regions arranged to form a FcRH5 binding moiety in binding arm A, a CD3 binding moiety in binding arm B, a BCMA binding moiety in binding arm C, and a GPRC5D binding moiety in binding arm D.

FIG. 11 shows a diagram of a tetraspecific binder molecule formed from six polypeptide sequences in an IgG-Fab configuration. Two light chains are paired with each of two modified heavy chains with the respective VH and VL regions arranged to form a GPRC5D binding moiety in binding arm A, a CD3 binding moiety in binding arm B, a FcRH5 binding moiety in binding arm C, and a BCMA binding moiety in binding arm D.

FIG. 12 shows a diagram of a tetraspecific binder molecule formed from six polypeptide sequences in an IgG-Fab configuration. Two light chains are paired with each of two modified heavy chains with the respective VH and VL regions arranged to form a GPRC5D binding moiety in binding arm A, a CD3 binding moiety in binding arm B, a BCMA binding moiety in binding arm C, and a FcRH5 binding moiety in binding arm D.

### DETAILED DESCRIPTION

Cancers originating in the blood and bone marrow make up a significant portion of diseases in which there is either an unmet medical need or treatment options offering limited success. Multiple myeloma (MM) is the second most common hematological malignancy in the United States accounting for approximately 10% of hematological cancers. Multiple myeloma is characterized by uncontrolled clonal expansion of neoplastic plasma cells in and outside of the bone marrow (BM). These neoplastic plasma cells undergo excessive proliferation while surrounded by a permissive and protective tumor microenvironment. A dependence of MM plasma cells on BM microenvironment cells, each of which express different sets of cell surface receptors and adhesion molecules, has been demonstrated indicating a critical role for cross-talk involving receptors and adhesion in establishing and maintaining the permissive and protective role of BM microenvironment in MM progression. CD38, BCMA, and SLAMF7 are highly expressed in MM plasma cells pointing to their utility as selective targets for delivering therapeutics to malignant cells. BCMA is expressed at significantly higher levels in MM plasma cells than in healthy plasma cells, but is not expressed in other hematological cells types. CD38 and SLAMF7, while being highly expressed in MM plasma cells, also maintain expression in other immune cell types including immune effector cells and other hematopoietic cell lineages. GPRC5D is another marker strongly expressed in MM plasma cells, and restricted to expression among immune cell types to plasma cells. FcRH5 is expressed exclusively in B cell lineages and expression of FcRH5 is retained in plasma cells, including MM plasma cells. This expression pattern for FcRH5 makes it a promising candidate to use for tumor antigen targeting in MM and other hematological malignancies derived from B cell lineages.

Chemotherapy, stem cell transplant, or gene therapy are currently used where treatment in a subject is indicated in myeloma with symptoms. Yet, the majority of patients treated for multiple myeloma, including those having undergone either autologous hematopoietic stem-cell transplantation (auto-SCT) or allogeneic hematopoietic stem-cell transplantation, do suffer relapse after initial treatment. Relapse within the first 18 months of initial multiple myeloma diagnosis is considered to be functional high-risk multiple myeloma. Treatment options for relapsed disease can include retreatment using the original treatment modality, use of a different treatment modality, or a first or second auto-SCT. As relapsed multiple myeloma progresses, the disease can become refractory to one or more formerly effective treatments. This stage of multiple myeloma is referred to as relapsed/refractory multiple myeloma (RRMM).

Immunotherapeutic approaches to elicit or amplify an immune response are classified as activation immunotherapies and can be effective as therapeutics for cancer. Immune effector cells such as lymphocytes, macrophages, natural killer (NK) cells, and cytotoxic T lymphocytes participate in the defense against cancer by targeting abnormal antigens expressed on the surface of tumor cells. Compositions and methods to activate immune responses in these cells to target tumor cells can provide an effective therapy for various cancers.

Treatment modalities currently used to treat RRMM in patients include dexamethasone, proteasome inhibitors, nuclear export inhibitors, immunomodulatory drugs such as thalidomide or pomalidomide, or antibody treatments. Yet, despite recent advances in the treatment of MM, acute forms of MM remain almost incurable for the majority of patients since treatment-resistant clones eventually emerge and evolve leading to a low 5-year overall survival (OS) of about 50%. Clinical outcomes for subjects with RRMM are poor due to the diminishing performance and the gradual decreased durability of response with successive lines of MM therapy. High risk patients suffer shorter survival times, partly due to weakened immune systems. Immunotherapy has provided improved response rate, patient survival, and a favorable safety profile offering hope of significant long term treatment efficacy and durability for patients suffering from MM. An obstacle to effective immunotherapy is the immunosuppressive nature of the BM microenvironment. Malignant MM cells interact with surrounding BM accessory cells, including BM stromal cells, osteoclasts, regulatory T cells, regulatory B cells, myeloid-derived suppressor cells, tumor-associated macrophages, and plasmacytoid dendritic cells within the BM microenvironment. These surrounding BM accessory cells promote the growth and chemoresistance of MM plasma cells and also promote tumor cells immune escape, inhibiting tumor-specific T effector cells, inducing T-cell anergy, and increasing regulatory T cell numbers, fostering an immunosuppressive BM microenvironment. Therapies aimed at targeting specific MM antigens and also reversing or overcoming the immunosuppressive BM microenvironment provide a path to developing more effective therapies. Clinical success using anti-BCMA CAR-T cell therapy has demonstrated a significance of and a need to develop improved T cell-directing immunotherapies. The choice of tumor antigen(s) that is targeted is critical in effectiveness of a T cell-directing immunotherapy.

Described herein are multispecific binder molecules targeting tumor associated antigens which can be used in novel treatments for MM. A multispecific binder molecule targeted to at least three MM tumor antigens and having an immune cell engager component, offers a novel approach to immunotherapy in MM. The multispecific binder molecule can bring an immune effector cell or immune effector cells in proximity to a pre-malignant or malignant MM cell. The multispecific binder molecule can simultaneously bind to a pre-malignant or malignant MM cell and an immune effector cell, creating an immune synapse between the two cells. This interaction encourages T cell activation, promotes tumor cell lysis, enhances T cell proliferation, or any combination thereof. The cytotoxic effect of this treatment involves polyclonal T cell responses. The multispecific binder molecule modulates general T cell function, such as differentiation of naïve T cells to T cells with memory phenotypes (e.g., central memory and effector memory T cells) which contributes to improved occurrence of minimal residual disease (MRD) negativity in treated patients.

Multispecific binder molecules described herein and methods for use thereof target at least three tumor associated antigens selected from GPRC5D, BCMA, CD38, FcRH5 and SLAMF7 to effective target cancerous plasma cells, including pre-malignant and malignant plasma cells. The multispecific binder molecule additionally has an immune cell engager which comprises either a T cell binding moiety, an NK cell binding moiety, and/or an NKT cell binding moiety. The immune cell engager component of the multispecific binder molecule allows for binding to immune cells which can then elicit an immune response and the tumor associated antigen binding moieties allow for binding to pre-malignant and/or malignant plasma cells. Contacting a pre-malignant or a malignant plasma cell with the multispecific binding molecule can bring cytotoxic immune cells, which may be activated, in close proximity thereby eliciting a targeted immune response against pre-malignant and/or malignant plasma cells. Administering the multispecific binding molecule to a subject in need thereof can similarly induce a targeted immune response against pre-malignant and/or malignant plasma cells present in the subject. In some embodiments, the multispecific binder molecule has specific binding affinity for at least two tumor associated antigens and at least one T cell binding moiety, NK cell binding moiety, or NKT cell binding moiety. In some embodiments, the multispecific binder molecule has specific binding affinity for at least three tumor associated antigens and at least one T cell binding moiety, NK cell binding moiety, or NKT cell binding moiety. In some instances, the multispecific binder molecule having specific binding affinity for at least three tumor associated antigens and at least one T cell binding moiety, NK cell binding moiety, or NKT cell binding moiety is referred to as a tetraspecific binder molecule or a tetraspecific binding molecule.

Disclosed herein, in certain aspects, are multispecific binding molecules that bind to a combination of the targets: B-cell maturation antigen (BCMA), G-protein coupled receptor, class C, group 5, member D (GPRC5D), CD38 molecule (CD38), Fc receptor-homolog 5 (FcRH5), SLAM family member 7 (SLAMF7), and/or cluster determinant 3 (CD3). In some aspects, the combination of targets comprise an amino acid sequence and/or a carbohydrate, lipid, or other chemical entity of BCMA, GPRC5D, CD38, FcRH5, SLAMF7, and/or CD3. In some embodiments, the targets comprise one or more antigens comprising a portion of a target molecule that is able to be bound by an adaptive immune molecule such as an antibody or antigen-binding fragment thereof, B-cell receptor, or T-cell receptor. Disclosed herein, in certain aspects, are multispecific binding molecules that bind to a combination of the antigens: B-cell maturation antigen (BCMA), G-protein coupled receptor, class C, group 5, member D (GPRC5D), CD38 molecule (CD38), Fc receptor-homolog 5 (FcRH5), SLAM family member 7 (SLAMF7), and/or cluster determinant 3 (CD3). In some aspects, also disclosed herein are methods used for inducing an immune response against a tumor cells by contacting the tumor cell with a multispecific binding molecule described herein. In some aspects, also disclosed herein are methods for treating a cancer in a subject in need thereof by administering a therapeutically effective amount of a multispecific binding molecule described herein.

G protein-coupled receptor class C group 5 member D (GPRC5D) is a cell surface receptor exhibiting highly variable levels of expression in patients with MM. Low levels of GPRC5D are found in normal tissues. High levels of GPRC5D mRNA are positively correlated with high plasma cell count in the BM of MM patients indicating its utility as an MM-associated antigen. Human GPRC5D is described in the NCBI Gene database under Gene ID: 55507 and in the UniProt database under Entry: Q9NZD1. GPRC5D can be expressed in high quantities in plasma B cells. GPRC5D can be expressed in high quantities in malignant plasma B cells. GPRC5D can be expressed in memory B-cells. GPRC5D can be expressed in class switched memory B cells. GPRC5D can be expressed in exhausted memory B cells. GPRC5D can be expressed in plasmablasts. GPRC5D can be expressed in peripheral blood mononuclear cells (PBMCs). In some embodiments, GPRC5D expression serves as a marker for plasma B cells. In some embodiments of multispecific binder molecules described herein, GPRC5D is a tumor associated antigen to which the multispecific binder molecule binds.

BCMA encodes a member of the TNF-receptor superfamily and is preferentially expressed in mature B lymphocytes including plasma cells. BCMA specifically binds to the signaling ligand BAFF (TNFSF13B) and this signaling event can lead to NF-kappaB and MAPK8/JNK activation. BCMA can be expressed in high quantities in plasma B cells. BCMA can be expressed in high quantities in malignant plasma B cells. BCMA can be expressed in germinal center B cells. BCMA can be expressed in memory B cells. BCMA can be expressed in naïve B cells. BCMA can be expressed in class switched memory B cells. BCMA can be expressed in exhausted memory B cells. BCMA can be expressed in plasmablasts. BCMA can be expressed in plasmacytoid dendritic cells. BCMA can be expressed in PBMCs. In some embodiments, BCMA expression serves as a marker for plasma B cells. Human BCMA (TNFRSF17) is described in the NCBI Gene database under Gene ID: 608 and in the UniProt database under Entry: Q02223. In some embodiments of multispecific binder molecules described herein, BCMA is a tumor associated antigen to which the multispecific binder molecule binds.

Fc receptor-homolog 5 (FcRH5) is a single-pass type I membrane protein containing 8 immunoglobulin-like C2-type domains that is exclusively expressed in B cell lineages. Two membrane-bound isoforms of FcRH5 protein and one soluble isoform of FcRH5 protein have been identified. FcRH5, also known as FCRL5 or CD307, has elevated expression in MM cells as compared to normal B cells or non-cancerous plasma cells. High levels of soluble FcRH5 has been in detected in serum from patients with MM, chronic lymphocytic leukemia (CLL), and mantle cell lymphoma (MCL). In some embodiments, FcRH5 serves as a marker for plasma B cells. FcRH5 can be expressed in high quantities in plasma B cells. FcRH5 can be expressed in high quantities in malignant plasma B cells. FcRH5 can be expressed in germinal center B cells. FcRH5 can be expressed in memory B cells. FcRH5 can be expressed in class switched memory B cells. FcRH5 can be expressed in exhausted memory B cells. Human FcRH5 is described in the NCBI Gene database under Gene ID: 83416 and in the UniProt database under Entry: Q96RD9. In some embodiments of multispecific binder molecules described herein, FcRH5 is a tumor associated antigen to which the multispecific binder molecule binds.

Cyclic ADP ribose hydrolase is a transmembrane glycoprotein also known as CD38. CD38 is widely expressed in lymphoid and myeloid cell lineages. For example, CD38 is expressed by T cell precursors such as CD4+CD8+ double-positive thymocytes, in a subset of memory T cells, in CD4+/CD45RA+ naïve T cells, and CD38 is a marker for activated T cells. Among CD8+ T cells, CD38 is strongly expressed during period of chronic infection. CD38 expression is modulated during different stages of B cell differentiation being expressed at high levels on BM B cell precursors and then downregulated in mature B cells. CD38 is then expressed at high levels in terminally differentiated plasma cells. CD38 is also strongly expressed in several pathological cells including chronic lymphocytic leukemia cells and on malignant MM cells. Detailed analysis of CD38 expression within the BM microenvironment has indicated that only plasma cell types express high levels of CD38 demonstrating its utility as a specific antigen to target plasma cells. CD38 can be expressed in plasmablasts at a high level of expression. CD38 can be expressed in NK cells at a high level of expression. CD38 can be expressed in memory B cells. CD38 can be expressed in naïve B cells. CD38 can be expressed in plasmacytoid dendritic cells. CD38 can be expressed in exhausted memory B cells. Human CD38 is described in the NCBI Gene database under Gene ID: 952 and in the UniProt database under Entry: P28907. In some embodiments of multispecific binder molecules described herein, CD38 is a tumor associated antigen to which the multispecific binder molecule binds. In some embodiments of multispecific binder molecules described herein, a CD38 binding moiety is an immune cell engager comprising an NK cell binding moiety. In some embodiments of multispecific binder molecules described herein, a CD38 binding moiety is an immune cell engager comprising a T cell binding moiety. In some embodiments of multispecific binder molecules described herein, a CD38 binding moiety is an immune cell engager comprising an NKT cell binding moiety.

The SLAM family member 7 (SLAMF7), also known as CD319 or CS1, is a robustly expressed marker of normal plasma cells and malignant MM plasma cells. SLAMF7 plays roles in several cell functions including maintaining plasma cell survival, cell adhesion, and NK cell and CD8+ T cell-mediated cytotoxicity. In NK cells, SLAMF7 functions as a positive regulatory of NK cell activation. Primary myeloma cells and human-derived multiple myeloma cell lines express higher levels of SLAMF7 than normal cells. SLAMF7 has a demonstrated role in mediating proliferation of MM cells. High levels of soluble SLAMF7 protein and elevated SLAMF7 mRNA levels in purified plasma cells have been documented in subjects affected by monoclonal gammopathies across the entire clinical spectrum, (e.g., monoclonal gammopathy of undetermined significance (MGUS), smoldering MM, active MM, and RRMM). In recently diagnosed MM patients, increased soluble SLAMF7 was found to be positively associated with active MM and was further increased in the most aggressive presentations of the disease. SLAMF7 can be expressed at a high level in plasma B cells. SLAMF7 can be expressed at a high level in malignant plasma B cells. SLAMF7 can be expressed in classical, non-classical, and intermediate monocytes. SLAMF7 can be expressed in a variety of T cells including Gamma-Delta T cells, mucosal-associated invariant T cells, memory CD4+ T cells, memory CD8+ T cells, central memory CD8+ T cells, effector memory CD8+ T cells, naïve CD4+ T cells, naïve CD8+ T cells, or terminal effector memory CD4+ T cells, or any combination thereof. SLAMF7 can be expressed in plasmacytoid dendritic cells. SLAMF7 can be expressed in myeloid dendritic cells. SLAMF7 can be expressed in NK cells. SLAMF7 can be expressed in plasmablasts. SLAMF7 can be expressed in PBMCs. Human SLAMF7 is described in the NCBI Gene database under Gene ID: 57823 and in the UniProt database under Entry: Q9NQ25. In some embodiments of multispecific binder molecules described herein, SLAMF7 is a tumor associated antigen to which the multispecific binder molecule binds. In some embodiments of multispecific binder molecules described herein, a SLAMF7 binding moiety is an immune cell engager comprising an NK cell binding moiety. In some embodiments of multispecific binder molecules described herein, a SLAMF7 binding moiety is an immune cell engager comprising a T cell binding moiety. In some embodiments of multispecific binder molecules described herein, a SLAMF7 binding moiety is an immune cell engager comprising an NKT cell binding moiety.

Cluster of differentiation 3 (CD3) is a protein complex and T cell co-receptor. The protein complex contains a CD3γ chain, a CD38 chain, and two CD3& chains. These chains in the protein complex associate with the T-cell receptor (TCR) and the CD3 zeta chain in order to generate an activation signal in T lymphocytes. Together, the CD3 zeta chain, the TCR, and the other CD3 chains in the protein complex constitute the TCR complex. The CD3 complex serves as a T cell co-receptor that associates noncovalently with TCR. CD3Y, CD38, and CD38 are highly related immunoglobulin superfamily cell-surface proteins each having a single extracellular immunoglobulin domain. CD3 is initially expressed in the cytoplasm of pro-lymphocytes. CD3 is bound to the plasma membrane of all mature T-cells, and virtually no other cell types indicating its specificity for use as an immune cell engager target. The CD3 protein complex is a defining feature of the T cell lineage, making binders that bind to CD3 a T cell marker. CD38 (CD3D) can be expressed in regulatory T cells, mucosal-associated invariant T cells, naïve CD4+ T cells, delta gamma T cells, memory CD4+ T cells, terminal effector memory CD4+ T cells, naïve CD8+ T cells, central memory CD8+ T cells, effector memory CD8+ T cells, terminal effector memory CD8+ T cells, naïve regulatory T cells, memory regulatory T cells, activated T cells, or cytotoxic T cells, or any combination thereof. CD3D can be expressed in NKT cells. CD3D can be expressed in NK cells. CD38 (CD3E) can be expressed in regulatory T cells, mucosal-associated invariant T cells, naïve CD4+ T cells, delta gamma T cells, memory CD4+ T cells, naïve CD8+ T cells, memory CD4+ T cells, terminal effector memory CD4+ T cells, central memory CD8+ T cells, effector memory CD8+ T cells, terminal effector memory CD8+ T cells, naïve regulatory T cells, memory regulatory T cells, activated T cells, or cytotoxic T cells, or any combination thereof. CD3E can be expressed in NKT cells. CD3E can be expressed in NK cells. CD3E can be expressed in PBMCs. CD3γ (CD3G) can be expressed in regulatory T cells, mucosal-associated invariant T cells, naïve CD4+ T cells, delta gamma T cells, memory CD4+ T cells, naïve CD8+ T cells, memory CD4+ T cells, terminal effector memory CD4+ T cells, central memory CD8+ T cells, effector memory CD8+ T cells, terminal effector memory CD8+ T cells, naïve regulatory T cells, memory regulatory T cells, activated T cells, or cytotoxic T cells, or any combination thereof. CD3G can be expressed in NKT cells. CD3G can be expressed in NK cells. CD3G can be expressed in PBMCs. CD3 zeta (CD247) can be expressed in regulatory T cells, mucosal-associated invariant T cells, naïve CD4+ T cells, delta gamma T cells, memory CD4+ T cells, naïve CD8+ T cells, memory CD4+ T cells, terminal effector memory CD4+ T cells, central memory CD8+ T cells, effector memory CD8+ T cells, terminal effector memory CD8+ T cells, naïve regulatory T cells, memory regulatory T cells, activated T cells, or cytotoxic T cells, or any combination thereof. CD247 can be expressed in NKT cells. CD247 can be expressed in NK cells. CD247 can be expressed in PBMCs. CD247 can be expressed in intermediate monocytes. Human CD3γ is described in the NCBI Gene database under Gene ID: 917 and in the UniProt database under Entry: P09693. Human CD3δ is described in the NCBI Gene database under Gene ID: 915 and in the UniProt database under Entry: P04234. Human CD3ε is described in the NCBI Gene database under Gene ID: 916 and in the UniProt database under Entry: P07766. Human CD3 zeta (CD247) is described in the NCBI Gene database under Gene ID: 919 and in the UniProt database under Entry: P20963. In some embodiments of multispecific binder molecules described herein, one or more sites within the CD3 receptor complex are targets to which the multispecific binder molecule binds. In some embodiments, by binding to the CD3 receptor complex or by binding to one of the elements comprising the CD3 receptor complex, the multispecific binder molecule functions as an immune cell engager. In some embodiments, the immune cell engager comprises a T cell binding moiety, an NK cell binding moiety, or an NKT cell binding moiety, or any combination thereof. In some embodiments, the T cell binding moiety, the NK cell binding moiety, the NKT cell binding moiety, or any combination thereof binds to CD3 or binds to one of the elements comprising the CD3 receptor complex.

### Antibody-Based Multi-Specific Binders

In some aspects, the multispecific binder molecule comprises an antibody-based multispecific binder. In some aspects, the multispecific binder is an antibody-based multispecific binder. In some aspects, the antibody-based multispecific binder comprises a binding moiety. In some aspects, the antibody-based multispecific binder comprises at least one, at least two, at least three, or at least four antibody-based binding moieties. In some embodiments, at least one antibody-based binding moiety binds to a tumor associated antigen. In some embodiments, at least one, at least two, at least three, or at least four antibody-based binding moieties bind to one or more tumor associated antigens. In some embodiments, at least three antibody-based binding moieties bind to three distinct tumor antigens. In some embodiments, each of at least one, at least two, at least three, or at least four antibody-based binding moieties bind to a different tumor associated antigen. In some instances, the tumor associated antigen is GPRC5D. In some instances, the tumor associated antigen is BCMA. In some instances, the tumor associated antigen is FcRH5. In some instances, the tumor associated antigen is CD38. In some instances, the tumor associated antigen is SLAMF7. In some instances, the tumor associated antigen is an extracellular portion of GPRC5D, BCMA, FcRH5, CD38, or SLAMF7. In some instances, the tumor associated antigen is present on the surface of a plasma cell. In some instances, the tumor associated antigen is present on the surface of a pre-malignant plasma cell. In some instances, the tumor associated antigen is present on the surface of a malignant plasma cell. In some instances, the tumor associated antigen is present in the blood of the subject. In some instances, the tumor associated antigen is present in a serum sample taken from the subject. In some instances, the tumor associated antigen is present in a BM aspirate from the subject. In some instances, the tumor associated antigen is present in a BM biopsy from the subject. In some instances, at least one antibody-based binding moiety binds to an immune cell. In some instances, at least one antibody-based binding moiety binds to an immune effector cell. In some instances, at least one antibody-based binding moiety binds to a T cell. In some instances, at least one antibody-based binding moiety binds to an NK cell. In some instances, at least one antibody-based binding moiety binds to an NKT cell. In some instances, at least one antibody-based binding moiety binds to a T cell, an NK cell, an NKT cell, or any combination thereof. In some instances, the at least one antibody-based binding moiety that binds to an immune cell functions as an immune cell engager. In some instances, the immune cell engager comprises a CD3 binding moiety. In some instances, the immune cell engager binds to one or more components of the CD3 co-receptor.

In some aspects, the multispecific binder molecule comprises at least three binding moieties selected from the group consisting of: a GPRC5D-binding moiety, a BCMA-binding moiety, an FcRH5-binding moiety, a CD38-binding moiety, a SLAMF7-binding moiety, and a CD3-binding moiety. In some embodiments, the multispecific binder molecule comprises at least four binding moieties selected from the group consisting of: a GPRC5D-binding moiety, a BCMA-binding moiety, an FcRH5-binding moiety, a CD38-binding moiety, a SLAMF7-binding moiety, and a CD3-binding moiety. In some embodiments, at least one of the at least three binding moieties is an antibody or antigen binding fragment thereof. In some embodiments, at least one of the at least four binding moieties is an antibody or antigen binding fragment thereof. In some embodiments, at least two of the at least three binding moieties are antibodies or antigen binding fragments thereof. In some embodiments, at least two of the at least four binding moieties are antibodies or antigen binding fragments thereof. In some embodiments, three of the at least three binding moieties are antibodies or antigen binding fragments thereof. In some embodiments, at least three of the at least four binding moieties are antibodies or antigen binding fragments thereof. In some embodiments, four of the at least four binding moieties are antibodies or antigen binding fragments thereof. In some embodiments, the multispecific binder molecule comprises one or more antibody-based binders and one or more non-antibody based binders.

In some aspects, the multispecific binder molecule comprises at least one antibody-based multispecific binder. In some aspects, the multispecific binder molecule comprises at least one antibody-based binder. In some aspects, the multispecific binder molecule comprises at least two or at least three antibody-based binders that bind different tumor associated antigens. In some embodiments, the multispecific binder molecule comprises an immune cell engager. In some embodiments, one or more antibody-based binders function as an immune cell engager. In some embodiments, the immune cell engager comprises a T cell binding moiety, an NK cell binding moiety, or an NKT cell binding moiety, an any combination thereof. In some embodiments, the immune cell engager comprises a T cell binding moiety. In some embodiments, the multispecific binder molecule comprises at least two antibody-based binding moieties that bind to different tumor associated antigens and at least one antibody-based immune cell binder. In some embodiments, the multispecific binder molecule comprises at least three antibody-based binding moieties that bind to different tumor associated antigens and at least one antibody-based immune cell binder. In some aspects, the antibody-based multispecific binder molecule comprises a GPRC5D binding moiety. GPRC5D is a tumor associated antigen in several forms of tumors or cancers. In some instances, GPRC5D is a tumor associated antigen for a hematological cancer. In some instances, GPRC5D is a myeloma tumor associated antigen. In some instances, GPRC5D is a multiple myeloma tumor associated antigen. In some embodiments, the antibody-based GPRC5D binding moiety of the multispecific binder molecule binds to a tumor associated antigen.

In some aspects, the antibody-based multispecific binder molecule comprises a BCMA binding moiety. BCMA is a tumor associated antigen in several forms of tumors or cancers. In some instances, BCMA is a tumor associated antigen for a hematological cancer. In some instances, BCMA is a myeloma tumor associated antigen. In some instances, BCMA is a multiple myeloma tumor associated antigen. In some embodiments, the antibody-based BCMA binding moiety of the multispecific binder molecule binds to a tumor associated antigen.

In some aspects, the antibody-based multispecific binder molecule comprises an FcRH5 binding moiety. FcRH5 is a tumor associated antigen in several forms of tumors or cancers. In some instances, FcRH5 is a tumor associated antigen for a hematological cancer. In some instances, FcRH5 is a myeloma tumor associated antigen. In some instances, FcRH5 is a multiple myeloma tumor associated antigen. In some instances, FcRH5 is a CLL tumor associated antigen. In some instances, FcRH5 is an MCL tumor associated antigen. In some embodiments, the antibody-based FcRH5 binding moiety of the multispecific binder molecule binds to a tumor associated antigen.

In some aspects, the antibody-based multispecific binder molecule comprises a CD38 binding moiety. CD38 is a tumor associated antigen in several forms of tumors or cancers. In some instances, CD38 is a tumor associated antigen for a hematological cancer. In some instances, CD38 is a myeloma tumor associated antigen. In some instances, CD38 is a multiple myeloma tumor associated antigen. In some instances, the antibody-based CD38 binding moiety of the multispecific binder molecule binds to a tumor associated antigen. In some instances, CD38 is a marker expressed in T cells, NK cells, or NKT cells, or any combination thereof.

In some aspects, the antibody-based multispecific binder molecule comprises a SLAMF7 binding moiety. SLAMF7 is a tumor associated antigen in several forms of tumors or cancers. In some instances, SLAMF7 is a tumor associated antigen for a hematological cancer. In some instances, SLAMF7 is a myeloma tumor associated antigen. In some instances, SLAMF7 is a multiple myeloma tumor associated antigen. In some instances, the antibody-based SLAMF7 binding moiety of the multispecific binder molecule binds to a tumor associated antigen. In some instances, SLAMF7 is a marker expressed in T cells, NK cells, or NKT cells, or any combination thereof.

In some aspects, the antibody-based multispecific binder molecule comprises a CD3 binding moiety. In some embodiments, the CD3 binding moiety binds to one or more components of the CD3 co-receptor complex. CD3 is an antigen expressed by several types of immune cells. In some instances, CD3 expression marks immune effector cells. In some instances, CD3 is a marker expressed in T cells, or NKT cells, or T cells and NKT cells. In some instances, CD3 is a marker expressed in effector T cells. In some instances, CD3 is a marker expressed in cytotoxic T cells. In some embodiments, the CD3 binding moiety is an antibody-based CD3 binding moiety. In some embodiments, the antibody-based CD3 binding moiety functions as an immune cell engager.

In some aspects, the multispecific binder molecule comprises four antibody-based binding moieties. In some instances, the multispecific binder molecule comprises an antibody-based GPRC5D binding moiety, an antibody-based BCMA binding moiety, an antibody-based CD38 binding moiety, and an antibody-based immune cell engager. In some instances, the multispecific binder molecule comprises an antibody-based GPRC5D binding moiety, an antibody-based BCMA binding moiety, an antibody-based SLAMF7 binding moiety, and an antibody-based immune cell engager. In some instances, the multispecific binder molecule comprises an antibody-based GPRC5D binding moiety, an antibody-based SLAMF7 binding moiety, an antibody-based CD38 binding moiety, and an antibody-based immune cell engager. In some instances, the multispecific binder molecule comprises an antibody-based CD38 binding moiety, an antibody-based SLAMF7 binding moiety, an antibody-based BCMA binding moiety, and an antibody-based immune cell engager. In some instances, the multispecific binder molecule comprises an antibody-based GPRC5D binding moiety, an antibody-based BCMA binding moiety, an antibody-based FcRH5 binding moiety, and an antibody-based immune cell engager. In some instances, the multispecific binder molecule comprises an antibody-based GPRC5D binding moiety, an antibody-based CD38 binding moiety, an antibody-based FcRH5 binding moiety, and an antibody-based immune cell engager. In some instances, the multispecific binder molecule comprises an antibody-based GPRC5D binding moiety, an antibody-based SLAMF7 binding moiety, an antibody-based FcRH5 binding moiety, and an antibody-based immune cell engager. In some instances, the multispecific binder molecule comprises an antibody-based BCMA binding moiety, an antibody-based CD38 binding moiety, an antibody-based FcRH5 binding moiety, and an antibody-based immune cell engager. In some instances, the multispecific binder molecule comprises an antibody-based BCMA binding moiety, an antibody-based SLAMF7 binding moiety, an antibody-based FcRH5 binding moiety, and an antibody-based immune cell engager. In some instances, the multispecific binder molecule comprises an antibody-based CD38 binding moiety, an antibody-based SLAMF7 binding moiety, an antibody-based FcRH5 binding moiety, and an antibody-based immune cell engager. In some embodiments, one or more non-antibody based binding moieties replace one or more antibody-based binding moieties in a configuration of a multispecific binder molecule.

In some embodiments, one or more of the antibody-based binding moieties enhances binding of the multispecific binder molecule to plasma B cells. In some embodiments, one or more of the antibody-based binding moieties enhances binding of the multispecific binder molecule to pre-malignant plasma B cells. In some embodiments, one or more of the antibody-based binding moieties enhances binding of the multispecific binder molecule to malignant plasma B cells. In some embodiments, one or more of the antibody-based binding moieties enhances binding of the multispecific binder molecule to plasma B cells from a patient diagnosed with MGUS. In some embodiments, one or more of the antibody-based binding moieties enhances binding of the multispecific binder molecule to plasma B cells from a patient diagnosed with MM. In some embodiments, one or more of the antibody-based binding moieties enhances binding of the multispecific binder molecule to plasma B cells derived from a patient diagnosed with MM. In some embodiments, one or more of the antibody-based binding moieties enhances binding of the multispecific binder molecule to plasma B cells from a patient diagnosed with smoldering MM. In some embodiments, one or more of the antibody-based binding moieties enhances binding of the multispecific binder molecule to plasma B cells from a patient diagnosed with acute MM. In some embodiments, one or more of the antibody-based binding moieties enhances binding of the multispecific binder molecule to plasma B cells from a patient diagnosed with RRMM. In some embodiments, one or more of the antibody-based binding moieties enhances binding of the multispecific binder molecule to plasma B cells derived from a patient diagnosed with RRMM. In some embodiments, one or more of the antibody-based binding moieties enhances binding of the multispecific binder molecule to plasma B cells that have undergone deletion of one or more tumor associated antigens. In some embodiments, one or more of the antibody-based binding moieties enhances binding of the multispecific binder molecule to plasma B cells that have undergone downregulation of one or more tumor associated antigens. In some embodiments, SLAMF7 has undergone deletion or downregulation in plasma B cells or MM plasma B cells. In some embodiments, CD38 has undergone deletion or downregulation in plasma B cells or MM plasma B cells. In some embodiments, BCMA has undergone deletion or downregulation in plasma B cells or MM plasma B cells. In some embodiments, GPRC5D has undergone deletion or downregulation in plasma B cells or MM plasma B cells. In some embodiments, FcRH5 has undergone deletion or downregulation in plasma B cells or MM plasma B cells. In some embodiments, the multispecific binder molecule maintains therapeutic efficacy in a subject in which any one of CD38, BCMA, GPRC5D, FcRH5, or SLAMF7 have undergone deletion or downregulation in plasma B cells or MM plasma B cells by targeting a retained tumor antigen. In some embodiments, the multispecific binder molecule maintains therapeutic efficacy in a subject in which any two of CD38, BCMA, GPRC5D, FcRH5, or SLAMF7 have undergone deletion or downregulation in plasma B cells or MM plasma B cells by targeting a retained tumor antigen. In some embodiments, the multispecific binder molecule maintains therapeutic efficacy in a subject in which BCMA and GPRC5D have undergone deletion or downregulation in plasma B cells or MM plasma B cells by targeting a retained tumor antigen. In some embodiments, the multispecific binder molecule maintains therapeutic efficacy in a subject in which BCMA and FcRH5 have undergone deletion or downregulation in plasma B cells or MM plasma B cells by targeting a retained tumor antigen. In some embodiments, the multispecific binder molecule maintains therapeutic efficacy in a subject in which BCMA and CD38 have undergone deletion or downregulation in plasma B cells or MM plasma B cells by targeting a retained tumor antigen. In some embodiments, the multispecific binder molecule maintains therapeutic efficacy in a subject in which BCMA and SLAMF7 have undergone deletion or downregulation in plasma B cells or MM plasma B cells by targeting a retained tumor antigen. In some embodiments, the multispecific binder molecule maintains therapeutic efficacy in a subject in which GPRC5D and FcRH5 have undergone deletion or downregulation in plasma B cells or MM plasma B cells by targeting a retained tumor antigen. In some embodiments, the multispecific binder molecule maintains therapeutic efficacy in a subject in which GPRC5D and CD38 have undergone deletion or downregulation in plasma B cells or MM plasma B cells by targeting a retained tumor antigen. In some embodiments, the multispecific binder molecule maintains therapeutic efficacy in a subject in which GPRC5D and SLAMF7 have undergone deletion or downregulation in plasma B cells or MM plasma B cells by targeting a retained tumor antigen. In some embodiments, the multispecific binder molecule maintains therapeutic efficacy in a subject in which CD38 and FcRH5 have undergone deletion or downregulation in plasma B cells or MM plasma B cells by targeting a retained tumor antigen. In some embodiments, the multispecific binder molecule maintains therapeutic efficacy in a subject in which SLAMF7 and FcRH5 have undergone deletion or downregulation in plasma B cells or MM plasma B cells by targeting a retained tumor antigen. In some embodiments, the multispecific binder molecule maintains therapeutic efficacy in a subject in which CD38 and SLAMF7 have undergone deletion or downregulation in plasma B cells or MM plasma B cells by targeting a retained tumor antigen.

In some embodiments, one or more of the antibody-based binding moieties enhances binding of the multispecific binder molecule to plasma B cells in a subject that was determined to have a very good partial response (VGPR) to a previous MM treatment. In some embodiments, one or more of the antibody-based binding moieties enhances binding of the multispecific binder molecule to plasma B cells in a subject that was determined to have a partial response (PR) to a previous MM treatment. In some embodiments, one or more of the antibody-based binding moieties enhances binding of the multispecific binder molecule to plasma B cells in a subject that was determined to be non-responsive to a previous MM treatment. In some embodiments, one or more of the antibody-based binding moieties enhances binding of the multispecific binder molecule to plasma B cells that possess significant upregulation of expression of one or more tumor associated antigens. In some embodiments, the antibody-based GPRC5D binding moiety enhances binding to plasma B cells. In some embodiments, the antibody-based BCMA binding moiety enhances binding to plasma B cells. In some embodiments, the antibody-based FcRH5 binding moiety enhances binding to plasma B cells. In some embodiments, the antibody-based CD38 binding moiety enhances binding to plasma B cells. In some embodiments, the antibody-based SLAMF7 binding moiety enhances binding to plasma B cells. In some embodiments, the antibody-based CD3 binding moiety enhances binding to immune effector cells.

In some aspects, an antibody-based binder of the multispecific binding molecule comprises an antibody or antigen binding fragment thereof. In some embodiments, the antibody or antigen binding fragment thereof is a human antibody or antigen binding fragment thereof. In some embodiments, the antibody or antigen binding fragment thereof is a humanized antibody or antigen binding fragment thereof. In some embodiments, the antibody or antigen binding fragment thereof comprises a chimeric antibody, a monoclonal antibody, a monovalent Fab′, a divalent Fab2, a single-chain variable fragment (scFv), a diabody, a minibody, a nanobody, a single-domain antibody (sdAb), a camelid antibody, or antigen binding fragment thereof. In some embodiments, a GPRC5D-binding moiety, a BCMA-binding moiety, an FcRH5-binding moiety, a CD38-binding moiety, a SLAMF7-binding moiety, a CD3-binding moiety, or any combination thereof comprise an antibody or antigen binding fragment thereof, wherein the antibody or antigen binding fragment thereof comprises Fab′, scFv, IgG-scFv, nanobody, mini-antibody, minibody, scFv-CH3 KiH, or scFv-KiH. In some embodiments, an antibody-based binder that binds to GPRC5D is derived from a monoclonal antibody. In some embodiments, an antibody-based binder that binds to BCMA is derived from a monoclonal antibody. In some embodiments, an antibody-based binder that binds to FcRH5 is derived from a monoclonal antibody. In some embodiments, an antibody-based binder that binds to CD38 is derived from a monoclonal antibody. In some embodiments, an antibody-based binder that binds to SLAMF7 is derived from a monoclonal antibody. In some embodiments, an antibody-based binder that binds to CD3 is derived from a monoclonal antibody.

In certain aspects, the multispecific binder molecule comprises a first immunoglobulin chain constant region and a second immunoglobulin chain constant region. In some embodiments, at least three of the antibody-based binding moieties of the multispecific binder molecule: i) the CD3 binding moiety, ii) the GPRC5D binding moiety, iii) the TNFRSF17 (BCMA) binding moiety, iv) the CD38 binding moiety, v) the SLAMF7 (CD319) binding moiety, and vi) the FcRH5 binding moiety, are linked to either the first immunoglobulin chain constant region or the second immunoglobulin chain constant region. In some embodiments, the TNFRSF17 (BCMA) binding moiety and the GPRC5D binding moiety are linked to the first immunoglobulin chain constant region and the CD3 binding moiety and optionally one of the CD38 binding moiety or the SLAMF7 (CD319) binding moiety are linked to the second immunoglobulin chain constant region. In some embodiments, the TNFRSF17 (BCMA) binding moiety and the CD3 binding moiety are linked to the first immunoglobulin chain constant region and the GPRC5D binding moiety and optionally one of the CD38 binding moiety or the SLAMF7 (CD319) binding moiety are linked to the second immunoglobulin chain constant region. In some embodiments, the CD3 binding moiety and the GPRC5D binding moiety are linked to the first immunoglobulin chain constant region and the TNFRSF17 (BCMA) binding moiety and optionally one of the CD38 binding moiety or the SLAMF7 (CD319) binding moiety are linked to the second immunoglobulin chain constant region. In some embodiments, the CD3 binding moiety and the GPRC5D binding moiety are linked to the first immunoglobulin chain constant region and the TNFRSF17 (BCMA) binding moiety and the FcRH5 binding moiety are linked to the second immunoglobulin chain constant region. In some embodiments, the CD3 binding moiety and the TNFRSF17 (BCMA) binding moiety are linked to the first immunoglobulin chain constant region and the GPRC5D binding moiety and the FcRH5 binding moiety are linked to the second immunoglobulin chain constant region. In some embodiments, the CD3 binding moiety and the FcRH5binding moiety are linked to the first immunoglobulin chain constant region and the GPRC5D binding moiety and the TNFRSF17 (BCMA) binding moiety are linked to the second immunoglobulin chain constant region. In some embodiments, the linked binding moieties are covalently coupled to a single polypeptide chain. In some embodiments, the linked binding moieties are conjugated to a single polypeptide chain. In some embodiments, the i) CD3 binding moiety, ii) GPRC5D binding moiety, iii) TNFRSF17 (BCMA) binding moiety, iv) CD38 binding moiety, v) SLAMF7 (CD319) binding moiety, and vi) FcRH5 binding moiety comprises an antibody or antigen binding fragment thereof, wherein the antibody or antigen binding fragment thereof comprises Fab′, scFv, IgG-scFv, nanobody, mini-antibody, minibody, scFv-CH3 KiH, or scFv-KiH. In some embodiments, the first or second immunoglobulin chain constant region comprises a heavy chain constant region of IgG1, IgG2, and IgG4, or a variant thereof. In some embodiments, the first or second immunoglobulin chain constant region form an IgG heavy chain, or variant thereof.

In some aspects, the multispecific binder molecule comprises one or more antibody heavy chains. In some embodiments, the multispecific binder molecule comprises a first heavy chain and a second heavy chain. In some embodiments, i) the first heavy chain; ii) the second heavy chain; or iii) the first and second heavy chains, are modified heavy chains. In some embodiments, i) the first heavy chain or first modified heavy chain; ii) the second heavy chain or second modified heavy chain; iii) the first heavy chain and second heavy chain; iv) the first modified heavy chain and second heavy chain; v) the first heavy chain and second modified heavy chain; or vi) the first modified heavy chain and second modified heavy chain, are variant heavy chains. In some embodiments, a variant heavy chain comprises one or more amino acid substitutions, insertions, deletions, or any combination thereof in a CH1 domain, a CH2 domain, a CH3 domain, or any combination of CH1, CH2, and CH3 domains. In some embodiments, a variant heavy chain comprises a CH1 domain. In some embodiments, a variant heavy chain comprises a CH1 domain and a CH2 domain. In some embodiments, a heavy chain or a variant heavy chain comprises a CH1 domain, a CH2 domain, and a CH3 domain. In some embodiments, a heavy chain or a variant heavy chain comprises a CH1 domain, a CH2 domain, or a CH3 domain, or any combination thereof. In some embodiments, a modified heavy chain comprises a modification to the N-terminus of the heavy chain. In some embodiments, a modified heavy chain comprises a CH1 domain, a CH2 domain, and a CH3 domain with a modification of the N-terminus of the heavy chain. In some embodiments, a modified heavy chain comprises a modification to the C-terminus of the heavy chain. In some embodiments, a modified heavy chain comprises a CH1 domain, a CH2 domain, and a CH3 domain with a modification of the C-terminus of the heavy chain. In some embodiments, a modified heavy chain comprises a CH1 domain, a CH2 domain, and a CH3 domain with a modification of the N-terminus and the C-terminus of the heavy chain. In some embodiment, a heavy chain or a modified heavy chain comprises a VH domain at the N-terminus. In some embodiments, a heavy chain is modified by coupling one or more protein domains to the N-terminus, C-terminus, or N-terminus and C-terminus of the heavy chain to form a modified heavy chain. In some embodiments, the modification to the N-terminus or C-terminus of the modified heavy chain comprises a binding moiety coupled to the modified heavy chain. In some embodiments, the binding moiety coupled to the modified heavy chain comprises an antibody-based binding moiety. In some embodiments, the antibody-based binding moiety comprises a VH domain, a nanobody, a biparatopic nanobody, a camelid heavy-chain variable domain (VHH), an scFv, a dimeric scFv, a bispecific-scFv, a diabody, a triabody, a tetrabody, an scAb, a V-NAR domain, a minibody, an sdAb, a disulfide stabilized Fv protein (“dsFv”), or antigen binding fragment thereof. In some embodiments, the binding moiety comprises a non-antibody-based binding moiety.

In some embodiments, the first heavy chain or first modified heavy chain comprises a first immunoglobulin chain constant region and the second heavy chain or second modified heavy chain comprises a second immunoglobulin chain constant region. In some embodiments, the first heavy chain or first modified heavy chain comprises a CD3 binding moiety at the N-terminus of the first heavy chain linked to the first immunoglobulin chain constant region. In some embodiments, the first heavy chain or first modified heavy chain comprises a GPRC5D binding moiety at the N-terminus of the first heavy chain linked to the first immunoglobulin chain constant region. In some embodiments, the first heavy chain or first modified heavy chain comprises a BCMA binding moiety at the N-terminus of the first heavy chain linked to the first immunoglobulin chain constant region. In some embodiments, the first heavy chain or first modified heavy chain comprises a CD38 binding moiety at the N-terminus of the first heavy chain linked to the first immunoglobulin chain constant region. In some embodiments, the first heavy chain or first modified heavy chain comprises a SLAMF7 binding moiety at the N-terminus of the first heavy chain linked to the first immunoglobulin chain constant region. In some embodiments, the first modified heavy chain comprises a CD3 binding moiety at the C-terminus of the first heavy chain. In some embodiments, the first modified heavy chain comprises a GPRC5D binding moiety at the C-terminus of the first heavy chain. In some embodiments, the first modified heavy chain comprises a BCMA binding moiety at the C-terminus of the first heavy chain. In some embodiments, the first modified heavy chain comprises an FcRH5 binding moiety at the N-terminus of the first heavy chain. In some embodiments, the first modified heavy chain comprises an FcRH5 binding moiety at the C-terminus of the first heavy chain. In some embodiments, the second modified heavy chain comprises an FcRH5 binding moiety at the N-terminus of the second heavy chain. In some embodiments, the second modified heavy chain comprises an FcRH5 binding moiety at the C-terminus of the second heavy chain. In some embodiments, the first modified heavy chain comprises a CD38 binding moiety at the C-terminus of the first heavy chain. In some embodiments, the first modified heavy chain comprises a SLAMF7 binding moiety at the C-terminus of the first heavy chain. In some embodiments, the second heavy chain or second modified heavy chain comprises a CD3 binding moiety at the N-terminus of the second heavy chain linked to the second a immunoglobulin chain constant region. In some embodiments, the second heavy chain or second modified heavy chain comprises a GPRC5D binding moiety at the N-terminus of the second heavy chain linked to the second immunoglobulin chain constant region. In some embodiments, the second heavy chain or second modified heavy chain comprises a BCMA binding moiety at the N-terminus of the second heavy chain linked to the second immunoglobulin chain constant region. In some embodiments, the second heavy chain or second modified heavy chain comprises a CD38 binding moiety at the N-terminus of the second heavy chain linked to the second immunoglobulin chain constant region. In some embodiments, the second heavy chain or second modified heavy chain comprises a SLAMF7 binding moiety at the N-terminus of the second heavy chain linked to the second immunoglobulin chain constant region. In some embodiments, the second modified heavy chain comprises a CD3 binding moiety at the C-terminus of the second heavy chain. In some embodiments, the second modified heavy chain comprises a GPRC5D binding moiety at the C-terminus of the second heavy chain. In some embodiments, the second modified heavy chain comprises a BCMA binding moiety at the C-terminus of the second heavy chain. In some embodiments, the second modified heavy chain comprises a CD38 binding moiety at the C-terminus of the second heavy chain. In some embodiments, the second modified heavy chain comprises a SLAMF7 binding moiety at the C-terminus of the second heavy chain. In some embodiments, the TNFRSF17 (BCMA) binding moiety is coupled to the C-terminus of a modified IgG1 heavy chain or a variant thereof. In some embodiments, the CD38 binding moiety is coupled to the C-terminus of a modified IgG1 heavy chain or a variant thereof. In some embodiments, the GPRC5D binding moiety is coupled to the C-terminus of a modified IgG1 heavy chain or a variant thereof. In some embodiments, the CD3 binding moiety is coupled to the C-terminus of a modified IgG1 heavy chain or a variant thereof. In some embodiments, the SLAMF7 binding moiety is coupled to the C-terminus of a modified IgG1 heavy chain or a variant thereof.

In some embodiments, a first modified heavy chain, a second modified heavy chain, or a first and a second modified heavy chain comprise a binding moiety at the N-terminus and the C-terminus of a heavy chain. In some embodiments, the first or second modified heavy chain comprises a GPRC5D binding moiety at the N-terminus and a CD3 binding moiety coupled to the C-terminus. In some embodiments, the first or second modified heavy chain comprises a GPRC5D binding moiety at the N-terminus and a BCMA binding moiety coupled to the C-terminus. In some embodiments, the first or second modified heavy chain comprises a GPRC5D binding moiety at the N-terminus and a CD38 binding moiety coupled to the C-terminus. In some embodiments, the first or second modified heavy chain comprises a GPRC5D binding moiety at the N-terminus and a SLAMF7 binding moiety coupled to the C-terminus. In some embodiments, the first or second modified heavy chain comprises a BCMA binding moiety at the N-terminus and a CD3 binding moiety coupled to the C-terminus. In some embodiments, the first or second modified heavy chain comprises a BCMA binding moiety at the N-terminus and a GPRC5D binding moiety coupled to the C-terminus. In some embodiments, the first or second modified heavy chain comprises a BCMA binding moiety at the N-terminus and a CD38 binding moiety coupled to the C-terminus. In some embodiments, the first or second modified heavy chain comprises a BCMA binding moiety at the N-terminus and a SLAMF7 binding moiety coupled to the C-terminus. In some embodiments, the first or second modified heavy chain comprises a CD38 binding moiety at the N-terminus and a CD3 binding moiety coupled to the C-terminus. In some embodiments, the first or second modified heavy chain comprises a CD38 binding moiety at the N-terminus and a GPRC5D binding moiety coupled to the C-terminus. In some embodiments, the first or second modified heavy chain comprises a CD38 binding moiety at the N-terminus and a BCMA binding moiety coupled to the C-terminus. In some embodiments, the first or second modified heavy chain comprises a CD38 binding moiety at the N-terminus and a SLAMF7 binding moiety coupled to the C-terminus. In some embodiments, the first or second modified heavy chain comprises a SLAMF7 binding moiety at the N-terminus and a CD3 binding moiety coupled to the C-terminus. In some embodiments, the first or second modified heavy chain comprises a SLAMF7 binding moiety at the N-terminus and a GPRC5D binding moiety coupled to the C-terminus. In some embodiments, the first or second modified heavy chain comprises a SLAMF7 binding moiety at the N-terminus and a BCMA binding moiety coupled to the C-terminus. In some embodiments, the first or second modified heavy chain comprises a SLAMF7 binding moiety at the N-terminus and a CD38 binding moiety coupled to the C-terminus. In some embodiments, the first or second modified heavy chain comprises a CD3 binding moiety at the N-terminus and a SLAMF7 binding moiety coupled to the C-terminus. In some embodiments, the first or second modified heavy chain comprises a CD3 binding moiety at the N-terminus and a GPRC5D binding moiety coupled to the C-terminus. In some embodiments, the first or second modified heavy chain comprises a CD3 binding moiety at the N-terminus and a BCMA binding moiety coupled to the C-terminus. In some embodiments, the first or second modified heavy chain comprises a CD3 binding moiety at the N-terminus and a CD38 binding moiety coupled to the C-terminus. In some embodiments, the first or second modified heavy chain comprises an FcRH5 binding moiety at the N-terminus and a CD3 binding moiety coupled to the C-terminus. In some embodiments, the first or second modified heavy chain comprises an FcRH5 binding moiety at the N-terminus and a BCMA binding moiety coupled to the C-terminus. In some embodiments, the first or second modified heavy chain comprises an FcRH5 binding moiety at the N-terminus and a GPRC5D binding moiety coupled to the C-terminus. In some embodiments, the first or second modified heavy chain comprises an FcRH5 binding moiety at the N-terminus and a CD38 binding moiety coupled to the C-terminus. In some embodiments, the first or second modified heavy chain comprises an FcRH5 binding moiety at the N-terminus and a SLAMF7 binding moiety coupled to the C-terminus. In some embodiments, the first or second modified heavy chain comprises an FcRH5 binding moiety at the C-terminus and a CD3 binding moiety coupled to the N-terminus. In some embodiments, the first or second modified heavy chain comprises an FcRH5 binding moiety at the C-terminus and a BCMA binding moiety coupled to the N-terminus. In some embodiments, the first or second modified heavy chain comprises an FcRH5 binding moiety at the C-terminus and a GPRC5D binding moiety coupled to the N-terminus. In some embodiments, the first or second modified heavy chain comprises an FcRH5 binding moiety at the C-terminus and a CD38 binding moiety coupled to the N-terminus. In some embodiments, the first or second modified heavy chain comprises an FcRH5 binding moiety at the C-terminus and a SLAMF7 binding moiety coupled to the N-terminus.

In some embodiments, a first heavy chain comprising at least two binding moieties is paired with a second heavy chain comprising at least two binding moieties to form a tetraspecific binding molecule comprising a CD3 binding moiety, a GPRC5D binding moiety, a BCMA binding moiety, and a CD38 binding moiety. In some embodiments, a first heavy chain comprising at least two binding moieties is paired with a second heavy chain comprising at least two binding moieties to form a tetraspecific binding molecule comprising a CD3 binding moiety, a GPRC5D binding moiety, a BCMA binding moiety, and a SLAMF7 binding moiety. In some embodiments, a first heavy chain comprising at least two binding moieties is paired with a second heavy chain comprising at least two binding moieties to form a tetraspecific binding molecule comprising a CD3 binding moiety, a GPRC5D binding moiety, a CD38 binding moiety, and a SLAMF7 binding moiety. In some embodiments, a first heavy chain comprising at least two binding moieties is paired with a second heavy chain comprising at least two binding moieties to form a tetraspecific binding molecule comprising a CD3 binding moiety, a CD38 binding moiety, a BCMA binding moiety, and a SLAMF7 binding moiety. In some embodiments, a first heavy chain comprising at least two binding moieties is paired with a second heavy chain comprising at least two binding moieties to form a tetraspecific binding molecule comprising a CD3 binding moiety, a GPRC5D binding moiety, a BCMA binding moiety, and an FcRH5 binding moiety. In some embodiments, a first heavy chain comprising at least two binding moieties is paired with a second heavy chain comprising at least two binding moieties to form a tetraspecific binding molecule comprising a CD3 binding moiety, a CD38 binding moiety, a BCMA binding moiety, and an FcRH5 binding moiety. In some embodiments, a first heavy chain comprising at least two binding moieties is paired with a second heavy chain comprising at least two binding moieties to form a tetraspecific binding molecule comprising a CD3 binding moiety, a SLAMF7 binding moiety, a BCMA binding moiety, and an FcRH5 binding moiety. In some embodiments, a first heavy chain comprising at least two binding moieties is paired with a second heavy chain comprising at least two binding moieties to form a tetraspecific binding molecule comprising a CD3 binding moiety, a GPRC5D binding moiety, a CD38 binding moiety, and an FcRH5 binding moiety. In some embodiments, a first heavy chain comprising at least two binding moieties is paired with a second heavy chain comprising at least two binding moieties to form a tetraspecific binding molecule comprising a CD3 binding moiety, a GPRC5D binding moiety, a SLAMF7 binding moiety, and an FcRH5 binding moiety. In some embodiments, a first heavy chain comprising at least two binding moieties is paired with a second heavy chain comprising at least two binding moieties to form a tetraspecific binding molecule comprising a CD3 binding moiety, a CD38 binding moiety, a SLAMF7 binding moiety, and an FcRH5 binding moiety. In some embodiments, a VH domain residing within a heavy chain is paired with a VL domain residing within an antibody light chain to form a binding moiety. In some embodiments, a VH domain and VL domain that form a binding moiety are present in a single antibody chain.

In some embodiments, the multispecific binder molecule comprises one or more heavy chains paired with one or more antibody light chains. In some embodiments, the one or more light chains are variant or modified light chains. In some embodiments, the one or more light chains comprise one or more binding moieties. In some embodiments, the one or more light chains comprises one or more VL domains. In some embodiments, a VL domain pairs with a VH domain in a heavy chain to form a binding moiety. In some embodiments, the light chain or modified light chain comprises a CD3 binding moiety at the N-terminus or C-terminus. In some embodiments, the light chain or modified light chain comprises a GPRC5D binding moiety at the N-terminus or C-terminus. In some embodiments, the light chain or modified light chain comprises a BCMA binding moiety at the N-terminus or C-terminus. In some embodiments, the light chain or modified light chain comprises an FcRH5 binding moiety at the N-terminus or C-terminus. In some embodiments, the light chain or modified light chain comprises a CD38 binding moiety at the N-terminus or C-terminus. In some embodiments, the light chain or modified light chain comprises a SLAMF7 binding moiety at the N-terminus or C-terminus. In some embodiments, the multispecific binder molecule comprises two modified heavy chains and at least one, at least two, at least three, or at least four light chains. In some embodiments, the multispecific binder molecule comprises two modified heavy chains and four light chains. In some embodiments, the multispecific binder molecule comprises two modified heavy chains, wherein the modified heavy chains comprise different amino acid sequences. In some embodiments, the multispecific binder molecule comprises four light chains, wherein the four light chains each comprise at least one portion of non-identical amino acid sequence when compared to each other. In some embodiments, the multispecific binder molecule comprises four light chains, wherein two of the four light chains comprise an identical amino acid protein sequence. In some embodiments, wherein two of the four light chains comprise an identical amino acid protein sequence, the identical light chains are CD3 light chains, each CD3 light chain comprising a CD3 VL domain. In some embodiments, the multispecific binder molecule comprises four light chains, wherein two of the four light chains consist of identical amino acid protein sequences. In some embodiments, the multispecific binder molecule comprises four light chains, wherein three of the four light chains comprise an identical amino acid protein sequence. In some embodiments, wherein three of the four light chains comprise an identical amino acid protein sequence, the identical light chains are CD3 light chains, each CD3 light chain comprising a CD3 VL domain. In some embodiments, the multispecific binder molecule comprises four light chains, wherein three of the four light chains consist of identical amino acid protein sequences. In some embodiments wherein the multispecific binder molecules comprises two identical CD3 light chain, a first CD3 light chain is paired with a region comprising a CD3 VH domain and a CH1 domain, and a second CD3 light chain is paired with a region comprising: i) a BCMA VH domain and a CH1 domain, ii) a CD38 VH domain and a CH1 domain, or iii) a SLAMF7 VH domain and a CH1 domain. In some embodiments wherein the multispecific binder molecules comprises three identical CD3 light chains, a first CD3 light chain is paired with a region comprising a CD3 VH domain and a CH1 domain, and a second CD3 light chain and a third CD3 light chain are paired respectively with regions selected from: i) a BCMA VH domain and a CH1 domain, ii) a CD38 VH domain and a CH1 domain, and iii) a SLAMF7 VH domain and a CH1 domain. In some embodiments wherein the multispecific binder molecules comprises two identical CD3 light chain, a first CD3 light chain is paired with a region comprising a CD3 VH domain and a CH1 domain, and a second CD3 light chain is paired with a region comprising: i) a BCMA VH domain and a CH1 domain, or ii) an FcRH5 VH domain and a CH1 domain.

In some embodiments, the multispecific binder molecule comprises four light chains, wherein two of the four light chains comprise an identical amino acid amino acid sequences. In some embodiments, a disulfide bond formed between a CH1 on a heavy chain or modified heavy chain and a CL region on a light chain aids in the stabilization of the structure of the multispecific binder molecules.

In some embodiments, the antibody-based binders bind to mammalian antigens. In some embodiments, the anti-GPRC5D antibody-based binder specifically binds to mammalian GPRC5D. In some embodiments, the anti-GPRC5D antibody-based binder specifically binds to mouse GPRC5D. In some embodiments, the anti-GPRC5D antibody-based binder specifically binds to cynomolgus monkey GPRC5D. In some embodiments, the anti-GPRC5D antibody-based binder specifically binds to human GPRC5D. In some embodiments, the anti-GPRC5D antibody-based binder specifically binds to one or more extracellular portions of GPRC5D. In some embodiments, the anti-GPRC5D antibody-based binder specifically binds to one or more extracellular portions of human GPRC5D. In some embodiments, the anti-BCMA antibody-based binder specifically binds to mammalian BCMA. In some embodiments, the anti-BCMA antibody-based binder specifically binds to mouse BCMA. In some embodiments, the anti-BCMA antibody-based binder specifically binds to cynomolgus monkey BCMA. In some embodiments, the anti-BCMA antibody-based binder specifically binds to human BCMA. In some embodiments, the anti-BCMA antibody-based binder specifically binds to one or more extracellular portions of BCMA. In some embodiments, the anti-BCMA antibody-based binder specifically binds to one or more extracellular portions of human BCMA. In some embodiments, the anti-FcRH5 antibody-based binder specifically binds to mammalian FcRH5. In some embodiments, the anti-FcRH5 antibody-based binder specifically binds to mouse FcRH5. In some embodiments, the anti-FcRH5 antibody-based binder specifically binds to cynomolgus monkey FcRH5. In some embodiments, the anti-FcRH5 antibody-based binder specifically binds to human FcRH5. In some embodiments, the anti-FcRH5 antibody-based binder specifically binds to one or more extracellular portions of FcRH5. In some embodiments, the anti-FcRH5 antibody-based binder specifically binds to one or more extracellular portions of human FcRH5. In some embodiments, the anti-FcRH5 antibody-based binder specifically binds to soluble human FcRH5. In some embodiments, the anti-CD38 antibody-based binder specifically binds to mammalian CD38. In some embodiments, the anti-CD38 antibody-based binder specifically binds to mouse CD38. In some embodiments, the anti-CD38 antibody-based binder specifically binds to cynomolgus monkey CD38. In some embodiments, the anti-CD38 antibody-based binder specifically binds to human CD38. In some embodiments, the anti-CD38 antibody-based binder specifically binds to one or more extracellular portions of CD38. In some embodiments, the anti-CD38 antibody-based binder specifically binds to one or more extracellular portions of human CD38. In some embodiments, the anti-SLAMF7 antibody-based binder specifically binds to mammalian SLAMF7. In some embodiments, the anti-SLAMF7 antibody-based binder specifically binds to mouse SLAMF7. In some embodiments, the anti-SLAMF7 antibody-based binder specifically binds to cynomolgus monkey SLAMF7. In some embodiments, the anti-SLAMF7 antibody-based binder specifically binds to human SLAMF7. In some embodiments, the anti-SLAMF7 antibody-based binder specifically binds to one or more extracellular portions of SLAMF7. In some embodiments, the anti-SLAMF7 antibody-based binder specifically binds to one or more extracellular portions of human SLAMF7. In some embodiments, the anti-CD3 antibody-based binder specifically binds to mammalian CD3. In some embodiments, the anti-CD3 antibody-based binder specifically binds to mouse CD3. In some embodiments, the anti-CD3 antibody-based binder specifically binds to cynomolgus monkey CD3. In some embodiments, the anti-CD3 antibody-based binder specifically binds to human CD3. In some embodiments, the anti-CD3 antibody-based binder specifically binds to one or more extracellular portions of CD3. In some embodiments, the anti-CD3 antibody-based binder specifically binds to one or more extracellular portions of human CD3. In some embodiments, the anti-CD3 antibody-based binder specifically binds to one or more epitopes of CD3. In some embodiments, the anti-CD3 antibody-based binder specifically binds to a CD3 gamma chain. In some embodiments, the anti-CD3 antibody-based binder specifically binds to a CD3 delta chain. In some embodiments, the anti-CD3 antibody-based binder specifically binds to a CD3 epsilon chain. In some embodiments, the anti-CD3 antibody-based binder specifically binds to a CD3 zeta chain. In some embodiments, the anti-CD3 antibody-based binder specifically binds to any combination of a CD3 gamma chain, a CD3 delta chain, a CD3 epsilon chain, a CD3 zeta chain, and a T-cell receptor.

In some embodiments, the antibody-based binders of the multispecific binder molecule comprise an antibody or antigen binding fragment thereof. In some embodiments, the anti-GPRC5D antibody-based binder is derived from a human antibody, a rat antibody, a mouse antibody, a rabbit antibody, a camelid antibody, a humanized antibody, or a chimeric antibody. In some embodiments, the anti-BCMA antibody-based binder is derived from a human antibody, a rat antibody, a mouse antibody, a rabbit antibody, a camelid antibody, a humanized antibody, or a chimeric antibody. In some embodiments, the anti-FcRH5 antibody-based binder is derived from a human antibody, a rat antibody, a mouse antibody, a rabbit antibody, a camelid antibody, a humanized antibody, or a chimeric antibody. In some embodiments, the anti-CD38 antibody-based binder is derived from a human antibody, a rat antibody, a mouse antibody, a rabbit antibody, a camelid antibody, a humanized antibody, or a chimeric antibody. In some embodiments, the anti-SLAMF7 antibody-based binder is derived from a human antibody, a rat antibody, a mouse antibody, a rabbit antibody, a camelid antibody, a humanized antibody, or a chimeric antibody. In some embodiments, the anti-CD3 antibody-based binder is derived from a human antibody, a rat antibody, a mouse antibody, a rabbit antibody, a camelid antibody, a humanized antibody, or a chimeric antibody.

In some embodiments, the antibody-based binders of the multispecific binder molecule are derived from chimeric amino acid sequences. In some embodiments, one or more of the antibody-based binders are made of chimeric amino acid sequences some of which are murine-derived and some of which are human-derived. In some embodiments, one or more of the antibody-based binders are made with complementarity-determining regions (CDRs) that have been incorporated into an antibody scaffold. In some embodiments, one or more of the antibody-based binders are made with complementarity-determining regions (CDRs) incorporated into a human antibody variable region framework. In some instances, the human antibody variable region framework has been sequence-optimized to retain GPRC5D affinity with the engrafted mouse CDR sequences. In some instances, the human antibody variable region framework has been sequence-optimized to retain BCMA affinity with the engrafted mouse CDR sequences. In some instances, the human antibody variable region framework has been sequence-optimized to retain FcRH5 affinity with the engrafted mouse CDR sequences. In some instances, the human antibody variable region framework has been sequence-optimized to retain CD38 affinity with the engrafted mouse CDR sequences. In some instances, the human antibody variable region framework has been sequence-optimized to retain SLAMF7 affinity with the engrafted mouse CDR sequences. In some instances, the human antibody variable region framework has been sequence-optimized to retain CD3 affinity with the engrafted mouse CDR sequences.

In some embodiments, amino acids sequences corresponding to CDR sequences of an antibody variable domain described herein are used in a binding moiety within the multispecific binder molecule. In some embodiments, a set of HCDR sequences listed Table 1 are used in a VH domain that forms a CD3 binding moiety, a GPRC5D binding moiety, a BCMA binding moiety, an FcRH5 binding moiety, a CD38 binding moiety, or a SLAMF7 binding moiety. In some embodiments, a set of LCDR sequences listed Table 2 are used in a VL domain that forms a CD3 binding moiety, a GPRC5D binding moiety, a BCMA binding moiety, an FcRH5 binding moiety, a CD38 binding moiety, or a SLAMF7 binding moiety. In some embodiments, a VH domain comprising a set of CDR sequences listed in Table 1 is paired with a VL domain comprising a set of CDR sequences listed in Table 2, wherein the paired VH and VL domain form a CD3 binding moiety, a GPRC5D binding moiety, a BCMA binding moiety, an FcRH5 binding moiety, a CD38 binding moiety, or a SLAMF7 binding moiety.

In some embodiments, the VH and VL domains are formatted in a configuration to form a binding moiety. In some embodiments, the GPRC5D-binding moiety comprises a VH and a VL in the format of a single-chain variable fragment (scFv) domain. In some embodiments, the BCMA-binding moiety comprises a VH and a VL in the format of a single-chain variable fragment (scFv) domain. In some embodiments, the FcRH5-binding moiety comprises a VH and a VL in the format of a single-chain variable fragment (scFv) domain. In some embodiments, the CD38-binding moiety comprises a VH and a VL in the format of a single-chain variable fragment (scFv) domain. In some embodiments, the SLAMF7-binding moiety comprises a VH and a VL in the format of a single-chain variable fragment (scFv) domain. In some embodiments, the CD3-binding moiety comprises a VH and a VL in the format of a single-chain variable fragment (scFv) domain. In some embodiments, the GPRC5D-binding scFv domain is paired with a second scFv domain to form a tandem scFv. In some embodiments, the BCMA-binding scFv domain is paired with a second scFv domain to form a tandem scFv. In some embodiments, the FcRH5-binding scFv domain is paired with a second scFv domain to form a tandem scFv. In some embodiments, the CD38-binding scFv domain is paired with a second scFv domain to form a tandem scFv. In some embodiments, the SLAMF7-binding scFv domain is paired with a second scFv domain to form a tandem scFv. In some embodiments, the CD3-binding scFv domain is paired with a second scFv domain to form a tandem scFv. In some embodiments, the tandem scFv comprises a bivalent GPRC5D-binding moiety. In some embodiments, the tandem scFv comprises a bivalent BCMA-binding moiety. In some embodiments, the tandem scFv comprises a bivalent FcRH5-binding moiety. In some embodiments, the tandem scFv comprises a bivalent CD38-binding moiety. In some embodiments, the tandem scFv comprises a bivalent SLAMF7-binding moiety. In some embodiments, the tandem scFv comprises a bivalent CD3-binding moiety. In some embodiments, the tandem scFv comprises a bi-specific binding moiety comprising a GPRC5D-binding moiety and a second binding moiety. In some embodiments, the tandem scFv comprises a bi-specific binding moiety comprising a BCMA-binding moiety and a second binding moiety. In some embodiments, the tandem scFv comprises a bi-specific binding moiety comprising an FcRH5 binding moiety and a second binding moiety. In some embodiments, the tandem scFv comprises a bi-specific binding moiety comprising a CD38-binding moiety and a second binding moiety. In some embodiments, the tandem scFv comprises a bi-specific binding moiety comprising a SLAMF7-binding moiety and a second binding moiety. In some embodiments, the tandem scFv comprises a bi-specific binding moiety comprising a CD3-binding moiety and a second binding moiety. In some embodiments, a noncovalent dimer of scFv fragments comprised of a heavy chain variable and a light chain variable regions connected by a small peptide linker form a diabody that functions as a GPRC5D-binding moiety. In some embodiments, a noncovalent dimer of scFv fragments comprised of a heavy chain variable and a light chain variable regions connected by a small peptide linker form a diabody that functions as a BCMA-binding moiety. In some embodiments, a noncovalent dimer of scFv fragments comprised of a heavy chain variable and a light chain variable regions connected by a small peptide linker form a diabody that functions as an FcRH5-binding moiety. In some embodiments, a noncovalent dimer of scFv fragments comprised of a heavy chain variable and a light chain variable regions connected by a small peptide linker form a diabody that functions as a CD38-binding moiety. In some embodiments, a noncovalent dimer of scFv fragments comprised of a heavy chain variable and a light chain variable regions connected by a small peptide linker form a diabody that functions as a SLAMF7-binding moiety. In some embodiments, a noncovalent dimer of scFv fragments comprised of a heavy chain variable and a light chain variable regions connected by a small peptide linker form a diabody that functions as a CD3-binding moiety. In some embodiments, the diabody is a bivalent GPRC5D-binding moiety, a bivalent BCMA-binding moiety, a bivalent FcRH5-binding moiety, a bivalent CD38-binding moiety, a bivalent SLAMF7-binding moiety, or a bivalent CD3-binding moiety. In some embodiments, the diabody is a bi-specific binding moiety comprising a GPRC5D-binding moiety and a second binding moiety. In some embodiments, the diabody is a bi-specific binding moiety comprising a BCMA-binding moiety and a second binding moiety. In some embodiments, the diabody is a bi-specific binding moiety comprising an FcRH5-binding moiety and a second binding moiety. In some embodiments, the diabody is a bi-specific binding moiety comprising a CD38-binding moiety and a second binding moiety. In some embodiments, the diabody is a bi-specific binding moiety comprising a SLAMF7-binding moiety and a second binding moiety. In some embodiments, the diabody is a bi-specific binding moiety comprising a CD3-binding moiety and a second binding moiety. In some embodiments, three scFv fragments are linked together to form a triabody that functions as a GPRC5D-binding moiety, a BCMA-binding moiety, a CD38-binding moiety, an FcRH5-binding moiety, a SLAMF7-binding moiety, or a CD3-binding moiety. In some embodiments, three scFv fragments are linked together to form a triabody that functions as a GPRC5D-binding moiety and a binding moiety for a second binding moiety. In some embodiments, three scFv fragments are linked together to form a triabody that functions as a BCMA-binding moiety and a binding moiety for a second binding moiety. In some embodiments, three scFv fragments are linked together to form a triabody that functions as an FcRH5-binding moiety and a binding moiety for a second binding moiety. In some embodiments, three scFv fragments are linked together to form a triabody that functions as a CD38-binding moiety and a binding moiety for a second binding moiety. In some embodiments, three scFv fragments are linked together to form a triabody that functions as a SLAMF7-binding moiety and a binding moiety for a second binding moiety. In some embodiments, three scFv fragments are linked together to form a triabody that functions as a CD3-binding moiety and a binding moiety for a second binding moiety. In some embodiments, three scFv fragments are linked together to form a triabody that functions as a GPRC5D-binding moiety and a binding moiety for a second binding moiety and a third binding moiety. In some embodiments, three scFv fragments are linked together to form a triabody that functions as a BCMA-binding moiety and a binding moiety for a second binding moiety and a third binding moiety. In some embodiments, three scFv fragments are linked together to form a triabody that functions as an FcRH5-binding moiety and a binding moiety for a second binding moiety and a third binding moiety. In some embodiments, three scFv fragments are linked together to form a triabody that functions as a CD38-binding moiety and a binding moiety for a second binding moiety and a third binding moiety. In some embodiments, three scFv fragments are linked together to form a triabody that functions as a SLAMF7-binding moiety and a binding moiety for a second binding moiety and a third binding moiety. In some embodiments, three scFv fragments are linked together to form a triabody that functions as a CD3-binding moiety and a binding moiety for a second binding moiety and a third binding moiety. In some embodiments, two scFv fragments are covalently linked to each other to from a single-chain (Fv) 2 that functions as a GPRC5D-binding moiety, a BCMA-binding moiety, an FcRH5-binding moiety, a CD38-binding moiety, a SLAMF7-binding moiety, or a CD3-binding moiety. In some embodiments, the scFv is a disulfide-bond stabilized scFv. In some embodiments, the GPRC5D-binding moiety is in the format of a tandem diabody (bispecific tetravalent antibody). In some embodiments, the BCMA-binding moiety is in the format of a tandem diabody (bispecific tetravalent antibody). In some embodiments, the FcRH5-binding moiety is in the format of a tandem diabody (bispecific tetravalent antibody). In some embodiments, the CD38-binding moiety is in the format of a tandem diabody (bispecific tetravalent antibody). In some embodiments, the SLAMF7-binding moiety is in the format of a tandem diabody (bispecific tetravalent antibody). In some embodiments, the CD3-binding moiety is in the format of a tandem diabody (bispecific tetravalent antibody). In some embodiments, the tandem diabody comprises two VH/VL pairs from two distinct Fv regions. In some embodiments, the GPRC5D-binding moiety is in the format of a dual affinity retargeting (DART) format comprising two Fv fragments, wherein Fv1 comprises a VH domain from an anti-GPRC5D antibody and a VL from a second antibody, and Fv2 comprises a VH domain from a second antibody and a VL domain from an anti-GPRC5D antibody. In some embodiments, the BCMA-binding moiety is in the format of a dual affinity retargeting (DART) format comprising two Fv fragments, wherein Fv1 comprises a VH domain from an anti-BCMA antibody and a VL from a second antibody, and Fv2 comprises a VH domain from a second antibody and a VL domain from an anti-BCMA antibody. In some embodiments, the FcRH5-binding moiety is in the format of a dual affinity retargeting (DART) format comprising two Fv fragments, wherein Fv1 comprises a VH domain from an anti-FcRH5 antibody and a VL from a second antibody, and Fv2 comprises a VH domain from a second antibody and a VL domain from an anti-FcRH5 antibody. In some embodiments, the CD38-binding moiety is in the format of a dual affinity retargeting (DART) format comprising two Fv fragments, wherein Fv1 comprises a VH domain from an anti-CD38 antibody and a VL from a second antibody, and Fv2 comprises a VH domain from a second antibody and a VL domain from an anti-CD38 antibody. In some embodiments, the SLAMF7-binding moiety is in the format of a dual affinity retargeting (DART) format comprising two Fv fragments, wherein Fv1 comprises a VH domain from an anti-SLAMF7 antibody and a VL from a second antibody, and Fv2 comprises a VH domain from a second antibody and a VL domain from an anti-SLAMF7 antibody. In some embodiments, the CD3-binding moiety is in the format of a dual affinity retargeting (DART) format comprising two Fv fragments, wherein Fv1 comprises a VH domain from an anti-CD3 antibody and a VL from a second antibody, and Fv2 comprises a VH domain from a second antibody and a VL domain from an anti-CD3 antibody. In some embodiments, the GPRC5D-binding moiety is in the format of a bi-specific T-cell engager (BiTE) wherein an scFv domain comprising a VH domain and VL domain from an anti-GPRC5D antibody is linked in a single polypeptide chain to a second scFv domain comprising a VH domain and VL domain derived from a T-cell binding antibody. In some embodiments, the BCMA-binding moiety is in the format of a bi-specific T-cell engager (BiTE) wherein an scFv domain comprising a VH domain and VL domain from an anti-BCMA antibody is linked in a single polypeptide chain to a second scFv domain comprising a VH domain and VL domain derived from a T-cell binding antibody. In some embodiments, the FcRH5-binding moiety is in the format of a bi-specific T-cell engager (BiTE) wherein an scFv domain comprising a VH domain and VL domain from an anti-FcRH5 antibody is linked in a single polypeptide chain to a second scFv domain comprising a VH domain and VL domain derived from a T-cell binding antibody. In some embodiments, the CD38-binding moiety is in the format of a bi-specific T-cell engager (BiTE) wherein an scFv domain comprising a VH domain and VL domain from an anti-CD38 antibody is linked in a single polypeptide chain to a second scFv domain comprising a VH domain and VL domain derived from a T-cell binding antibody. In some embodiments, the SLAMF7-binding moiety is in the format of a bi-specific T-cell engager (BiTE) wherein an scFv domain comprising a VH domain and VL domain from an anti-SLAMF7 antibody is linked in a single polypeptide chain to a second scFv domain comprising a VH domain and VL domain derived from a T-cell binding antibody. In some embodiments, the CD3-binding moiety is in the format of a bi-specific T-cell engager (BiTE) wherein an scFv domain comprising a VH domain and VL domain from an anti-CD3 antibody is linked in a single polypeptide chain to a second scFv domain comprising a VH domain and VL domain derived from a T-cell binding antibody.

In some embodiments, VH and VL domains are located on separate polypeptides and form a binding moiety in the multispecific binder molecule. In some embodiments, the GPRC5D VH and GPRC5D VL are near the N-terminus of polypeptides which are paired together to form a GPRC5D-binding moiety. In some embodiments, the BCMA VH and BCMA VL are near the N-terminus of polypeptides which are paired together to form a BCMA-binding moiety. In some embodiments, the FcRH5 VH and RcRH5 VL are near the N-terminus of polypeptides which are paired together to form an FcRH5-binding moiety. In some embodiments, the CD38 VH and CD38 VL are near the N-terminus of polypeptides which are paired together to form a CD38-binding moiety. In some embodiments, the SLAMF7 VH and SLAMF7 VL are near the N-terminus of polypeptides which are paired together to form a SLAMF7-binding moiety. In some embodiments, the CD3 VH and CD3 VL are near the N-terminus of polypeptides which are paired together to form a CD3-binding moiety. In some embodiments, the GPRC5D VH and GPRC5D VL are near the C-terminus of polypeptides which are paired together to form a GPRC5D-binding moiety. In some embodiments, the BCMA VH and BCMA VL are near the C-terminus of polypeptides which are paired together to form a BCMA-binding moiety. In some embodiments, the FcRH5 VH and FcRH5 VL are near the C-terminus of polypeptides which are paired together to form an FcRH5-binding moiety. In some embodiments, the CD38 VH and CD38 VL are near the C-terminus of polypeptides which are paired together to form a CD38-binding moiety. In some embodiments, the SLAMF7 VH and SLAMF7 VL are near the C-terminus of polypeptides which are paired together to form a SLAMF7-binding moiety. In some embodiments, the CD3 VH and CD3 VL are near the C-terminus of polypeptides which are paired together to form a CD3-binding moiety. In some embodiments, an scFv comprising a GPRC5D VH and a GPRC5D VL that forms a GPRC5D-binding moiety is located at the N-terminus of a modified heavy chain. In some embodiments, an scFv comprising a BCMA VH and a BCMA VL that forms a BCMA-binding moiety is located at the N-terminus of a modified heavy chain. In some embodiments, an scFv comprising an FcRH5 VH and an FcRH5 VL that forms an FcRH5-binding moiety is located at the N-terminus of a modified heavy chain. In some embodiments, an scFv comprising a CD38 VH and a CD38 VL that forms a CD38-binding moiety is located at the N-terminus of a modified heavy chain. In some embodiments, an scFv comprising a SLAMF7 VH and a SLAMF7 VL that forms a SLAMF7-binding moiety is located at the N-terminus of a modified heavy chain. In some embodiments, an scFv comprising a CD3 VH and a CD3 VL that forms a CD3-binding moiety is located at the N-terminus of a modified heavy chain. In some embodiments, an scFv comprising a GPRC5D VH and a GPRC5D VL that forms a GPRC5D-binding moiety is located at the C-terminus of a modified heavy chain. In some embodiments, an scFv comprising a BCMA VH and a BCMA VL that forms a BCMA-binding moiety is located at the C-terminus of a modified heavy chain. In some embodiments, an scFv comprising an FcRH5 VH and an FcRH5 VL that forms an FcRH5-binding moiety is located at the C-terminus of a modified heavy chain. In some embodiments, an scFv comprising a CD38 VH and a CD38 VL that forms a CD38-binding moiety is located at the C-terminus of a modified heavy chain. In some embodiments, an scFv comprising a SLAMF7 VH and a SLAMF7 VL that forms a SLAMF7-binding moiety is located at the C-terminus of a modified heavy chain. In some embodiments, an scFv comprising a CD3 VH and a CD3 VL that forms a CD3-binding moiety is located at the C-terminus of a modified heavy chain. In some embodiments, an scFv comprising a GPRC5D VH and a GPRC5D VL that forms a GPRC5D-binding moiety is located at the N-terminus of a modified light chain. In some embodiments, an scFv comprising a BCMA VH and a BCMA VL that forms a BCMA-binding moiety is located at the N-terminus of a modified light chain. In some embodiments, an scFv comprising an FcRH5 VH and an FcRH5 VL that forms an FcRH5-binding moiety is located at the N-terminus of a modified light chain. In some embodiments, an scFv comprising a CD38 VH and a CD38 VL that forms a CD38-binding moiety is located at the N-terminus of a modified light chain. In some embodiments, an scFv comprising a SLAMF7 VH and a SLAMF7 VL that forms a SLAMF7-binding moiety is located at the N-terminus of a modified light chain. In some embodiments, an scFv comprising a CD3 VH and a CD3 VL that forms a CD3-binding moiety is located at the N-terminus of a modified light chain. In some embodiments, an scFv comprising a GPRC5D VH and a GPRC5D VL that forms a GPRC5D-binding moiety is coupled to the C-terminus of a modified light chain. In some embodiments, an scFv comprising a BCMA VH and a BCMA VL that forms a BCMA-binding moiety is coupled to the C-terminus of a modified light chain. In some embodiments, an scFv comprising an FcRH5 VH and an FcRH5 VL that forms an FcRH5-binding moiety is coupled to the C-terminus of a modified light chain. In some embodiments, an scFv comprising a CD38 VH and a CD38 VL that forms a CD38-binding moiety is coupled to the C-terminus of a modified light chain. In some embodiments, an scFv comprising a SLAMF7 VH and a SLAMF7 VL that forms a SLAMF7-binding moiety is coupled to the C-terminus of a modified light chain. In some embodiments, an scFv comprising a CD3 VH and a CD3 VL that forms a CD3-binding moiety is coupled to the C-terminus of a modified light chain. In some embodiments, coupled binding moieties are covalently coupled to a polypeptide chain. In some embodiments, coupled binding moieties are conjugated to a polypeptide chain.

In some aspects, VH and VL domains are located on separate polypeptides and form a binding moiety in the multispecific binder molecule. In some embodiments, the VH and VL domains located on separate polypeptides are paired together and form a binding moiety. In some embodiments, the VH domain comprises HCDR sequences which confer antigen binding specificity for either GPRC5D, BCMA, FcRH5, CD38, SLAMF7, or CD3. In some embodiments, the GPRC5D VH domain comprising HCDR sequences which confers antigen binding specificity for GPRC5D is paired with a GPRC5D VL domain comprising GPRC5D LCDR sequences which when paired together with the GPRC5D VH, confers high affinity antigen binding specificity for GPRC5D. In some embodiments, the BCMA VH domain comprising HCDR sequences which confers antigen binding specificity for BMCA is paired with a BCMA VL domain comprising BCMA LCDR sequences which when paired together with the BCMA VH, confers high affinity antigen binding specificity for BCMA. In some embodiments, the BCMA VH domain comprising HCDR sequences which confers antigen binding specificity for BMCA is paired with a CD3 VL domain comprising CD3 LCDR sequences which when paired together with the BCMA VH, confers high affinity antigen binding specificity for BCMA. In some embodiments, the FcRH5 VH domain comprising HCDR sequences which confers antigen binding specificity for FcRH5 is paired with an FcRH5 VL domain comprising FcRH5 LCDR sequences which when paired together with the FcRH5 VH, confers high affinity antigen binding specificity for FcRH5. In some embodiments, the FcRH5 VH domain comprising HCDR sequences which confers antigen binding specificity for FcRH5 is paired with a CD3 VL domain comprising CD3 LCDR sequences which when paired together with the FcRH5 VH, confers high affinity antigen binding specificity for FcRH5. In some embodiments, the CD38 VH domain comprising HCDR sequences which confers antigen binding specificity for CD38 is paired with a CD38 VL domain comprising CD38 LCDR sequences which when paired together with the CD38 VH, confers high affinity antigen binding specificity for CD38. In some embodiments, the CD38 VH domain comprising HCDR sequences which confers antigen binding specificity for CD38 is paired with a CD3 VL domain comprising CD3 LCDR sequences which when paired together with the CD38 VH, confers high affinity antigen binding specificity for CD38. In some embodiments, the SLAMF7 VH domain comprising HCDR sequences which confers antigen binding specificity for SLAMF7 is paired with a SLAMF7 VL domain comprising SLAMF7 LCDR sequences which when paired together with the SLAMF7 VH, confers high affinity antigen binding specificity for CD38. In some embodiments, the SLAMF7 VH domain comprising HCDR sequences which confers antigen binding specificity for SLAMF7 is paired with a CD3 VL domain comprising CD3 LCDR sequences which when paired together with the CD38 VH, confers high affinity antigen binding specificity for CD38. In some embodiments, the CD3 VH domain comprising HCDR sequences which confers antigen binding specificity for CD3 is paired with a CD3 VL domain comprising CD3 LCDR sequences which when paired together with the CD3 VH, confers high affinity antigen binding specificity for CD3.

In some embodiments, the multispecific binder molecule comprises a first immunoglobulin chain constant region on a first heavy chain and a second immunoglobulin chain constant region on a second heavy chain. In some embodiments, the first and second heavy chains are paired together in the multispecific binder molecule. In some embodiments, the first heavy chain comprises a modified heavy chain comprising two VH domains. In some embodiments, the configuration of the modified heavy chain comprises a GPRC5D VH located at the N-terminus, followed by CH1, CH2, CH3, CD3 VH, and CH1 domains. In some embodiments, the configuration of the modified heavy chain comprises a GPRC5D VH located at the N-terminus, followed by CH1, CH2, CH3, CH1, and CD3 VH domains. In some embodiments, the configuration of the modified heavy chain comprises a GPRC5D VH located at the N-terminus, followed by CH1, CH2, CH3, BCMA VH, and CH1 domains. In some embodiments, the configuration of the modified heavy chain comprises a GPRC5D VH located at the N-terminus, followed by CH1, CH2, CH3, CH1, and BCMA VH domains. In some embodiments, the configuration of the modified heavy chain comprises a GPRC5D VH located at the N-terminus, followed by CH1, CH2, CH3, CD38 VH, and CH1 domains. In some embodiments, the configuration of the modified heavy chain comprises a GPRC5D VH located at the N-terminus, followed by CH1, CH2, CH3, CH1, and CD38 VH domains. In some embodiments, the configuration of the modified heavy chain comprises a GPRC5D VH located at the N-terminus, followed by CH1, CH2, CH3, SLAMF7 VH, and CH1 domains. In some embodiments, the configuration of the modified heavy chain comprises a GPRC5D VH located at the N-terminus, followed by CH1, CH2, CH3, CH1, and SLAMF7 VH domains. In some embodiments, the configuration of the modified heavy chain comprises a GPRC5D VH located at the N-terminus, followed by CH1, CH2, CH3, FcRH5 VH, and CH1 domains. In some embodiments, the configuration of the modified heavy chain comprises a GPRC5D VH located at the N-terminus, followed by CH1, CH2, CH3, CH1, and FcRH5 VH domains. In some embodiments, the configuration of the modified heavy chain comprises a BCMA VH located at the N-terminus, followed by CH1, CH2, CH3, CD3 VH, and CH1 domains. In some embodiments, the configuration of the modified heavy chain comprises a BCMA VH located at the N-terminus, followed by CH1, CH2, CH3, CH1, and CD3 VH domains. In some embodiments, the configuration of the modified heavy chain comprises a BCMA VH located at the N-terminus, followed by CH1, CH2, CH3, GPRC5D VH, and CH1 domains. In some embodiments, the configuration of the modified heavy chain comprises a BCMA VH located at the N-terminus, followed by CH1, CH2, CH3, CH1, and GPRC5D VH domains. In some embodiments, the configuration of the modified heavy chain comprises a BCMA VH located at the N-terminus, followed by CH1, CH2, CH3, CD38 VH, and CH1 domains. In some embodiments, the configuration of the modified heavy chain comprises a BCMA VH located at the N-terminus, followed by CH1, CH2, CH3, CH1, and CD38 VH domains. In some embodiments, the configuration of the modified heavy chain comprises a BCMA VH located at the N-terminus, followed by CH1, CH2, CH3, SLAMF7 VH, and CH1 domains. In some embodiments, the configuration of the modified heavy chain comprises a BCMA VH located at the N-terminus, followed by CH1, CH2, CH3, CH1, and SLAMF7 VH domains. In some embodiments, the configuration of the modified heavy chain comprises a BCMA VH located at the N-terminus, followed by CH1, CH2, CH3, FcRH5 VH, and CH1 domains. In some embodiments, the configuration of the modified heavy chain comprises a BCMA VH located at the N-terminus, followed by CH1, CH2, CH3, CH1, and FcRH5 VH domains. In some embodiments, the configuration of the modified heavy chain comprises an FcRH5 VH located at the N-terminus, followed by CH1, CH2, CH3, GPRC5D VH, and CH1 domains. In some embodiments, the configuration of the modified heavy chain comprises an FcRH5 VH located at the N-terminus, followed by CH1, CH2, CH3, CH1, and GPRC5D VH domains. In some embodiments, the configuration of the modified heavy chain comprises an FcRH5 VH located at the N-terminus, followed by CH1, CH2, CH3, BCMA VH, and CH1 domains. In some embodiments, the configuration of the modified heavy chain comprises an FcRH5 VH located at the N-terminus, followed by CH1, CH2, CH3, CH1, and BCMA VH domains. In some embodiments, the configuration of the modified heavy chain comprises a FcRH5 VH located at the N-terminus, followed by CH1, CH2, CH3, CD3 VH, and CH1 domains. In some embodiments, the configuration of the modified heavy chain comprises an FcRH5 VH located at the N-terminus, followed by CH1, CH2, CH3, CH1, and CD3 VH domains. In some embodiments, the configuration of the modified heavy chain comprises a FcRH5 VH located at the N-terminus, followed by CH1, CH2, CH3, CD38 VH, and CH1 domains. In some embodiments, the configuration of the modified heavy chain comprises an FcRH5 VH located at the N-terminus, followed by CH1, CH2, CH3, CH1, and CD38 VH domains. In some embodiments, the configuration of the modified heavy chain comprises a FcRH5 VH located at the N-terminus, followed by CH1, CH2, CH3, SLAMF7 VH, and CH1 domains. In some embodiments, the configuration of the modified heavy chain comprises an FcRH5 VH located at the N-terminus, followed by CH1, CH2, CH3, CH1, and SLAMF7 VH domains. In some embodiments, the configuration of the modified heavy chain comprises a CD38 VH located at the N-terminus, followed by CH1, CH2, CH3, CD3 VH, and CH1 domains. In some embodiments, the configuration of the modified heavy chain comprises a CD38 VH located at the N-terminus, followed by CH1, CH2, CH3, CH1, and CD3 VH domains. In some embodiments, the configuration of the modified heavy chain comprises a CD38 VH located at the N-terminus, followed by CH1, CH2, CH3, GPRC5D VH, and CH1 domains. In some embodiments, the configuration of the modified heavy chain comprises a CD38 VH located at the N-terminus, followed by CH1, CH2, CH3, CH1, and GPRC5D VH domains. In some embodiments, the configuration of the modified heavy chain comprises a CD38 VH located at the N-terminus, followed by CH1, CH2, CH3, BCMA VH, and CH1 domains. In some embodiments, the configuration of the modified heavy chain comprises a CD38 VH located at the N-terminus, followed by CH1, CH2, CH3, CH1, and BCMA VH domains. In some embodiments, the configuration of the modified heavy chain comprises a CD38 VH located at the N-terminus, followed by CH1, CH2, CH3, SLAMF7 VH, and CH1 domains. In some embodiments, the configuration of the modified heavy chain comprises a CD38 VH located at the N-terminus, followed by CH1, CH2, CH3, CH1, and SLAMF7 VH domains. In some embodiments, the configuration of the modified heavy chain comprises a CD38 VH located at the N-terminus, followed by CH1, CH2, CH3, FcRH5 VH, and CH1 domains. In some embodiments, the configuration of the modified heavy chain comprises a CD38 VH located at the N-terminus, followed by CH1, CH2, CH3, CH1, and FcRH5 VH domains. In some embodiments, the configuration of the modified heavy chain comprises a SLAMF7 VH located at the N-terminus, followed by CH1, CH2, CH3, CD3 VH, and CH1 domains. In some embodiments, the configuration of the modified heavy chain comprises a SLAMF7 VH located at the N-terminus, followed by CH1, CH2, CH3, CH1, and CD3 VH domains. In some embodiments, the configuration of the modified heavy chain comprises a SLAMF7 VH located at the N-terminus, followed by CH1, CH2, CH3, GPRC5D VH, and CH1 domains. In some embodiments, the configuration of the modified heavy chain comprises a SLAMF7 VH located at the N-terminus, followed by CH1, CH2, CH3, CH1, and GPRC5D VH domains. In some embodiments, the configuration of the modified heavy chain comprises a SLAMF7 VH located at the N-terminus, followed by CH1, CH2, CH3, BCMA VH, and CH1 domains. In some embodiments, the configuration of the modified heavy chain comprises a SLAMF7 VH located at the N-terminus, followed by CH1, CH2, CH3, CH1, and BCMA VH domains. In some embodiments, the configuration of the modified heavy chain comprises a SLAMF7 VH located at the N-terminus, followed by CH1, CH2, CH3, CD38 VH, and CH1 domains. In some embodiments, the configuration of the modified heavy chain comprises a SLAMF7 VH located at the N-terminus, followed by CH1, CH2, CH3, CH1, and CD38 VH domains. In some embodiments, the configuration of the modified heavy chain comprises a SLAMF7 VH located at the N-terminus, followed by CH1, CH2, CH3, FcRH5 VH, and CH1 domains. In some embodiments, the configuration of the modified heavy chain comprises a SLAMF7 VH located at the N-terminus, followed by CH1, CH2, CH3, CH1, and FcRH5 VH domains. In some embodiments, the configuration of the modified heavy chain comprises a CD3 VH located at the N-terminus, followed by CH1, CH2, CH3, SLAMF7 VH, and CH1 domains. In some embodiments, the configuration of the modified heavy chain comprises a CD3 VH located at the N-terminus, followed by CH1, CH2, CH3, CH1, and SLAMF7 VH domains. In some embodiments, the configuration of the modified heavy chain comprises a CD3 VH located at the N-terminus, followed by CH1, CH2, CH3, GPRC5D VH, and CH1 domains. In some embodiments, the configuration of the modified heavy chain comprises a CD3 VH located at the N-terminus, followed by CH1, CH2, CH3, CH1, and GPRC5D VH domains. In some embodiments, the configuration of the modified heavy chain comprises a CD3 VH located at the N-terminus, followed by CH1, CH2, CH3, BCMA VH, and CH1 domains. In some embodiments, the configuration of the modified heavy chain comprises a CD3 VH located at the N-terminus, followed by CH1, CH2, CH3, CH1, and BCMA VH domains. In some embodiments, the configuration of the modified heavy chain comprises a CD3 VH located at the N-terminus, followed by CH1, CH2, CH3, CD38 VH, and CH1 domains. In some embodiments, the configuration of the modified heavy chain comprises a CD3 VH located at the N-terminus, followed by CH1, CH2, CH3, CH1, and CD38 VH domains. In some embodiments, the configuration of the modified heavy chain comprises a CD3 VH located at the N-terminus, followed by CH1, CH2, CH3, FcRH5 VH, and CH1 domains. In some embodiments, the configuration of the modified heavy chain comprises a CD3 VH located at the N-terminus, followed by CH1, CH2, CH3, CH1, and FcRH5 VH domains.

In some embodiments, the multispecific binder molecule comprises a first immunoglobulin chain constant region on a first modified heavy chain and a second immunoglobulin chain constant region on a second modified heavy chain. In some embodiments, the first and second modified heavy chains are paired together in the multispecific binder molecule. In some embodiments, the first immunoglobulin chain constant region and the second immunoglobulin chain constant region each comprise a CH1 domain. In some embodiments, a BCMA VH domain is positioned N-terminal to the CH1 domain of the first immunoglobulin chain constant region and a CD3 VH domain is positioned N-terminal to the CH1 domain of the second immunoglobulin chain constant region. In some embodiments, the first modified heavy chain comprises a CH2 domain or a mutated CH2 domain C-terminal to the CH1 domain. In some embodiments, the second modified heavy chain comprises a CH2 domain or a mutated CH2 domain C-terminal to the CH1 domain. In some embodiments, the first modified heavy chain comprises a CH3 domain or a mutated CH3 domain C-terminal to the CH2 domain. In some embodiments, the second modified heavy chain comprises a CH3 domain or a mutated CH3 domain C-terminal to the CH2 domain. In some embodiments, the first modified heavy chain comprises an FcRH5 VH domain C-terminal to the CH3 domain or mutated CH3 domain. In some embodiments, the second modified heavy chain comprises a GPRC5D VH domain C-terminal to the CH3 domain or mutated CH3 domain. In some embodiments, the mutated CH3 domain or domains comprises one or more KiH substitutions. In some embodiments, the first modified heavy chain comprises two VH domains. In some embodiments, the configuration of the first modified heavy chain comprises a BCMA VH located at the N-terminus, followed by CH1, CH2, CH3, FcRH5 VH, and CH1 domains. In some embodiments, the second modified heavy chain comprises two VH domains. In some embodiments, the configuration of the second modified heavy chain comprises a CD3 VH located at the N-terminus, followed by CH1, CH2, CH3, GPRC5D VH, and CH1 domains. In some embodiments, the CH3 of the first modified heavy chain comprises one or more KiH knob substitutions. In some embodiments, the one or more KiH knob substitutions comprise either S354C, T366W, or S354C and T366W. In some embodiments, the one or more KiH knob substitutions comprise S354C and T366W. In some embodiments, the CH3 of the first modified heavy chain comprises one or more KiH hole substitutions. In some embodiments, the one or more KiH hole substitutions comprise Y349C, T366S, L368A, Y407V, or any combination thereof. In some embodiments, the one or more KiH hole substitutions comprise Y349C, T366S, L368A, and Y407V. In some embodiments, the CH3 of the second modified heavy chain comprises one or more KiH knob substitutions. In some embodiments, the one or more KiH knob substitutions comprise either S354C, T366W, or S354C and T366W. In some embodiments, the one or more KiH knob substitutions comprise S354C and T366W. In some embodiments, the CH3 of the second modified heavy chain comprises one or more KiH hole substitutions. In some embodiments, the one or more KiH hole substitutions comprise Y349C, T366S, L368A, Y407V, or any combination thereof. In some embodiments, the one or more KiH hole substitutions comprise Y349C, T366S, L368A, and Y407V. In some embodiments, the multispecific binder molecule comprises two modified heavy chains comprising KiH knob substitutions and KiH hole substitutions respectively which aid in heterodimeric pairing of the two distinct modified heavy chains. In some embodiments, the first modified heavy chain comprising KiH knob mutations is paired with the second modified heavy chain comprising KiH hole mutations to aid in heterodimerization of the separate modified heavy chains. In some embodiments, the multispecific binder molecule comprises four light chains. In some embodiments, each of the four light chains is paired with a distinct VH domain to form a binding moiety. In some embodiments, the multispecific binder molecule comprises three light chains each comprising a CD3 binding moiety and a fourth light chain comprising a GPRC5D binding moiety. In some embodiments, the multispecific binder molecule comprises four distinct light chains, wherein the first light chain comprises a CD3 binding moiety, the second light chain comprises a BCMA binding moiety, the third light chain comprises an FcRH5 binding moiety, and the fourth light chain comprises a GPRC5D binding moiety. In some embodiments, a first light chain comprising a CD3 VL domain is paired with a CD3 VH domain on a modified heavy chain to form a CD3 binding moiety. In some embodiments, a second light chain comprising a CD3 VL domain is paired with a BCMA VH domain on a modified heavy chain to form a BCMA binding moiety. In some embodiments, a second light chain comprising a BCMA VL domain is paired with a BCMA VH domain on a modified heavy chain to form a BCMA binding moiety. In some embodiments, a third light chain comprising a CD3 VL domain is paired with an FcRH5 VH domain on a modified heavy chain to form an FcRH5 binding moiety. In some embodiments, a third light chain comprising a FcRH5 VL domain is paired with an FcRH5 VH domain on a modified heavy chain to form an FcRH5 binding moiety. In some embodiments, a fourth light chain comprising a GPRC5D VL domain is paired with a GPRC5D VH domain on a modified heavy chain to form a GPRC5D binding moiety. In some embodiments, the multispecific binder molecule comprises: i) a first modified heavy chain comprising an N-terminal BCMA binding moiety and a C-terminal FcRH5 binding moiety, ii) a second modified heavy chain comprising an N-terminal CD3 binding moiety and a C-terminal GPRC5D binding moiety, iii) a first light chain comprising a CD3 VL domain, iv) a second light chain comprising a BCMA VL domain or a CD3 VL domain, v) a third light chain comprising a FcRH5 VL domain or a CD3 VL domain, and vi), a fourth light chain comprising a GPRC5D VL domain.

In some embodiments, the multispecific binder molecule comprises one or more linker sequences. In some embodiments, one or more linker sequences separate peptide domains within a single heavy chain polypeptide. In some embodiments, one or more linker sequences separate peptide domains within a single light chain polypeptide. In some embodiments, the linker sequence is a rigid linker sequence. In some embodiments, the rigid linker sequence comprises a proline-rich sequence. In some embodiments, the linker sequence is a flexible linker sequence. In some embodiments, the flexible linker sequence comprises a glycine-rich sequence. In some embodiments, the flexible linker sequence comprises a glycine-serine linker sequence. In some embodiments, the glycine-serine linker sequence comprises an amino sequence of GS₍ₙ₎ wherein n is 1-10 (SEQ ID NO: 86). In some embodiments, the glycine-serine linker sequence comprises an amino sequence of GGGGS₍ₙ₎ wherein n is 1-6 (SEQ ID NO: 87). In some embodiments, a linker sequence positioned between a binding moiety and a heavy chain antibody constant region comprises a sequence listed in Table 21. In some embodiments, the linker sequence positioned between a binding moiety and a light chain antibody constant region comprises a sequence listed in Table 21.

In some embodiments, the multispecific binder molecule comprises at least three binding moieties selected from the group consisting of: a GPRC5D-binding moiety, a BCMA-binding moiety, an FcRH5-binding moiety, a CD38-binding moiety, a SLAMF7-binding moiety, and a CD3-binding moiety. In some embodiments, the multispecific binder molecule comprises at least four binding moieties selected from the group consisting of: a GPRC5D-binding moiety, a BCMA-binding moiety, an FcRH5-binding moiety, a CD38-binding moiety, a SLAMF7-binding moiety, and a CD3-binding moiety. In some embodiments, at least one of the at least three binding moieties is coupled to a heavy chain. In some embodiments, at least one of the at least four binding moieties is coupled to a heavy chain. In some embodiments, the GPRC5D-binding moiety is coupled to the N-terminus of an IgG1 heavy chain. In some embodiments, the BCMA-binding moiety is coupled to the N-terminus of an IgG1 heavy chain. In some embodiments, the FcRH5-binding moiety is coupled to the N-terminus of an IgG1 heavy chain. In some embodiments, the CD38-binding moiety is coupled to the N-terminus of an IgG1 heavy chain. In some embodiments, the SLAMF7-binding moiety is coupled to the N-terminus of an IgG1 heavy chain. In some embodiments, the CD3-binding moiety is coupled to the N-terminus of an IgG1 heavy chain. In some embodiments, the GPRC5D-binding moiety is coupled to the C-terminus of an IgG1 heavy chain. In some embodiments, the BCMA-binding moiety is coupled to the C-terminus of an IgG1 heavy chain. In some embodiments, the FcRH5-binding moiety is coupled to the C-terminus of an IgG1 heavy chain. In some embodiments, the CD38-binding moiety is coupled to the C-terminus of an IgG1 heavy chain. In some embodiments, the SLAMF7-binding moiety is coupled to the C-terminus of an IgG1 heavy chain. In some embodiments, the CD3-binding moiety is coupled to the C-terminus of an IgG1 heavy chain. In some embodiments, the IgG1 heavy chain is a modified IgG1 heavy chain. In some embodiments, at least one of the at least three binding moieties is coupled to a light chain. In some embodiments, at least one of the at least four binding moieties is coupled to a light chain. In some embodiments, the GPRC5D-binding moiety is coupled to an immunoglobulin kappa light chain domain. In some embodiments, the BCMA-binding moiety is coupled to an immunoglobulin kappa light chain domain. In some embodiments, the FcRH5-binding moiety is coupled to an immunoglobulin kappa light chain domain. In some embodiments, the CD38-binding moiety is coupled to an immunoglobulin kappa light chain domain. In some embodiments, the SLAMF7-binding moiety is coupled to an immunoglobulin kappa light chain domain. In some embodiments, the CD3-binding moiety is coupled to an immunoglobulin kappa light chain domain. In some embodiments, the GPRC5D-binding moiety is coupled to an immunoglobulin lambda light chain domain. In some embodiments, the BCMA-binding moiety is coupled to an immunoglobulin lambda light chain domain. In some embodiments, the FcRH5-binding moiety is coupled to an immunoglobulin lambda light chain domain. In some embodiments, the CD38-binding moiety is coupled to an immunoglobulin lambda light chain domain. In some embodiments, the SLAMF7-binding moiety is coupled to an immunoglobulin lambda light chain domain. In some embodiments, the CD3-binding moiety is coupled to an immunoglobulin lambda light chain domain. In some embodiments, one of the at least three binding moieties is coupled to an immunoglobulin lambda light chain domain and another of the at least three binding moieties is coupled to an immunoglobulin kappa light chain domain. In some embodiments, one of the at least four binding moieties is coupled to an immunoglobulin lambda light chain domain and another of the at least three binding moieties is coupled to an immunoglobulin kappa light chain domain. In some embodiments, the light chain is a modified light chain. In some embodiments, the immunoglobulin kappa light chain domain resides on a modified light chain. In some embodiments, the immunoglobulin lambda light chain domain resides on a modified light chain. In some embodiments, the GPRC5D-binding moiety is a feature of a tetraspecific binder molecule that also comprises a CD3-binding moiety, a SLAMF7-binding moiety, and a BCMA-binding moiety. In some embodiments, the GPRC5D-binding moiety is a feature of a tetraspecific binder molecule that also comprises a CD3-binding moiety, a CD38-binding moiety, and a BCMA-binding moiety. In some embodiments, the GPRC5D-binding moiety is a feature of a tetraspecific binder molecule that also comprises a CD3-binding moiety, a CD38-binding moiety, and a SLAMF7-binding moiety. In some embodiments, the BCMA-binding moiety is a feature of a tetraspecific binder molecule that also comprises a CD3-binding moiety, a SLAMF7-binding moiety, and a GPRC5D-binding moiety. In some embodiments, the BCMA-binding moiety is a feature of a tetraspecific binder molecule that also comprises a CD3-binding moiety, a CD38-binding moiety, and a GPRC5D-binding moiety. In some embodiments, the BCMA-binding moiety is a feature of a tetraspecific binder molecule that also comprises a CD3-binding moiety, a CD38-binding moiety, and a SLAMF7-binding moiety. In some embodiments, the FcRH5-binding moiety is a feature of a tetraspecific binder molecule that also comprises a CD3-binding moiety, a BCMA-binding moiety, and a GPRC5D-binding moiety. In some embodiments, the FcRH5-binding moiety is a feature of a tetraspecific binder molecule that also comprises a CD3-binding moiety, a CD38-binding moiety, and a GPRC5D-binding moiety. In some embodiments, the FcRH5-binding moiety is a feature of a tetraspecific binder molecule that also comprises a CD3-binding moiety, a CD38-binding moiety, and a SLAMF7-binding moiety. In some embodiments, the FcRH5-binding moiety is a feature of a tetraspecific binder molecule that also comprises a CD3-binding moiety, a CD38-binding moiety, and a BCMA-binding moiety. In some embodiments, the FcRH5-binding moiety is a feature of a tetraspecific binder molecule that also comprises a CD3-binding moiety, a BCMA-binding moiety, and a SLAMF7-binding moiety. In some embodiments, the CD38-binding moiety is a feature of a tetraspecific binder molecule that also comprises a CD3-binding moiety, a SLAMF7-binding moiety, and a GPRC5D-binding moiety. In some embodiments, the CD38-binding moiety is a feature of a tetraspecific binder molecule that also comprises a CD3-binding moiety, a SLAMF7-binding moiety, and a BCMA-binding moiety. In some embodiments, the CD38-binding moiety is a feature of a tetraspecific binder molecule that also comprises a CD3-binding moiety, a GPRC5D-binding moiety, and a BCMA-binding moiety. In some embodiments, the SLAMF7-binding moiety is a feature of a tetraspecific binder molecule that also comprises a CD3-binding moiety, a BCMA-binding moiety, and a GPRC5D-binding moiety. In some embodiments, the SLAMF7-binding moiety is a feature of a tetraspecific binder molecule that also comprises a CD3-binding moiety, a CD38-binding moiety, and a BCMA-binding moiety. In some embodiments, the SLAMF7-binding moiety is a feature of a tetraspecific binder molecule that also comprises a CD3-binding moiety, a GPRC5D-binding moiety, and a CD38-binding moiety. In some embodiments, the multispecific binder molecule at least two immune cell binders. In some embodiments, the multispecific binder molecule at least three immune cell binders. In some embodiments, the multispecific binder molecule four immune cell binders.

### Antibody-Based Immune Cell Engagers

Provided herein are antibody-based binders that bind to CD3. In some embodiments, the antibody-based binder comprises an immune cell engager. In some instances, the immune cell engager is a T cell binding moiety. In some instances, the immune cell engager binds to a cell surface marker of the T cell. In some instances, the immune cell engager comprises a CD3 binding moiety. In some embodiments, the immune cell engager comprises an NK cell binding moiety. In some instances, the NK cell binding moiety is selected from a moiety that binds to CD56, CD161, CD94, CD16, NKG2A, NKG2C, NKG2D, MF1, NKp30, NKp44, NKp46, TBR1, +CD158a or CD158b. In some embodiments, the immune cell engager comprises an NKT cell binding moiety. In some instances, the NKT cell binding moiety is selected from a moiety that binds to CD161, CD16, CD158a, CD158b, NKG2A, NKG2C, NKp30, NKp44, or NKp46. In some embodiments, the multispecific binder molecule comprises the immune cell engager, a GPRC5D binding moiety, a BCMA binding moiety, and a CD38 binding moiety. In some embodiments, the multispecific binder molecule comprises the immune cell engager, a GPRC5D binding moiety, a BCMA binding moiety, and a SLAMF7 binding moiety. In some embodiments, the multispecific binder molecule comprises the immune cell engager, a GPRC5D binding moiety, a SLAMF7 binding moiety, and a CD38 binding moiety. In some embodiments, the multispecific binder molecule comprises the immune cell engager, a SLAMF7 binding moiety, a BCMA binding moiety, and a CD38 binding moiety. In some embodiments, the multispecific binder molecule comprises the immune cell engager, a GPRC5D binding moiety, a BCMA binding moiety, and an FcRH5 binding moiety. In some embodiments, the multispecific binder molecule comprises the immune cell engager, a GPRC5D binding moiety, a CD38 binding moiety, and an FcRH5 binding moiety. In some embodiments, the multispecific binder molecule comprises the immune cell engager, a GPRC5D binding moiety, a SLAMF7 binding moiety, and an FcRH5 binding moiety. In some embodiments, the multispecific binder molecule comprises the immune cell engager, a CD38 binding moiety, a SLAMF7 binding moiety, and an FcRH5 binding moiety.

In some embodiments, the CD3 binding moiety enhances binding of the multispecific binder molecule to T cells. In some embodiments, the CD3 binding moiety enhances binding of the multispecific binder molecule to NKT cells. In some embodiments, the CD3 binding moiety enhances binding of the multispecific binder molecule to T cells and NKT cells. In some embodiments, the CD3 binding moiety engages one or more classes of immune cells. In some embodiments, the CD3 binding moiety allows the multispecific binder molecule to bind one or more T cells simultaneously as binding one or more plasma cells. In some embodiments, the CD3 binding moiety allows the multispecific binder molecule to bind one or more T cells simultaneously as binding one or more malignant plasma cells. In some embodiments, the CD3 binding moiety allows the multispecific binder molecule to bind one or more NKT cells simultaneously as binding one or more malignant plasma cells. In some embodiments, the CD3 binding moiety allows the multispecific binder molecule to simultaneously bind to a T cell or an NKT cell and a tumor cells expressing any one of BCMA, CD38, SLAMF7, or GPRC5D. In some embodiments, the CD3 binding moiety allows the multispecific binder molecule to simultaneously bind to a T cell or an NKT cell and a tumor cells expressing any two of BCMA, CD38, SLAMF7, or GPRC5D. In some embodiments, the CD3 binding moiety allows the multispecific binder molecule to simultaneously bind to a T cell or an NKT cell and a tumor cells expressing BCMA, CD38, and SLAMF7.

In some embodiments, the CD3 binding moiety allows the multispecific binder molecule to simultaneously bind to a T cell or an NKT cell and a tumor cells expressing any one of BCMA, CD38, SLAMF7, GPRC5D, or FcRH5. In some embodiments, the CD3 binding moiety allows the multispecific binder molecule to simultaneously bind to a T cell or an NKT cell and a tumor cells expressing any two of BCMA, CD38, SLAMF7, GPRC5D, or FcRH5. In some embodiments, the CD3 binding moiety allows the multispecific binder molecule to simultaneously bind to a T cell or an NKT cell and a tumor cells expressing BCMA, GPRC5D, and FcRH5. In some embodiments, the CD3 binding moiety allows the multispecific binder molecule to simultaneously bind to a T cell or an NKT cell and a tumor cells expressing BCMA, CD38, and FcRH5. In some embodiments, the CD3 binding moiety allows the multispecific binder molecule to simultaneously bind to a T cell or an NKT cell and a tumor cells expressing BCMA, SLAMF7, and FcRH5. In some embodiments, the CD3 binding moiety allows the multispecific binder molecule to simultaneously bind to a T cell or an NKT cell and a tumor cells expressing CD38, GPRC5D, and FcRH5. In some embodiments, the CD3 binding moiety allows the multispecific binder molecule to simultaneously bind to a T cell or an NKT cell and a tumor cells expressing SLAMF7, GPRC5D, and FcRH5. In some embodiments, the CD3 binding moiety allows the multispecific binder molecule to simultaneously bind to a T cell or an NKT cell and a tumor cells expressing CD38, SLAMF7, and FcRH5. In some embodiments, the CD3 binding moiety allows the multispecific binder molecule to simultaneously bind to a T cell or an NKT cell and a tumor cells expressing BCMA, CD38, and GPRC5D. In some embodiments, the CD3 binding moiety allows the multispecific binder molecule to simultaneously bind to a T cell or an NKT cell and a tumor cells expressing BCMA, SLAMF7, and GPRC5D. In some embodiments, the CD3 binding moiety allows the multispecific binder molecule to simultaneously bind to a T cell or an NKT cell and a tumor cells expressing CD38, SLAMF7, and GPRC5D. In some embodiments, the tumor cells are from a hematological cancer. In some embodiments, the tumor cells are from myeloma. In some embodiments, the tumor cells are from multiple myeloma. In some embodiments, the multispecific binder molecule brings T cells closer in proximity to tumor cells. In some embodiments, the binding of the multispecific binder molecule potentiates unstimulated T cells or unstimulated NKT cells and induces direct cytotoxicity against tumor cells expressing BCMA, CD38, FcRH5, SLAMF7, or GPRC5D, or any combination thereof.

In some embodiments, the multispecific binder molecules comprises at least two immune cell engagers. In some embodiments, the multispecific binder molecules comprises at least two T cell engagers. In some embodiments, the at least two T cell engagers comprise a CD3-binding moiety and a second binding moiety selected from the group consisting of a CD2-binding moiety, a CD5-binding moiety, a CD8-binding moiety, a CD25-binding moiety, a CD27-binding moiety, and a CD28-binding moiety.

### Targeted Therapeutics

Various targeted therapeutics have been developed to create immunotherapies that can be applied to the treatment of hematological cancers including MM. Bispecific targeted therapeutics eliciting binding to both BCMA and CD3 include AMG 701, Alnuctamab, Elranatamab, Linvoseltamab, Teclistamab, ABBV-383, and HPN217. Bispecific targeted therapeutics eliciting binding to both CD38 and CD3 include XmAb969 and ISB1347. Bispecific targeted therapeutics eliciting binding to both GPRC5D and CD3 include Talquetamab and RG6234. Bispecific targeted therapeutics eliciting binding to both FcRH5 and CD3 include Cevostamab. A trispecific targeted therapeutic eliciting binding to CD38, CD28, and CD3 is SAR442257. A trispecific targeted therapeutic eliciting binding to BCMA, CD38, and CD3 is ISB 2001. Binding moieties from previously developed targeted therapeutics can be derived from them and adapted for use in targeting the multispecific binder molecule. In some embodiments, the multispecific binder molecule comprises a GPRC5D binding moiety derived from Talquetamab or RG6234. In some embodiments, the multispecific binder molecule comprises a TNFRSF17 (BCMA) binding moiety derived from AMG 701 (pavurutamab), Alnuctamab (EM 801), Elranatamab, Linvoseltamab (REGN5458), Teclistamab (Tecvayli™), ABBV-383 (TNB383B), HPN217, ISB 2001, GSK287916, MEDI2228, or AMG 420. In some embodiments, the multispecific binder molecule comprises a SLAMF7 (CD319) binding moiety derived from ABBV-838, Luc63, or Elotuzumab. In some embodiments, the multispecific binder molecule comprises an FcRH5 binding moiety derived from Cevostamab. In some embodiments, the multispecific binder molecule comprises an FcRH5 binding moiety derived from DFRF4539A. In some embodiments, the multispecific binder molecule comprises a CD38 binding moiety derived from Daratumumab, Isatuximab, I S B 2001, or MOR202. In some embodiments, the multispecific binder molecule comprises an immune cell engager. In some embodiments, an immune cell engager from previously developed targeted therapeutics can be derived from one of them and adapted for use in engaging immune cells with the targeted tumor cells. In some embodiments, the immune cell engager binds to one or more CD3 chains. In some embodiments, the immune cell engager binds to CD3 gamma chain. In some embodiments, the immune cell engager binds to CD3 delta chain. In some embodiments, the immune cell engager binds to CD3 epsilon chain. In some embodiments, the immune cell engager binds to CD3 zeta chain. In some embodiments, multispecific binder molecule comprises an immune cell engager comprising a CD3 binding moiety derived from AMG 701, Alnuctamab, Elranatamab, Linvoseltamab, Teclistamab, ABBV-383, HPN217, XmAb969, ISB1347, Talquetamab, RG6234, Cevostamab, SAR442257, Cevostamab, or ISB 2001. In some embodiments, the multispecific binder molecule comprises a tetraspecific binder molecule.

In some embodiments, the tetraspecific binder molecule comprises a CD38-binding moiety derived from Daratumumab, a SLAMF7-binding moiety derived from Elotuzumab, a BCMA-binding moiety derived from Teclistamab, and a CD3-binding moiety. In some embodiments, the tetraspecific binder molecule comprises a CD38-binding moiety derived from Daratumumab, a SLAMF7-binding moiety derived from Elotuzumab, a GPRC5D-binding moiety derived from Talquetamab, and a CD3-binding moiety. In some embodiments, the tetraspecific binder molecule comprises a CD38-binding moiety derived from Daratumumab, a BCMA-binding moiety derived from Teclistamab, a GPRC5D-binding moiety derived from Talquetamab, and a CD3-binding moiety. In some embodiments, the tetraspecific binder molecule comprises a SLAMF7-binding moiety derived from Elotuzumab, a BCMA-binding moiety derived from Teclistamab, a GPRC5D-binding moiety derived from Talquetamab, and a CD3-binding moiety. In some embodiments, a binding moiety derived from a targeted therapeutic listed herein comprises amino acids of a CDR series described herein. In some embodiments, the CDR series is located in a VH domain and described in Table 1. In some embodiments, the CDR series is located in a VH domain and described in Table 1 and comprises an HCDR1 sequence, an HCDR2 sequence, and an HCDR3 sequence from a CDR series listed in Table 1 and found within antibody sequences from one of SEQ ID NOs: 21, 22, 1, 2, 23, 24, 3, 4, 5, 6, 25, 26, 27, 28, 7, 29, 30, 31, 32, 48, 8, 9, 33, or 34. In some embodiments, the CDR series is located in a VL domain and described in Table 2. In some embodiments, the CDR series is located in a VL domain and described in Table 2 and comprises an LCDR1 sequence, an LCDR2 sequence, and an LCDR3 sequence from a CDR series listed in Table 2 and found within antibody sequences from one of SEQ ID NOs: 35, 36, 12, 13, 37, 38, 14, 15, 16, 17, 39, 40, 41, 42, 43, 44, 48, 45, or 46. In some embodiments, a binding moiety derived from a targeted therapeutic listed herein comprises amino acids of a VH domain described herein. In some embodiments, the VH domain sequence is listed in Table 3. In some embodiments, a binding moiety derived from a targeted therapeutic listed herein comprises amino acids of a VL domain described herein. In some embodiments, the VL domain sequence is listed in Table 4.

Heavy chain CDR sequences listed according to different antibody numbering schemes derived from targeted therapeutics are listed in Table 1. Heavy chain CDR sequences listed in Table 1 are adapted for use in generating a GPRC5D binding moiety in the multispecific binder molecule. Heavy chain CDR sequences listed in Table 1 are adapted for use in generating a TNFRSF17 (BCMA) binding moiety in the multispecific binder molecule. Heavy chain CDR sequences listed in Table 1 are adapted for use in generating an FcRH5 binding moiety in the multispecific binder molecule. Heavy chain CDR sequences listed in Table 1 are adapted for use in generating a CD38 binding moiety in the multispecific binder molecule. Heavy chain CDR sequences listed in Table 1 are adapted for use in generating a SLAMF7 (CD319) binding moiety in the multispecific binder molecule. Heavy chain CDR sequences listed in Table 1 are adapted for use in generating a T cell binding moiety that binds to CD3 in the multispecific binder molecule. Heavy chain CDR sequences listed in Table 1 are adapted for use in generating a NK cell binding moiety that binds to CD3 in the multispecific binder molecule. Heavy chain CDR sequences listed in Table 1 are adapted for use in generating a NKT cell binding moiety that binds to CD3 in the multispecific binder molecule. Light chain CDR sequences listed according to different antibody numbering schemes derived from targeted therapeutics are listed in Table 2. Light chain CDR sequences listed in Table 2 are adapted for use in generating a GPRC5D binding moiety in the multispecific binder molecule. Light chain CDR sequences listed in Table 2 are adapted for use in generating a TNFRSF17 (BCMA) binding moiety in the multispecific binder molecule. Light chain CDR sequences listed in Table 2 are adapted for use in generating an FcRH5 binding moiety in the multispecific binder molecule. Light chain CDR sequences listed in Table 2 are adapted for use in generating a CD38 binding moiety in the multispecific binder molecule. Light chain CDR sequences listed in Table 2 are adapted for use in generating a SLAMF7 (CD319) binding moiety in the multispecific binder molecule. Light chain CDR sequences listed in Table 2 are adapted for use in generating a T cell binding moiety that binds to CD3 in the multispecific binder molecule. Light chain CDR sequences listed in Table 2 are adapted for use in generating a NK cell binding moiety that binds to CD3 in the multispecific binder molecule. Light chain CDR sequences listed in Table 2 are adapted for use in generating a NKT cell binding moiety that binds to CD3 in the multispecific binder molecule.

TABLE 1
HCDR Sequences Derived from Targeted Therapeutics

| Found | | | | | | | SEQ | |
|---|---|---|---|---|---|---|---|---|
| Within | | | SEQ | | SEQ | | ID | |
| SEQ ID | Numbering | | ID | | ID | | NO: | Targeted |
| NO: | Scheme | HCDR1 | NO: | HCDR2 | NO: | HCDR3 | | Therapeutic |
| 21 | IMGT | GFTFNS | 88 | ISGSGG | 186 | AKDKIL | 280 | Daratumumab |
| | | FA | | GT | | WFGEPV | | |
| | | | | | | FDY | | |
| | Kabat | SFAMS | 89 | AISGSG | 187 | DKILWF | 281 | |
| | | | | GGTYYA | | GEPVFD | | |
| | | | | DSVKG | | Y | | |
| | AbM | GFTFNS | 90 | AISGSG | 188 | DKILWF | 281 | |
| | | FAMS | | GGTY | | GEPVFD | | |
| | | | | | | Y | | |
| | Chothia | GFTFNS | 91 | SGSGG | 189 | DKILWF | 281 | |
| | | F | | G | | GEPVFD | | |
| | | | | | | Y | | |
| | Contact | NSFAMS | 92 | WVSAIS | 190 | AKDKIL | 282 | |
| | | | | GSGGG | | WFGEPV | | |
| | | | | TY | | FD | | |
| 22 | IMGT | GYTFTD | 93 | IYPGDG | 191 | ARGDYY | 283 | Isatuximab |
| | | YW | | DT | | GSNSLD | | |
| | | | | | | Y | | |
| | Kabat | DYWMQ | 94 | TIYPGD | 192 | GDYYGS | 284 | |
| | | | | GDTGYA | | NSLDY | | |
| | | | | QKFQG | | | | |
| | AbM | GYTFTD | 95 | TIYPGD | 193 | GDYYGS | 284 | |
| | | YWMQ | | GDTG | | NSLDY | | |
| | Chothia | GYTFTD | 96 | YPGDGD | 194 | GDYYGS | 284 | |
| | | Y | | | | NSLDY | | |
| | Contact | TDYWM | 97 | WIGTIYP | 195 | ARGDYY | 285 | |
| | | Q | | GDGDTG | | GSNSLD | | |
| 1 | IMGT | GFTFSS | 98 | ISGDPS | 196 | ARDLPL | 286 | MOR202 |
| | | YY | | NT | | VYTGFA | | |
| | | | | | | Y | | |
| | Kabat | SYYMN | 99 | GISGDP | 197 | DLPLVY | 287 | |
| | | | | SNTYYA | | TGFAY | | |
| | | | | DSVKG | | | | |
| | AbM | GFTFSS | 100 | GISGDP | 198 | DLPLVY | 287 | |
| | | YYMN | | SNTY | | TGFAY | | |
| | Chothia | GFTFSS | 101 | SGDPSN | 199 | DLPLVY | 287 | |
| | | Y | | | | TGFAY | | |
| | Contact | SSYYMN | 102 | WVSGIS | 200 | ARDLPL | 288 | |
| | | | | GDPSNT | | VYTGFA | | |
| | | | | Y | | | | |
| 2 | IMGT | GFDFSR | 103 | INPDSST | 201 | ARPDGN | 289 | Luc63 |
| | | YW | | 1 | | YWYFDV | | |
| | Kabat | RYWMS | 104 | EINPDSS | 202 | PDGNY | 290 | |
| | | | | TINYTPS | | WYFDV | | |
| | | | | LKD | | | | |
| | AbM | GFDFSR | 105 | EINPDSS | 203 | PDGNY | 290 | |
| | | YWMS | | TIN | | WYFDV | | |
| | Chothia | GFDFSR | 106 | NPDSST | 204 | PDGNY | 290 | |
| | | Y | | | | WYFDV | | |
| | Contact | SRYWM | 107 | WIGEINP | 205 | ARPDGN | 291 | |
| | | S | | DSSTIN | | YWYFD | | |
| 23 | IMGT | GFTFSD | 108 | INYDGS | 206 | ARDRGY | 292 | ABBV-838 |
| | | YY | | ST | | YFDY | | |
| | Kabat | DYYMA | 109 | SINYDG | 207 | DRGYYF | 293 | |
| | | | | SSTYYV | | DY | | |
| | | | | DSVKG | | | | |
| | AbM | GFTFSD | 110 | SINYDG | 208 | DRGYYF | 293 | |
| | | YYMA | | SSTY | | DY | | |
| | Chothia | GFTFSD | 111 | NYDGSS | 209 | DRGYYF | 293 | |
| | | Y | | | | DY | | |
| | Contact | SDYYMA | 112 | WVASIN | 210 | ARDRGY | 294 | |
| | | | | YDGSST | | YFD | | |
| | | | | Y | | | | |
| 24 | IMGT | GFDFSR | 103 | INPDSST | 201 | ARPDGN | 289 | Elotuzumab |
| | | YW | | | | YWYFDV | | |
| | Kabat | RYWMS | 104 | EINPDSS | 211 | PDGNY | 290 | |
| | | | | TINYAPS | | WYFDV | | |
| | | | | LKD | | | | |
| | AbM | GFDFSR | 105 | EINPDSS | 203 | PDGNY | 290 | |
| | | YWMS | | TIN | | WYFDV | | |
| | Chothia | GFDFSR | 106 | NPDSST | 204 | PDGNY | 290 | |
| | | Y | | | | WYFDV | | |
| | Contact | SRYWM | 107 | WIGEINP | 205 | ARPDGN | 291 | |
| | | S | | DSSTIN | | YWYFD | | |
| 3 | IMGT | GYTFTN | 113 | INPYPG | 212 | ARDGYY | 295 | AMG 701 |
| | | HI | | YH | | RDTDVL | | (BCMA) |
| | | | | | | DY | | |
| | Kabat | NHIIH | 114 | YINPYP | 213 | DGYYRD | 296 | |
| | | | | GYHAYN | | TDVLDY | | |
| | | | | EKFQG | | | | |
| | AbM | GYTFTN | 115 | YINPYP | 214 | DGYYRD | 296 | |
| | | HIIH | | GYHA | | TDVLDY | | |
| | Chothia | GYTFTN | 116 | NPYPGY | 215 | DGYYRD | 296 | |
| | | H | | | | TDVLDY | | |
| | Contact | TNHIIH | 117 | WMGYIN | 216 | ARDGYY | 297 | |
| | | | | PYPGYH | | RDTDVL | | |
| | | | | A | | D | | |
| 4 | IMGT | GFTFNK | 118 | IRSKYN | 217 | VRHGNF | 298 | AMG 701 |
| | | YA | | NYAT | | GNSYIS | | (CD3) |
| | | | | | | YWAY | | |
| | Kabat | KYAMN | 119 | RIRSKY | 218 | HGNFGN | 299 | |
| | | | | NNYATY | | SYISYW | | |
| | | | | YADSVK | | AY | | |
| | | | | D | | | | |
| | AbM | GFTFNK | 120 | RIRSKY | 219 | HGNFGN | 299 | |
| | | YAMN | | NNYATY | | SYISYW | | |
| | | | | | | AY | | |
| | Chothia | GFTFNK | 121 | RSKYNN | 220 | HGNFGN | 299 | |
| | | Y | | YA | | SYISYW | | |
| | | | | | | AY | | |
| | Contact | NKYAMN | 122 | WVARIR | 221 | VRHGNF | 300 | |
| | | | | SKYNNY | | GNSYIS | | |
| | | | | ATY | | YWA | | |
| 5 | IMGT | GFTFSD | 123 | ISGPGS | 222 | AKVLGW | 301 | Alnuctamab |
| | | NA | | ST | | FDY | | (EM 801) |
| | Kabat | DNAMG | 124 | AISGPG | 223 | VLGWFD | 302 | (BCMA) |
| | | | | SSTYYA | | Y | | |
| | | | | DSVKG | | | | |
| | AbM | GFTFSD | 125 | AISGPG | 224 | VLGWFD | 302 | |
| | | NAMG | | SSTY | | Y | | |
| | Chothia | GFTFSD | 126 | SGPGSS | 225 | VLGWFD | 302 | |
| | | N | | | | Y | | |
| | Contact | SDNAMG | 127 | WVSAIS | 226 | AKVLGW | 303 | |
| | | | | GPGSST | | FD | | |
| | | | | Y | | | | |
| 6 | IMGT | GFTFST | 128 | IRSKYN | 217 | VRHGNF | 304 | Alnuctamab |
| | | YA | | NYAT | | GNSYVS | | (EM 801) |
| | | | | | | WFAY | | (CD3) |
| | Kabat | TYAMN | 129 | RIRSKY | 227 | HGNFGN | 305 | |
| | | | | NNYATY | | SYVSWF | | |
| | | | | YADSVK | | AY | | |
| | | | | G | | | | |
| | AbM | GFTFST | 130 | RIRSKY | 219 | HGNFGN | 305 | |
| | | YAMN | | NNYATY | | SYVSWF | | |
| | | | | | | AY | | |
| | Chothia | GFTFST | 131 | RSKYNN | 220 | HGNFGN | 305 | |
| | | Y | | YA | | SYVSWF | | |
| | | | | | | AY | | |
| | Contact | STYAMN | 132 | WVSRIR | 228 | VRHGNF | 306 | |
| | | | | SKYNNY | | GNSYVS | | |
| | | | | ATY | | WFA | | |
| 25 | IMGT | GFTFSD | 108 | IRNRAR | 229 | ARDRPS | 307 | Elranatamab |
| | | YY | | GYTS | | YYVLDY | | (CD3e) |
| | Kabat | DYYMT | 133 | FIRNRA | 230 | DRPSYY | 308 | |
| | | | | RGYTSD | | VLDY | | |
| | | | | HNPSVK | | | | |
| | | | | G | | | | |
| | AbM | GFTFSD | 134 | FIRNRA | 231 | DRPSYY | 308 | |
| | | YYMT | | RGYTSD | | VLDY | | |
| | Chothia | GFTFSD | 111 | RNRARG | 232 | DRPSYY | 308 | |
| | | Y | | YT | | VLDY | | |
| | Contact | SDYYMT | 135 | WVAFIR | 233 | ARDRPS | 309 | |
| | | | | NRARGY | | YYVLD | | |
| | | | | TSD | | | | |
| 26 | IMGT | GFTFSS | 136 | IGGSGG | 234 | ARYWP | 310 | Elranatamab |
| | | YP | | SL | | MDI | | (BCMA) |
| | Kabat | SYPMS | 137 | AIGGSG | 235 | YWPMDI | 311 | |
| | | | | GSLPYA | | | | |
| | | | | DIVKG | | | | |
| | AbM | GFTFSS | 138 | AIGGSG | 236 | YWPMDI | 311 | |
| | | YPMS | | GSLP | | | | |
| | Chothia | GFTFSS | 101 | GGSGG | 237 | YWPMDI | 311 | |
| | | Y | | S | | | | |
| | Contact | SSYPMS | 139 | WVSAIG | 238 | ARYWP | 312 | |
| | | | | GSGGSL | | MD | | |
| | | | | P | | | | |
| 27 | IMGT | GFTFSN | 140 | MNQDG | 239 | ARDREY | 313 | Linvoseltamab |
| | | FW | | SEK | | CISTSCY | | (REGN5458) |
| | | | | | | DDFDY | | Heavy chain 1 |
| | Kabat | NFWMT | 141 | NMNQD | 240 | DREYCI | 314 | (CD3 or |
| | | | | GSEKYY | | STSCYD | | BCMA) |
| | | | | VDSVKG | | DFDY | | |
| | AbM | GFTFSN | 142 | NMNQD | 241 | DREYCI | 314 | |
| | | FWMT | | GSEKY | | STSCYD | | |
| | | | | | | DFDY | | |
| | Chothia | GFTFSN | 143 | NQDGSE | 242 | DREYCI | 314 | |
| | | F | | | | STSCYD | | |
| | | | | | | DFDY | | |
| | Contact | SNFWMT | 144 | WVANM | 243 | ARDREY | 315 | |
| | | | | NQDGSE | | CISTSCY | | |
| | | | | KY | | DDFD | | |
| 28 | IMGT | GFTFDD | 145 | ISWNSG | 244 | AKYGSG | 316 | Linvoseltamab |
| | | YS | | SK | | YGKFYH | | (REGN5458) |
| | | | | | | YGLDV | | Heavy chain 2 |
| | Kabat | DYSMH | 146 | GISWNS | 245 | YGSGYG | 317 | (CD3 or |
| | | | | GSKGYA | | KFYHYG | | BCMA) |
| | | | | DSVKG | | LDV | | |
| | AbM | GFTFDD | 147 | GISWNS | 246 | YGSGYG | 317 | |
| | | YSMH | | GSKG | | KFYHYG | | |
| | | | | | | LDV | | |
| | Chothia | GFTFDD | 148 | SWNSG | 247 | YGSGYG | 317 | |
| | | Y | | S | | KFYHYG | | |
| | | | | | | LDV | | |
| | Contact | DDYSMH | 149 | WVSGIS | 248 | AKYGSG | 318 | |
| | | | | WNSGS | | YGKFYH | | |
| | | | | KG | | YGLD | | |
| 7 | IMGT | GFTFNT | 150 | IRSKYN | 217 | VRHGNF | 304 | SP34 CD3 |
| | | YA | | NYAT | | GNSYVS | | |
| | | | | | | WFAY | | |
| | Kabat | TYAMN | 129 | RIRSKY | 218 | HGNFGN | 305 | |
| | | | | NNYATY | | SYVSWF | | |
| | | | | YADSVK | | AY | | |
| | | | | D | | | | |
| | AbM | GFTFNT | 151 | RIRSKY | 219 | HGNFGN | 305 | |
| | | YAMN | | NNYATY | | SYVSWF | | |
| | | | | | | AY | | |
| | Chothia | GFTFNT | 152 | RSKYNN | 220 | HGNFGN | 305 | |
| | | Y | | YA | | SYVSWF | | |
| | | | | | | AY | | |
| | Contact | NTYAMN | 153 | WVARIR | 221 | VRHGNF | 306 | |
| | | | | SKYNNY | | GNSYVS | | |
| | | | | ATY | | WFA | | |
| 29 | IMGT | GGSISS | 154 | IYYSGIT | 249 | ARHDGA | 319 | Teclistamab |
| | | GSYF | | | | VAGLFD | | (anti-BCMA |
| | | | | | | Y | | heavy chain) |
| | Kabat | SGSYFW | 155 | SIYYSGI | 250 | HDGAVA | 320 | |
| | | G | | TYYNPS | | GLFDY | | |
| | | | | LKS | | | | |
| | AbM | GGSISS | 156 | SIYYSGI | 251 | HDGAVA | 320 | |
| | | GSYFW | | TY | | GLFDY | | |
| | | G | | | | | | |
| | Chothia | GGSISS | 157 | YYSGI | 252 | HDGAVA | 320 | |
| | | GSY | | | | GLFDY | | |
| | Contact | SSGSYF | 158 | WIGSIYY | 253 | ARHDGA | 321 | |
| | | WG | | SGITY | | VAGLFD | | |
| 30 | IMGT | GFTFNT | 150 | IRSKYN | 217 | ARHGNF | 322 | Teclistamab |
| | | YA | | NYAT | | GNSYVS | | (anti-CD3 |
| | | | | | | WFAY | | heavy chain) |
| | Kabat | TYAMN | 129 | RIRSKY | 254 | HGNFGN | 305 | |
| | | | | NNYATY | | SYVSWF | | |
| | | | | YAASVK | | AY | | |
| | | | | G | | | | |
| | AbM | GFTFNT | 151 | RIRSKY | 219 | HGNFGN | 305 | |
| | | YAMN | | NNYATY | | SYVSWF | | |
| | | | | | | AY | | |
| | Chothia | GFTFNT | 152 | RSKYNN | 220 | HGNFGN | 305 | |
| | | Y | | YA | | SYVSWF | | |
| | | | | | | AY | | |
| | Contact | NTYAMN | 153 | WVARIR | 221 | ARHGNF | 323 | |
| | | | | SKYNNY | | GNSYVS | | |
| | | | | ATY | | WFA | | |
| 31 | IMGT | GFTVSS | 159 | IRGSDG | 255 | AKQGEN | 324 | ABBV-383 |
| | | YG | | ST | | DGPFDH | | BCMA heavy |
| | Kabat | SYGMS | 160 | GIRGSD | 256 | QGEND | 325 | chain |
| | | | | GSTYYA | | GPFDH | | |
| | | | | DSVKG | | | | |
| | AbM | GFTVSS | 161 | GIRGSD | 257 | QGEND | 325 | |
| | | YGMS | | GSTY | | GPFDH | | |
| | Chothia | GFTVSS | 162 | RGSDGS | 258 | QGEND | 325 | |
| | | Y | | | | GPFDH | | |
| | Contact | SSYGMS | 163 | WVSGIR | 259 | AKQGEN | 326 | |
| | | | | GSDGST | | DGPFD | | |
| | | | | Y | | | | |
| 32 | IMGT | GFTFDD | 164 | ISWNSG | 260 | AKDSRG | 327 | ABBV-383 |
| | | YA | | SI | | YGDYRL | | CD3 heavy |
| | | | | | | GGAY | | chain |
| | Kabat | DYAMH | 165 | GISWNS | 261 | DSRGYG | 328 | |
| | | | | GSIGYA | | DYRLGG | | |
| | | | | DSVKG | | AY | | |
| | AbM | GFTFDD | 166 | GISWNS | 262 | DSRGYG | 328 | |
| | | YAMH | | GSIG | | DYRLGG | | |
| | | | | | | AY | | |
| | Chothia | GFTFDD | 148 | SWNSG | 247 | DSRGYG | 328 | |
| | | Y | | S | | DYRLGG | | |
| | | | | | | AY | | |
| | Contact | DDYAMH | 167 | WVSGIS | 263 | AKDSRG | 329 | |
| | | | | WNSGSI | | YGDYRL | | |
| | | | | G | | GGA | | |
| 48 | IMGT | TNIFSIS | 168 | IHGTST | 264 | NKVPW | 330 | HPN217 |
| | | P | | | | GDYHPG | | (BCMA) |
| | | | | | | NVY | | |
| | Kabat | ISPYG | 169 | AIHGTST | 265 | VPWGD | 331 | |
| | | | | LYADSV | | YHPGNV | | |
| | | | | KG | | Y | | |
| | AbM | TNIFSIS | 170 | AIHGTST | 266 | VPWGD | 331 | |
| | | PYG | | L | | YHPGNV | | |
| | | | | | | Y | | |
| | Chothia | TNIFSIS | 171 | HGTS | 267 | VPWGD | 331 | |
| | | | | | | YHPGNV | | |
| | | | | | | Y | | |
| | Contact | SISPYG | 172 | LVAAIHG | 268 | NKVPW | 332 | |
| | | | | TSTL | | GDYHPG | | |
| | | | | | | NV | | |
| 48 | IMGT | GFTFNK | 118 | IRSKYN | 217 | VRHANF | 333 | HPN217 (CD3) |
| | | YA | | NYAT | | GNSYIS | | |
| | | | | | | YWAY | | |
| | Kabat | KYAIN | 173 | RIRSKY | 269 | HANFGN | 334 | |
| | | | | NNYATY | | SYISYW | | |
| | | | | YADQVK | | AY | | |
| | | | | D | | | | |
| | AbM | GFTFNK | 174 | RIRSKY | 219 | HANFGN | 334 | |
| | | YAIN | | NNYATY | | SYISYW | | |
| | | | | | | AY | | |
| | Chothia | GFTFNK | 121 | RSKYNN | 220 | HANFGN | 334 | |
| | | Y | | YA | | SYISYW | | |
| | | | | | | AY | | |
| | Contact | NKYAIN | 175 | WVARIR | 221 | VRHANF | 335 | |
| | | | | SKYNNY | | GNSYIS | | |
| | | | | ATY | | YWA | | |
| 8 | IMGT | GFTFSN | 140 | MNQDG | 239 | ARDREY | 313 | REGN5459 |
| | | FW | | SEK | | CISTSCY | | (BCMA) |
| | | | | | | DDFDY | | |
| | Kabat | NFWMT | 141 | NMNQD | 240 | DREYCI | 314 | |
| | | | | GSEKYY | | STSCYD | | |
| | | | | VDSVKG | | DFDY | | |
| | AbM | GFTFSN | 142 | NMNQD | 241 | DREYCI | 314 | |
| | | FWMT | | GSEKY | | STSCYD | | |
| | | | | | | DFDY | | |
| | Chothia | GFTFSN | 143 | NQDGSE | 242 | DREYCI | 314 | |
| | | F | | | | STSCYD | | |
| | | | | | | DFDY | | |
| | Contact | SNFWMT | 144 | WVANM | 243 | ARDREY | 315 | |
| | | | | NQDGSE | | CISTSCY | | |
| | | | | KY | | DDFD | | |
| 9 | IMGT | GFTFDD | 145 | ISWNSG | 260 | AKYGSG | 336 | REGN5459 |
| | | YS | | SI | | YGKFYY | | (CD3) |
| | | | | | | YGMDV | | |
| | Kabat | DYSMH | 146 | GISWNS | 261 | YGSGYG | 337 | |
| | | | | GSIGYA | | KFYYYG | | |
| | | | | DSVKG | | MDV | | |
| | AbM | GFTFDD | 147 | GISWNS | 262 | YGSGYG | 337 | |
| | | YSMH | | GSIG | | KFYYYG | | |
| | | | | | | MDV | | |
| | Chothia | GFTFDD | 148 | SWNSG | 247 | YGSGYG | 337 | |
| | | Y | | S | | KFYYYG | | |
| | | | | | | MDV | | |
| | Contact | DDYSMH | 149 | WVSGIS | 263 | AKYGSG | 338 | |
| | | | | WNSGSI | | YGKFYY | | |
| | | | | G | | YGMD | | |
| 33 | IMGT | GFSLTR | 176 | IWRGGS | 270 | SNHYYG | 339 | Cevostamab |
| | | FG | | T | | SSDYAL | | (FcRH5) |
| | | | | | | DN | | |
| | Kabat | RFGVH | 177 | VIWRGG | 271 | HYYGSS | 340 | |
| | | | | STDYNA | | DYALDN | | |
| | | | | AFVS | | | | |
| | AbM | GFSLTR | 178 | VIWRGG | 272 | HYYGSS | 340 | |
| | | FGVH | | STD | | DYALDN | | |
| | Chothia | GFSLTR | 179 | WRGGS | 273 | HYYGSS | 340 | |
| | | F | | | | DYALDN | | |
| | Contact | TRFGVH | 180 | WLGVIW | 274 | SNHYYG | 341 | |
| | | | | RGGSTD | | SSDYAL | | |
| | | | | | | D | | |
| 34 | IMGT | GFTFTS | 181 | IYPEND | 275 | ARDGYS | 342 | Cevostamab |
| | | YY | | NT | | RYYFDY | | (CD3) |
| | Kabat | SYYIH | 182 | WIYPEN | 276 | DGYSRY | 343 | |
| | | | | DNTKYN | | YFDY | | |
| | | | | EKFKD | | | | |
| | AbM | GFTFTS | 183 | WIYPEN | 277 | DGYSRY | 343 | |
| | | YYIH | | DNTK | | YFDY | | |
| | Chothia | GFTFTS | 184 | YPENDN | 278 | DGYSRY | 343 | |
| | | Y | | | | YFDY | | |
| | Contact | TSYYIH | 185 | WIGWIY | 279 | ARDGYS | 344 | |
| | | | | PENDNT | | RYYFD | | |
| | | | | K | | | | |

In some embodiments, the VH region of the GPRC5D binding moiety comprises HCDR1, HCDR2, and HCDR3 sequences selected from the sequences listed in Table 1. In some embodiments, the VH region of the TNFRSF17 (BCMA) binding moiety comprises HCDR1, HCDR2, and HCDR3 sequences selected from the sequences listed in Table 1. In some embodiments, the VH region of the FcRH5 binding moiety comprises HCDR1, HCDR2, and HCDR3 sequences selected from the sequences listed in Table 1. In some embodiments, the VH region of the CD38 binding moiety comprises HCDR1, HCDR2, and HCDR3 sequences selected from the sequences listed in Table 1. In some embodiments, the VH region of the SLAMF7 (CD319) binding moiety HCDR1, HCDR2, and HCDR3 sequences selected from the sequences listed in Table 1. In some embodiments, the VH region of the immune cell engager CD3 binding moiety comprises HCDR1, HCDR2, and HCDR3 sequences selected from the sequences listed in Table 1.

In some embodiments, the VL region of the GPRC5D binding moiety comprises LCDR1, LCDR2, and LCDR3 sequences selected from the sequences listed in Table 2. In some embodiments, the VL region of the TNFRSF17 (BCMA) binding moiety comprises LCDR1, LCDR2, and LCDR3 sequences selected from the sequences listed in Table 2. In some embodiments, the VL region of the FcRH5 binding moiety comprises LCDR1, LCDR2, and LCDR3 sequences selected from the sequences listed in Table 2. In some embodiments, the VL region of the CD38 binding moiety comprises LCDR1, LCDR2, and LCDR3 sequences selected from the sequences listed in Table 2. In some embodiments, the VL region of the SLAMF7 (CD319) binding moiety comprises LCDR1, LCDR2, and LCDR3 sequences selected from the sequences listed in Table 2. In some embodiments, the VL region of the immune cell engager CD3 binding moiety comprises LCDR1, LCDR2, and LCDR3 sequences selected from the sequences listed in Table 2.

TABLE 2
LCDR Sequences Derived from Targeted Therapeutics

| Found | | | | | | | | |
|---|---|---|---|---|---|---|---|---|
| Within | | | SEQ | | SEQ | | SEQ | |
| SEQ ID | Numbering | | ID | | ID | | ID | Targeted |
| NO: | Scheme | LCDR1 | NO: | LCDR2 | NO: | LCDR3 | NO: | Therapeutic |
| 35 | IMGT | QSVSSY | 345 | DAS | | QQRSN | 430 | Daratumumab |
| | | | | | | WPPT | | |
| | Kabat | RASQSV | 346 | DASNRA | 395 | QQRSN | 430 | |
| | | SSYLA | | T | | WPPT | | |
| | AbM | RASQSV | 346 | DASNRA | 395 | QQRSN | 430 | |
| | | SSYLA | | T | | WPPT | | |
| | Chothia | RASQSV | 346 | DASNRA | 395 | QQRSN | 430 | |
| | | SSYLA | | T | | WPPT | | |
| | Contact | SSYLAW | 347 | LLIYDAS | 396 | QQRSN | 431 | |
| | | Y | | NRA | | WPP | | |
| 36 | IMGT | QDVSTV | 348 | SAS | | QQHYSP | 432 | Isatuximab |
| | | | | | | PYT | | |
| | Kabat | KASQDV | 349 | SASYRYI | 397 | QQHYSP | 432 | |
| | | STWVA | | | | PYT | | |
| | AbM | KASQDV | 349 | SASYRYI | 397 | QQHYSP | 432 | |
| | | STVVA | | | | PYT | | |
| | Chothia | KASQDV | 349 | SASYRYI | 397 | QQHYSP | 432 | |
| | | STWVA | | | | PYT | | |
| | Contact | STWVAW | 350 | RLIYSAS | 398 | QQHYSP | 433 | |
| | | Y | | YRY | | PY | | |
| 12 | IMGT | NLRHYY | 351 | GDS | | QTYTGG | 434 | MOR202 |
| | | | | | | ASLV | | |
| | Kabat | SGDNLR | 352 | GDSKRP | 399 | QTYTGG | 434 | |
| | | HYYVY | | S | | ASLV | | |
| | AbM | SGDNLR | 352 | GDSKRP | 399 | QTYTGG | 434 | |
| | | HYYVY | | S | | ASLV | | |
| | Chothia | SGDNLR | 352 | GDSKRP | 399 | QTYTGG | 434 | |
| | | HYYVY | | S | | ASLV | | |
| | Contact | HYYVYW | 353 | LVIYGDS | 400 | QTYTGG | 435 | |
| | | Y | | KRP | | ASL | | |
| 13 | IMGT | QDVGIA | 354 | WAS | | QQYSSY | 436 | Luc63 |
| | | | | | | PYT | | |
| | Kabat | KASQDV | 355 | WASTRH | 401 | QQYSSY | 436 | |
| | | GIAVA | | T | | PYT | | |
| | AbM | KASQDV | 355 | WASTRH | 401 | QQYSSY | 436 | |
| | | GIAVA | | T | | PYT | | |
| | Chothia | KASQDV | 355 | WASTRH | 401 | QQYSSY | 436 | |
| | | GIAVA | | T | | PYT | | |
| | Contact | GIAVAW | 356 | LLIYWAS | 402 | QQYSSY | 437 | |
| | | Y | | TRH | | PY | | |
| 37 | IMGT | QSLVHS | 357 | KVS | | SQSTHV | 438 | ABBV-838 |
| | | NGNTY | | | | PPFT | | |
| | Kabat | RSSQSL | 358 | KVSNRF | 403 | SQSTHV | 438 | |
| | | VHSNGN | | S | | PPFT | | |
| | | TYLH | | | | | | |
| | AbM | RSSQSL | 358 | KVSNRF | 403 | SQSTHV | 438 | |
| | | VHSNGN | | S | | PPFT | | |
| | | TYLH | | | | | | |
| | Chothia | RSSQSL | 358 | KVSNRF | 403 | SQSTHV | 438 | |
| | | VHSNGN | | S | | PPFT | | |
| | | TYLH | | | | | | |
| | Contact | VHSNGN | 359 | LLIYKVS | 404 | SQSTHV | 439 | |
| | | TYLHWY | | NRF | | PPF | | |
| 38 | IMGT | QDVGIA | 354 | WAS | | QQYSSY | 436 | Elotuzumab |
| | | | | | | PYT | | |
| | Kabat | KASQDV | 355 | WASTRH | 401 | QQYSSY | 436 | |
| | | GIAVA | | T | | PYT | | |
| | AbM | KASQDV | 355 | WASTRH | 401 | QQYSSY | 436 | |
| | | GIAVA | | T | | PYT | | |
| | Chothia | KASQDV | 355 | WASTRH | 401 | QQYSSY | 436 | |
| | | GIAVA | | T | | PYT | | |
| | Contact | GIAVAW | 356 | LLIYWAS | 402 | QQYSSY | 437 | |
| | | Y | | TRH | | PY | | |
| 14 | IMGT | QDISNY | 360 | YTS | | QQGNTL | 440 | AMG 701 |
| | | | | | | PWT | | (BCMA) |
| | Kabat | QASQDI | 361 | YTSRLH | 405 | QQGNTL | 440 | |
| | | SNYLN | | T | | PWT | | |
| | AbM | QASQDI | 361 | YTSRLH | 405 | QQGNTL | 440 | |
| | | SNYLN | | T | | PWT | | |
| | Chothia | QASQDI | 361 | YTSRLH | 405 | QQGNTL | 440 | |
| | | SNYLN | | T | | PWT | | |
| | Contact | SNYLNW | 362 | LLIYYTS | 406 | QQGNTL | 441 | |
| | | Y | | RLH | | PW | | |
| 15 | IMGT | TGAVTS | 363 | GTK | | VLWYSN | 442 | AMG 701 |
| | | GNY | | | | RWV | | (CD3) |
| | Kabat | GSSTGA | 364 | GTKFLA | 407 | VLWYSN | 442 | |
| | | VTSGNY | | P | | RWV | | |
| | | PN | | | | | | |
| | AbM | GSSTGA | 364 | GTKFLA | 407 | VLWYSN | 442 | |
| | | VTSGNY | | P | | RWV | | |
| | | PN | | | | | | |
| | Chothia | GSSTGA | 364 | GTKFLA | 407 | VLWYSN | 442 | |
| | | VTSGNY | | P | | RWV | | |
| | | PN | | | | | | |
| | Contact | VTSGNY | 365 | GLIGGT | 408 | VLWYSN | 443 | |
| | | PNWV | | KFLA | | RW | | |
| 16 | IMGT | QSVSDE | 366 | SAS | | QQYGYP | 444 | Alnuctamab |
| | | Y | | | | PDFT | | (EM 801) |
| | Kabat | RASQSV | 367 | SASTRA | 409 | QQYGYP | 444 | (BCMA) |
| | | SDEYLS | | T | | PDFT | | |
| | AbM | RASQSV | 367 | SASTRA | 409 | QQYGYP | 444 | |
| | | SDEYLS | | T | | PDFT | | |
| | Chothia | RASQSV | 367 | SASTRA | 409 | QQYGYP | 444 | |
| | | SDEYLS | | T | | PDFT | | |
| | Contact | SDEYLS | 368 | LLIHSAS | 410 | QQYGYP | 445 | |
| | | WY | | TRA | | PDF | | |
| 17 | IMGT | TGAVTT | 369 | GTN | | ALWYSN | 446 | Alnuctamab |
| | | SNY | | | | LWV | | (EM 801) |
| | Kabat | GSSTGA | 370 | GTNKRA | 411 | ALWYSN | 446 | (CD3) |
| | | VTTSNY | | P | | LWV | | |
| | | AN | | | | | | |
| | AbM | GSSTGA | 370 | GTNKRA | 411 | ALWYSN | 446 | |
| | | VTTSNY | | P | | LWV | | |
| | | AN | | | | | | |
| | Chothia | GSSTGA | 370 | GTNKRA | 411 | ALWYSN | 446 | |
| | | VTTSNY | | P | | LWV | | |
| | | AN | | | | | | |
| | Contact | VTTSNY | 371 | GLIGGT | 412 | ALWYSN | 447 | |
| | | ANWV | | NKRA | | LW | | |
| 39 | IMGT | QSLFNV | 372 | WAS | | KQSYDL | 448 | Elranatamab |
| | | RSRKNY | | | | FT | | (CD3e) |
| | Kabat | KSSQSL | 373 | WASTRE | 413 | KQSYDL | 448 | |
| | | FNVRSR | | S | | FT | | |
| | | KNYLA | | | | | | |
| | AbM | KSSQSL | 373 | WASTRE | 413 | KQSYDL | 448 | |
| | | FNVRSR | | S | | FT | | |
| | | KNYLA | | | | | | |
| | Chothia | KSSQSL | 373 | WASTRE | 413 | KQSYDL | 448 | |
| | | FNVRSR | | S | | FT | | |
| | | KNYLA | | | | | | |
| | Contact | FNVRSR | 374 | LLISWAS | 414 | KQSYDL | 449 | |
| | | KNYLAW | | TRE | | F | | |
| | | Y | | | | | | |
| 40 | IMGT | QSVSSS | 375 | DAS | | QQYQS | 450 | Elranatamab |
| | | Y | | | | WPLT | | (BCMA) |
| | Kabat | RASQSV | 376 | DASIRAT | 415 | QQYQS | 450 | |
| | | SSSYLA | | | | WPLT | | |
| | AbM | RASQSV | 376 | DASIRAT | 415 | QQYQS | 450 | |
| | | SSSYLA | | | | WPLT | | |
| | Chothia | RASQSV | 376 | DASIRAT | 415 | QQYQS | 450 | |
| | | SSSYLA | | | | WPLT | | |
| | Contact | SSSYLA | 377 | LLMYDA | 416 | QQYQS | 451 | |
| | | WY | | SIRA | | WPL | | |
| 41 | IMGT | QSISSY | 378 | AAS | | QQSYST | 452 | Linvoseltamab |
| | | | | | | PPIT | | (REGN5458) |
| | Kabat | RASQSI | 379 | AASSLQ | 417 | QQSYST | 452 | light chain |
| | | SSYLN | | S | | PPIT | | |
| | AbM | RASQSI | 379 | AASSLQ | 417 | QQSYST | 452 | |
| | | SSYLN | | S | | PPIT | | |
| | Chothia | RASQSI | 379 | AASSLQ | 417 | QQSYST | 452 | |
| | | SSYLN | | S | | PPIT | | |
| | Contact | SSYLNW | 380 | LLIYAAS | 418 | QQSYST | 453 | |
| | | Y | | SLQ | | PPI | | |
| 18 | IMGT | TGAVTT | 369 | GTN | | ALWYSN | 446 | SP34 CD3 |
| | | SNY | | | | LWV | | |
| | Kabat | RSSTGA | 381 | GTNKRA | 411 | ALWYSN | 446 | |
| | | VTTSNY | | P | | LWV | | |
| | | AN | | | | | | |
| | AbM | RSSTGA | 381 | GTNKRA | 411 | ALWYSN | 446 | |
| | | VTTSNY | | P | | LWV | | |
| | | AN | | | | | | |
| | Chothia | RSSTGA | 381 | GTNKRA | 411 | ALWYSN | 446 | |
| | | VTTSNY | | P | | LWV | | |
| | | AN | | | | | | |
| | Contact | VTTSNY | 371 | GLIGGT | 412 | ALWYSN | 447 | |
| | | ANWV | | NKRA | | LW | | |
| 42 | IMGT | NIGSKS | 382 | DDS | | QVWDS | 454 | Teclistamab |
| | | | | | | SSDHVV | | (anti-BCMA |
| | Kabat | GGNNIG | 383 | DDSDRP | 419 | QVWDS | 454 | light chain) |
| | | SKSVH | | S | | SSDHVV | | |
| | AbM | GGNNIG | 383 | DDSDRP | 419 | QVWDS | 454 | |
| | | SKSVH | | S | | SSDHVV | | |
| | Chothia | GGNNIG | 383 | DDSDRP | 419 | QVWDS | 454 | |
| | | SKSVH | | S | | SSDHVV | | |
| | Contact | SKSVHW | 384 | WWVYDD | 420 | QVWDS | 455 | |
| | | Y | | SDRP | | SSDHV | | |
| 43 | IMGT | TGAVTT | 369 | GTN | | ALWYSN | 446 | Teclistamab |
| | | SNY | | | | LWV | | (anti-CD3 |
| | Kabat | RSSTGA | 381 | GTNKRA | 411 | ALWYSN | 446 | light |
| | | VTTSNY | | P | | LWV | | chain) |
| | | AN | | | | | | |
| | AbM | RSSTGA | 381 | GTNKRA | 411 | ALWYSN | 446 | |
| | | VTTSNY | | P | | LWV | | |
| | | AN | | | | | | |
| | Chothia | RSSTGA | 381 | GTNKRA | 411 | ALWYSN | 446 | |
| | | VTTSNY | | P | | LWV | | |
| | | AN | | | | | | |
| | Contact | VTTSNY | 371 | GLIGGT | 412 | ALWYSN | 447 | |
| | | ANWV | | NKRA | | LW | | |
| 44 | IMGT | QSVSSN | 385 | GAS | | QQYNN | 456 | ABBV-383 |
| | | | | | | WPWT | | CD3 light |
| | Kabat | RASQSV | 386 | GASTRA | 421 | QQYNN | 456 | chain |
| | | SSNLA | | T | | WPWT | | |
| | AbM | RASQSV | 386 | GASTRA | 421 | QQYNN | 456 | |
| | | SSNLA | | T | | WPWT | | |
| | Chothia | RASQSV | 386 | GASTRA | 421 | QQYNN | 456 | |
| | | SSNLA | | T | | WPWT | | |
| | Contact | SSNLAW | 387 | LLIYGAS | 422 | QQYNN | 457 | |
| | | Y | | TRA | | WPW | | |
| 48 | IMGT | TGAVTS | 363 | GTKF | 423 | TLWYSN | 458 | HPN217 (CD3) |
| | | GNY | | | | RWV | | |
| | Kabat | ASSTGA | 388 | GTKFLV | 424 | TLWYSN | 458 | |
| | | VTSGNY | | P | | RWV | | |
| | | PN | | | | | | |
| | AbM | ASSTGA | 388 | GTKFLV | 424 | TLWYSN | 458 | |
| | | VTSGNY | | P | | RWV | | |
| | | PN | | | | | | |
| | Chothia | ASSTGA | 388 | GTKFLV | 424 | TLWYSN | 458 | |
| | | VTSGNY | | P | | RWV | | |
| | | PN | | | | | | |
| | Contact | VTSGNY | 365 | GLIGGT | 425 | TLWYSN | 459 | |
| | | PNWV | | KFLV | | RW | | |
| 45 | IMGT | QDVRNL | 389 | SGS | | QQHYSP | 432 | Cevostamab |
| | | | | | | PYT | | (FcRH5) |
| | Kabat | KASQDV | 390 | SGSYRY | 426 | QQHYSP | 432 | |
| | | RNLVV | | S | | PYT | | |
| | AbM | KASQDV | 390 | SGSYRY | 426 | QQHYSP | 432 | |
| | | RNLVV | | S | | PYT | | |
| | Chothia | KASQDV | 390 | SGSYRY | 426 | QQHYSP | 432 | |
| | | RNLVV | | S | | PYT | | |
| | Contact | RNLVVW | 391 | LLIYSGS | 427 | QQHYSP | 433 | |
| | | F | | YRY | | PY | | |
| 46 | IMGT | QSLLNS | 392 | WTS | | KQSFILR | 460 | Cevostamab |
| | | RTRKNY | | | | T | | (CD3) |
| | Kabat | KSSQSL | 393 | WTSTRK | 428 | KQSFILR | 460 | |
| | | LNSRTR | | S | | T | | |
| | | KNYLA | | | | | | |
| | AbM | KSSQSL | 393 | WTSTRK | 428 | KQSFILR | 460 | |
| | | LNSRTR | | S | | T | | |
| | | KNYLA | | | | | | |
| | Chothia | KSSQSL | 393 | WTSTRK | 428 | KQSFILR | 460 | |
| | | LNSRTR | | S | | T | | |
| | | KNYLA | | | | | | |
| | Contact | LNSRTR | 394 | LLIYWTS | 429 | KQSFILR | 461 | |
| | | KNYLAW | | TRK | | | | |
| | | Y | | | | | | |

In some aspects, the multispecific binding molecule comprises a VH domain disclosed herein. In some embodiments, the VH domain comprises a CDR sequence listed in Table 1. In some embodiments, the VH domain comprises a first binding moiety to a first tumor associated antigen. In some embodiments, the VH domain comprises a second binding moiety to a second tumor associated antigen. In some embodiments, the VH domain comprises a third binding moiety to a third tumor associated antigen. In some embodiments, the VH domain comprises an immune cell engager. In some embodiments, the immune cell engager is a T cell binding moiety, an NK cell binding moiety, an NKT cell binding moiety, or a combination thereof. In some embodiments, the first, second, and third binding moieties comprise a GPRC5D binding moiety, a TNFRSF17 (BCMA) binding moiety, and a CD38 binding moiety. In some embodiments, the first, second, and third binding moieties comprise a GPRC5D binding moiety, a TNFRSF17 (BCMA) binding moiety, and a SLAMF7 (CD319) binding moiety. In some embodiments, the first, second, and third binding moieties comprise a GPRC5D binding moiety, a CD38 binding moiety, and a SLAMF7 (CD319) binding moiety. In some embodiments, the multispecific binding molecule comprises an immune cell engager comprising a CD3 binding moiety, and a first, second, and third binding moieties comprising a GPRC5D binding moiety, a TNFRSF17 (BCMA) binding moiety, and a CD38 binding moiety. In some embodiments, the multispecific binding molecule comprises an immune cell engager comprising a CD3 binding moiety, and a first, second, and third binding moieties comprising a GPRC5D binding moiety, a TNFRSF17 (BCMA) binding moiety, and a SLAMF7 (CD319) binding moiety. In some embodiments, the multispecific binding molecule comprises an immune cell engager comprising a CD3 binding moiety, and a first, second, and third binding moieties comprising a GPRC5D binding moiety, a CD38 binding moiety, and a SLAMF7 (CD319) binding moiety. In some embodiments, the multispecific binding molecule comprises an immune cell engager comprising a CD3 binding moiety, and a first, second, and third binding moieties comprising a BCMA binding moiety, a CD38 binding moiety, and a SLAMF7 (CD319) binding moiety.

In some embodiments, the multispecific binding molecule comprises an immune cell engager comprising a CD3 binding moiety, and a first, second, and third binding moieties comprising a GPRC5D binding moiety, a BCMA binding moiety, and an FcRH5 binding moiety. In some embodiments, the multispecific binding molecule comprises an immune cell engager comprising a CD3 binding moiety, and a first, second, and third binding moieties comprising a GPRC5D binding moiety, a CD38 binding moiety, and an FcRH5 binding moiety. In some embodiments, the multispecific binding molecule comprises an immune cell engager comprising a CD3 binding moiety, and a first, second, and third binding moieties comprising a GPRC5D binding moiety, a SLAMF7 binding moiety, and an FcRH5 binding moiety. In some embodiments, the multispecific binding molecule comprises an immune cell engager comprising a CD3 binding moiety, and a first, second, and third binding moieties comprising a CD38 binding moiety, a BCMA binding moiety, and an FcRH5 binding moiety. In some embodiments, the multispecific binding molecule comprises an immune cell engager comprising a CD3 binding moiety, and a first, second, and third binding moieties comprising a SLAMF7 binding moiety, a BCMA binding moiety, and an FcRH5 binding moiety. In some embodiments, the multispecific binding molecule comprises an immune cell engager comprising a CD3 binding moiety, and a first, second, and third binding moieties comprising a CD38 binding moiety, a SLAMF7 binding moiety, and an FcRH5 binding moiety.

In some embodiments, the multispecific binding molecule comprises one or more VH domains listed in Table 3. In some embodiments, the multispecific binding molecule comprises one or more VH domains derived from one or more sequences listed in Table 5. In some embodiments, the multispecific binding molecule comprises one or more VH domains derived from one or more sequences listed in Table 5 or Table 3. Exemplary VH domains for use in the multispecific binding molecule are listed in Table 3. Exemplary heavy chains for use in the multispecific binding molecule are listed in Table 5. In some embodiments, the multispecific binding molecule comprises one or more VH domains derived from one or more sequences listed in Table 7.

In some aspects, the multispecific binding molecule comprises a VL domain disclosed herein. In some embodiments, the VL domain comprises a CDR sequence listed in Table 2. In some embodiments, the VL domain comprises a first binding moiety to a first tumor associated antigen. In some embodiments, the VL domain comprises a second binding moiety to a second tumor associated antigen. In some embodiments, the VL domain comprises a third binding moiety to a third tumor associated antigen. In some embodiments, the VL domain comprises an immune cell engager. In some embodiments, the immune cell engager is a T cell binding moiety, an NK cell binding moiety, an NKT cell binding moiety, or a combination thereof. In some embodiments, the first, second, and third binding moieties comprise a GPRC5D binding moiety, a TNFRSF17 (BCMA) binding moiety, and a CD38 binding moiety. In some embodiments, the first, second, and third binding moieties comprise a GPRC5D binding moiety, a TNFRSF17 (BCMA) binding moiety, and a SLAMF7 (CD319) binding moiety. In some embodiments, the first, second, and third binding moieties comprise a GPRC5D binding moiety, a TNFRSF17 (BCMA) binding moiety, and a TNFRSF17 (BCMA) binding moiety. In some embodiments, the first, second, and third binding moieties comprise a GPRC5D binding moiety, a TNFRSF17 (BCMA) binding moiety, and an FcRH5 binding moiety. In some embodiments, the first, second, and third binding moieties comprise a GPRC5D binding moiety, a CD38 binding moiety, and an FcRH5 binding moiety. In some embodiments, the first, second, and third binding moieties comprise a GPRC5D binding moiety, a SLAMF7 binding moiety, and an FcRH5 binding moiety. In some embodiments, the first, second, and third binding moieties comprise a CD38 binding moiety, a TNFRSF17 (BCMA) binding moiety, and an FcRH5 binding moiety. In some embodiments, the first, second, and third binding moieties comprise a SLAMF7 binding moiety, a TNFRSF17 (BCMA) binding moiety, and an FcRH5 binding moiety. In some embodiments, the first, second, and third binding moieties comprise a CD38 binding moiety, a SLAMF7 binding moiety, and an FcRH5 binding moiety. In some embodiments, the multispecific binding molecule comprises an immune cell engager comprising a CD3 binding moiety, and a first, second, and third binding moieties comprising a GPRC5D binding moiety, a TNFRSF17 (BCMA) binding moiety, and a CD38 binding moiety. In some embodiments, the multispecific binding molecule comprises an immune cell engager comprising a CD3 binding moiety, and a first, second, and third binding moieties comprising a GPRC5D binding moiety, a TNFRSF17 (BCMA) binding moiety, and a SLAMF7 (CD319) binding moiety. In some embodiments, the multispecific binding molecule comprises an immune cell engager comprising a CD3 binding moiety, and a first, second, and third binding moieties comprising a GPRC5D binding moiety, a CD38 binding moiety, and a SLAMF7 (CD319) binding moiety. In some embodiments, the multispecific binding molecule comprises an immune cell engager comprising a CD3 binding moiety, and a first, second, and third binding moieties comprising a BCMA binding moiety, a CD38 binding moiety, and a SLAMF7 (CD319) binding moiety. In some embodiments, the multispecific binding molecule comprises an immune cell engager comprising a CD3 binding moiety, and a first, second, and third binding moieties comprising a GPRC5D binding moiety, a TNFRSF17 (BCMA) binding moiety, and an FcRH5 binding moiety. In some embodiments, the multispecific binding molecule comprises an immune cell engager comprising a CD3 binding moiety, and a first, second, and third binding moieties comprising a GPRC5D binding moiety, a CD38 binding moiety, and an FcRH5 binding moiety. In some embodiments, the multispecific binding molecule comprises an immune cell engager comprising a CD3 binding moiety, and a first, second, and third binding moieties comprising a GPRC5D binding moiety, a SLAMF7, and an FcRH5 binding moiety. In some embodiments, the multispecific binding molecule comprises an immune cell engager comprising a CD3 binding moiety, and a first, second, and third binding moieties comprising a CD38 binding moiety, a TNFRSF17 (BCMA) binding moiety, and an FcRH5 binding moiety. In some embodiments, the multispecific binding molecule comprises an immune cell engager comprising a CD3 binding moiety, and a first, second, and third binding moieties comprising a SLAMF7 binding moiety, a TNFRSF17 (BCMA) binding moiety, and an FcRH5 binding moiety. In some embodiments, the multispecific binding molecule comprises an immune cell engager comprising a CD3 binding moiety, and a first, second, and third binding moieties comprising a CD38 binding moiety, a SLAMF7 binding moiety, and an FcRH5 binding moiety.

In some embodiments, the multispecific binding molecule comprises one or more VL domains listed in Table 4. In some embodiments, the multispecific binding molecule comprises one or more VL domains derived from one or more sequences listed in Table 6. In some embodiments, the multispecific binding molecule comprises one or more VL domains derived from one or more sequences listed in Table 4 or Table 6. In some embodiments, the multispecific binding molecule comprises one or more VL domains derived from one or more sequences listed in Table 7. In some embodiments, a VH domain comprising a GPRC5D binding moiety is paired with a VL domain comprising a GPRC5D binding moiety in the multispecific binding molecule. In some embodiments, a VH domain comprising a BCMA binding moiety is paired with a VL domain comprising a BCMA binding moiety in the multispecific binding molecule. In some embodiments, a VH domain comprising an FcRH5 binding moiety is paired with a VL domain comprising an FcRH5 binding moiety in the multispecific binding molecule. In some embodiments, a VH domain comprising a SLAMF7 binding moiety is paired with a VL domain comprising a SLAMF7 binding moiety in the multispecific binding molecule. In some embodiments, a VH domain comprising a CD38 binding moiety is paired with a VL domain comprising a CD38 binding moiety in the multispecific binding molecule. In some embodiments, a VH domain comprising a CD3 binding moiety is paired with a VL domain comprising a CD3 binding moiety in the multispecific binding molecule. In some embodiments, a VH domain comprising a BCMA binding moiety is paired with a VL domain comprising a CD3 binding moiety in the multispecific binding molecule. In some embodiments, a VH domain comprising an FcRH5 binding moiety is paired with a VL domain comprising a CD3 binding moiety in the multispecific binding molecule. In some embodiments, a VH domain comprising a CD38 binding moiety is paired with a VL domain comprising a CD3 binding moiety in the multispecific binding molecule. In some embodiments, a VH domain comprising a SLAMF7 binding moiety is paired with a VL domain comprising a CD3 binding moiety in the multispecific binding molecule. Exemplary VL domains for use in the multispecific binding molecule are listed in Table 4. Exemplary light chains for use in the multispecific binding molecule are listed in Table 6.

TABLE 3
Heavy chain variable domain (VH) Sequences

| SEQ | | Targeted |
|---|---|---|
| ID NO: | VH Sequence | Therapeutic |
| 1 | QVQLVESGGGLVQPGGSLRLSCAASGFTFSSYYMNWVRQAPGKGLEWVSGISGDPSN | MOR202 |
| | TYYADSVKGRFTISRDNSKNTLYLQMNSLRAEDTAVYYCARDLPLVYTGFAYWGQGTLV | |
| | TVSS | |
| 2 | EVKLLESGGGLVQPGGSLKLSCAASGFDFSRYWMSWVRQAPGKGLEWIGEINPDSSTI | Luc63 |
| | NYTPSLKDKFIISRDNAKNTLYLQMSKVRSEDTALYYCARPDGNYWYFDVWGAGTTVTV | |
| | SS | |
| 3 | QVQLVQSGAEVKKPGASVKVSCKASGYTFTNHIIHWVRQAPGQCLEWMGYINPYPGYH | AMG 701 |
| | AYNEKFQGRATMTSDTSTSTVYMELSSLRSEDTAVYYCARDGYYRDTDVLDYWGQGTL | (BCMA) |
| | VTVSS | |
| 4 | EVQLVESGGGLVQPGGSLKLSCAASGFTFNKYAMNWVRQAPGKGLEWVARIRSKYNN | AMG 701 |
| | YATYYADSVKDRFTISRDDSKNTAYLQMNNLKTEDTAVYYCVRHGNFGNSYISYWAYW | (CD3) |
| | GQGTLVTVSS | |
| 5 | EVQLLESGGGLVQPGGSLRLSCAASGFTFSDNAMGWVRQAPGKGLEWVSAISGPGSS | Alnuctamab |
| | TYYADSVKGRFTISRDNSKNTLYLQMNSLRAEDTAVYYCAKVLGWFDYWGQGTLVTVS | (EM 801) |
| | S | (BCMA) |
| 6 | EVQLLESGGGLVQPGGSLRLSCAASGFTFSTYAMNWVRQAPGKGLEWVSRIRSKYNN | Alnuctamab |
| | YATYYADSVKGRFTISRDDSKNTLYLQMNSLRAEDTAVYYCVRHGNFGNSYVSWFAYW | (EM 801) |
| | GQGTLVTVSS | (CD3) |
| 7 | EVQLVESGGGLVQPGGSLRLSCAASGFTFNTYAMNWVRQAPGKGLEWVARIRSKYNN | SP34 CD3 |
| | YATYYADSVKDRFTISRDDSKNTLYLQMNSLRAEDTAVYYCVRHGNFGNSYVSWFAYW | |
| | GQGTLVTVSS | |
| 8 | EVQLVESGGGLVQPGGSLRLSCAASGFTFSNFWMTWVRQAPGKGLEWVANMNQDGS | REGN5459 |
| | EKYYVDSVKGRFTISRDNAKSSLYLQMNSLRAEDTAVYYCARDREYCISTSCYDDFDYW | (BCMA) |
| | GQGTLVTVSS | |
| 9 | EVQLVESGGGLVQPGRSLRLSCAASGFTFDDYSMHWVRQAPGKGLEWVSGISWNSGS | REGN5459 |
| | IGYADSVKGRFTISRDNAKNSLYLQMNSLRAEDTALYYCAKYGSGYGKFYYYGMDVWG | (CD3) |
| | QGTTVTVSS | |
| 10 | EVQLVESGPGLVKPSETLSLTCTVSGFSLTRFGVHWVRQPPGKGLEWLGVIWRGGSTD | Cevostamab |
| | YNAAFVSRLTISKDNSKNQVSLKLSSVTAADTAVYYCSNHYYGSSDYALDNWGQGTLVT | (FcRH5) |
| | VSS | |
| 11 | EVQLVQSGAEVKKPGASVKVSCKASGFTFTSYYIHWVRQAPGQGLEWIGWIYPENDNT | Cevostamab |
| | KYNEKFKDRVTITADTSTSTAYLELSSLRSEDTAVYYCARDGYSRYYFDYWGQGTLVTV | (CD3E) |
| | SS | |

TABLE 4
Light chain variable domain (VL) Sequences

| SEQ | | Targeted |
|---|---|---|
| ID NO: | VL Sequence | Therapeutic |
| 12 | DIELTQPPSVSVAPGQTARISCSGDNLRHYYVYWYQQKPGQAPVLVIYGDSKRP | MOR202 |
| | SGIPERFSGSNSGNTATLTISGTQAEDEADYYCQTYTGGASLVFGGGTKLTVLG | |
| | Q | |
| 13 | DIVMTQSHKFMSTSVGDRVSITCKASQDVGIAVAWYQQKPGQSPKLLIYWASTR | Luc63 |
| | HTGVPDRFTGSGSGTDFTLTISNVQSEDLADYFCQQYSSYPYTFGGGTKLEIK | |
| 14 | DIQMTQSPSSLSASVGDRVTITCQASQDISNYLNWYQQKPGKAPKLLIYYTSRL | AMG 701 |
| | HTGVPSRFSGSGSGTDFTFTISSLEPEDIATYYCQQGNTLPWTFGCGTKVEIK | (BCMA) |
| 15 | QTVVTQEPSLTVSPGGTVTLTCGSSTGAVTSGNYPNWVQQKPGQAPRGLIGGTK | AMG 701 |
| | FLAPGTPARFSGSLLGGKAALTLSGVQPEDEAEYYCVLWYSNRWVFGGGTKLT | (CD3) |
| | VL | |
| 16 | EIVLTQSPGTLSLSPGERATLSCRASQSVSDEYLSWYQQKPGQAPRLLIHSAS | Alnuctamab |
| | TRATGIPDRFSGSGSGTDFTLAISRLEPEDFAVYYCQQYGYPPDFTFGQGTKV | (EC 801) |
| | EIK | (BCMA) |
| 17 | QAWTQEPSLTVSPGGTVTLTCGSSTGAVTTSNYANWVQEKPGQAFRGLIGGT | Alnuctamab |
| | NKRAPGTPARFSGSLLGGKAALTLSGAQPEDEAEYYCALWYSNLWVFGGGTK | (EC 801) |
| | LTVL | (CD3) |
| 18 | QAVVTQEPSLTVSPGGTVTLTCRSSTGAVTTSNYANWVQQKPDHLFRGLIGGT | SP34 CD3 |
| | NKRAPGTPARFSGSLLGDKAALTISGAQPEDEAEYYCALWYSNLWVFGGGTK | |
| | LTVL | |
| 19 | EVQLVESGPGLVKPSETLSLTCTVSGFSLTRFGVHWVRQPPGKGLEWLGVIWR | Cevostamab |
| | GGSTDYNAAFVSRLTISKDNSKNQVSLKLSSVTAADTAVYYCSNHYYGSSDYA | (FcRH5) |
| | LDNWGQGTLVTVSS | |
| 20 | EVQLVQSGAEVKKPGASVKVSCKASGFTFTSYYIHWVRQAPGQGLEWIGWIYP | Cevostamab |
| | ENDNTKYNEKFKDRVTITADTSTSTAYLELSSLRSEDTAVYYCARDGYSRYYF | (CD3E) |
| | DYWGQGTLVTVSS | |

In some embodiments, an antibody-based multispecific binder molecule described herein comprises a full-length antibody. In other aspects, the antibody-based multispecific binder molecule comprises an antigen-binding fragment thereof. In some embodiments, the antibody-based multispecific binder molecule comprises an intact IgG1. In some embodiments, the antibody-based multispecific binder molecule comprises an intact IgG2. In some embodiments, the antibody-based multispecific binder molecule comprises an intact IgG3. In some embodiments, the antibody-based multispecific binder molecule comprises an intact IgG4. In some embodiments, the antibody-based multispecific binder molecule comprises an intact IgA. In some embodiments, the antibody-based multispecific binder molecule comprises an intact IgM. In some embodiments, the antibody-based multispecific binder molecule comprises an intact IgE. In some embodiments, the antibody-based multispecific binder molecule comprises an intact IgD. In some cases, the antibody-based multispecific binder molecule comprises a humanized antibody or an antigen-binding fragment thereof, a chimeric antibody or an antigen-binding fragment thereof, a monoclonal antibody or an antigen-binding fragment thereof. In some cases, the antibody-based multispecific binder molecule comprises a monovalent Fab′, F(ab)′3 fragments, single-chain variable fragment (scFv), (scFv)2, minibody, nanobody, disulfide stabilized Fv protein (“dsFv”), single-domain antibody (sdAb), Ig NAR, camelid antibody or an antigen-binding fragment thereof, or a chemically-modified derivative thereof. In some embodiments, the antibody-based multispecific binder molecule comprises an arrangement of binding moieties in an IgG-scFv format. In some embodiments, the antibody-based multispecific binder molecule comprises an arrangement of binding moieties in an scFv-Fc-scFv format. In some embodiments, the antibody-based multispecific binder molecule comprises an arrangement of binding moieties in a IgG-dAb format. In some embodiments, the antibody-based multispecific binder molecule comprises an arrangement of binding moieties in a bispecific antibody format. In some embodiments, the antibody-based multispecific binder molecule comprises an arrangement of binding moieties in a bispecific killer engager (BiKE) format. In some embodiments, the antibody-based multispecific binder molecule comprises an arrangement of binding moieties in a trispecific killer engager (TriKE) format. In some embodiments, the antibody-based multispecific binder molecule comprises an arrangement of binding moieties in a tetraspecific killer engager (TetraKE) format.

In some cases, the antibody-based multispecific binder molecule comprises one or more mutations in a framework region, e.g., in the CH1 domain, CH2 domain, CH3 domain, hinge region, or a combination thereof. In some instances, the one or more mutations are to stabilize the antibody-based multispecific binder molecule. In some instances, the one or more mutations are to increase half-life. In some instances, the one or more mutations are to modulate Fc receptor interactions. In some instances, the one or more mutations are to reduce or eliminate Fc effector functions such as FcyR, antibody-dependent cell-mediated cytotoxicity (ADCC), or complement-dependent cytotoxicity (CDC). In additional instances, the one or more mutations are to modulate glycosylation.

The term Fc region is used herein to specify a C-terminal region of an immunoglobulin heavy chain. The Fc region of the antibodies described herein may be a wild-type sequence. In some embodiments, the Fc region is human Fc region. In some embodiments, the Fc region is a mouse Fc region. Although the boundaries of the Fc region of an immunoglobulin heavy chain might vary, the human IgG heavy chain Fc region is typically defined to stretch from an amino acid residue at position Cys226, or from Pro230, to the carboxyl-terminus thereof. The Fc region of an immunoglobulin generally comprises two constant domains, CH2 and CH3. An Fc region can be present in a monomeric form or a dimeric form. The Fc region can bind to various cell receptors, such as Fc receptors, and other immune molecules, including complement proteins. In some embodiments, the one or more mutations are located in the Fc region.

In some embodiments, the human IgG constant region is modified to alter antibody-dependent cellular cytotoxicity (ADCC) and/or complement-dependent cytotoxicity (CDC), e.g., with an amino acid modification described in Natsume et al., 2008 Cancer Res, 68 (10): 3863-72; Idusogie et al., 2001 J Immunol, 166 (4): 2571-5; Moore et al., 2010 mAbs, 2 (2): 181-189; Lazar et al., 2006 PNAS, 103 (11): 4005-4010, Shields et al., 2001 JBC, 276 (9): 6591-6604; Stavenhagen et al., 2007 Cancer Res, 67 (18): 8882-8890; Stavenhagen et al., 2008 Advan. Enzyme Regul., 48:152-164; Alegre et al, 1992 J Immunol, 148:3461-3468; Urban et al., 2021 Front Immunol. November 25; 12:724361; Zhou et al., 2020 MAbs. Jan-Dec; 12 (1): 1814583; Reviewed in Kaneko and Niwa, 2011 Biodrugs, 25 (1): 1-11.

In some embodiments, an antibody-based multispecific binder molecule described herein comprises a full-length antibody, comprising a heavy chain (HC) and a light chain (LC). In some embodiments, the HC comprises about 80%, 85%, 90%, 95%, 96% 97%, 98%, 99%, or 100% sequence identity to a sequence listed in Table 5. In some embodiments, the LC comprises about 80%, 85%, 90%, 95%, 96% 97%, 98%, 99%, or 100% sequence identity to a sequence listed in Table 6. In some cases, the heavy chain (HC) comprises a sequence selected from the sequences listed in Table 5. In some cases, the light chain (LC) comprises a sequence selected from the sequences listed in Table 6.

TABLE 5
Antibody heavy chain sequences

| SEQ ID | | Targeted |
|---|---|---|
| NO: | Heavy Chain sequence | Therapeutic |
| 21 | (X)n = 14-40 | Daratumumab |
| | EVQLLESGGGLVQPGGSLRLSCAVSGFTFNSFAMSWVRQAPGKGLEWVSAI | |
| | SGSGGGTYYADSVKGRFTISRDNSKNTLYLQMNSLRAEDTAVYFCAKDKILW | |
| | FGEPVFDYWGQGTLVTVSSASTKGPSVFPLAPSSKSTSGGTAALGCLVKDYF | |
| | PEPVTVSWNSGALTSGVHTFPAVLQSSGLYSLSSWVTVPSSSLGTQTYICNV | |
| | NHKPSNTKVDKRVEPKSCDKTHTCPPCPAPELLGGPSVFLFPPKPKDTLMIS | |
| | RTPEVTCWVDVSHEDPEVKFNWYVDGVEVHNAKTKPREEQYNSTYRVVSV | |
| | LTVLHQDWLNGKEYKCKVSNKALPAPIEKTISKAKGQPREPQVYTLPPSREE | |
| | MTKNQVSLTCLVKGFYPSDIAVEWESNGQPENNYKTTPPVLDSDGSFFLYSK | |
| | LTVDKSRWQQGNVFSCSVMHEALHNHYTQKSLSLSPGK | |
| 22 | (X)_{n = 14-40} | Isatuximab |
| | QVQLVQSGAEVAKPGTSVKLSCKASGYTFTDYWMQWVKQRPGQGLEWIGTI | |
| | YPGDGDTGYAQKFQGKATLTADKSSKTVYMHLSSLASEDSAVYYCARGDYY | |
| | GSNSLDYWGQGTSVTVSSASTKGPSVFPLAPSSKSTSGGTAALGCLVKDYF | |
| | PEPVTVSWNSGALTSGVHTFPAVLQSSGLYSLSSWVTVPSSSLGTQTYICNV | |
| | NHKPSNTKVDKKVEPKSCDKTHTCPPCPAPELLGGPSVFLFPPKPKDTLMISR | |
| | TPEVTCVWVDVSHEDPEVKFNWYVDGVEVHNAKTKPREEQYNSTYRVVSVL | |
| | TVLHQDWLNGKEYKCKVSNKALPAPIEKTISKAKGQPREPQVYTLPPSRDELT | |
| | KNQVSLTCLVKGFYPSDIAVEWESNGQPENNYKTTPPVLDSDGSFFLYSKLT | |
| | VDKSRWQQGNVFSCSVMHEALHNHYTQKSLSLSPGK | |
| 23 | (X)_{n = 14-40} | ABBV-838 |
| | EVQLVESGGGLVQPGGSLRLSCAASGFTFSDYYMAWVRQAPGKGLEWVASI | |
| | NYDGSSTYYVDSVKGRFTISRDNAKNSLYLQMNSLRAEDTAVYYCARDRGYY | |
| | FDYWGQGTTVTVSSASTKGPSVFPLAPSSKSTSGGTAALGCLVKDYFPEPVT | |
| | VSWNSGALTSGVHTFPAVLQSSGLYSLSSWVTVPSSSLGTQTYICNVNHKPS | |
| | NTKVDKKVEPKSCDKTHTCPPCPAPELLGGPSVFLFPPKPKDTLMISRTPEVT | |
| | CWWVDVSHEDPEVKFNWYVDGVEVNAKTKPREEQYNSTYRVVSVLTVLHQD | |
| | WLNGKEYKCKVSNKALPAPIEKTISKAKGQPREPQVYTLPPSREEMTKNQVS | |
| | LTCLVKGFYPSDIAVEWESNGQPENNYKTTPPVLDSDGSFFLYSKLTVDKSR | |
| | WQQGNVFSCSVMHEALHNHYTQKSLSLSPGK | |
| 24 | (X)_{n = 14-40} | Elotuzumab |
| | EVQLVESGGGLVQPGGSLRLSCAASGFDFSRYWMSWVRQAPGKGLEWIGEI | |
| | NPDSSTINYAPSLKDKFIISRDNAKNSLYLQMNSLRAEDTAVYYCARPDGNYW | |
| | YFDVWGQGTLVTVSSASTKGPSVFPLAPSSKSTSGGTAALGCLVKDYFPEPV | |
| | TVSWNSGALTSGVHTFPAVLQSSGLYSLSSVVTVPSSSLGTQTYICNVNHKP | |
| | SNTKVDKKVEPKSCDKTHTCPPCPAPELLGGPSVFLFPPKPKDTLMISRTPEV | |
| | TCWWVDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDELTKNQ | |
| | VSLTCLVKGFYPSDIAVEWESNGQPENNYKTTPPVLDSDGSFFLYSKLTVDKS | |
| | RWQQGNVFSCSVMHEALHNHYTQKSLSLSPGK | |
| 25 | (X)_{n = 14-40} | Elranatamab |
| | EVQLVESGGGLVQPGGSLRLSCAASGFTFSDYYMTWVRQAPGKGLEWVAFI | (CD3e) |
| | RNRARGYTSDHNPSVKGRFTISRDNAKNSLYLQMNSLRAEDTAVYYCARDR | |
| | PSYYVLDYWGQGTTVTVSSASTKGPSVFPLAPCSRSTSESTAALGCLVKDYF | |
| | PEPVTVSWNSGALTSGVHTFPAVLQSSGLYSLSSVVTVPSSNFGTQTYTCNV | |
| | DHKPSNTKVDKTVERKCRVRCPRCPAPPVAGPSVFLFPPKPKDTLMISRTPE | |
| | VTCWWVAVSHEDPEVQFNWYVDGVEVHNAKTKPREEQFNSTFRVVSVLTWV | |
| | HQDWLNGKEYKCKVSNKGLPSSIEKTISKTKGQPREPQVYTLPPSREEMTKN | |
| | QVSLTCLVKGFYPSDIAVEWESNGQPENNYKTTPPMLDSDGSFFLYSRLTVD | |
| | KSRWQQGNVFSCSVMHEALHNHYTQKSLSLSPGK | |
| 26 | (X)_{n = 14-40} | Elranatamab |
| | EVQLLESGGGLVQPGGSLRLSCAASGFTFSSYPMSWVRQAPGKGLEWVSAI | (BCMA) |
| | GGSGGSLPYADIVKGRFTISRDNSKNTLYLQMNSLRAEDTAVYYCARYWPMD | |
| | IWGQGTLVTVSSASTKGPSVFPLAPCSRSTSESTAALGCLVKDYFPEPVTVS | |
| | WNSGALTSGVHTFPAVLQSSGLYSLSSVVTVPSSNFGTQTYTCNVDHKPSNT | |
| | KVDKTVERKCEVECPECPAPPVAGPSVFLFPPKPKDTLMISRTPEVTCVVVAV | |
| | SHEDPEVQFNWYVDGVEVHNAKTKPREEQFNSTFRVVSVLTVVHQDWLNG | |
| | KEYKCKVSNKGLPSSIEKTISKTKGQPREPQVYTLPPSREEMTKNQVSLTCEV | |
| | KGFYPSDIAVEWESNGQPENNYKTTPPMLDSDGSFFLYSKLTVDKSRWQQG | |
| | NVFSCSVMHEALHNHYTQKSLSLSPGK | |
| 27 | (X)_{n = 14-40} | Linvoseltamab |
| | EVQLVESGGGLVQPGGSLRLSCAASGFTFSNFWMTWVRQAPGKGLEWVAN | (REGN5458) |
| | MNQDGSEKYYVDSVKGRFTISRDNAKSSLYLQMNSLRAEDTAVYYCARDRE | Heavy chain 1 |
| | YCISTSCYDDFDYWGQGTLTVSSASTKGPSVFPLAPCSRSTSESTAALGCLV | (CD3 or BCMA) |
| | KDYFPEPVTVSWNSGALTSGVHTFPALQSSGLYSLSSVVTVPSSSLGTKTYT | |
| | CNVDHKPSNTKVDKRVESKYGPPCPPCPAPPVAGPSVFLFPPKPKDTLMISR | |
| | TPEVTCVVVDVSQEDPEVQFNWYVDGVEVHNAKTKPREEQFNSTYRVVSVL | |
| | TVLHQDWLNGKEYKCKVSNKGLPSSIEKTISKAKGQPREPQVYTLPPSQEEM | |
| | TKNQVSLTCLVKGFYPSDIAVEWESNGQPENNYKTTPPVLDSDGSFFLYSRL | |
| | TVDKSRWQEGNVFSCSVMHEALHNHYTQKSLSLSLGK | |
| 28 | (X)_{n = 14-40} | Linvoseltamab |
| | EVQLVESGGGLVQPGRSLRLSCAASGFTFDDYSMHWVRQAPGKGLEWVSG | (REGN5458) |
| | ISWNSGSKGYADSVKGRFTISRDNAKNSLYLQMNSLRAEDTALYYCAKYGSG | Heavy chain 2 |
| | YGKFYHYGLDVWGQGTTVTVSSASTKGPSVFPLAPCSRSTSESTAALGCLVK | (CD3 or BCMA) |
| | DYFPEPVTVSWNSGALTSGVHTFPAVLQSSGLYSLSSWTVPSSSLGTKTYT | |
| | CNVDHKPSNTKVDKRVESKYGPPCPPCPAPPVAGPSVFLFPPKPKDTLMISR | |
| | TPEVTCVVVDVSQEDPEVQFNWYVDGVEVHNAKTKPREEQFNSTYRVVSVL | |
| | TVLHQDWLNGKEYKCKVSNKGLPSSIEKTISKAKGQPREPQVYTLPPSQEEM | |
| | TKNQVSLTCLVKGFYPSDIAVEWESNGQPENNYKTTPPVLDSDGSFFLYSRL | |
| | TVDKSRWQEGNVFSCSVMHEALHNRFTQKSLSLSPGK | |
| 29 | (X)_{n = 14-40} | Teclistamab |
| | QLQLQESGPGLVKPSETLSLTCTVSGGSISSGSYFWGWIRQPPGKGLEWIGS | (anti-BCMA |
| | IYYSGITYYNPSLKSRVTISVDTSKNQFSLKLSSVTAADTAVYYCARHDGAVAG | heavy chain) |
| | LFDYWGQGTLVTVSSASTKGPSVFPLAPCSRSTSESTAALGCLVKDYFPEPV | |
| | TVSWNSGALTSGVHTFPAVLQSSGLYSLSSVVTVPSSSLGTKTYTCNVDHKP | |
| | SNTKVDKRVESKYGPPCPPCPAPEAAGGPSVFLFPPKPKDTLMISRTPEVTC | |
| | WWVDVSQEDPEVQFNWYVDGVEVHNAKTKPREEQFNSTYRVVSVLTVLHQD | |
| | WLNGKEYKCKVSNKGLPSSIEKTISKAKGQPREPQVYTLPPSQEEMTKNQVS | |
| | LTCLVKGFYPSDIAVEWESNGQPENNYKTTPPVLDSDGSFFLYSRLTVDKSR | |
| | WQEGNVFSCSVMHEALHNHYTQKSLSLSLGK | |
| 30 | (X)_{n = 14-40} | Teclistamab |
| | EVQLVESGGGLVQPGGSLRLSCAASGFTFNTYAMNWVRQAPGKGLEWVARI | (anti-CD3 heavy |
| | RSKYNNYATYYAASVKGRFTISRDDSKNSLYLQMNSLKTEDTAVYYCARHGN | chain) |
| | FGNSYVSWFAYWGQGTLVTVSSASTKGPSVFPLAPCSRSTSESTAALGCLV | |
| | KDYFPEPVTVSWNSGALTSGVHTFPAVLQSSGLYSLSSWTVPSSSLGTKTY | |
| | TCNVDHKPSNTKVDKRVESKYGPPCPPCPAPEAAGGPSVFLFPPKPKDTLMI | |
| | SRTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPSQE | |
| | EMTKNQVSLTCLVKGFYPSDIAVEWESNGQPENNYKTTPPVLDSDGSFLLYS | |
| | KLTVDKSRWQEGNVFSCSVMHEALHNHYTQKSLSLSLGK | |
| 31 | (X)_{n = 14-40} | ABBV-383 |
| | EVQLLESGGGLVQPGGSLRLSCAASGFTVSSYGMSWVRQAPGKGPEWVSG | BCMA heavy |
| | IRGSDGSTYYADSVKGRFTISRDNSKNTLYLQMNSLRAEDTAVYYCAKQGEN | chain |
| | DGPFDHRGQGTLVTVSSGGGGSEVQLLESGGGLVQPGGSLRLSCAASGFTV | |
| | SSYGMSWVRQAPGKGPEWVSGIRGSDGSTYYADSVKGRFTISRDNSKNTLY | |
| | LQMNSLRAEDTAVYYCAKQGENDGPFDHRGQGTLVTVSSESKYGPPCPPCP | |
| | APEAAGGPSVFLFPPKPKDTLMISRTPEVTCVVVDVSQEDPEVQFNWYVDGV | |
| | EVHNAKTKPREEQFNSTYRVVSVLTVLHQDWLNGKEYKCKVSNKGLPSSIEK | |
| | TISKAKGQPREPQVYTLPPSQEEMTKNQVSLSCAVKGFYPSDIAVEWESNGQ | |
| | PENNYKTTPPVLDSDGSFFLVSRLTVDKSRWQEGNVFSCSVMHEALHNHYT | |
| | QKSLSLSLGK | |
| 32 | (X)_{n = 14-40} | ABBV-383 CD3 |
| | EVQLVESGGGLVQPGRSLRLSCAASGFTFDDYAMHWVRQAPGKGLEWVSG | heavy chain |
| | ISWNSGSIGYADSVKGRFTISRDNAKNSLYLQMNSLRAEDTALYYCAKDSRG | |
| | YGDYRLGGAYWGQGTLVTVSSASTKGPSVFPLAPCSRSTSESTAALGCLVK | |
| | DYFPEPVTVSWNSGALTSGVHTFPAVLQSSGLYSLSSVVTVPSSSLGTKTYT | |
| | CNVDHKPSNTKVDKRVESKYGPPCPPCPAPEAAGGPSVFLFPPKPKDTLMIS | |
| | RTPEVTCVVVDVSQEDPEVQFNWYVDGVEVHNAKTKPREEQFNSTYRVVSV | |
| | LTVLHQDWLNGKEYKCKVSNKGLPSSIEKTISKAKGQPREPQVYTLPPSQEE | |
| | MTKNQVSLWCLVKGFYPSDIAVEWESNGQPENNYKTTPPVLDSDGSFFLYS | |
| | RLTVDKSRWQEGNVFSCSVMHEALHNHYTQKSLSLSLGK | |
| 33 | (X)_{n = 14-40} | Cevostamab- |
| | EVQLVESGPGLVKPSETLSLTCTVSGFSLTRFGVHWVRQPPGKGLEWLGVI | Chain A |
| | WRGGSTDYNAAFVSRLTISKDNSKNQVSLKLSSVTAADTAVYYCSNHYYGSS | (FcRH5) heavy |
| | DYALDNWGQGTLVTVSSASTKGPSVFPLAPSSKSTSGGTAALGCLVKDYFPE | chain |
| | PVTVSWNSGALTSGVHTFPAVLQSSGLYSLSSVVTVPSSSLGTQTYICNVNH | |
| | KPSNTKVDKKVEPKSCDKTHTCPPCPAPELLGGPSVFLFPPKPKDTLMISRTP | |
| | EVTCWWVDVSHEDPEVKFNWYVDGVEVHNAKTKPREEQYGSTYRVVSVLTV | |
| | LHQDWLNGKEYKCKVSNKALPAPIEKTISKAKGQPREPQVYTLPPSREEMTK | |
| | NQVSLWCLVKGFYPSDIAVEWESNGQPENNYKTTPPVLDSDGSFFLYSKLTV | |
| | DKSRWQQGNVFSCSVMHEALHNHYTQKSLSLSPGK | |
| 34 | (X)_{n = 14-40} | Cevostamab- |
| | EVQLVQSGAEVKKPGASVKVSCKASGFTFTSYYIHWVRQAPGQGLEWIGWIY | Chain B (CD3E) |
| | PENDNTKYNEKFKDRVTITADTSTSTAYLELSSLRSEDTAVYYCARDGYSRYY | heavy chain |
| | FDYWGQGTLVTVSSASTKGPSVFPLAPSSKSTSGGTAALGCLVKDYFPEPVT | |
| | VSWNSGALTSGVHTFPAVLQSSGLYSLSSVTVPSSSLGTQTYICNVNHKPS | |
| | NTKVDKKVEPKSCDKTHTCPPCPAPELLGGPSVFLFPPKPKDTLMISRTPEVT | |
| | CWWVDVSHEDPEVKFNWYVDGVEVHNAKTKPREEQYGSTYRVVSVLTVLHQ | |
| | DWLNGKEYKCKVSNKALPAPIEKTISKAKGQPREPQVYTLPPSREEMTKNQV | |
| | SLSCAVKGFYPSDIAVEWESNGQPENNYKTTPPVLDSDGSFFLVSKLTVDKS | |
| | RWQQGNVFSCSVMHEALHNHYTQKSLSLSPGK | |

(X)_{n = 14-40} is a leader sequence

TABLE 6
Antibody light chain sequences

| SEQ ID | | Targeted |
|---|---|---|
| NO: | Light Chain sequence | Therapeutic |
| 35 | (X)_{n = 14-40} | Daratumumab |
| | EIVLTQSPATLSLSPGERATLSCRASQSVSSYLAWYQQKPGQAPRLLIYDASN | |
| | RATGIPARFSGSGSGTDFTLTISSLEPEDFAVYYCQQRSNWPPTFGQGTKVEI | |
| | KRTVAAPSVFIFPPSDEQLKSGTASVVCLLNNFYPREAKVQWKVDNALQSGN | |
| | SQESVTEQDSKDSTYSLSSTLTLSKADYEKHKVYACEVTHQGLSSPVTKSFN | |
| | RGEC | |
| 36 | (X)_{n = 14-40} | Isatuximab |
| | DIVMTQSHLSMSTSLGDPVSITCKASQDVSTWAWYQQKPGQSPRRLIYSAS | |
| | YRYIGVPDRFTGSGAGTDFTFTISSVQAEDLAVYYCQQHYSPPYTFGGGTKL | |
| | EIKRTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTKSF | |
| | NRGEC | |
| 37 | (X)_{n = 14-40} | ABBV-838 |
| | DWMTQTPLSLSVTPGQPASISCRSSQSLVHSNGNTYLHWYLQKPGQSPQLL | |
| | IYKVSNRFSGVPDRFSGSGSGTDFTLKISRVEAEDVGVYFCSQSTHVPPFTFG | |
| | GGTKVEIKRTVAAPSVFIFPPSDEQLKSGTASVVCLLNNFYPREAKVQWKVDN | |
| | ALQSGNSQESVTEQDSKDSTYSLSSTLTLSKADYEKHKVYACEVTHQGLSSP | |
| | VTKSFNRGEC | |
| 38 | (X)_{n = 14-40} | Elotuzumab |
| | DIQMTQSPSSLSASVGDRVTITCKASQDVGIAVAWYQQKPGKVPKLLIYWAST | |
| | RHTGVPDRFSGSGSGTDFTLTISSLQPEDVATYYCQQYSSYPYTFGQGTKVE | |
| | IKRTVAAPSVFIFPPSDEQLKSGTASVVCLLNNFYPREAKVQWKVDNALQSGN | |
| | SQESVTEQDSKDSTYSLSSTLTLSKADYEKHKVYACEVTHQGLSSPVTKSFN | |
| | RGEC | |
| 39 | (X)_{n = 14-40} | Elranatamab |
| | DIVMTQSPDSLAVSLGERATINCKSSQSLFNVRSRKNYLAWYQQKPGQPPKL | light chain 1 |
| | LISWASTRESGVPDRFSGSGSGTDFTLTISSLQAEDVAVYYCKQSYDLFTFGS | |
| | GTKLEIKRTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQGLSSPV | |
| | TKSFNRGEC | |
| 40 | (X)_{n = 14-40} | Elranatamab |
| | EIVLTQSPGTLSLSPGERATLSCRASQSVSSSYLAWYQQKPGQAPRLLMYDA | light chain 2 |
| | SIRATGIPDRFSGSGSGTDFTLTISRLEPEDFAVYYCQQYQSWPLTFGQGTKV | |
| | EIKRTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTKSF | |
| | NRGEC | |
| 41 | (X)_{n = 14-40} | Linvoseltamab |
| | DIQMTQSPSSLSASVGDRVTITCRASQSISSYLNWYQQKPGKAPKLLIYAASS | (REGN5458) |
| | LQSGVPSRFSGSGSGTDFTLTISSLQPEDFATYYCQQSYSTPPITFGQGTRLE | (CD3 or BCMA) |
| | IKRTVAAPSVFIFPPSDEQLKSGTASWVCLLNNFYPREAKVQWKVDNALQSGN | |
| | SQESVTEQDSKDSTYSLSSTLTLSKADYEKHKVYACEVTHQGLSSPVTKSFN | |
| | RGEC | |
| 42 | (X)_{n = 14-40} | Teclistamab |
| | SYVLTQPPSVSVAPGQTARITCGGNNIGSKSVHWYQQPPGQAPWWWVYDDS | (anti-BCMA light |
| | DRPSGIPERFSGSNSGNTATLTISRVEAGDEAVYYCQVWDSSSDHVVFGGG | chain) |
| | TKLTVLGQPKAAPSVTLFPPSSEELQANKATLVCLISDFYPGAVTVAWKGDSS | |
| | PVKAGVETTTPSKQSNNKYAASSYLSLTPEQWKSHRSYSCQVTHEGSTVEK | |
| | TVAPTECS | |
| 43 | (X)_{n = 14-40} | Teclistamab |
| | QTVVTQEPSLTVSPGGTVTLTCRSSTGAVTTSNYANWVQQKPGQAPRGLIG | (anti-CD3 light |
| | GTNKRAPGTPARFSGSLLGGKAALTLSGVQPEDEAEYYCALWYSNLWVFGG | chain) |
| | GTKLTVLGQPKAAPSVTLFPPSSEELQANKATLVCLISDFYPGAVTVAWKADS | |
| | SPVKAGVETTTPSKQSNNKYAASSYLSLTPEQWKSHRSYSCQVTHEGSTVE | |
| | KTVAPTECS | |
| 44 | (X)_{n = 14-40} | ABBV-383 CD3 |
| | EIVMTQSPATLSVSPGERATLSCRASQSVSSNLAWYQQKPGQAPRLLIYGAS | light chain |
| | TRATGIPARFSGSGSGTEFTLTISSLQSEDFAVYYCQQYNNWPWTFGQGTKV | |
| | EIKRTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTKSF | |
| | NRGEC | |
| 45 | (X)_{n = 14-40} | Cevostamab- |
| | DIQMTQSPSSLSASVGDRVTITCKASQDVRNLVVWFQQKPGKAPKLLIYSGS | Chain C |
| | YRYSGVPSRFSGSGSGTDFTLTISSLQPEDFATYYCQQHYSPPYTFGQGTKV | (FcRH5) light |
| | EIKRTVAAPSVFIFPPSDEQLKSGTASWVCLLNNFYPREAKVQWKVDNALQSG | chain |
| | NSQESVTEQDSKDSTYSLSSTLTLSKADYEKHKVYACEVTHQGLSSPVTKSF | |
| | NRGEC | |
| 46 | (X)_{n = 14-40} | Cevostamab- |
| | DIVMTQSPDSLAVSLGERATINCKSSQSLLNSRTRKNYLAWYQQKPGQSPKL | Chain D (CD3E) |
| | LIYWTSTRKSGVPDRFSGSGSGTDFTLTISSLQAEDVAVYYCKQSFILRTFGQ | light chain |
| | GTKVEIKRTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQGLSSPV | |
| | TKSFNRGEC | |

(X)_{n = 14-40} is a leader sequence

TABLE 7
Sequences of antibody-based targeted therapeutics in non-
conventional antibody formats

| SEQ ID | | Targeted |
|---|---|---|
| NO: | Amino Acid Sequence | Therapeutic |
| 47 | QVQLVQSGAEVKKPGASVKVSCKASGYTFTNHIIHWVRQAPGQCLEWMGYI | AMG 701 |
| | NPYPGYHAYNEKFQGRATMTSDTSTSTVYMELSSLRSEDTAVYYCARDGYY | (BITE) |
| | RDTDVLDYWGQGTLVTVSSGGGGSGGGGSGGGGSDIQMTQSPSSLSASVG | |
| | DRVTITCQASQDISNYLNWYQQKPGKAPKLLIYYTSRLHTGVPSRFSGSGSGT | |
| | DFTFTISSLEPEDIATYYCQQGNTLPWTFGCGTKVEIKSGGGGSEVQLVESG | |
| | GGLVQPGGSLKLSCAASGFTFNKYAMNWVRQAPGKGLEWVARIRSKYNNYA | |
| | TYYADSVKDRFTISRDDSKNTAYLQMNNLKTEDTAVYYCVRHGNFGNSYISY | |
| | WAYWGQGTLVTVSSGGGGSGGGGSGGGGSQTVVTQEPSLTVSPGGTVTL | |
| | TCGSSTGAVTSGNYPNWVQQKPGQAPRGLIGGTKFLAPGTPARFSGSLLGG | |
| | KAALTLSGVQPEDEAEYYCVLWYSNRWVFGGGTKLTVLGGGGDKTHTCPPC | |
| | PAPELLGGPSVFLFPPKPKDTLMISRTPEVTCVVVDVSHEDPEVKFNWYVDG | |
| | VEVHNAKTKPCEEQYGSTYRCVSVLTVLHQDWLNGKEYKCKVSNKALPAPIE | |
| | KTISKAKGQPREPQVYTLPPSREEMTKNQVSLTCLVKGFYPSDIAVEWESNG | |
| | QPENNYKTTPPVLDSDGSFFLYSKLTVDKSRWQQGNVFSCSVMHEALHNHY | |
| | TQKSLSLSPGKGGGGSGGGGSGGGGSGGGGSGGGGSGGGGSDKTHTCP | |
| | PCPAPELLGGPSVFLFPPKPKDTLMISRTPEVTCVVVDVSHEDPEVKFNWYV | |
| | DGVEVHNAKTKPCEEQYGSTYRCVSVLTVLHQDWLNGKEYKCKVSNKALPA | |
| | PIEKTISKAKGQPREPQVYTLPPSREEMTKNQVSLTCLVKGFYPSDIAVEWES | |
| | NGQPENNYKTTPPVLDSDGSFFLYSKLTVDKSRWQQGNVFSCSVMHEALHN | |
| | HYTQKSLSLSPGK | |
| 48 | EVQLVESGGGLVQPGRSLTLSCAASTNIFSISPYGWYRQAPGKQRELVAAIH | HPN217 |
| | GTSTLYADSVKGRFTISRDNAKNSIYLQMNSLRPEDTALYYCNKVPWGDYHP | (TriTAC) |
| | GNVYWGQGTQVTVSSGGGGSGGGSEVQLVESGGGLVQPGNSLRLSCAAS | |
| | GFTFSKFGMSWVRQAPGKGLEWVSSISGSGRDTLYADSVKGRFTISRDNAK | |
| | TTLYLQMNSLRPEDTAVYYCTIGGSLSVSSQGTLVTVSSGGGGSGGGSEVQL | |
| | VESGGGLVQPGGSLKLSCAASGFTFNKYAINWVRQAPGKGLEWVARIRSKY | |
| | NNYATYYADQVKDRFTISRDDSKNTAYLQMNNLKTEDTAVYYCVRHANFGNS | |
| | YISYWAYWGQGTLVTVSSGGGGSGGGGSGGGGSQTVVTQEPSLTVSPGGT | |
| | VTLTCASSTGAVTSGNYPNWVQQKPGQAPRGLIGGTKFLVPGTPARFSGSLL | |
| | GGKAALTLSGVQPEDEAEYYCTLWYSNRWVFGGGTKLTVLHHHHHH | |

In some embodiments, the antibody-based multispecific binding molecules have a leader sequence near an amino-terminal region of each polypeptide. In some embodiments, the leader sequence is N-terminal to an antibody heavy chain sequence or modified heavy chain sequence. In some embodiments, the leader sequence is N-terminal to an antibody heavy chain sequence provided in Table 5. In some embodiments, the leader sequence is N-terminal to an antibody light chain sequence or modified light chain sequence. In some embodiments, the leader sequence is N-terminal to an antibody light chain sequence provided in Table 6. In some embodiments, (X)ₙ₌₁₄₋₄₀ is used to represent amino acids of a leader sequence at the amino-terminal end of an antibody heavy chain sequence. In some embodiments, (X)ₙ₌₁₄₋₄₀ is used to represent amino acids of a leader sequence at the amino-terminal end of an antibody light chain sequence. In some embodiments, the leader sequence is a length of between 14-40 amino acids. In some embodiments, the leader sequence is a length of between 15-35 amino acids. In some embodiments, the leader sequence of an antibody heavy chain and an antibody light chain paired to form multispecific binding molecule are not identical to each other. In some embodiments, the leader sequence comprises an amino acid sequence listed in Table 20.

In some embodiments, the antibody-based multispecific binder molecule comprises a full-length antibody. In other aspects, the antibody-based multispecific binder molecule comprises an antigen-binding fragment thereof. In some cases, the antibody-based multispecific binder molecule comprises a humanized antibody or an antigen-binding fragment thereof, a chimeric antibody or an antigen-binding fragment thereof, a monoclonal antibody or an antigen-binding fragment thereof. In some cases, the antibody-based multispecific binder molecule comprises a monovalent Fab′, F(ab)′3 fragments, single-chain variable fragment (scFv), (scFv)2, minibody, nanobody, disulfide stabilized Fv protein (“dsFv”), single-domain antibody (sdAb), Ig NAR, camelid antibody or an antigen-binding fragment thereof, or a chemically-modified derivative thereof.

In some embodiments, the multispecific binder molecule comprises one or more immunoglobulin chain constant regions. In some embodiments, the one or more immunoglobulin chain constant regions are derived from IgG1, IgG2, and/or IgG4 antibodies. In some embodiments, the one or more immunoglobulin chain constant regions are derived from human IgG1, IgG2, and/or IgG4 antibodies. In some embodiments, the one or more immunoglobulin chain constant regions is a CH1, CH2, or CH3 domain. In some embodiments, the one or more immunoglobulin chain constant regions is a CL domain. In some embodiments, the one or more immunoglobulin heavy chain constant regions in the multispecific binding molecule is derived from one or more sequences listed in Table 8. In some embodiments, the one or more immunoglobulin heavy chain constant regions in the multispecific binding molecule is selected from one or more sequences listed in Table 8. In some embodiments, the one or more immunoglobulin light chain constant regions in the multispecific binding molecule is derived from a sequence listed in Table 9. In some embodiments, the one or more immunoglobulin light chain constant regions in the multispecific binding molecule is selected from one or more sequences listed in Table 9. In some embodiments, a CH1 domain listed in a portion of sequence in Table 8 is positioned C-terminal to a GPRC5D binding moiety in the multispecific binder molecule. In some embodiments, a CH1 domain listed in a portion of sequence in Table 8 is positioned C-terminal to a BCMA binding moiety in the multispecific binder molecule. In some embodiments, a CH1 domain listed in a portion of sequence in Table 8 is positioned C-terminal to an FcRH5 binding moiety in the multispecific binder molecule. In some embodiments, a CH1 domain listed in a portion of sequence in Table 8 is positioned C-terminal to a CD38 binding moiety in the multispecific binder molecule. In some embodiments, a CH1 domain listed in a portion of sequence in Table 8 is positioned C-terminal to a SLAMF7 binding moiety in the multispecific binder molecule. In some embodiments, a CH1 domain listed in a portion of sequence in Table 8 is positioned C-terminal to a CD3 binding moiety in the multispecific binder molecule. In some embodiments, a CH2 and CH3 domain listed in a portion of sequence in Table 8 is positioned C-terminal to a GPRC5D binding moiety in the multispecific binder molecule. In some embodiments, a CH2 and CH3 domain listed in a portion of sequence in Table 8 is positioned C-terminal to a BCMA binding moiety in the multispecific binder molecule. In some embodiments, a CH2 and CH3 domain listed in a portion of sequence in Table 8 is positioned C-terminal to an FcRH5 binding moiety in the multispecific binder molecule. In some embodiments, a CH2 and CH3 domain listed in a portion of sequence in Table 8 is positioned C-terminal to a CD38 binding moiety in the multispecific binder molecule. In some embodiments, a CH2 and CH3 domain listed in a portion of sequence in Table 8 is positioned C-terminal to a SLAMF7 binding moiety in the multispecific binder molecule. In some embodiments, a CH2 and CH3 domain listed in a portion of sequence in Table 8 is positioned C-terminal to a CD3 binding moiety in the multispecific binder molecule. In some embodiments, a CH2 and CH3 domain listed in a portion of sequence in Table 8 is positioned C-terminal to a CH1 domain. In some embodiments, the multispecific binder molecule comprises a sequence at least 80%, 85%, 90%, 92%, 94%, 95%, 96%, 97%, 98%, or 99% identical to a sequence listed in Table 8. In some embodiments, the multispecific binder molecule comprises a sequence selected from a sequence listed in Table 8. In some embodiments, the multispecific binder molecule comprises a sequence at least 80%, 85%, 90%, 92%, 94%, 95%, 96%, 97%, 98%, or 99% identical to a sequence listed in Table 9. In some embodiments, the multispecific binder molecule comprises a sequence selected from a sequence listed in Table 9. In some embodiments, the multispecific binder molecule comprises two modified heavy chains with different sequences which are paired together to form a multispecific binding format. In some embodiments, the multispecific binder molecule comprises two modified heavy chains comprising KiH knob substitutions and KiH hole substitutions respectively which aid in heterodimeric pairing of the two distinct modified heavy chains. In some embodiments, the multispecific binder molecule comprises four light chains. In some embodiments, the multispecific binder molecule comprises three light chains each comprising a CD3 binding moiety and a fourth light chain comprising a GPRC5D binding moiety. In some embodiments, the multispecific binder molecule comprises three light chains each comprising a CD3 binding moiety and a fourth light chain comprising an FcRH5 binding moiety. In some embodiments, the multispecific binder molecule comprises two modified heavy chains and four light chains. In some embodiments, the multispecific binder molecule is produced in single cells. In some embodiments, the multispecific binder molecule is assembled in vitro.

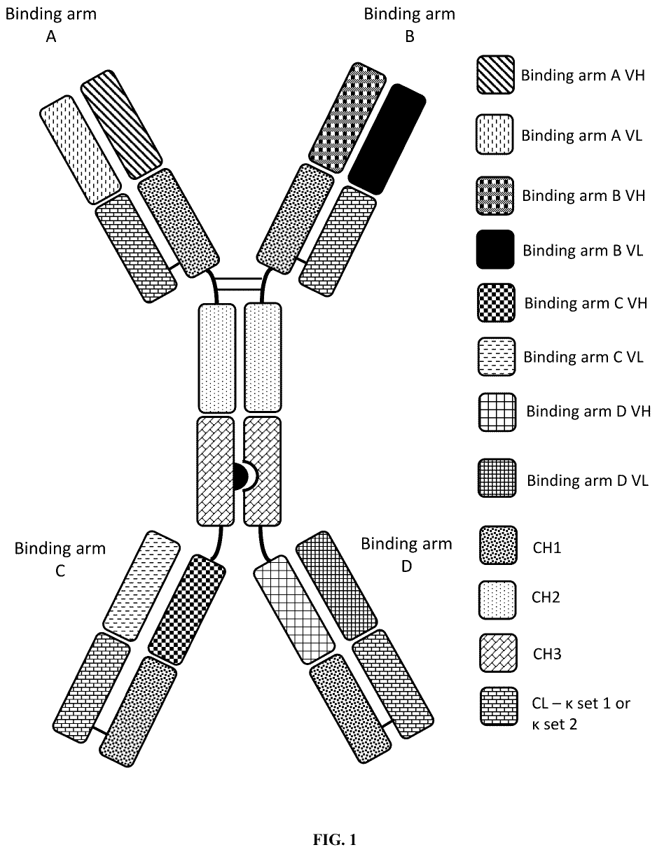

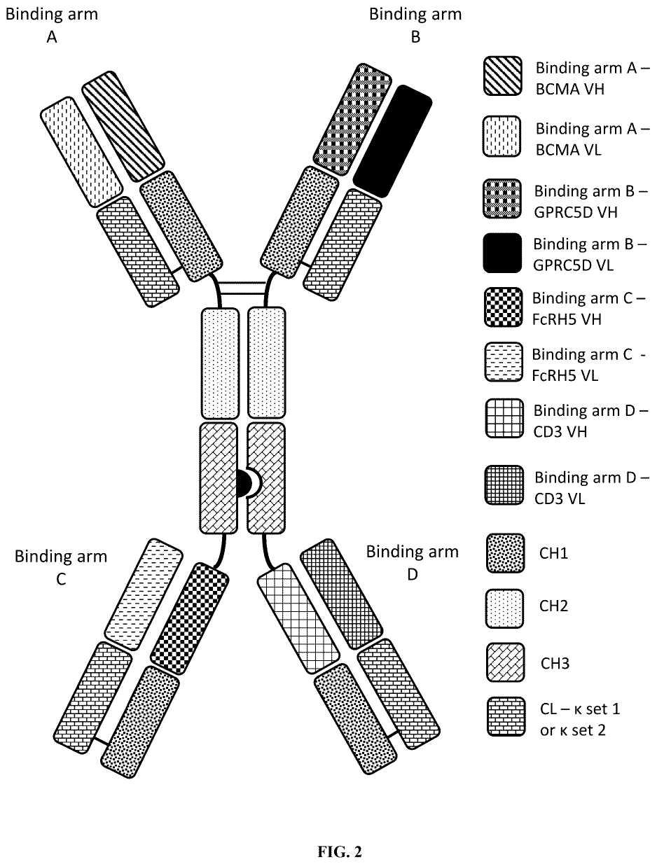

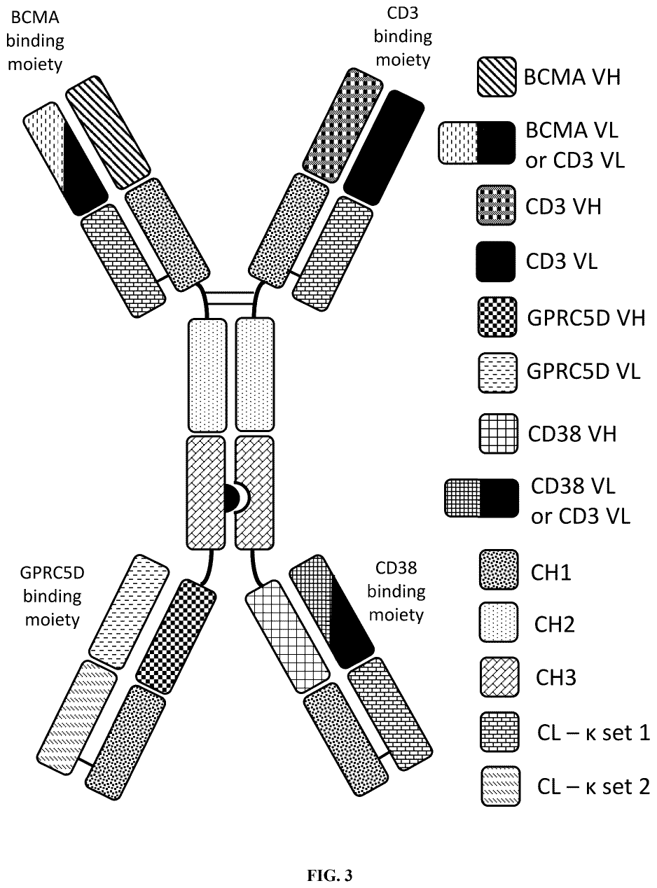

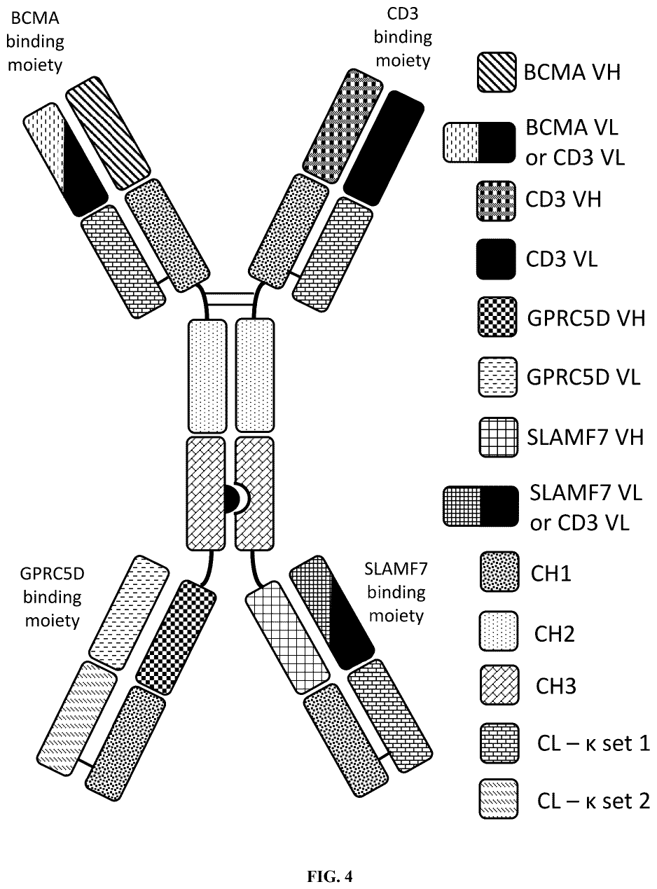

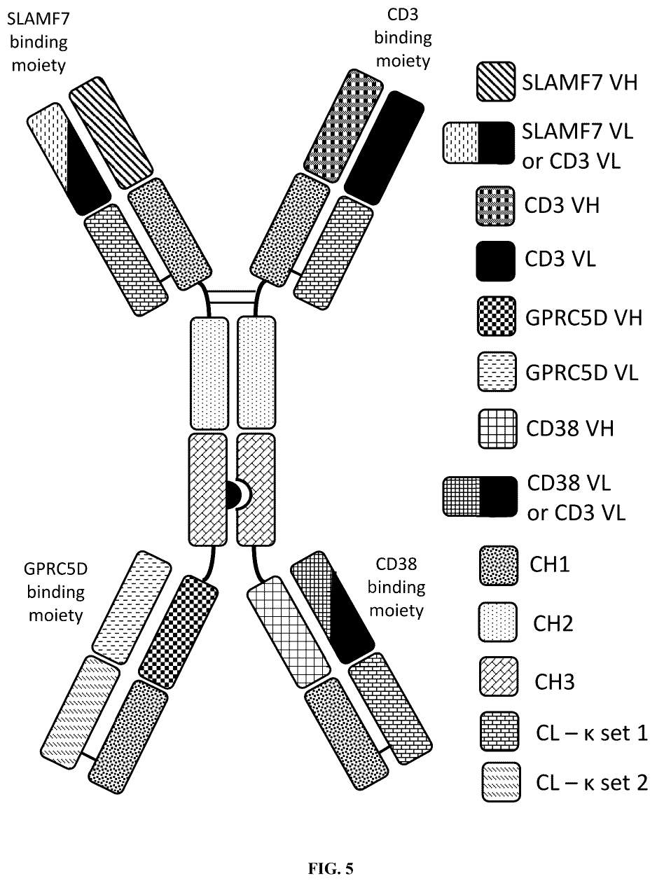

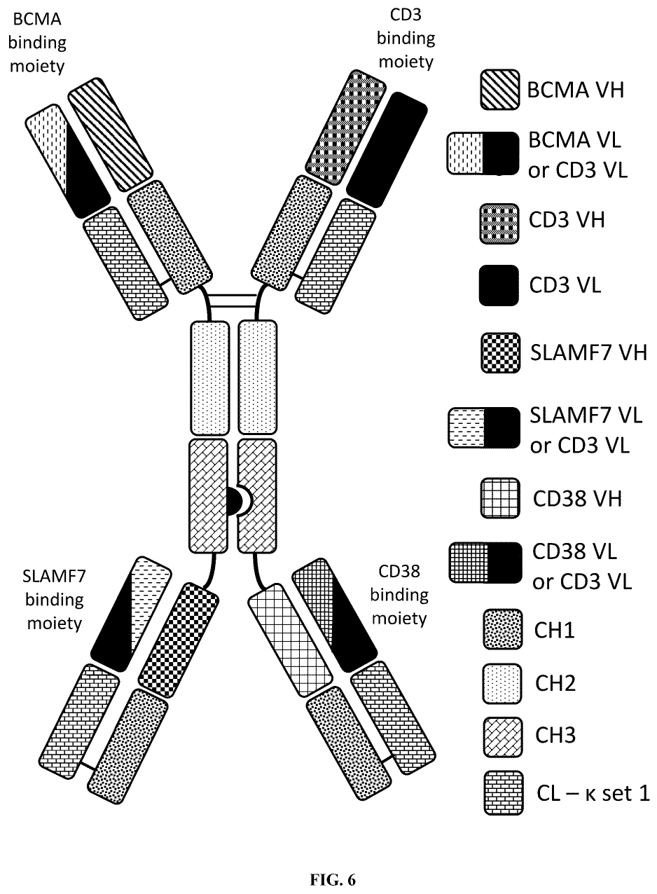

In some aspects, the multispecific binder molecule comprises one or more antibody heavy chains. In some embodiments, the one or more antibody heavy chains are modified antibody heavy chains. In some embodiments, the multispecific binder molecule is organized as a tetraspecific binding molecule capable of high specificity and high affinity binding to four distinct antigens. In some embodiments, the tetraspecific binding molecule is configured with four binding arms (A-D) linked in two modified heavy chains as illustrated in FIG. 1. In some embodiments, each of binding arms A-D is configured to elicit high specificity and high affinity binding to a distinct antigen. In some embodiments, the format of the multispecific binder molecule comprising a tetraspecific binder molecule is listed in FIG. 2. In some embodiments, the format of the multispecific binder molecule comprising a tetraspecific binder molecule is listed in FIG. 3. In some embodiments, the format of the multispecific binder molecule comprising a tetraspecific binder molecule is listed in FIG. 4. In some embodiments, the format of the multispecific binder molecule comprising a tetraspecific binder molecule is listed in FIG. 5. In some embodiments, the format of the multispecific binder molecule comprising a tetraspecific binder molecule is listed in FIG. 6. In some embodiments, the formats of the multispecific binder molecule in FIG. 1-FIG. 12 are formed with six polypeptides. In some embodiments, the six polypeptides comprises two modified heavy chains and four light chains. In some embodiments, the multispecific binder molecule comprises six polypeptides comprising two modified heavy chains and four light chains. In some embodiments, the tetraspecific binder molecule comprises six polypeptides comprising two modified heavy chains and four light chains. In some embodiments, the multispecific binder molecule consists of six polypeptides comprising two modified heavy chains and four light chains. In some embodiments, the tetraspecific binder molecule consists of six polypeptides comprising two modified heavy chains and four light chains.

In some embodiments, the binding arms in the tetraspecific binder molecule are configured to create four distinct binding moieties according to FIG. 1, labeled as binding arm 1, binding arm 2, binding arm 3, and binding arm 4. In some embodiments, the tetraspecific binder molecule is configured according to one of the twenty-four possible configurations for an anti-CD3/BCMA/GPRC5D/FCRH5 tetraspecific binder molecule indicated as Configurations A1-A24 in Table 10.

Exemplary configurations of tetraspecific binder molecules selected from Table 10 are listed below.

Configuration A6 of tetraspecific binder molecule: In some embodiments, the tetraspecific binding molecule is configured according to the configuration for an anti-CD3/BCMA/GPRC5D/FCRH5 tetraspecific binder molecule indicated in FIG. 2. FIG. 2 shows a diagram of a tetraspecific binder molecule formed from six polypeptide sequences in an IgG-Fab configuration. A first modified heavy chain is configured from N-terminal to C-terminal as: i) Binding arm A-BCMA heavy chain variable region (VH), ii) a first constant Ig domain of a heavy chain (CH1), iii) a second constant Ig domain of a heavy chain (CH2), iv) a mutated third Ig domain of a heavy chain (CH3) comprising KiH knob substitutions, v) Binding arm C-FcRH5 VH, and vi) CH1. A second modified heavy chain is configured from N-terminal to C-terminal as: i) Binding arm B-GPRC5D VH, ii) CH1, iii) CH2 iv) a mutated CH3 comprising KiH hole substitutions, v) Binding arm D-CD3 VH, and vi) CH1. A first light chain is configured from N-terminal to C-terminal as: i) Binding arm A-BMCA light chain variable region (VL). and ii) a constant Ig domain of a light chain (CL-κ set 1 or CL-κ set 2). Paired Binding arm A VH and Binding arm A VL form a BCMA binding moiety. A second light chain is configured from N-terminal to C-terminal as: i) Binding arm B-GPRC5D VL and ii) CL-κ set 1 or CL-κ set 2. Paired Binding arm B VH and Binding arm B VL form a GPRC5D binding moiety. A third light chain is configured from N-terminal to C-terminal as: i) Binding arm C-FcRH5 VL and ii) CL-κ set 1 or CL-κ set 2. Paired Binding arm C VH and Binding arm C VL form an FcRH5 binding moiety. A fourth light chain is configured from N-terminal to C-terminal as: i) Binding arm D-CD3 VL, and ii) CL-κ set 1 or CL-κ set 2. Paired Binding arm D VH and Binding arm D VL form a CD3 binding moiety. In some embodiments, the tetraspecific binder molecule comprises four light chains wherein each light chain comprises a CL-κ set 1. In some embodiments, the tetraspecific binder molecule comprises four light chains wherein each light chain comprises a CL-κ set 2. In some embodiments, the tetraspecific binder molecule comprises four light chains wherein one light chain comprises a CL-κ set 1 and three light chains comprise a CL-κ set 2. In some embodiments, the tetraspecific binder molecule comprises four light chains wherein two light chains comprise a CL-κ set 1 and two light chains comprise a CL-κ set 2. In some embodiments, the tetraspecific binder molecule comprises four light chains wherein three light chains comprise a CL-κ set 1 and one light chain comprises a CL-κ set 2.

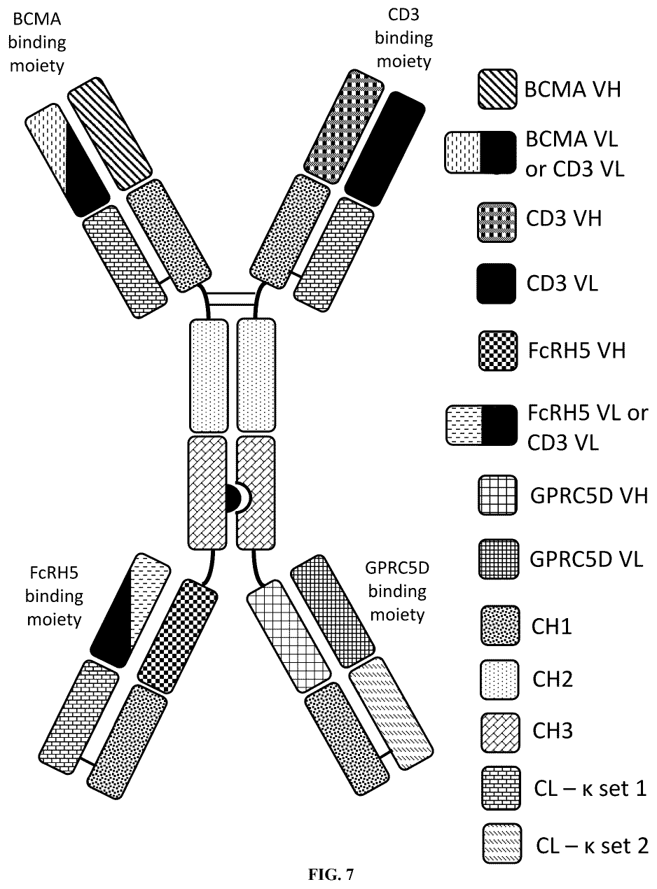

Configuration A2 of tetraspecific binder molecule: In some embodiments, the tetraspecific binding molecule is configured according to the configuration for an anti-CD3/BCMA/GPRC5D/FCRH5 tetraspecific binder molecule indicated in FIG. 7. FIG. 7 shows a diagram of a tetraspecific binder molecule formed from six polypeptide sequences in an IgG-Fab configuration. A first modified heavy chain is configured from N-terminal to C-terminal as: i) BCMA VH, ii) CH1, iii) CH2, iv) a mutated CH3 comprising KiH knob substitutions, v) FcRH5 VH, and vi) CH1. A second modified heavy chain is configured from N-terminal to C-terminal with: i) CD3 VH, ii) CH1, iii) CH2 iv) a mutated CH3 comprising KiH hole substitutions, v) GPRC5D VH, and vi) CH1. A first light chain in binding arm A is configured from N-terminal to C-terminal as either: i-a) BCMA VL or i-b) CD3 VL, and ii) CL-κ set 1. A second light chain in binding arm B is configured from N-terminal to C-terminal as: i) CD3 VL and ii) CL-κ set 1. A third light chain in binding arm C is configured from N-terminal to C-terminal as either: i-a) FcRH5 VL or i-b) CD3 VL, and ii) CL-κ set 1. A fourth light chain in binding arm D is configured from N-terminal to C-terminal as: i) GPRC5D and ii) CL-κ set 2. Two light chains are paired with each modified heavy chain with the respective VH and VL regions arranged to form a BCMA binding moiety in binding arm A, a CD3 binding moiety in binding arm B, a FcRH5 binding moiety in binding arm C, and a GPRC5D binding moiety in binding arm D.

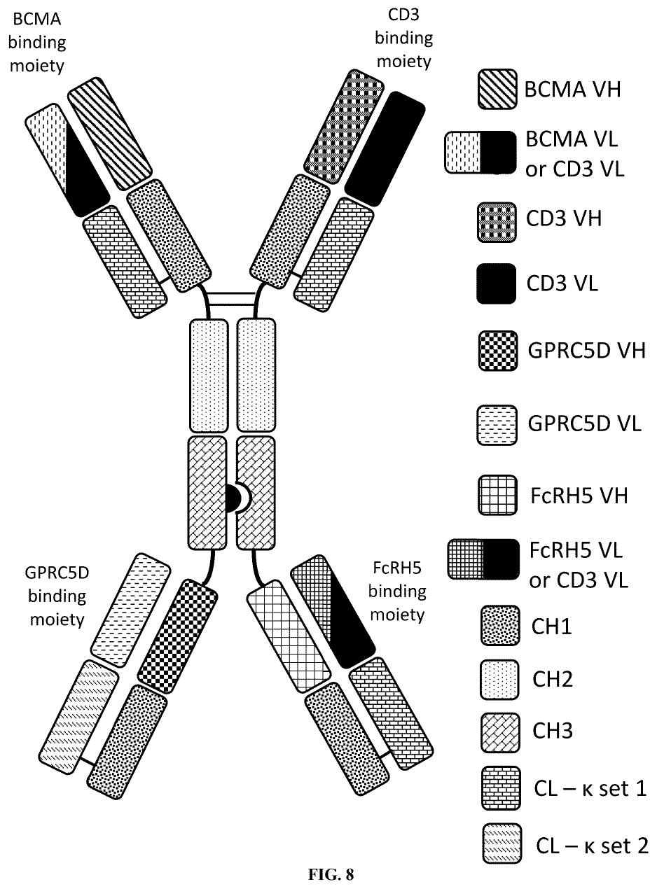

Configuration A1 of tetraspecific binder molecule: In some embodiments, the tetraspecific binding molecule is configured according to the configuration for an anti-CD3/BCMA/GPRC5D/FCRH5 tetraspecific binder molecule indicated in FIG. 8. FIG. 8 shows a diagram of a tetraspecific binder molecule formed from six polypeptide sequences in an IgG-Fab configuration. A first modified heavy chain is configured from N-terminal to C-terminal as: i) BCMA VH, ii) CH1, iii) CH2, iv) a mutated CH3 comprising KiH knob substitutions, v) GPRC5D VH, and vi) CH1. A second modified heavy chain is configured from N-terminal to C-terminal with: i) CD3 VH, ii) CH1, iii) CH2 iv) a mutated CH3 comprising KiH hole substitutions, v) FcRH5 VH, and vi) CH1. A first light chain in binding arm A is configured from N-terminal to C-terminal as either: i-a) BCMA VL or i-b) CD3 VL, and ii) CL-κ set 1. A second light chain in binding arm B is configured from N-terminal to C-terminal as: i) CD3 VL and ii) CL-κ set 1. A third light chain in binding arm C is configured from N-terminal to C-terminal as: i) GPRC5D VL, and ii) CL-κ set 2. A fourth light chain in binding arm D is configured from N-terminal to C-terminal as either: i-a) FcRH5 VL or i-b) CD3 VL and ii) CL-κ set 1. Two light chains are paired with each modified heavy chain with the respective VH and VL regions arranged to form a BCMA binding moiety in binding arm A, a CD3 binding moiety in binding arm B, a GPRC5D binding moiety in binding arm C, and a FcRH5 binding moiety in binding arm D.

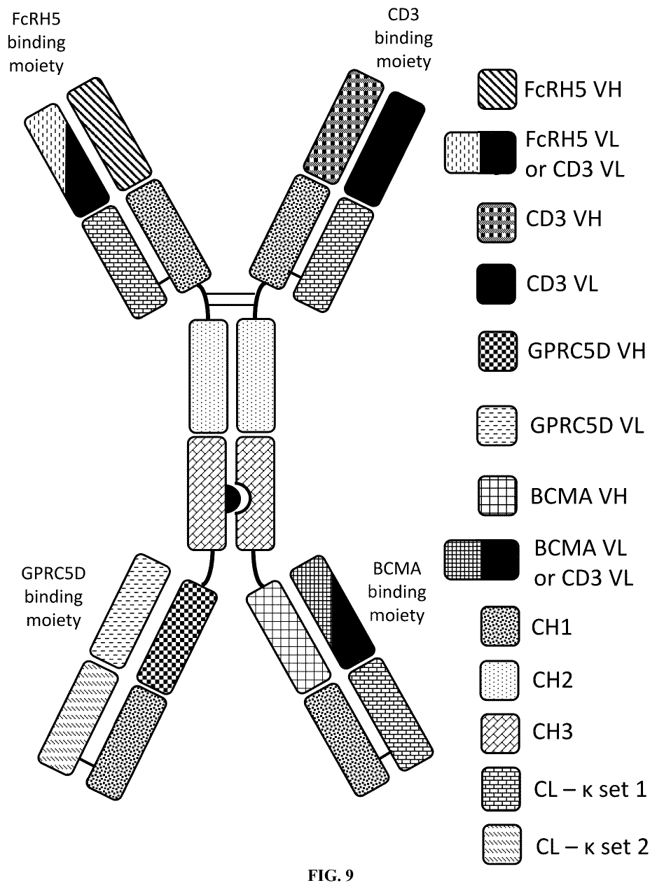

Configuration A16 of tetraspecific binder molecule: In some embodiments, the tetraspecific binding molecule is configured according to the configuration for an anti-CD3/BCMA/GPRC5D/FCRH5 tetraspecific binder molecule indicated in FIG. 9. FIG. 9 shows a diagram of a tetraspecific binder molecule formed from six polypeptide sequences in an IgG-Fab configuration. A first modified heavy chain is configured from N-terminal to C-terminal as: i) FcRH5 VH, ii) CH1, iii) CH2, iv) a mutated CH3 comprising KiH knob substitutions, v) GPRC5D VH, and vi) CH1. A second modified heavy chain is configured from N-terminal to C-terminal with: i) CD3 VH, ii) CH1, iii) CH2 iv) a mutated CH3 comprising KiH hole substitutions, v) BCMA VH, and vi) CH1. A first light chain in binding arm A is configured from N-terminal to C-terminal as either: i-a) FcRH5 VL or i-b) CD3 VL, and ii) CL-κ set 1. A second light chain in binding arm B is configured from N-terminal to C-terminal as: i) CD3 VL and ii) CL-κ set 1. A third light chain in binding arm C is configured from N-terminal to C-terminal as: i) GPRC5D VL, and ii) CL-κ set 2. A fourth light chain in binding arm D is configured from N-terminal to C-terminal as either: i-a) BCMA VL or i-b) CD3 VL and ii) CL-κ set 1. Two light chains are paired with each modified heavy chain with the respective VH and VL regions arranged to form a FcRH5 binding moiety in binding arm A, a CD3 binding moiety in binding arm B, a GPRC5D binding moiety in binding arm C, and a BCMA binding moiety in binding arm D.

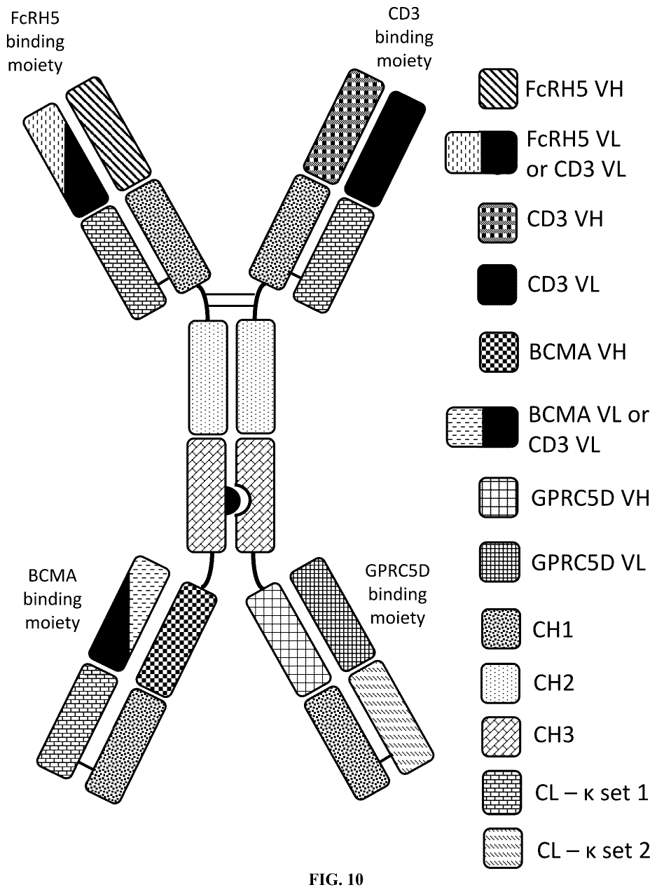

Configuration A15 of tetraspecific binder molecule: In some embodiments, the tetraspecific binding molecule is configured according to the configuration for an anti-CD3/BCMA/GPRC5D/FCRH5 tetraspecific binder molecule indicated in FIG. 10. FIG. 10 shows a diagram of a tetraspecific binder molecule formed from six polypeptide sequences in an IgG-Fab configuration. A first modified heavy chain is configured from N-terminal to C-terminal as: i) FcRH5 VH, ii) CH1, iii) CH2, iv) a mutated CH3 comprising KiH knob substitutions, v) BCMA VH, and vi) CH1. A second modified heavy chain is configured from N-terminal to C-terminal with: i) CD3 VH, ii) CH1, iii) CH2 iv) a mutated CH3 comprising KiH hole substitutions, v) GPRC5D VH, and vi) CH1. A first light chain in binding arm A is configured from N-terminal to C-terminal as either: i-a) FcRH5 VL or i-b) CD3 VL, and ii) CL-κ set 1. A second light chain in binding arm B is configured from N-terminal to C-terminal as: i) CD3 VL and ii) CL-κ set 1. A third light chain in binding arm C is configured from N-terminal to C-terminal as either: i-a) BCMA VL or i-b) CD3 VL and ii) CL-κ set 1. A fourth light chain in binding arm D is configured from N-terminal to C-terminal as: i) GPRC5D VL and ii) CL-κ set 2. Two light chains are paired with each modified heavy chain with the respective VH and VL regions arranged to form a FcRH5 binding moiety in binding arm A, a CD3 binding moiety in binding arm B, a BCMA binding moiety in binding arm C, and a GPRC5D binding moiety in binding arm D.

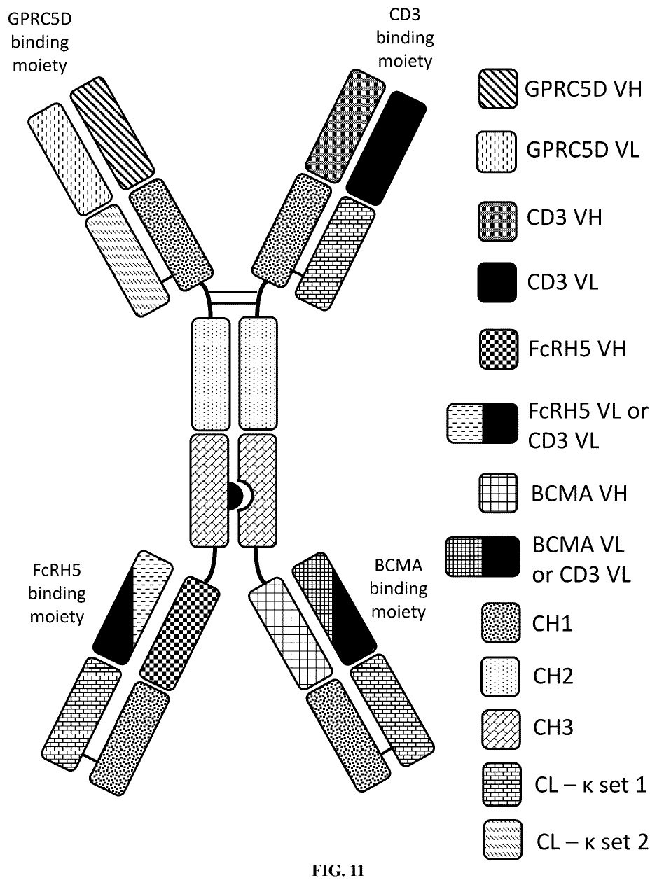

Configuration A22 of tetraspecific binder molecule: In some embodiments, the tetraspecific binding molecule is configured according to the configuration for an anti-CD3/BCMA/GPRC5D/FCRH5 tetraspecific binder molecule indicated in FIG. 11. FIG. 11 shows a diagram of a tetraspecific binder molecule formed from six polypeptide sequences in an IgG-Fab configuration. A first modified heavy chain is configured from N-terminal to C-terminal as: i) GPRC5D VH, ii) CH1, iii) CH2, iv) a mutated CH3 comprising KiH knob substitutions, v) FcRH5 VH, and vi) CH1. A second modified heavy chain is configured from N-terminal to C-terminal with: i) CD3 VH, ii) CH1, iii) CH2 iv) a mutated CH3 comprising KiH hole substitutions, v) BCMA VH, and vi) CH1. A first light chain in binding arm A is configured from N-terminal to C-terminal as: i) GPRC5D VL and ii) CL-κ set 2. A second light chain in binding arm B is configured from N-terminal to C-terminal as: i) CD3 VL and ii) CL-κ set 1. A third light chain in binding arm C is configured from N-terminal to C-terminal as either: i-a) FcRH5 VL or i-b) CD3 VL, and ii) CL-κ set 1. A fourth light chain in binding arm D is configured from N-terminal to C-terminal as either: i-a) BCMA VL or i-b) CD3 VL, and ii) CL-κ set 1. Two light chains are paired with each modified heavy chain with the respective VH and VL regions arranged to form a GPRC5D binding moiety in binding arm A, a CD3 binding moiety in binding arm B, a FcRH5 binding moiety in binding arm C, and a BCMA binding moiety in binding arm D.

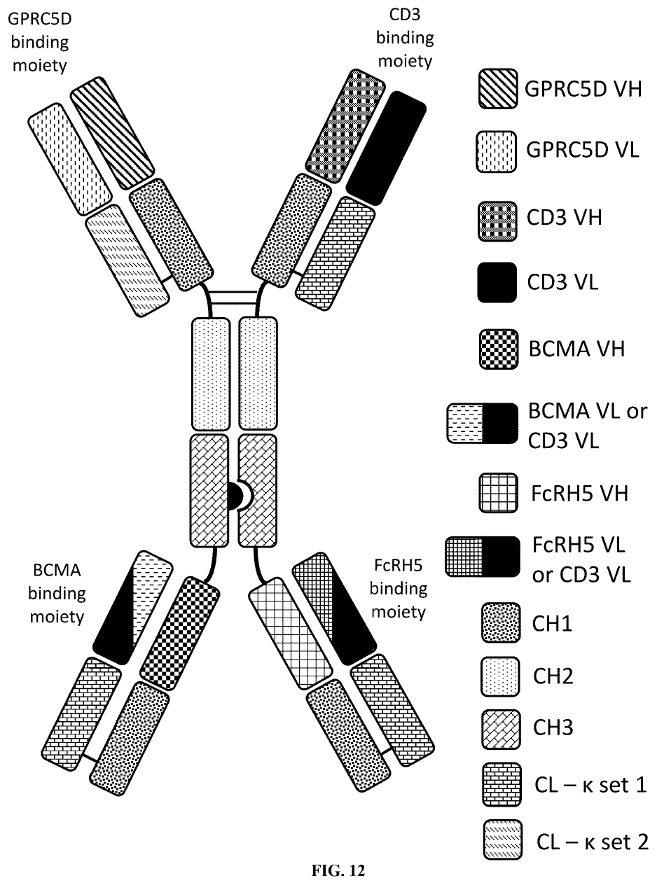

Configuration A21 of tetraspecific binder molecule: In some embodiments, the tetraspecific binding molecule is configured according to the configuration for an anti-CD3/BCMA/GPRC5D/FCRH5 tetraspecific binder molecule indicated in FIG. 12. FIG. 12 shows a diagram of a tetraspecific binder molecule formed from six polypeptide sequences in an IgG-Fab configuration. A first modified heavy chain is configured from N-terminal to C-terminal as: i) GPRC5D VH, ii) CH1, iii) CH2, iv) a mutated CH3 comprising KiH knob substitutions, v) BCMA VH, and vi) CH1. A second modified heavy chain is configured from N-terminal to C-terminal with: i) CD3 VH, ii) CH1, iii) CH2 iv) a mutated CH3 comprising KiH hole substitutions, v) FcRH5 VH, and vi) CH1. A first light chain in binding arm A is configured from N-terminal to C-terminal as: i) GPRC5D VL and ii) CL-κ set 2. A second light chain in binding arm B is configured from N-terminal to C-terminal as: i) CD3 VL and ii) CL-κ set 1. A third light chain in binding arm C is configured from N-terminal to C-terminal as either: i-a) BCMA VL or i-b) CD3 VL, and ii) CL-κ set 1. A fourth light chain in binding arm D is configured from N-terminal to C-terminal as either: i-a) FcRH5 VL or i-b) CD3 VL, and ii) CL-κ set 1. Two light chains are paired with each modified heavy chain with the respective VH and VL regions arranged to form a GPRC5D binding moiety in binding arm A, a CD3 binding moiety in binding arm B, a BCMA binding moiety in binding arm C, and a FcRH5 binding moiety in binding arm D.

In some embodiments, the tetraspecific binding molecule is configured according to one of the twenty-four possible configurations for an anti-CD3/BCMA/GPRC5D/CD38 tetraspecific indicated as Configurations B1-B24 in Table 11. In some embodiments, the tetraspecific binding molecule is configured according to the configuration for an anti-CD3/BCMA/GPRC5D/CD38 tetraspecific binder molecule indicated in FIG. 3. Configuration B1: FIG. 3 shows a diagram of a tetraspecific binder molecule formed from six polypeptide sequences in an IgG-Fab configuration. A first modified heavy chain is configured from N-terminal to C-terminal as: i) BCMA heavy chain variable region (VH), ii) a first constant Ig domain of a heavy chain (CH1), iii) a second constant Ig domain of a heavy chain (CH2), iv) a mutated third Ig domain of a heavy chain (CH3) comprising KiH knob substitutions, v) GPRC5D VH, and vi) CH1. A second modified heavy chain is configured from N-terminal to C-terminal as: i) CD3 VH, ii) CH1, iii) CH2 iv) a mutated CH3 comprising KiH hole substitutions, v) CD38 VH, and vi) CH1. A first light chain is configured from N-terminal to C-terminal as either: i-a) BCMA light chain variable region (VL) or i-b) CD3 VL, and ii) a constant Ig domain of a light chain (CL-κ set 1). A second light chain is configured from N-terminal to C-terminal as: i) GPRC5D VL and ii) CL-κ set 2. A third light chain is configured from N-terminal to C-terminal as: i) CD3 VL and ii) CL-κ set 1. A fourth light chain is configured from N-terminal to C-terminal as either: i-a) CD38 VL or i-b) CD3 VL, and ii) CL-κ set 1. Two light chains are paired with each modified heavy chain with the respective VH and VL regions arranged to form a BCMA binding moiety, a GPRC5D binding moiety, a CD3 binding moiety, and a CD38 binding moiety. In some embodiments, the tetraspecific binding molecule is configured according to one of the twenty-four possible configurations for an anti-CD3/BCMA/GPRC5D/SLAMF7 tetraspecific indicated as Configurations C1-C24 in Table 12. In some embodiments, the tetraspecific binding molecule is configured according to the configuration for an anti-CD3/BCMA/GPRC5D/SLAMF7 tetraspecific indicated in FIG. 4. Configuration C1: FIG. 4 shows a diagram of a tetraspecific binder molecule formed from six polypeptide sequences in an IgG-Fab configuration. A first modified heavy chain is configured from N-terminal to C-terminal as: i) BCMA VH, ii) CH1, iii) CH2, iv) a mutated CH3 comprising KiH knob substitutions, v) GPRC5D VH, and vi) CH1. A second modified heavy chain is configured from N-terminal to C-terminal as: i) CD3 VH, ii) CH1, iii) CH2 iv) a mutated CH3 comprising KiH hole substitutions, v) SLAMF7 VH, and vi) CH1. A first light chain is configured from N-terminal to C-terminal as either: i-a) BCMA VL or i-b) CD3 VL, and ii) CL-κ set 1. A second light chain is configured from N-terminal to C-terminal as: i) GPRC5D VL and ii) CL-κ set 2. A third light chain is configured from N-terminal to C-terminal as: i) CD3 VL and ii) CL-κ set 1. A fourth light chain is configured from N-terminal to C-terminal as either: i-a) SLAMF7 VL or i-b) CD3 VL, and ii) CL-κ set 1. Two light chains are paired with each modified heavy chain with the respective VH and VL regions arranged to form a BCMA binding moiety, a GPRC5D binding moiety, a CD3 binding moiety, and a SLAMF7 binding moiety. In some embodiments, the tetraspecific binding molecule is configured according to one of the twenty-four possible configurations for an anti-CD3/BCMA/CD38/SLAMF7 tetraspecific indicated as Configurations D1-D24 in Table 13. In some embodiments, the tetraspecific binding molecule is configured according to the configuration for an anti-CD3/BCMA/CD38/SLAMF7 tetraspecific indicated in FIG. 6. Configuration D2: FIG. 6 shows a diagram of a tetraspecific binder molecule formed from six polypeptide sequences in an IgG-Fab configuration. A first modified heavy chain is configured from N-terminal to C-terminal as: i) BCMA VH, ii) CH1, iii) CH2, iv) a mutated CH3 comprising KiH knob substitutions, v) SLAMF7 VH, and vi) CH1. A second modified heavy chain is configured from N-terminal to C-terminal with: i) CD3 VH, ii) CH1, iii) CH2 iv) a mutated CH3 comprising KiH hole substitutions, v) CD38 VH, and vi) CH1. A first light chain is configured from N-terminal to C-terminal as either: i-a) BCMA VL or i-b) CD3 VL, and ii) CL-κ set 1. A second light chain is configured from N-terminal to C-terminal as either: i-a) SLAMF7 VL or i-b) CD3 VL, and ii) CL-κ set 1. A third light chain is configured from N-terminal to C-terminal as: i) CD3 VL and ii) CL-κ set 1. A fourth light chain is configured from N-terminal to C-terminal as either: i) CD38 VL or CD3 VL, and ii) CL-κ set 1. Two light chains are paired with each modified heavy chain with the respective VH and VL regions arranged to form a BCMA binding moiety, a SLAMF7 binding moiety, a CD3 binding moiety, and a CD38 binding moiety. In some embodiments, the tetraspecific binding molecule is configured according to one of the twenty-four possible configurations for an anti-CD3/GPRC5D/SLAMF7/CD38 tetraspecific indicated as Configurations E1-E24 in Table 14. In some embodiments, the tetraspecific binding molecule is configured according to the configuration for an anti-CD3/GPRC5D/SLAMF7/CD38 tetraspecific indicated in FIG. 5. Configuration E15: FIG. 5 shows a diagram of a tetraspecific binder molecule formed from six polypeptide sequences in an IgG-Fab configuration. A first modified heavy chain is configured from N-terminal to C-terminal as: i) SLAMF7 VH, ii) CH1, iii) CH2, iv) a mutated CH3 comprising KiH knob substitutions, v) GPRC5D VH, and vi) CH1. A second modified heavy chain is configured from N-terminal to C-terminal as: i) CD3 VH, ii) CH1, iii) CH2 iv) a mutated CH3 comprising KiH hole substitutions, v) CD38 VH, and vi) CH1. A first light chain is configured from N-terminal to C-terminal as either: i-a) SLAMF7 VL or i-b) CD3 VL, and ii) CL-κ set 1. A second light chain is configured from N-terminal to C-terminal as: i) GPRC5D VL and ii) CL-κ set 2. A third light chain is configured from N-terminal to C-terminal as: i) CD3 VL and ii) CL-κ set 1. A fourth light chain is configured from N-terminal to C-terminal as either: i-a) CD38 VL or i-b) CD3 VL, and ii) CL-κ set 1. Two light chains are paired with each modified heavy chain with the respective VH and VL regions arranged to form a SLAMF7 binding moiety, a GPRC5D binding moiety, a CD3 binding moiety, and a CD38 binding moiety. In some embodiments, the tetraspecific binding molecule is configured according to one of the twenty-four possible configurations for an anti-CD3/BCMA/CD38/FCRH5 tetraspecific indicated as Configurations F1-F24 in Table 15. In some embodiments, the tetraspecific binding molecule is configured according to the configuration for an anti-CD3/BCMA/GPRC5D/FCRH5 tetraspecific indicated in FIG. 7. In some embodiments, the tetraspecific binding molecule is configured according to one of the twenty-four possible configurations for an anti-CD3/BCMA/SLAMF7/FCRH5 tetraspecific indicated as Configurations G1-G24 in Table 16. In some embodiments, the tetraspecific binding molecule is configured according to one of the twenty-four possible configurations for an anti-CD3/GPRC5D/CD38/FCRH5 tetraspecific indicated as Configurations H1-H24 in Table 17. In some embodiments, the tetraspecific binding molecule is configured according to one of the twenty-four possible configurations for an anti-CD3/GPRC5D/SLAMF7/FCRH5 tetraspecific indicated as Configurations I1-I24 in Table 18. In some embodiments, the tetraspecific binding molecule is configured according to one of the twenty-four possible configurations for an anti-CD3/CD38/SLAMF7/FCRH5 tetraspecific indicated as Configurations J1-J24 in Table 19.

In some embodiments, the two modified heavy chains are connected in a hinge region by a variable number of disulfide bonds: 2 for IgG1 and IgG4, 4 for IgG2 and 11 for IgG3. In some embodiments, a light chain of the IgG1-format multispecific binder molecule is connected to the modified heavy chain by a disulfide bond between the last cysteine residue of the light chain and the fifth cysteine residue of the modified heavy chain. In some embodiments, the light chain comprising a GPRC5D VL domain comprises a CL-κ set 2 sequence. In some embodiments, the light chain comprising a BCMA VL domain comprises a CL-κ set 1 sequence. In some embodiments, the light chain comprising an FcRH5 VL domain comprises a CL-κ set 1 sequence. In some embodiments, the light chain comprising a CD38 VL domain comprises a CL-κ set 1 sequence. In some embodiments, the light chain comprising a SLAMF7 VL domain comprises a CL-κ set 1 sequence. In some embodiments, the light chain comprising a CD3 VL domain comprises a CL-κ set 1 sequence. In some embodiments, the CL-κ set 1 sequence of: i) the light chain comprising the BCMA VL domain, ii) the light chain comprising the CD38 VL domain, iii) the light chain comprising the SLAMF7 VL domain, and iv) the light chain comprising the CD3 VL domain, comprise identical CL-κ set 1 sequences. In some embodiments, the CL-κ set 1 sequence of: i) the light chain comprising the BCMA VL domain, ii) the light chain comprising the FcRH5 VL domain, and iii) the light chain comprising the CD3 VL domain, comprise identical CL-κ set 1 sequences.

In some embodiments, a high affinity GPRC5D binding moiety is formed by pairing the GPRC5D VH domain with a GPRC5D VL domain. In some embodiments, a high affinity BCMA binding moiety is formed by pairing the BCMA VH domain with a BCMA VL domain. In some embodiments, a high affinity BCMA binding moiety is formed by pairing the BCMA VH domain with a CD3 VL domain. In some embodiments, a high affinity FcRH5 binding moiety is formed by pairing the FcRH5 VH domain with an FcRH5VL domain. In some embodiments, a high affinity FcRH5 binding moiety is formed by pairing the FcRH5 VH domain with a CD3 VL domain. In some embodiments, a high affinity CD38 binding moiety is formed by pairing the CD38 VH domain with a CD38 VL domain. In some embodiments, a high affinity CD38 binding moiety is formed by pairing the CD38 VH domain with a CD3 VL domain. In some embodiments, a high affinity SLAMF7 binding moiety is formed by pairing the SLAMF7 VH domain with a SLAMF7 VL domain. In some embodiments, a high affinity SLAMF7 binding moiety is formed by pairing the SLAMF7 VH domain with a CD3 VL domain. In some embodiments, a high affinity CD3 binding moiety is formed by pairing the CD3 VH domain with a CD3 VL domain. In some embodiments, as illustrated as non-limiting examples in FIG. 1-FIG. 12, multispecific binder molecules comprise six polypeptide chains, each polypeptide chain comprising one or more regions of amino acids sequence which are not identical to any of the other five polypeptide chain sequences. In some embodiments, the six polypeptide chains comprise one CD3 light chain. In some embodiments, the six polypeptide chains comprise two CD3 light chains. In some embodiments, the two CD3 light chains comprises identical amino acid sequences. In some embodiments, the six polypeptide chains comprise three CD3 light chains. In some embodiments, the three CD3 light chains comprises identical amino acid sequences. In some embodiments, two CD3 light chains, or the three CD3 light chains are identical. In some embodiments, the multispecific binder molecule comprises a CD3 light chain that pairs a CD3 VL domain together with a CD3 VH domain residing on a modified heavy chain to form a CD3 binding moiety. In some embodiments, the multispecific binder molecule comprises a first CD3 light chain that pairs a CD3 VL domain together with a CD3 VH domain residing on a first modified heavy chain to form a CD3 binding moiety, and a second CD3 light chain that pairs a CD3 VL domain together with a BCMA VH domain residing on a second modified heavy chain to form a BCMA binding moiety. In some embodiments, the multispecific binder molecule comprises a first CD3 light chain that pairs a CD3 VL domain together with a CD3 VH domain residing on a modified heavy chain to form a CD3 binding moiety, and a second CD3 light chain that pairs a CD3 VL domain together with a CD38 VH domain residing on a modified heavy chain to form a CD38 binding moiety. In some embodiments, the multispecific binder molecule comprises a first CD3 light chain that pairs a CD3 VL domain together with a CD3 VH domain residing on a modified heavy chain to form a CD3 binding moiety, and a second CD3 light chain that pairs a CD3 VL domain together with a SLAMF7 VH domain residing on a modified heavy chain to form a SLAMF7 binding moiety. In some embodiments, the multispecific binder molecule comprises two modified heavy chains, two identical CD3 light chains, and two light chains not identical to CD3 light chains. In some embodiments, the multispecific binder molecule comprises two modified heavy chains, three identical CD3 light chains, and one light chain not identical to CD3 light chains. In some embodiments, the CD3 light chains comprising a CD3 VL domain may be paired with any of: i) a CD3 VH domain, ii) a BCMA VH domain, iii) a CD38 VH domain, iv) a SLAMF7 VH domain, or v) an FcRH5 VH domain.

In some cases, an antibody-based multispecific binder molecule comprises one or more mutations in a framework region, e.g., in the CH1 domain, CH2 domain, CH3 domain, hinge region, or a combination thereof. In some instances, the one or more mutations are to stabilize the antibody. In some instances, the one or more mutations are to increase half-life. In some instances, the one or more mutations are to modulate Fc receptor interactions. In some instances, the one or more mutations are to reduce or eliminate Fc effector functions such as FcyR, antibody-dependent cell-mediated cytotoxicity (ADCC), or complement-dependent cytotoxicity (CDC). In additional instances, the one or more mutations are to modulate glycosylation.

In some embodiments, the one or more mutations are located in the Fc region. In some embodiments, the one or more mutations are located in the CH1 region. In some embodiments, the one or more mutations are located in the CH2 region. In some embodiments, the one or more mutations are located in the CH3 region.

In some embodiments, the human IgG constant region is modified to alter antibody-dependent cellular cytotoxicity (ADCC) and/or complement-dependent cytotoxicity (CDC), e.g., with an amino acid modification described in Natsume et al., 2008 Cancer Res, 68 (10): 3863-72; Idusogie et al., 2001 J Immunol, 166 (4): 2571-5; Moore et al., 2010 mAbs, 2 (2): 181-189; Lazar et al., 2006 PNAS, 103 (11): 4005-4010, Shields et al., 2001 JBC, 276 (9): 6591-6604; Stavenhagen et al., 2007 Cancer Res, 67 (18): 8882-8890; Stavenhagen et al., 2008 Advan. Enzyme Regul., 48:152-164; Alegre et al, 1992 J Immunol, 148:3461-3468; Urban et al., 2021 Front Immunol. November 25; 12:724361; Zhou et al., 2020 MAbs. Jan-Dec; 12 (1): 1814583; Reviewed in Kaneko and Niwa, 2011 Biodrugs, 25 (1): 1-11.

In some embodiments, the heavy chain constant regions of a multispecific binding molecule antibody heavy chain described herein is categorized as IgG1, IgG2, or IgG4. In some embodiments, the IgG1, IgG2, or IgG4 is human IgG1, human IgG2, or human IgG4. In some embodiments, the IgG1, IgG2, or IgG4 is mouse IgG1, mouse IgG2, or mouse IgG4. In some embodiments, the IgG1, IgG2, or IgG4 is rat IgG1, rat IgG2, or rat IgG4. In some embodiments, the constant regions of the multispecific binding molecule antibody heavy chain are human IgG4 wild-type. In some embodiments, the constant regions of the multispecific binding molecule antibody heavy chain are human IgG1 wild-type. In some embodiments, the constant regions of the multispecific binding molecule antibody heavy chain are human IgG1 wild-type having a REEM allotype sequence. In some embodiments, the constant regions of the multispecific binding molecule antibody heavy chain are human IgG2 wild-type. In some embodiments, the constant regions of the multispecific binding molecule antibody heavy chain are human IgG1-3M wild-type. In some embodiments, the constant regions of the multispecific binding molecule antibody heavy chain are human IgG1-3M wild-type having a REEM allotype sequence.

In some embodiments, constant regions of the multispecific binding molecule antibody heavy chain have one or more mutations. In some embodiments, the one or more mutations comprise one or more amino acid substitutions, insertions, or deletions. In some embodiments, a variant or modified heavy chain comprises a mutation that reduced FcγR binding. In some embodiments, a variant or modified IgG1 heavy chain comprises a mutation that reduced FcγR binding. In some embodiments, the constant regions of the multispecific binding molecule antibody heavy chain are human IgG4 having a S228P substitution. In some embodiments, the constant regions of the multispecific binding molecule antibody heavy chain are human IgG1 having a L234A substitution. In some embodiments, the constant regions of the multispecific binding molecule antibody heavy chain are human IgG1 having a L235A substitution. In some embodiments, the constant regions of the multispecific binding molecule antibody heavy chain are human IgG1 having L234A and L235A substitutions. In some embodiments, the constant regions of the multispecific binding molecule antibody heavy chain are human IgG1 having L234A, L235A, and P329G substitutions. In some embodiments, the constant regions of the multispecific binding molecule antibody heavy chain are human IgG1 having L234A, L235A, and P329S substitutions. In some embodiments, the constant regions of the multispecific binding molecule antibody heavy chain are human IgG1 having L234A, L235A, and P329G substitutions. In some embodiments, the constant regions of the multispecific human IgG1 wild-type having a REEM allotype sequence and L234A, L235A, and P329G substitutions. In some embodiments, the CH2 domain of the multispecific binder molecule comprises L234A, L235A, and P329G substitutions. In some embodiments, the L234A, L235A, and P329G substitutions decrease binding of the multispecific binder molecule to Fc gamma receptors and to complement components preventing Fc gamma receptor-mediated activation of innate immune effector cells including NK cells, monocytes, macrophages, and neutrophils. In some embodiments, the CH2 domain of the multispecific binder molecule comprises L234A, L235A, and P329S substitutions. In some embodiments, the CH2 domain of the multispecific binder molecule comprises an N297A substitution. In some embodiments, a substitution in a heavy chain constant region in the multispecific binder molecule is referred to as a mutation. In some embodiments, the multispecific binder molecule comprises a first heavy chain and a second heavy chain. In some embodiments, the first heavy chain, the second heavy chain, or the first and second heavy chains are modified heavy chains or variant heavy chains. In some embodiments, one or more amino acid substitutions in the first, second, or first and second heavy chains aid in heterodimer formation of the multispecific binder molecule comprising at least two heavy chains. In some embodiments, the CH3 domain of the multispecific binder molecule comprises substitutions which contribute to forming a knob structure for knob-in-hole (KiH) technology for use in aiding in heterodimerization of modified heavy chains. In some embodiments, the knob substitutions comprise either S354C, T366W, or S354C and T366W. In some embodiments, the CH3 domain of the multispecific binder molecule comprises substitutions which contribute to forming a hole structure for KiH technology for use in aiding in heterodimerization of modified heavy chains. In some embodiments, the knob substitutions comprise Y349C, T366S, L368A, Y407V, or any combination thereof. In some embodiments, a first heavy chain comprising KiH knob mutations is paired with a second heavy chain comprising KiH hole mutations to aid in heterodimerization of the separate modified heavy chains. Representative heavy chain constant region sequences used in antibody heavy chains described herein are listed in Table 8. In some embodiments, the heavy chain constant region sequences of the multispecific binder molecule heavy chain comprise an amino acid sequence selected from a sequence of SEQ ID NOs: 49-68. In some embodiments, a CH1 region present in Table 8 is positioned C-terminal to a VH domain described here in the multispecific binder molecule. In some embodiments, a CH2 region present in Table 8 is positioned C-terminal to a CH1 domain in the multispecific binder molecule. In some embodiments, a CH3 region present in Table 8 is positioned C-terminal to a CH2 domain in the multispecific binder molecule. A representative light chain constant region sequence used in antibody light chains described herein are listed in Table 9.

In some embodiments, the binding moieties of the multispecific binding molecule are linked to other elements of the molecule by linker sequences. In some embodiments, a linker sequence is positioned between a first binding moiety and a second binding moiety. In some embodiments, a linker sequence is positioned between a first binding moiety and a second unit of the first binding moiety. In some embodiments, a linker sequence is positioned between a first binding moiety and an antibody constant region. In some embodiments, a linker sequence is positioned between a second binding moiety and an antibody constant region. In some embodiments, a linker sequence is positioned between a third binding moiety and an antibody constant region. In some embodiments, a linker sequence is positioned between an immune cell engager and an antibody constant region. In some embodiments, a linker sequence is positioned between a first binding moiety and a CH1 region. In some embodiments, a linker sequence is positioned between a first binding moiety and a CH2 region. In some embodiments, a linker sequence is positioned between a second binding moiety and a CH1 region. In some embodiments, a linker sequence is positioned between a second binding moiety and a CH2 region. In some embodiments, a linker sequence is positioned between a third binding moiety and a CH1 region. In some embodiments, a linker sequence is positioned between a third binding moiety and a CH2 region. In some embodiments, a linker sequence is positioned between a first binding moiety and a CH1 region. In some embodiments, a linker sequence is positioned C-terminal to a CH3 domain. In some embodiments, a VH and CH1 domain are linked to a linker C-terminal to a CH3 domain comprising a single modified heavy chain. In some embodiments, the linker sequence positioned between a binding moiety and an antibody constant region comprises one or more sequences listed in Table 21.

TABLE 8
Heavy chain constant region sequences

| SEQ ID | | |
|---|---|---|
| NO: | Name | Amino Acid Sequence |
| 49 | Human | ASTKGPSVFPLAPCSRSTSESTAALGCLVKDYFPEPVTVSWNSGALTSGVHTFPAVLQ |
| | IgG4 wild- | SSGLYSLSSVVTVPSSSLGTKTYTCNVDHKPSNTKVDKRVESKYGPPCPSCPAPEFLG |
| | type | GPSVFLFPPKPKDTLMISRTPEVTCVVVDVSQEDPEVQFNWYVDGVEVHNAKTKPREE |
| | | QFNSTYRVVSVLTVLHQDWLNGKEYKCKVSNKGLPSSIEKTISKAKGQPREPQVYTLP |
| | | PSQEEMTKNQVSLTCLVKGFYPSDIAVEWESNGQPENNYKTTPPVLDSDGSFFLYSRL |
| | | TVDKSRWQEGNVFSCSVMHEALHNHYTQKSLSLSLGK |
| 50 | Human | ASTKGPSVFPLAPCSRSTSESTAALGCLVKDYFPEPVTVSWNSGALTSGVHTFPAVLQ |
| | IgG4 | SSGLYSLSSVVTVPSSSLGTKTYTCNVDHKPSNTKVDKRVESKYGPPCPPCPAPEFLG |
| | (S228P) | GPSVFLFPPKPKDTLMISRTPEVTCVVVDVSQEDPEVQFNWYVDGVEVHNAKTKPREE |
| | | QFNSTYRVVSVLTVLHQDWLNGKEYKCKVSNKGLPSSIEKTISKAKGQPREPQVYTLP |
| | | PSQEEMTKNQVSLTCLVKGFYPSDIAVEWESNGQPENNYKTTPPVLDSDGSFFLYSRL |
| | | TVDKSRWQEGNVFSCSVMHEALHNHYTQKSLSLSLGK |
| 51 | Human | ASTKGPSVFPLAPSSKSTSGGTAALGCLVKDYFPEPVTVSWNSGALTSGVHTFPAVLQ |
| | IgG1 wild- | SSGLYSLSSVVTVPSSSLGTQTYICNVNHKPSNTKVDKKVEPKSCDKTHTCPPCPAPEL |
| | type | LGGPSVFLFPPKPKDTLMISRTPEVTCVVVDVSHEDPEVKFNWYVDGVEVHNAKTKPR |
| | | EEQYNSTYRVVSVLTVLHQDWLNGKEYKCKVSNKALPAPIEKTISKAKGQPREPQVYTL |
| | | PPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGK |
| 52 | Human | ASTKGPSVFPLAPSSKSTSGGTAALGCLVKDYFPEPVTVSWNSGALTSGVHTFPAVLQ |
| | IgG1 wild- | SSGLYSLSSWTVPSSSLGTQTYICNVNHKPSNTKVDKKVEPKSCDKTHTCPPCPAPEL |
| | type | LGGPSVFLFPPKPKDTLMISRTPEVTCWWVDVSHEDPEVKFNWYVDGVEVHNAKTKPR |
| | N297A | EEQYASTYRVVSVLTVLHQDWLNGKEYKCKVSNKALPAPIEKTISKAKGQPREPQVYTL |
| | | PPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGK |
| 53 | Human | ASTKGPSVFPLAPSSKSTSGGTAALGCLVKDYFPEPVTVSWNSGALTSGVHTFPAVLQ |
| | IgG1 LALA | SSGLYSLSSWTVPSSSLGTQTYICNVNHKPSNTKVDKKVEPKSCDKTHTCPPCPAPE |
| | | AAGGPSVFLFPPKPKDTLMISRTPEVTCWWVDVSHEDPEVKFNWYVDGVEVHNAKTKP |
| | | REEQYNSTYRVVSVLTVLHQDWLNGKEYKCKVSNKALPAPIEKTISKAKGQPREPQVY |
| | | TLPPSRDELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 54 | Human | ASTKGPSVFPLAPSSKSTSGGTAALGCLVKDYFPEPVTVSWNSGALTSGVHTFPAVLQ |
| | IgG1 LALA | SSGLYSLSSWVTVPSSSLGTQTYICNVNHKPSNTKVDKKVEPKSCDKTHTCPPCPAPE |
| | P329G | AAGGPSVFLFPPKPKDTLMISRTPEVTCVVVDVSHEDPEVKFNWYVDGVEVHNAKTKP |
| | | REEQYNSTYRVVSVLTVLHQDWLNGKEYKCKVSNKALGAPIEKTISKAKGQPREPQVY |
| | | TLPPSRDELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 55 | Human | ASTKGPSVFPLAPSSKSTSGGTAALGCLVKDYFPEPVTVSWNSGALTSGVHTFPAVLQ |
| | IgG-3M | SSGLYSLSSVVTVPSSSLGTQTYICNVNHKPSNTKVDKKVEPKSCDKTHTCPPCPAPE |
| | | AAGAPSVFLFPPKPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTISKAKGQPREPQVY |
| | | TLPPSRDELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 56 | Human | ASTKGPSVFPLAPCSRSTSESTAALGCLVKDYFPEPVTVSWNSGALTSGVHTFPAVLQ |
| | IgG2 wild- | SSGLYSLSSWTVPSSNFGTQTYTCNVDHKPSNTKVDKTVERKCCVECPPCPAPPVAG |
| | type | PSVFLFPPKPKDTLMISRTPEVTCVVVDVSHEDPEVQFNWYVDGVEVHNAKTKPREEQ |
| | | FNSTFRVVSVLTWVHQDWLNGKEYKCKVSNKGLPAPIEKTISKTKGQPREPQVYTLPPS |
| | | REEMTKNQVSLTCLVKGFYPSDISVEWESNGQPENNYKTTPPMLDSDGSFFLYSKLTV |
| | | DKSRWQQGNVFSCSVMHEALHNHYTQKSLSLSPGK |
| 57 | Human | ASTKGPSVFPLAPSSKSTSGGTAALGCLVKDYFPEPVTVSWNSGALTSGVHTFPAVLQ |
| | IgG1 wild- | SSGLYSLSSWVTVPSSSLGTQTYICNVNHKPSNTKVDKKVEPKSCDKTHTCPPCPAPEL |
| | type REEM | LGGPSVFLFPPKPKDTLMISRTPEVTCVVVDVSHEDPEVKFNWYVDGVEVHNAKTKPR |
| | allotype | EEQYNSTYRVVSVLTVLHQDWLNGKEYKCKVSNKALPAPIEKTISKAKGQPREPQVYTL |
| | | PPSREEMTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGK |
| 58 | Human | ASTKGPSVFPLAPSSKSTSGGTAALGCLVKDYFPEPVTVSWNSGALTSGVHTFPAVLQ |
| | IgG1 wild- | SSGLYSLSSWVTVPSSSLGTQTYICNVNHKPSNTKVDKKVEPKSCDKTHTCPPCPAPE |
| | type REEM | AAGGPSVFLFPPKPKDTLMISRTPEVTCVVVDVSHEDPEVKFNWYVDGVEVHNAKTKP |
| | allotype | REEQYNSTYRVVSVLTVLHQDWLNGKEYKCKVSNKALGAPIEKTISKAKGQPREPQVY |
| | LALA | TLPPSREEMTKNQVSLTCLVKGFYPSDIAVEWESNGQPENNYKTTPPVLDSDGSFFLY |
| | P329G | SKLTVDKSRWQQGNVFSCSVMHEALHNHYTQKSLSLSPGK |
| 59 | Human | ASTKGPSVFPLAPSSKSTSGGTAALGCLVKDYFPEPVTVSWNSGALTSGVHTFPAVLQ |
| | IgG1-3M | SSGLYSLSSVVTVPSSSLGTQTYICNVNHKPSNTKVDKKVEPKSCDKTHTCPPCPAPE |
| | REEM | AAGAPSVFLFPPKPKDTLMISRTPEVTCWWVDVSHEDPEVKFNWYVDGVEVHNAKTKP |
| | allotype | REEQYNSTYRVVSVLTVLHQDWLNGKEYKCKVSNKALPAPIEKTISKAKGQPREPQVY |
| | | TLPPSREEMKNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| 60 | Human | APEAAGGPSVFLFPPKPKDTLMISRTPEVTCVVVDVSHEDPEVKFNWYVDGVEVHNAK |
| | IgG1-CH2 | TKPREEQYNSTYRVVSVLTVLHQDWLNGKEYKCKVSNKALGAPIEKTISKAK |
| | LALAPG | |
| 61 | Human | APEAAGGPSVFLFPPKPKDTLMISRTPEVTCVVVDVSHEDPEVKFNWYVDGVEVHNAK |
| | IgG1-CH2 | TKPREEQYNSTYRVVSVLTVLHQDWLNGKEYKCKVSNKALSAPIEKTISKAK |
| | LALAPS | |
| 62 | Human | GQPREPQVYTLPPCREEMTKNQVSLWCLVKGFYPSDIAVEWESNGQPENNYKTTPPV |
| | IgG1-CH3 | LDSDGSFFLYSKLTVDKSRWQQGNVFSCSVMHEALHNHYTQKSLSLSPGK |
| | KiH knob | |
| | S354C, | |
| | T366W | |
| 63 | Human | GQPREPQVCTLPPSREEMTKNQVSLSCAVKGFYPSDIAVEWESNGQPENNYKTTPPV |
| | IgG1-CH3 | LDSDGSFFLVSKLTVDKSRWQQGNVFSCSVMHEALHNHYTQKSLSLSPGK |
| | KiH hole | |
| | Y349C, | |
| | T366S, | |
| | L368A, | |
| | Y407V | |
| 64 | Human | DKTHTCPPCPAPEAAGGPSVFLFPPKPKDTLMISRTPEVTCVVVDVSHEDPEVKFNWY |
| | IgG1- | VDGVEVHNAKTKPREEQYNSTYRVVSVLTVLHQDWLNGKEYKCKVSNKALGAPIEKTI |
| | CH2: | SKAKGQPREPQVYTLPPCREEMTKNQVSLWCLVKGFYPSDIAVEWESNGQPENNYKT |
| | LALA-PG; | TPPVLDSDGSFFLYSKLTVDKSRWQQGNVFSCSVMHEALHNHYTQKSLSLSPGK |
| | CH3: KIH | |
| | knob | |
| 65 | Human | DKTHTCPPCPAPEAAGGPSVFLFPPKPKDTLMISRTPEVTCVVVDVSHEDPEVKFNWY |
| | IgG1- | VDGVEVHNAKTKPREEQYNSTYRVVSVLTVLHQDWLNGKEYKCKVSNKALGAPIEKTI |
| | CH2: | SKAKGQPREPQVCTLPPSREEMTKNQVSLSCAVKGFYPSDIAVEWESNGQPENNYKT |
| | LALA-PG; | TPPVLDSDGSFFLVSKLTVDKSRWQQGNVFSCSVMHEALHNHYTQKSLSLSPGK |
| | CH3: KIH | |
| | hole | |
| 66 | ACCESSION: | DKTHTCPPCPAPELLGGPSVFLFPPKPKDTLMISRTPEVTCVVVDVSHEDPEVKFNWY |
| | 7LUR_A | VDGVEVHNAKTKPCEEQYGSTYRCVSVLTVLHQDWLNGKEYKCKVSNKALPAPIEKTI |
| | Chain A, | SKAKGQPREPQVYTLPPSREEMTKNQVSLTCLVKGFYPSDIAVEWESNGQPENNYKT |
| | Immunoglobulin | TPPVLDSDGSFFLYSKLTVDKSRWQQGNVFSCSVMHEALHNHYTQKSLSLSPGK |
| | heavy | |
| | constant | |
| | gamma 1 | |
| | Homo | |
| | sapiens | |
| | CH1 | |
| 67 | CH1, CH2, | ASTKGPSVFPLAPSSKSTSGGTAALGCLVKDYFPEPVTVSWNSGALTSGVHTFPAVLQ |
| | CH3 from | SSGLYSLSSVTVPSSSLGTQTYICNVNHKPSNTKVDKKVEPKSCDKTHTCPPCPAPE |
| | subunit 1 | AAGGPSVFLFPPKPKDTLMISRTPEVTCVVVDVSHEDPEVKFNWYVDGVEVHNAKTKP |
| | Alnuctamab | REEQYNSTYRVVSVLTVLHQDWLNGKEYKCKVSNKALGAPIEKTISKAKGQPREPQVY |
| | (EM 801) | TLPPCRDELTKNQVSLWCLVKGFYPSDIAVEWESNGQPENNYKTTPPVLDSDGSFFLY |
| | | SKLTVDKSRWQQGNVFSCSVMHEALHNHYTQKSLSLSPGK |
| 68 | CH1, CH2, | ASTKGPSVFPLAPSSKSTSGGTAALGCLVEDYFPEPVTVSWNSGALTSGVHTFPAVLQ |
| | CH3 from | SSGLYSLSSVVTVPSSSLGTQTYICNVNHKPSNTKVDEKVEPKSCDKTHTCPPCPAPE |
| | subunit 2 | AAGGPSVFLFPPKPKDTLMISRTPEVTCVVVDVSHEDPEVKFNWYVDGVEVHNAKTKP |
| | Alnuctamab | REEQYNSTYRVVSVLTVLHQDWLNGKEYKCKVSNKALGAPIEKTISKAKGQPREPQVC |
| | (EM 801) | TLPPSRDELTKNQVSLSCAVKGFYPSDIAVEWESNGQPENNYKTTPPVLDSDGSFFLV |
| | | SKLTVDKSRWQQGNVFSCSVMHEALHNHYTQKSLSLSPGK |

TABLE 9
Light chain constant region sequences

| SEQ ID | | |
|---|---|---|
| NO: | Name | Amino Acid Sequence |
| 69 | Human kappa | RTVAAPSVFIFPPSDEQLKSGTASVVC |
| | light chain | LLNNFYPREAKVQWKVDNALQSGNSQE |
| | constant | SVTEQDSKDSTYSLSSTLTLSKADYEK |
| | | HKVYACEVTHQGLSSPVTKSFNRGEC |

TABLE 10
Anti-CD3/BCMA/GPRC5D/FCRH5 tetraspecific configurations

| | Binding arm | Binding arm | Binding arm | Binding arm |
|---|---|---|---|---|
| Configuration | in A | in B | in C | in D |
| A1 | BCMA | CD3 | GRPC5D | FcRH5 |
| A2 | BCMA | CD3 | FcRH5 | GRPC5D |
| A3 | BCMA | FcRH5 | CD3 | GRPC5D |
| A4 | BCMA | FcRH5 | GRPC5D | CD3 |
| A5 | BCMA | GRPC5D | CD3 | FcRH5 |
| A6 | BCMA | GRPC5D | FcRH5 | CD3 |
| A7 | CD3 | BCMA | GRPC5D | FcRH5 |
| A8 | CD3 | BCMA | FcRH5 | GRPC5D |
| A9 | CD3 | FcRH5 | GRPC5D | BCMA |
| A10 | CD3 | FcRH5 | BCMA | GRPC5D |
| A11 | CD3 | GRPC5D | FcRH5 | BCMA |
| A12 | CD3 | GRPC5D | BCMA | FcRH5 |
| A13 | FcRH5 | BCMA | CD3 | GPRC5D |
| A14 | FcRH5 | BCMA | GPRC5D | CD3 |
| A15 | FcRH5 | CD3 | BCMA | GPRC5D |
| A16 | FcRH5 | CD3 | GPRC5D | BCMA |
| A17 | FcRH5 | GRPC5D | BCMA | CD3 |
| A18 | FcRH5 | GRPC5D | CD3 | BCMA |
| A19 | GRPC5D | BCMA | CD3 | FcRH5 |
| A20 | GRPC5D | BCMA | FcRH5 | CD3 |
| A21 | GRPC5D | CD3 | BCMA | FcRH5 |
| A22 | GRPC5D | CD3 | FcRH5 | BCMA |
| A23 | GRPC5D | FcRH5 | BCMA | CD3 |
| A24 | GRPC5D | FcRH5 | CD3 | BCMA |

TABLE 11
Anti-CD3/BCMA/GPRC5D/CD38 tetraspecific configurations

| | Binding arm | Binding arm | Binding arm | Binding arm |
|---|---|---|---|---|
| Configuration | in A | in B | in C | in D |
| B1 | BCMA | CD3 | GRPC5D | CD38 |
| B2 | BCMA | CD3 | CD38 | GRPC5D |
| B3 | BCMA | CD38 | CD3 | GRPC5D |
| B4 | BCMA | CD38 | GRPC5D | CD3 |
| B5 | BCMA | GRPC5D | CD3 | CD38 |
| B6 | BCMA | GRPC5D | CD38 | CD3 |
| B7 | CD3 | BCMA | GRPC5D | CD38 |
| B8 | CD3 | BCMA | CD38 | GRPC5D |
| B9 | CD3 | CD38 | GRPC5D | BCMA |
| B10 | CD3 | CD38 | BCMA | GRPC5D |
| B11 | CD3 | GRPC5D | CD38 | BCMA |
| B12 | CD3 | GRPC5D | BCMA | CD38 |
| B13 | CD38 | BCMA | CD3 | GPRC5D |
| B14 | CD38 | BCMA | GPRC5D | CD3 |
| B15 | CD38 | CD3 | BCMA | GPRC5D |
| B16 | CD38 | CD3 | GPRC5D | BCMA |
| B17 | CD38 | GRPC5D | BCMA | CD3 |
| B18 | CD38 | GRPC5D | CD3 | BCMA |
| B19 | GRPC5D | BCMA | CD3 | CD38 |
| B20 | GRPC5D | BCMA | CD38 | CD3 |
| B21 | GRPC5D | CD3 | BCMA | CD38 |
| B22 | GRPC5D | CD3 | CD38 | BCMA |
| B23 | GRPC5D | CD38 | BCMA | CD3 |
| B24 | GRPC5D | CD38 | CD3 | BCMA |

TABLE 12
Anti-CD3/BCMA/GPRC5D/SLAMF7 tetraspecific configurations

| | Binding arm | Binding arm | Binding arm | Binding arm |
|---|---|---|---|---|
| Configuration | in A | in B | in C | in D |
| C1 | BCMA | CD3 | GRPC5D | SLAMF7 |
| C2 | BCMA | CD3 | SLAMF7 | GRPC5D |
| C3 | BCMA | SLAMF7 | CD3 | GRPC5D |
| C4 | BCMA | SLAMF7 | GRPC5D | CD3 |
| C5 | BCMA | GRPC5D | CD3 | SLAMF7 |
| C6 | BCMA | GRPC5D | SLAMF7 | CD3 |
| C7 | CD3 | BCMA | GRPC5D | SLAMF7 |
| C8 | CD3 | BCMA | SLAMF7 | GRPC5D |
| C9 | CD3 | SLAMF7 | GRPC5D | BCMA |
| C10 | CD3 | SLAMF7 | BCMA | GRPC5D |
| C11 | CD3 | GRPC5D | SLAMF7 | BCMA |
| C12 | CD3 | GRPC5D | BCMA | SLAMF7 |
| C13 | SLAMF7 | BCMA | CD3 | GPRC5D |
| C14 | SLAMF7 | BCMA | GPRC5D | CD3 |
| C15 | SLAMF7 | CD3 | BCMA | GPRC5D |
| C16 | SLAMF7 | CD3 | GPRC5D | BCMA |
| C17 | SLAMF7 | GRPC5D | BCMA | CD3 |
| C18 | SLAMF7 | GRPC5D | CD3 | BCMA |
| C19 | GRPC5D | BCMA | CD3 | SLAMF7 |
| C20 | GRPC5D | BCMA | SLAMF7 | CD3 |
| C21 | GRPC5D | CD3 | BCMA | SLAMF7 |
| C22 | GRPC5D | CD3 | SLAMF7 | BCMA |
| C23 | GRPC5D | SLAMF7 | BCMA | CD3 |
| C24 | GRPC5D | SLAMF7 | CD3 | BCMA |

TABLE 13
Anti-CD3/BCMA/CD38/SLAMF7 tetraspecific configurations

| | Binding arm | Binding arm | Binding arm | Binding arm |
|---|---|---|---|---|
| Configuration | in A | in B | in C | in D |
| D1 | BCMA | CD3 | CD38 | SLAMF7 |
| D2 | BCMA | CD3 | SLAMF7 | CD38 |
| D3 | BCMA | SLAMF7 | CD3 | CD38 |
| D4 | BCMA | SLAMF7 | CD38 | CD3 |
| D5 | BCMA | CD38 | CD3 | SLAMF7 |
| D6 | BCMA | CD38 | SLAMF7 | CD3 |
| D7 | CD3 | BCMA | CD38 | SLAMF7 |
| D8 | CD3 | BCMA | SLAMF7 | CD38 |
| D9 | CD3 | SLAMF7 | CD38 | BCMA |
| D10 | CD3 | SLAMF7 | BCMA | CD38 |
| D11 | CD3 | CD38 | SLAMF7 | BCMA |
| D12 | CD3 | CD38 | BCMA | SLAMF7 |
| D13 | SLAMF7 | BCMA | CD3 | CD38 |
| D14 | SLAMF7 | BCMA | CD38 | CD3 |
| D15 | SLAMF7 | CD3 | BCMA | CD38 |
| D16 | SLAMF7 | CD3 | CD38 | BCMA |
| D17 | SLAMF7 | CD38 | BCMA | CD3 |
| D18 | SLAMF7 | CD38 | CD3 | BCMA |
| D19 | CD38 | BCMA | CD3 | SLAMF7 |
| D20 | CD38 | BCMA | SLAMF7 | CD3 |
| D21 | CD38 | CD3 | BCMA | SLAMF7 |
| D22 | CD38 | CD3 | SLAMF7 | BCMA |
| D23 | CD38 | SLAMF7 | BCMA | CD3 |
| D24 | CD38 | SLAMF7 | CD3 | BCMA |

TABLE 14
Anti-CD3/GPRC5D/SLAMF7/CD38 tetraspecific configurations

| | Binding arm | Binding arm | Binding arm | Binding arm |
|---|---|---|---|---|
| Configuration | in A | in B | in C | in D |
| E1 | GPRC5D | CD3 | CD38 | SLAMF7 |
| E2 | GPRC5D | CD3 | SLAMF7 | CD38 |
| E3 | GPRC5D | SLAMF7 | CD3 | CD38 |
| E4 | GPRC5D | SLAMF7 | CD38 | CD3 |
| E5 | GPRC5D | CD38 | CD3 | SLAMF7 |
| E6 | GPRC5D | CD38 | SLAMF7 | CD3 |
| E7 | CD3 | GPRC5D | CD38 | SLAMF7 |
| E8 | CD3 | GPRC5D | SLAMF7 | CD38 |
| E9 | CD3 | SLAMF7 | CD38 | GPRC5D |
| E10 | CD3 | SLAMF7 | GPRC5D | CD38 |
| E11 | CD3 | CD38 | SLAMF7 | GPRC5D |
| E12 | CD3 | CD38 | GPRC5D | SLAMF7 |
| E13 | SLAMF7 | GPRC5D | CD3 | CD38 |
| E14 | SLAMF7 | GPRC5D | CD38 | CD3 |
| E15 | SLAMF7 | CD3 | GPRC5D | CD38 |
| E16 | SLAMF7 | CD3 | CD38 | GPRC5D |
| E17 | SLAMF7 | CD38 | GPRC5D | CD3 |
| E18 | SLAMF7 | CD38 | CD3 | GPRC5D |
| E19 | CD38 | GPRC5D | CD3 | SLAMF7 |
| E20 | CD38 | GPRC5D | SLAMF7 | CD3 |
| E21 | CD38 | CD3 | GPRC5D | SLAMF7 |
| E22 | CD38 | CD3 | SLAMF7 | GPRC5D |
| E23 | CD38 | SLAMF7 | GPRC5D | CD3 |
| E24 | CD38 | SLAMF7 | CD3 | GPRC5D |

TABLE 15
Anti-CD3/BCMA/CD38/FCRH5 tetraspecific configurations

| | Binding arm | Binding arm | Binding arm | Binding arm |
|---|---|---|---|---|
| Configuration | in A | in B | in C | in D |
| F1 | BCMA | CD3 | CD38 | FCRH5 |
| F2 | BCMA | CD3 | FCRH5 | CD38 |
| F3 | BCMA | FCRH5 | CD3 | CD38 |
| F4 | BCMA | FCRH5 | CD38 | CD3 |
| F5 | BCMA | CD38 | CD3 | FCRH5 |
| F6 | BCMA | CD38 | FCRH5 | CD3 |
| F7 | CD3 | BCMA | CD38 | FCRH5 |
| F8 | CD3 | BCMA | FCRH5 | CD38 |
| F9 | CD3 | FCRH5 | CD38 | BCMA |
| F10 | CD3 | FCRH5 | BCMA | CD38 |
| F11 | CD3 | CD38 | FCRH5 | BCMA |
| F12 | CD3 | CD38 | BCMA | FCRH5 |
| F13 | FCRH5 | BCMA | CD3 | CD38 |
| F14 | FCRH5 | BCMA | CD38 | CD3 |
| F15 | FCRH5 | CD3 | BCMA | CD38 |
| F16 | FCRH5 | CD3 | CD38 | BCMA |
| F17 | FCRH5 | CD38 | BCMA | CD3 |
| F18 | FCRH5 | CD38 | CD3 | BCMA |
| F19 | CD38 | BCMA | CD3 | FCRH5 |
| F20 | CD38 | BCMA | FCRH5 | CD3 |
| F21 | CD38 | CD3 | BCMA | FCRH5 |
| F22 | CD38 | CD3 | FCRH5 | BCMA |
| F23 | CD38 | FCRH5 | BCMA | CD3 |
| F24 | CD38 | FCRH5 | CD3 | BCMA |

TABLE 16
Anti-CD3/BCMA/SLAMF7/FCRH5 tetraspecific configurations

| | Binding arm | Binding arm | Binding arm | Binding arm |
|---|---|---|---|---|
| Configuration | in A | in B | in C | in D |
| G1 | BCMA | CD3 | SLAMF7 | FCRH5 |
| G2 | BCMA | CD3 | FCRH5 | SLAMF7 |
| G3 | BCMA | FCRH5 | CD3 | SLAMF7 |
| G4 | BCMA | FCRH5 | SLAMF7 | CD3 |
| G5 | BCMA | SLAMF7 | CD3 | FCRH5 |
| G6 | BCMA | SLAMF7 | FCRH5 | CD3 |
| G7 | CD3 | BCMA | SLAMF7 | FCRH5 |
| G8 | CD3 | BCMA | FCRH5 | SLAMF7 |
| G9 | CD3 | FCRH5 | SLAMF7 | BCMA |
| G10 | CD3 | FCRH5 | BCMA | SLAMF7 |
| G11 | CD3 | SLAMF7 | FCRH5 | BCMA |
| G12 | CD3 | SLAMF7 | BCMA | FCRH5 |
| G13 | FCRH5 | BCMA | CD3 | SLAMF7 |
| G14 | FCRH5 | BCMA | SLAMF7 | CD3 |
| G15 | FCRH5 | CD3 | BCMA | SLAMF7 |
| G16 | FCRH5 | CD3 | SLAMF7 | BCMA |
| G17 | FCRH5 | SLAMF7 | BCMA | CD3 |
| G18 | FCRH5 | SLAMF7 | CD3 | BCMA |
| G19 | SLAMF7 | BCMA | CD3 | FCRH5 |
| G20 | SLAMF7 | BCMA | FCRH5 | CD3 |
| G21 | SLAMF7 | CD3 | BCMA | FCRH5 |
| G22 | SLAMF7 | CD3 | FCRH5 | BCMA |
| G23 | SLAMF7 | FCRH5 | BCMA | CD3 |
| G24 | SLAMF7 | FCRH5 | CD3 | BCMA |

TABLE 17
Anti-CD3/GPRC5D/CD38/FCRH5 tetraspecific configurations

| | Binding arm | Binding arm | Binding arm | Binding arm |
|---|---|---|---|---|
| Configuration | in A | in B | in C | in D |
| H1 | GPRC5D | CD3 | CD38 | FCRH5 |
| H2 | GPRC5D | CD3 | FCRH5 | CD38 |
| H3 | GPRC5D | FCRH5 | CD3 | CD38 |
| H4 | GPRC5D | FCRH5 | CD38 | CD3 |
| H5 | GPRC5D | CD38 | CD3 | FCRH5 |
| H6 | GPRC5D | CD38 | FCRH5 | CD3 |
| H7 | CD3 | GPRC5D | CD38 | FCRH5 |
| H8 | CD3 | GPRC5D | FCRH5 | CD38 |
| H9 | CD3 | FCRH5 | CD38 | GPRC5D |
| H10 | CD3 | FCRH5 | GPRC5D | CD38 |
| H11 | CD3 | CD38 | FCRH5 | GPRC5D |
| H12 | CD3 | CD38 | GPRC5D | FCRH5 |
| H13 | FCRH5 | GPRC5D | CD3 | CD38 |
| H14 | FCRH5 | GPRC5D | CD38 | CD3 |
| H15 | FCRH5 | CD3 | GPRC5D | CD38 |
| H16 | FCRH5 | CD3 | CD38 | GPRC5D |
| H17 | FCRH5 | CD38 | GPRC5D | CD3 |
| H18 | FCRH5 | CD38 | CD3 | GPRC5D |
| H19 | CD38 | GPRC5D | CD3 | FCRH5 |
| H20 | CD38 | GPRC5D | FCRH5 | CD3 |
| H21 | CD38 | CD3 | GPRC5D | FCRH5 |
| H22 | CD38 | CD3 | FCRH5 | GPRC5D |
| H23 | CD38 | FCRH5 | GPRC5D | CD3 |
| H24 | CD38 | FCRH5 | CD3 | GPRC5D |

TABLE 18
Anti-CD3/GPRC5D/SLAMF7/FCRH5 tetraspecific configurations

| | Binding arm | Binding arm | Binding arm | Binding arm |
|---|---|---|---|---|
| Configuration | in A | in B | in C | in D |
| I1 | GPRC5D | CD3 | SLAMF7 | FCRH5 |
| I2 | GPRC5D | CD3 | FCRH5 | SLAMF7 |
| I3 | GPRC5D | FCRH5 | CD3 | SLAMF7 |
| I4 | GPRC5D | FCRH5 | SLAMF7 | CD3 |
| I5 | GPRC5D | SLAMF7 | CD3 | FCRH5 |
| I6 | GPRC5D | SLAMF7 | FCRH5 | CD3 |
| I7 | CD3 | GPRC5D | SLAMF7 | FCRH5 |
| I8 | CD3 | GPRC5D | FCRH5 | SLAMF7 |
| I9 | CD3 | FCRH5 | SLAMF7 | GPRC5D |
| I10 | CD3 | FCRH5 | GPRC5D | SLAMF7 |
| I11 | CD3 | SLAMF7 | FCRH5 | GPRC5D |
| I12 | CD3 | SLAMF7 | GPRC5D | FCRH5 |
| I13 | FCRH5 | GPRC5D | CD3 | SLAMF7 |
| I14 | FCRH5 | GPRC5D | SLAMF7 | CD3 |
| I15 | FCRH5 | CD3 | GPRC5D | SLAMF7 |
| I16 | FCRH5 | CD3 | SLAMF7 | GPRC5D |
| I17 | FCRH5 | SLAMF7 | GPRC5D | CD3 |
| I18 | FCRH5 | SLAMF7 | CD3 | GPRC5D |
| I19 | SLAMF7 | GPRC5D | CD3 | FCRH5 |
| I20 | SLAMF7 | GPRC5D | FCRH5 | CD3 |
| I21 | SLAMF7 | CD3 | GPRC5D | FCRH5 |
| I22 | SLAMF7 | CD3 | FCRH5 | GPRC5D |
| I23 | SLAMF7 | FCRH5 | GPRC5D | CD3 |
| I24 | SLAMF7 | FCRH5 | CD3 | GPRC5D |

TABLE 19
Anti-CD3/CD38/SLAMF7/FCRH5 tetraspecific configurations

| | Binding arm | Binding arm | Binding arm | Binding arm |
|---|---|---|---|---|
| Configuration | in A | in B | in C | in D |
| J1 | CD38 | CD3 | SLAMF7 | FCRH5 |
| J2 | CD38 | CD3 | FCRH5 | SLAMF7 |
| J3 | CD38 | FCRH5 | CD3 | SLAMF7 |
| J4 | CD38 | FCRH5 | SLAMF7 | CD3 |
| J5 | CD38 | SLAMF7 | CD3 | FCRH5 |
| J6 | CD38 | SLAMF7 | FCRH5 | CD3 |
| J7 | CD3 | CD38 | SLAMF7 | FCRH5 |
| J8 | CD3 | CD38 | FCRH5 | SLAMF7 |
| J9 | CD3 | FCRH5 | SLAMF7 | CD38 |
| J10 | CD3 | FCRH5 | CD38 | SLAMF7 |
| J11 | CD3 | SLAMF7 | FCRH5 | CD38 |
| J12 | CD3 | SLAMF7 | CD38 | FCRH5 |
| J13 | FCRH5 | CD38 | CD3 | SLAMF7 |
| J14 | FCRH5 | CD38 | SLAMF7 | CD3 |
| J15 | FCRH5 | CD3 | CD38 | SLAMF7 |
| J16 | FCRH5 | CD3 | SLAMF7 | CD38 |
| J17 | FCRH5 | SLAMF7 | CD38 | CD3 |
| J18 | FCRH5 | SLAMF7 | CD3 | CD38 |
| J19 | SLAMF7 | CD38 | CD3 | FCRH5 |
| J20 | SLAMF7 | CD38 | FCRH5 | CD3 |
| J21 | SLAMF7 | CD3 | CD38 | FCRH5 |
| J22 | SLAMF7 | CD3 | FCRH5 | CD38 |
| J23 | SLAMF7 | FCRH5 | CD38 | CD3 |
| J24 | SLAMF7 | FCRH5 | CD3 | CD38 |

TABLE 20
Leader Sequences for use with
heavy chains and light chains

| SEQ ID NO: | Leader Sequence |
|---|---|
| 70 | MDPKGSLSWRILLFLSLAFELSYG |
| 71 | METDTLLLWVLLLWVPGSTG |
| 72 | MGWSLILLFLVAVATRVHS |
| 73 | MRVPAQLLGLLLLWLPGARC |
| 74 | MKLWLNWVFLLTLLHGIQC |
| 75 | MDFQVQIFSFLLISASVIVSRG |

TABLE 21
Linker Sequences for use in modified heavy
chains and/or modified light chains

| SEQ ID NO: | Linker Sequence |
|---|---|
| 76 | GGGGS_{(n; wherein n is between 1-6)} |
| 77 | GGGGSGGGGSGGGGS |
| 78 | GGGGSGGGGSGGGGSGGGGS |
| 79 | GSGSGS |
| 80 | GGSGGSGGSGG |
| 81 | GGGGSLVPRGSGGGGS |
| 82 | GGSGGHMGSGG |
| 83 | GGSGG |
| 84 | GGSSG |
| 85 | GSGSGSGSGGSG |

In some embodiments, the antibody-based multispecific binder molecule comprises one, two, three, or four light chains. In some embodiments, the antibody-based multispecific binder molecule comprises four light chains. In some embodiments, the one or more light chains comprise one or more light chain constant regions. In some embodiments, the light chain constant region is a kappa light chain constant region. In some embodiments, the light chain constant region is a lambda light chain constant region. In some embodiments, the light chain constant region is a human kappa light chain constant region. In some embodiments, the light chain constant region is a human lambda light chain constant region. In some embodiments, the light chain constant region is a mouse kappa light chain constant region. In some embodiments, the light chain constant region is a mouse lambda light chain constant region. In some embodiments, the light chain constant region is a rat kappa light chain constant region. In some embodiments, the light chain constant region is a rat lambda light chain constant region. In some embodiments, amino acid sequences of the human kappa light chain constant region is listed in Table 9. In some embodiments, the human kappa light chain constant region comprises an amino acid sequence of SEQ ID NO: 69.

In some embodiments, an antibody-based multispecific binder molecule described herein comprises a full-length antibody, comprising a heavy chain (HC) and a light chain (LC). In some embodiments, the HC comprises about 80%, 85%, 90%, 95%, 96% 97%, 98%, 99%, or 100% sequence identity to a sequence selected from SEQ ID NOs: 21-34. In some embodiments, the HC comprises at least 80%, at least 85%, at least 90%, or at least 95% sequence identity sequence identity to a sequence selected from SEQ ID NOs: 28-34. In some embodiments, the LC comprises about 80%, 85%, 90%, 95%, 96% 97%, 98%, 99%, or 100% sequence identity to a sequence selected from SEQ ID NOs: 35-46. In some embodiments, the LC comprises at least 80%, at least 85%, at least 90%, or at least 95% sequence identity sequence identity to a sequence selected from SEQ ID NOs: 35-46. In some cases, the heavy chain (HC) comprises an amino acid sequence selected from a sequence listed in Table 5. In some cases, the light chain (LC) comprises an amino acid sequence selected from a sequence listed in Table 6. In some cases, two modified heavy chains comprising a sequence listed in Table 5 and at least one LC comprising a sequence listed in Table 6 are paired together to form the antibody-based multispecific binder molecule.

In some embodiments, an antibody-based multispecific binder molecule described herein comprises at least four VH domains and at least four VL domains. In some embodiments, at least one of the four VH domains comprises about 80%, 85%, 90%, 95%, 96% 97%, 98%, 99%, or 100% sequence identity to a sequence selected from SEQ ID NOs: 1-11. In some embodiments, at least two of the four VH domains each comprises about 80%, 85%, 90%, 95%, 96% 97%, 98%, 99%, or 100% sequence identity to a sequence selected from SEQ ID NOs: 1-11. In some embodiments, at least three of the four VH domains each comprises about 80%, 85%, 90%, 95%, 96% 97%, 98%, 99%, or 100% sequence identity to a sequence selected from SEQ ID NOs: 1-11. In some embodiments, at least one of the four VH domains comprises about 80%, 85%, 90%, 95%, 96% 97%, 98%, 99%, or 100% sequence identity to the VH domain region of a sequence selected from SEQ ID NOs: 21-34. In some embodiments, at least two of the four VH domains each comprise about 80%, 85%, 90%, 95%, 96% 97%, 98%, 99%, or 100% sequence identity to a VH domain region of a sequence selected from SEQ ID NOs: 21-34. In some embodiments, at least three of the four VH domains each comprise about 80%, 85%, 90%, 95%, 96% 97%, 98%, 99%, or 100% sequence identity to a VH domain region of a sequence selected from SEQ ID NOs: 21-34. In some embodiments, at least one of the four VL domains comprises about 80%, 85%, 90%, 95%, 96% 97%, 98%, 99%, or 100% sequence identity to a sequence selected from SEQ ID NOs: 12-20. In some embodiments, at least two of the four VL domains each comprise about 80%, 85%, 90%, 95%, 96% 97%, 98%, 99%, or 100% sequence identity to a sequence selected from SEQ ID NOs: 12-20. In some embodiments, at least three of the four VL domains each comprise about 80%, 85%, 90%, 95%, 96% 97%, 98%, 99%, or 100% sequence identity to a sequence selected from SEQ ID NOs: 12-20. In some embodiments, at least one of the four VL domains comprises about 80%, 85%, 90%, 95%, 96% 97%, 98%, 99%, or 100% sequence identity to a VL domain region of a sequence selected from SEQ ID NOs: 35-46. In some embodiments, at least two of the four VL domains each comprise about 80%, 85%, 90%, 95%, 96% 97%, 98%, 99%, or 100% sequence identity to a VL domain region of a sequence selected from SEQ ID NOs: 35-46. In some embodiments, at least three of the four VL domains each comprise about 80%, 85%, 90%, 95%, 96% 97%, 98%, 99%, or 100% sequence identity to a VL domain region of a sequence selected from SEQ ID NOs: 35-46. In some embodiments, an antigen binding regions of the multispecific binding molecule comprises a sequence of great than 80 amino acids that is 80%, 85%, 90%, 95%, 96% 97%, 98%, 99%, or 100% identical to a portion of a SEQ ID NO: 47. In some embodiments, an antigen binding regions of the multispecific binding molecule comprises a sequence of great than 80 amino acids that is 80%, 85%, 90%, 95%, 96% 97%, 98%, 99%, or 100% identical to a portion of a SEQ ID NO: 48.

### Production and Manufacture of Antibodies or Antigen-Binding Fragments Thereof

In some embodiments, polypeptides described herein (e.g., antibodies or antigen-binding fragments thereof) are produced using any method known in the art to be useful for the synthesis of polypeptides (e.g., antibodies), in particular, by chemical synthesis or by recombinant expression, and are preferably produced by use of a recombinant expression technique.

In some instances, an antibody or antigen-binding fragment thereof is expressed recombinantly, and the nucleic acid encoding the antibody or its antigen-binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 5′ and 3′ ends of the sequence or by cloning using an oligonucleotide specific for the particular nucleic acid sequence.

In some instances, an antibody or its binding is optionally made by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937.

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region, e.g., humanized antibodies.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, a nucleic acid sequence encodes the antibodies disclosed herein. In some embodiments, the polynucleotide sequence encoding the antibodies is operatively coupled to a eukaryotic regulatory sequence. In some embodiments, a cell comprises the nucleic acid sequence. In some embodiments, a cell comprises a nucleic acid encoding the antibodies disclosed herein. In some embodiments, the cell comprises a prokaryotic cell. In some embodiments, the prokaryotic cell is an Escherichia coli cell. In some embodiments, the cell comprises a eukaryotic cell. In some embodiments, the eukaryotic cell is a Chines Hamster Ovary (CHO) cell, a HEK293 cell, a BHK cell, an NS0 murine myeloma cell, or a PER.C6® human cell. In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific aspects, the expression of the antibody is regulated by a constitutive, an inducible or a tissue, specific promoter. Standard cell lines and methods for the production of antibodies from a large-scale cell culture are known in the art. See e.g., Li et al., “Cell culture processes for monoclonal antibody production.” Mabs. 2010 September-Oct; 2 (5): 466-477.

In certain aspects, described herein is a method of making antibodies comprising culturing a cell comprising a nucleic acid encoding a antibodies under conditions in vitro sufficient to allow production and secretion of the antibodies. In some embodiments, antibodies are harvested from the cell culture medium. The harvesting can further comprise one or more purification steps to remove live cells, cellular debris, non-antibody proteins or polypeptides, undesired salts, buffers, and medium components. In certain aspects, the additional purification step(s) include centrifugation, ultracentrifugation, protein A, protein G, protein A/G, or protein L purification, and/or ion exchange chromatography.

### Non-Antibody Based Multi-Specific Binders

In some aspects, the multi-specific binder comprises a non-antibody based binder that binds to a tumor associated antigen or binds to a molecule expressed on an immune effector cell. In some embodiments, the tumor associated antigen is GPRC5D, BCMA, FcRH5, CD38, or SLAMF7. In some embodiments, the molecule expressed on an immune effector cell is CD3. In some embodiments, the molecule expressed on an immune effector cell is one of the elements comprising the CD3 receptor complex. In some embodiments, the multi-specific binder comprises one or more non-antibody based binders and one or more antibody based binders. In some embodiments, the multi-specific binder comprises one non-antibody based binder and at least one antibody based binder. In some embodiments, the multi-specific binder comprises two non-antibody based binders. In some embodiments, the multi-specific binder comprises two non-antibody based binders and at least one antibody based binder. In some embodiments, the multi-specific binder comprises three non-antibody based binders. In some embodiments, the multi-specific binder comprises three non-antibody based binders and one antibody based binder. In some embodiments, the multi-specific binder comprises four non-antibody based binders. In some embodiments, the multi-specific binder comprises a non-antibody based binder that binds GPRC5D. In some embodiments, the multi-specific binder comprises a non-antibody based binder that binds BCMA. In some embodiments, the multi-specific binder comprises a non-antibody based binder that binds FcRH5. In some embodiments, the multi-specific binder comprises a non-antibody based binder that binds CD38. In some embodiments, the multi-specific binder comprises a non-antibody based binder that binds SLAMF7. In some embodiments, the multi-specific binder comprises a non-antibody based binder that binds CD3. In some embodiments, the multi-specific binder comprises two non-antibody based binders, each non-antibody based binder capable of binding specifically to any one of GPRC5D, BCMA, FcRH5, CD38, SLAMF7, or CD3. In some embodiments, the multi-specific binder comprises three non-antibody based binders, each non-antibody based binder capable of binding specifically to any one of GPRC5D, BCMA, FcRH5, CD38, SLAMF7, or CD3. In some embodiments, the multi-specific binder comprises four non-antibody based binders, each non-antibody based binder capable of binding specifically to any one of GPRC5D, BCMA, FcRH5, CD38, SLAMF7, or CD3. In some embodiments, the multi-specific binder comprises four non-antibody based binders comprising: i) a GPRC5D binding moiety, ii) a BCMA binding moiety, iii) an FcRH5 binding moiety, and iv) a CD3 binding moiety. In some embodiments, the multi-specific binder comprises four non-antibody based binders comprising: i) a GPRC5D binding moiety, ii) a BCMA binding moiety, iii) a CD38 binding moiety, and iv) a CD3 binding moiety. In some embodiments, the multi-specific binder comprises four non-antibody based binders comprising: i) a GPRC5D binding moiety, ii) a BCMA binding moiety, iii) a SLAMF7 binding moiety, and iv) a CD3 binding moiety. In some embodiments, the multi-specific binder comprises four non-antibody based binders comprising: i) a GPRC5D binding moiety, ii) a SLAMF7 binding moiety, iii) a CD38 binding moiety, and iv) a CD3 binding moiety. In some embodiments, the multi-specific binder comprises four non-antibody based binders comprising: i) a SLAMF7 binding moiety, ii) a BCMA binding moiety, iii) a CD38 binding moiety, and iv) a CD3 binding moiety. In some embodiments, the GPRC5D binding moiety enhances binding of the multispecific binder molecule to plasma B cells. In some embodiments, the GPRC5D binding moiety enhances binding of the multispecific binder molecule to malignant plasma B cells. In some embodiments, the BCMA binding moiety enhances binding of the multispecific binder molecule to plasma B cells. In some embodiments, the BCMA binding moiety enhances binding of the multispecific binder molecule to malignant plasma B cells. In some embodiments, the FcRH5 binding moiety enhances binding of the multispecific binder molecule to plasma B cells. In some embodiments, the FcRH5 binding moiety enhances binding of the multispecific binder molecule to malignant plasma B cells. In some embodiments, the CD38 binding moiety enhances binding of the multispecific binder molecule to plasma B cells. In some embodiments, the CD38 binding moiety enhances binding of the multispecific binder molecule to malignant plasma B cells. In some embodiments, the SLAMF7 binding moiety enhances binding of the multispecific binder molecule to plasma B cells. In some embodiments, the SLAMF7 binding moiety enhances binding of the multispecific binder molecule to malignant plasma B cells. In some embodiments, the immune cell binder binds to CD3. In some embodiments, the immune cell engager binds to CD3. In some embodiments, the immune cell binder binding to CD3 functions as an immune cell engager. In some embodiments, the BCMA binding moiety, the GPRC5D binding moiety, and the FcRH5 binding moiety enhance binding of the multispecific binder molecule to yield a higher binding affinity to a population of plasma B cells compared to a multispecific binder molecule comprising any two of a BCMA binding moiety, a GPRC5D binding moiety, or an FcRH5 binding moiety. In some embodiments, the BCMA binding moiety, the GPRC5D binding moiety, and the SLAMF7 binding moiety enhance binding of the multispecific binder molecule to yield a higher binding affinity to a population of plasma B cells compared to a multispecific binder molecule comprising any two of a BCMA binding moiety, a GPRC5D binding moiety, or a SLAMF7 binding moiety. In some embodiments, the BCMA binding moiety, the GPRC5D binding moiety, and the CD38 binding moiety enhance binding of the multispecific binder molecule to yield a higher binding affinity to a population of plasma B cells compared to a multispecific binder molecule comprising any two of a BCMA binding moiety, a GPRC5D binding moiety, or a CD38 binding moiety. In some embodiments, the BCMA binding moiety, the CD38 binding moiety, and the SLAMF7 binding moiety enhance binding of the multispecific binder molecule to yield a higher binding affinity to a population of plasma B cells compared to a multispecific binder molecule comprising any two of a BCMA binding moiety, a CD38 binding moiety, or a SLAMF7 binding moiety. In some embodiments, the CD38 binding moiety, the GPRC5D binding moiety, and the SLAMF7 binding moiety enhance binding of the multispecific binder molecule to yield a higher binding affinity to a population of plasma B cells compared to a multispecific binder molecule comprising any two of a CD38 binding moiety, a GPRC5D binding moiety, or a SLAMF7 binding moiety.

Provided herein are non-antibody-based binders that bind to CD3. In some embodiments, the CD3 binding moiety enhances binding of the multispecific binder molecule to T cells, NKT cells, or T cells and NKT cells. In some embodiments, the CD3 binding moiety engages one or more classes of immune cells. In some embodiments, the CD3 binding moiety allows the multispecific binder molecule to simultaneously bind one or more T cells or NKT cells as binding one or more plasma cells. In some embodiments, the CD3 binding moiety allows the multispecific binder molecule to simultaneously bind one or more T cells or NKT cells as binding one or more malignant plasma cells. In some embodiments, the CD3 binding moiety allows the multispecific binder molecule to simultaneously bind to a T cell or an NKT cell and a tumor cells expressing any one of BCMA, CD38, FcRH5, SLAMF7, or GPRC5D. In some embodiments, the CD3 binding moiety allows the multispecific binder molecule to simultaneously bind to a T cell or an NKT cell and a tumor cells expressing any two of BCMA, CD38, FcRH5, SLAMF7, or GPRC5D. In some embodiments, the CD3 binding moiety allows the multispecific binder molecule to simultaneously bind to a T cell or an NKT cell and a tumor cells expressing at least three of BCMA, CD38, FcRH5, SLAMF7, or GPRC5D. In some embodiments, the tumor cells are from a hematological cancer. In some embodiments, the tumor cells are from myeloma. In some embodiments, the tumor cells are from multiple myeloma. In some embodiments, the multispecific binder molecule brings T cells closer in proximity to tumor cells. In some embodiments, the multispecific binder molecule brings T cells closer in proximity to tumor cells in a BM microenvironment. In some embodiments, the multispecific binder molecule brings NK cells closer in proximity to tumor cells. In some embodiments, the multispecific binder molecule brings NKT cells closer in proximity to tumor cells.

Nucleic acid aptamers are short, single-stranded oligonucleotides (ssDNA, ssRNA, or ssXNA) able to bind selectively to a specific target such as a protein, a peptide, a carbohydrate, a toxin, or a live cell. In some embodiments, the tumor associated antigen binding moiety (e.g., GPRC5D binding moiety, BCMA binding moiety, FcRH5 binding moiety, SLAMF7 binding moiety, CD38 binding moiety) and/or the immune cell binding moiety (e.g., CD3 binding moiety) is a nucleic acid aptamer. In some embodiments, the nucleic acid aptamer is based on an oligonucleotide structure with specificity and affinity for GPRC5D, BCMA, FcRH5, CD38, SLAMF7, or CD3 similar to that of an anti-GPRC5D antibody, an anti-BCMA antibody, an anti-FcRH5 antibody, an anti-CD38 antibody, an anti-SLAMF7 antibody, an anti-CD3 antibody, respectively.

Peptide aptamers are small combinatorial proteins, usually between 5-20 amino acid residues in length, that are capable of selective binding to a specific site or sites on a target molecule. In some embodiments, the tumor associated antigen binding moiety (e.g., GPRC5D binding moiety, BCMA binding moiety, FcRH5 binding moiety, SLAMF7 binding moiety, CD38 binding moiety) and/or the immune cell binding moiety (e.g., CD3 binding moiety) is a peptide aptamer. In some embodiments, the peptide aptamer is based on an peptide structure with specificity and affinity for GPRC5D, BCMA, FcRH5, CD38, SLAMF7, or CD3 similar to that of an anti-GPRC5D antibody, an anti-BCMA antibody, an anti-FcRH5 antibody, an anti-CD38 antibody, an anti-SLAMF7 antibody, an anti-CD3 antibody, respectively. In some embodiments, the peptide aptamer is selected from a peptide aptamer library in which a randomized set of peptide sequences which have been engineered into a stable protein scaffold by screening the library using yeast or mammalian cells or by phage display technology. In some embodiments, the GPRC5D binding peptide aptamer is included as a part of a tetraspecific binder molecule that also binds to at least three of either BCMA, CD38, SLAMF7, or CD3. In some embodiments, the BCMA binding peptide aptamer is included as a part of a tetraspecific binder molecule that also binds to at least three of either GPRC5D, CD38, SLAMF7, or CD3. In some embodiments, the FcRH5 binding peptide aptamer is included as a part of a tetraspecific binder molecule that also binds to at least three of either GPRC5D, BCMA, CD38, SLAMF7, or CD3. In some embodiments, the CD38 binding peptide aptamer is included as a part of a tetraspecific binder molecule that also binds to at least three of either GPRC5D, BCMA, SLAMF7, or CD3. In some embodiments, the SLAMF7 binding peptide aptamer is included as a part of a tetraspecific binder molecule that also binds to at least three of either GPRC5D, CD38, BCMA, or CD3. In some embodiments, the CD3 binding peptide aptamer is included as a part of a tetraspecific binder molecule that also binds to at least three of either GPRC5D, FcRH5, CD38, SLAMF7, or BCMA.

Designed Ankyrin Repeat Proteins (DARPins) are engineered antibody mimetic proteins derived from an ankyrin repeat binding protein. In some embodiments, non-natural amino acids are incorporated into the DARPin. In some embodiments, the GPRC5D binding moiety is a Designed Ankyrin Repeat Protein (DARPin). In some embodiments, the BCMA binding moiety is a DARPin. In some embodiments, the FcRH5 binding moiety is a DARPin. In some embodiments, the SLAMF7 binding moiety is a DARPin. In some embodiments, the CD38 binding moiety is a DARPin. In some embodiments, the CD3 binding moiety is a DARPin. In some embodiments, the DARPin is selected using either phage or ribosome display. In some embodiments, the GPRC5D binding DARPin is included as a part of a tetraspecific binder molecule that also binds to CD3, and at least two of either BCMA, FcRH5, SLAMF7, or CD38. In some embodiments, the BCMA binding DARPin is included as a part of a tetraspecific binder molecule that also binds to CD3, and at least two of either GPRC5D, FcRH5, SLAMF7, or CD38. In some embodiments, the FcRH5 binding DARPin is included as a part of a tetraspecific binder molecule that also binds to CD3, and at least two of either GPRC5D, BCMA, SLAMF7, or CD38. In some embodiments, the SLAMF7 binding DARPin is included as a part of a tetraspecific binder molecule that also binds to CD3, and at least two of either BCMA, GPRC5D, or CD38. In some embodiments, the CD38 binding DARPin is included as a part of a tetraspecific binder molecule that also binds to CD3, and at least two of either BCMA, SLAMF7, or GPRC5D. In some embodiments, the CD3 binding DARPin is included as a part of a tetraspecific binder molecule that binds to at least three of either BCMA, SLAMF7, FcRH5, GPRC5D, or CD38.

Affimers are small, engineered proteins which bind to target proteins with affinity in the nanomolar range. In some embodiments, phage display technology is used to select for an affimer that binds to an extracellular region of either GPRC5D, BCMA, FcRH5, SLAMF7, CD38, or CD3. In some embodiments, selecting an affimer using yeast display provides affimer binders that possess one or more post-translational modifications. Identified affimers can be generated using recombinant technology. In some embodiments, use of a bacterial host system to produce a recombinant affimer can yield protein titers in the range of 200-400 mg/L. In some embodiments, one or more affimers are conjugated to a multispecific binder molecule. In some embodiments, one or more affimer sequences are included in a peptide that is used to form a multispecific binder molecule. In some embodiments, the GPRC5D binding moiety is an affimer. In some embodiments, the BCMA binding moiety is an affimer. In some embodiments, the FcRH5 binding moiety is an affimer. In some embodiments, the SLAMF7 binding moiety is an affimer. In some embodiments, the CD38 binding moiety is an affimer. In some embodiments, the CD3 binding moiety is an affimer. In some embodiments, the GPRC5D binding affimer is included as a part of a tetraspecific binder molecule that also binds to CD3, and at least two of either BCMA, FcRH5, SLAMF7, or CD38. In some embodiments, the BCMA binding affimer is included as a part of a tetraspecific binder molecule that also binds to CD3, and at least two of either GPRC5D, FcRH5, SLAMF7, or CD38. In some embodiments, the FcRH5 binding affimer is included as a part of a tetraspecific binder molecule that also binds to CD3, and at least two of either GPRC5D, BCMA, SLAMF7, or CD38. In some embodiments, the SLAMF7 binding affimer is included as a part of a tetraspecific binder molecule that also binds to CD3, and at least two of either BCMA, GPRC5D, or CD38. In some embodiments, the CD38 binding affimer is included as a part of a tetraspecific binder molecule that also binds to CD3, and at least two of either BCMA, SLAMF7, or GPRC5D. In some embodiments, the CD3 binding affimer is included as a part of a tetraspecific binder molecule that binds to at least three of either BCMA, SLAMF7, FcRH5, GPRC5D, or CD38.

An inhibitor cysteine knot (knottin) is a protein structural motif that contains of one of three folds in a cysteine knot motif. In some embodiments, the knottin comprises a loop region of variable length that is engineered to bind to either GPRC5D, BCMA, FcRH5, SLAMF7, CD38, or CD3. In some embodiments, the GPRC5D binding moiety is a knottin. In some embodiments, the BCMA binding moiety is a knottin. In some embodiments, the FcRH5 binding moiety is a knottin. In some embodiments, the SLAMF7 binding moiety is a knottin. In some embodiments, the CD38 binding moiety is a knottin. In some embodiments, the CD3 binding moiety is a knottin. In some embodiments, the GPRC5D binding knottin is included as a part of a tetraspecific binder molecule that also binds to CD3, and at least two of either BCMA, FcRH5, SLAMF7, or CD38. In some embodiments, the BCMA binding knottin is included as a part of a tetraspecific binder molecule that also binds to CD3, and at least two of either GPRC5D, FcRH5, SLAMF7, or CD38. In some embodiments, the FcRH5 binding knottin is included as a part of a tetraspecific binder molecule that also binds to CD3, and at least two of either GPRC5D, BCMA, SLAMF7, or CD38. In some embodiments, the SLAMF7 binding knottin is included as a part of a tetraspecific binder molecule that also binds to CD3, and at least two of either BCMA, GPRC5D, or CD38. In some embodiments, the CD38 binding knottin is included as a part of a tetraspecific binder molecule that also binds to CD3, and at least two of either BCMA, SLAMF7, or GPRC5D. In some embodiments, the CD3 binding knottin is included as a part of a tetraspecific binder molecule that binds to at least three of either BCMA, SLAMF7, FcRH5, GPRC5D, or CD38.

An avimer (short or avidity multimer) is an engineered protein capable of specifically binding to particular antigens via multiple binding sites. In some embodiments, the avimer is selected by selecting for optimal binders to either GPRC5D, BCMA, FcRH5, SLAMF7, CD38, or CD3 using phage display to screen an avimer library. In some embodiments, the GPRC5D binding moiety is an avimer. In some embodiments, the BCMA binding moiety is an avimer. In some embodiments, the FcRH5 binding moiety is an avimer. In some embodiments, the SLAMF7 binding moiety is an avimer. In some embodiments, the CD38 binding moiety is an avimer. In some embodiments, the CD3 binding moiety is an avimer. In some embodiments, the GPRC5D binding avimer is included as a part of a tetraspecific binder molecule that also binds to CD3, and at least two of either BCMA, FcRH5, SLAMF7, or CD38. In some embodiments, the BCMA binding avimer is included as a part of a tetraspecific binder molecule that also binds to CD3, and at least two of either GPRC5D, FcRH5, SLAMF7, or CD38. In some embodiments, the FcRH5 binding avimer is included as a part of a tetraspecific binder molecule that also binds to CD3, and at least two of either GPRC5D, BCMA, SLAMF7, or CD38. In some embodiments, the SLAMF7 binding avimer is included as a part of a tetraspecific binder molecule that also binds to CD3, and at least two of either BCMA, GPRC5D, or CD38. In some embodiments, the CD38 binding avimer is included as a part of a tetraspecific binder molecule that also binds to CD3, and at least two of either BCMA, SLAMF7, or GPRC5D. In some embodiments, the CD3 binding avimer is included as a part of a tetraspecific binder molecule that binds to at least three of either BCMA, FcRH5, SLAMF7, GPRC5D, or CD38.

Monobodies are synthetic, engineered binding proteins constructed using a fibronectin type III domain (FN3) as a scaffold structure. In some embodiments, a library of monobodies is screened for specific binding to either GPRC5D, BCMA, FcRH5, SLAMF7, CD38, or CD3 using either phage display or yeast display technology. In some embodiments, the monobody is expressed in Escherichia coli (E. coli). In some embodiments, the monobody is expressed in a mammalian cell. In some embodiments, the GPRC5D binding moiety is a monobody. In some embodiments, the BCMA binding moiety is a monobody. In some embodiments, the FcRH5 binding moiety is a monobody. In some embodiments, the SLAMF7 binding moiety is a monobody. In some embodiments, the CD38 binding moiety is a monobody. In some embodiments, the CD3 binding moiety is a monobody. In some embodiments, the GPRC5D binding monobody is included as a part of a tetraspecific binder molecule that also binds to CD3, and at least two of either BCMA, FcRH5, SLAMF7, or CD38. In some embodiments, the BCMA binding monobody is included as a part of a tetraspecific binder molecule that also binds to CD3, and at least two of either GPRC5D, FcRH5, SLAMF7, or CD38. In some embodiments, the FcRH5 binding monobody is included as a part of a tetraspecific binder molecule that also binds to CD3, and at least two of either GPRC5D, BCMA, SLAMF7, or CD38. In some embodiments, the SLAMF7 binding monobody is included as a part of a tetraspecific binder molecule that also binds to CD3, and at least two of either BCMA, GPRC5D, or CD38. In some embodiments, the CD38 binding monobody is included as a part of a tetraspecific binder molecule that also binds to CD3, and at least two of either BCMA, SLAMF7, or GPRC5D. In some embodiments, the CD3 binding monobody is included as a part of a tetraspecific binder molecule that binds to at least three of either BCMA, FcRH5, SLAMF7, GPRC5D, or CD38.

Anticalins are engineered artificial proteins, not structurally related to antibodies, capable of binding to protein antigens or small molecules. In some embodiments, the GPRC5D binding moiety is an anticalin. In some embodiments, the BCMA binding moiety is an anticalin. In some embodiments, the FcRH5 binding moiety is an anticalin. In some embodiments, the CD38 binding moiety is an anticalin. In some embodiments, the SLAMF7 binding moiety is an anticalin. In some embodiments, the CD3 binding moiety is an anticalin. In some embodiments, the GPRC5D binding anticalin is included as a part of a tetraspecific binder molecule that also binds to CD3, and at least two of either BCMA, FcRH5, SLAMF7, or CD38. In some embodiments, the BCMA binding anticalin is included as a part of a tetraspecific binder molecule that also binds to CD3, and at least two of either GPRC5D, FcRH5, SLAMF7, or CD38. In some embodiments, the FcRH5 binding anticalin is included as a part of a tetraspecific binder molecule that also binds to CD3, and at least two of either GPRC5D, BCMA, SLAMF7, or CD38. In some embodiments, the SLAMF7 binding anticalin is included as a part of a tetraspecific binder molecule that also binds to CD3, and at least two of either BCMA, GPRC5D, or CD38. In some embodiments, the CD38 binding anticalin is included as a part of a tetraspecific binder molecule that also binds to CD3, and at least two of either BCMA, SLAMF7, or GPRC5D. In some embodiments, the CD3 binding anticalin is included as a part of a tetraspecific binder molecule that binds to at least three of either BCMA, FcRH5, SLAMF7, GPRC5D, or CD38.

Fynomers are small binding proteins comprising a human tyrosine kinase Src Homology 3 (SH3) domain derived from human Fyn. In some embodiments, the GPRC5D binding moiety is a fynomer. In some embodiments, the BCMA binding moiety is a fynomer. In some embodiments, the FcRH5 binding moiety is a fynomer. In some embodiments, the SLAMF7 binding moiety is a fynomer. In some embodiments, the CD38 binding moiety is a fynomer. In some embodiments, the CD3 binding moiety is a fynomer. In some embodiments, the GPRC5D binding fynomer is included as a part of a tetraspecific binder molecule that also binds to CD3, and at least two of either BCMA, FcRH5, SLAMF7, or CD38. In some embodiments, the BCMA binding fynomer is included as a part of a tetraspecific binder molecule that also binds to CD3, and at least two of either GPRC5D, FcRH5, SLAMF7, or CD38. In some embodiments, the FcRH5 binding fynomer is included as a part of a tetraspecific binder molecule that also binds to CD3, and at least two of either GPRC5D, BCMA, SLAMF7, or CD38. In some embodiments, the SLAMF7 binding fynomer is included as a part of a tetraspecific binder molecule that also binds to CD3, and at least two of either BCMA, GPRC5D, or CD38. In some embodiments, the CD38 binding fynomer is included as a part of a tetraspecific binder molecule that also binds to CD3, and at least two of either BCMA, SLAMF7, or GPRC5D. In some embodiments, the CD3 binding fynomer is included as a part of a tetraspecific binder molecule that binds to at least three of either BCMA, FcRH5, SLAMF7, GPRC5D, or CD38.

Affibodies are small proteins engineered to bind to a target protein antigen with high affinity and selectivity. In some embodiments, affibodies are screened and selected for specific, high affinity binding to either GPRC5D, BCMA, FcRH5, SLAMF7, CD38, or CD3. In some embodiments, the GPRC5D binding moiety is an affibody. In some embodiments, the BCMA binding moiety is an affibody. In some embodiments, the FcRH5 binding moiety is an affibody. In some embodiments, the SLAMF7 binding moiety is an affibody. In some embodiments, the CD38 binding moiety is an affibody. In some embodiments, the CD3 binding moiety is an affibody. In some embodiments, the GPRC5D binding affibody is included as a part of a tetraspecific binder molecule that also binds to CD3, and at least two of either BCMA, FcRH5, SLAMF7, or CD38. In some embodiments, the BCMA binding affibody is included as a part of a tetraspecific binder molecule that also binds to CD3, and at least two of either GPRC5D, FcRH5, SLAMF7, or CD38. In some embodiments, the FcRH5 binding affibody is included as a part of a tetraspecific binder molecule that also binds to CD3, and at least two of either GPRC5D, BCMA, SLAMF7, or CD38. In some embodiments, the SLAMF7 binding affibody is included as a part of a tetraspecific binder molecule that also binds to CD3, and at least two of either BCMA, GPRC5D, or CD38. In some embodiments, the CD38 binding affibody is included as a part of a tetraspecific binder molecule that also binds to CD3, and at least two of either BCMA, SLAMF7, or GPRC5D. In some embodiments, the CD3 binding affibody is included as a part of a tetraspecific binder molecule that binds to at least three of either BCMA, FcRH5, SLAMF7, GPRC5D, or CD38.

In some embodiments, non-antibody based binders are selected by phage display. Using phage display technology, highly variable sequence library are presented as polypeptides on filamentous phage surfaces to enable selection of binders with specificity and high affinity toward a target protein (e.g., GPRC5D, BCMA, FcRH5, SLAMF7, CD38, or CD3). Selection of non-antibody-based binders using phage display involves biopanning. Biopanning includes the exposure of a phage library to a target or a selected region of the target followed by multiple steps of washing, elution, and amplification to obtain and then identify the most suitable binders for the target. In some embodiments, a non-antibody-based binder can be combined with antibody-based binders comprising a second binding moiety, a third binding moiety, and an immune cell engager to form the multispecific binder molecule.

### Methods for Multiple Myeloma (MM) Treatment Using a Multispecific Binder Molecule

Disclosed herein, in certain aspects, are multispecific binder molecules for use in methods for treating MM. Also disclosed herein, in certain aspects, are multispecific binder molecules for use in methods of inducing an immune response against a tumor cell. Current approaches to treat MM most often do not produce a desired clinical response or lead to a durable desired clinical outcome. In many instance, MM patients relapse following a treatment due to antigen escape, clonal evolution, development of tumor resistance, or any combination thereof. Antigen loss of a previously targeted tumor antigen can result in relapse and ineffectiveness of a previously effective treatment. Mechanisms that lead to tumor resistance can be categorized as either tumor intrinsic mechanisms or immune dependent mechanisms. Tumor intrinsic mechanisms include antigenic loss (either biallelic or functional loss) through deletions or mutations in one or more target genes, increased soluble levels of BCMA (in BCMA-targeting therapy), high tumor burden (bone marrow plasma cells (BMPC) of >60%), and extramedullary disease. Immune-mediated mechanisms of resistance are primarily dependent on T cell fitness and tolerant immune environment. In some embodiments, multispecific binder molecules described herein are able to facilitate immunological synapse formation in heterogeneous populations of MM cells. In some embodiments, multispecific binder molecules described herein are able to facilitate immunological synapse formation in populations of MM cells that are no longer treatment with single or dual targeting therapies. In some embodiments, multispecific binder molecules described herein are able to facilitate efficient immunological synapse formation in populations of MM cells by targeting two or more tumor-specific antigens to bring an engaged T cell in proximity to an MM cell.

In some embodiments, the multispecific binding molecule engages one or more immune effector cells and binds to a tumor cell. In some embodiments, the CD3 binding arm of the multispecific binder molecule binds to and engages T cells. In some embodiments, the CD3 binding arm of the multispecific binder molecule binds to and engages T cells in a chimeric antigen receptor therapy (CAR-T therapy). In some embodiments, the FcRH5 binding arm of the multispecific binder molecule binds to and engages B cells. In some embodiments, the FcRH5 binding arm of the multispecific binder molecule binds to and engages plasma cells. In some embodiments, the FcRH5 binding arm of the multispecific binder molecule binds to and engages malignant plasma cells. In some embodiments, the FcRH5 binding arm of the multispecific binder molecule binds to and engages MM cells. In some embodiments, the BCMA binding arm of the multispecific binder molecule binds to and engages plasma cells. In some embodiments, the BCMA binding arm of the multispecific binder molecule binds to and engages malignant plasma cells. In some embodiments, the BCMA binding arm of the multispecific binder molecule binds to and engages MM cells. In some embodiments, the multispecific binder molecule targets cell-surface BCMA preferentially versus soluble BCMA. In some embodiments, the GPRC5D binding arm of the multispecific binder molecule binds to and engages plasma cells. In some embodiments, the GPRC5D binding arm of the multispecific binder molecule binds to and engages malignant plasma cells. In some embodiments, the GPRC5D binding arm of the multispecific binder molecule binds to and engages MM cells. In some embodiments, the CD38 binding arm of the multispecific binder molecule binds to and engages plasma cells. In some embodiments, the CD38 binding arm of the multispecific binder molecule binds to and engages malignant plasma cells. In some embodiments, the CD38 binding arm of the multispecific binder molecule binds to and engages MM cells. In some embodiments, the SLAMF7 binding arm of the multispecific binder molecule binds to and engages plasma cells. In some embodiments, the SLAMF7 binding arm of the multispecific binder molecule binds to and engages malignant plasma cells. In some embodiments, the SLAMF7 binding arm of the multispecific binder molecule binds to and engages MM cells. In some embodiments, the binding to and engaging a T cell and a malignant plasma cell by the multispecific binding molecule forms an immunological synapse. In some embodiments, following formation of the immunological synapse, T cells become activated. In some embodiments, T cell activation results in multispecific binding molecule-dependent killing of plasma cells, MM cells, or both plasma cells and MM cells in the subject. In some embodiments, the multispecific binding molecule-dependent killing is the result of T cell mediated cytokine release. In some embodiments, the multispecific binding molecule-dependent killing is the result of T cell mediated cytotoxicity through granzyme and perforin-induced cell lysis. In some embodiments, the administering produces plasma cell depletion in the subject. In some embodiments, the administering produces MM cell depletion in the subject. In some embodiments, the administering produces plasma cell and MM cell depletion in the subject. In some embodiments, the administering produces a complete depletion of B cells and plasma cells in BM of the subject. In some embodiments, the administering produces a complete depletion of MM cells in BM of the subject. In some embodiments, the cytotoxic action of the multispecific binding molecule is due to T cell engagement. In some embodiments, Fc receptor-mediated ADCC does not contribute to multispecific binding molecule activity.

The efficacy of immunotherapy for MM treatment depends on the fitness of the immune effector cells. MM is characterized by progressive T-cell dysfunction. Marrow-infiltrating lymphocytes (MIL) in MM are heterogeneous T-cell populations that typically undergo exhaustion characterized by terminal differentiation, loss of effector functions, and expression of inhibitory receptors from suboptimal priming and chronic antigen stimulation in the immunosuppressive BM tumor microenvironment. Efficient targeting of MM cells by administering a multispecific binder molecule described herein to a subject in need thereof lessens the effect of progressive T-cell dysfunction and produces a more effective clinical outcome. In some embodiments, the methods of treatment described herein induce pooling of peripheral blood T cells to the BM-MM tumor microenvironment and enable the selective expansion of tumor-reactive clonotypic CD8+ T cells. In some embodiments, the methods of treatment described herein induce pooling of peripheral blood T cells to the BM-multiple myeloma tumor microenvironment and enable the selective expansion of tumor-reactive clonotypic CD4+ T cells. In some embodiments, the multispecific binding molecule targeting a combination of tumor antigens provides superior tumor selectivity in MM treatment compared to a single or dual targeting strategy. In some embodiments, superior tumor selectivity reduces inhibition of tumor-specific T effector cells activated by the multispecific binding molecule. In some embodiments, superior tumor selectivity reduces the induction of T cell anergy in the subject. In some embodiments, superior tumor selectivity leads to a lowered extent of induction of Tregs. In some embodiments, superior tumor selectivity produces a lowered extent of BM microenvironment immunosuppression. In some embodiments, the method reduces an extent of immunosuppression in the BM microenvironment. In some embodiments, the method induces T cell activation. In some embodiments, the method induces tumor cell lysis. In some embodiments, the method promotes T cell proliferation. In some embodiments, the method promote CD8+ T cell cytotoxicity toward MM cells. In some embodiments, the method promote CD4+ T cell cytotoxicity toward MM cells.

In some aspect, the methods for inducing an immune response against a tumor cell described herein comprise contacting the tumor cell with the multispecific binding molecule comprising three distinct tumor antigen binding moieties (a-c) comprising: a) a TNFRSF17 (BCMA) binding moiety, b) a GPRC5D binding moiety, and c) a FcRH5 binding moiety; and d) an immune cell engager. In some embodiments, the contacting induces proximity between the tumor cell and one or more immune effector cells. In some embodiments, the contacting comprises simultaneous or sequential binding of the multispecific binding molecule to an immune effector cell and a tumor cell. In some embodiments, the tumor cells is a myeloma cell. In some embodiments, the tumor cell is a multiple myeloma cell. In some embodiments, the contacting supports formation of a cytolytic immunological synapse between the tumor cell and one or more immune effector cells. In some embodiments, the one or more immune effector cells comprise one or more activated T cells. In some embodiments, the one or more activated T cells release primary cytokines that induce the activation of other immune cell types wherein the other immune cell types comprise macrophages, dendritic cells, monocytes, or any combination thereof. In some embodiments, the other immune cell types produce elevated levels of secondary cytokines, wherein the secondary cytokines comprise IL6, IL10, IL5, or any combination thereof. In some embodiments, the one or more immune effector cells induce apoptosis in a target tumor cell. In some embodiments, the one or more immune effector cells induce pyroptosis in a target tumor cell. In some embodiments, the one or more immune effector cells release one or more cytotoxic proteases of the granzyme family following the contacting. In some embodiments, the one or more cytotoxic proteases of the granzyme family comprise Granzyme B (GrB). In some embodiments, the one or more immune effector cells release one or more lytic proteins following the contacting. In some embodiments, the one or more lytic proteins comprise perforin. In some embodiments, the contacting engages a T cell receptor (TCR) on one or more T cells. In some embodiments, the one or more T cells comprise naïve T cells, helper T cells, cytotoxic T cells, or any combination thereof. In some embodiments, the one or more T cells receive a co-stimulatory signal resulting in T cell proliferation, differentiation, and survival. In some embodiments, the contacting induces co-stimulation independent polyclonal T-cell activation.

A subject having an exhausted T cell profile in both the peripheral blood and BM compartments, is less amenable to treatment efficacy with immunostimulatory therapies. In some embodiments, targeting three tumor-associated antigens with a T-cell engaging molecule improves treatment efficacy and prevents treatment-induced T cell exhaustion in the subject. In some embodiments, administering the multispecific binder molecule to the subject allows for a more durable therapeutic benefit while attenuating the risk of treatment-emergent adverse events.

In some embodiments, the methods for treatment comprise contacting a plurality of cells in the subject with a multispecific binder molecule described herein. In some embodiments, the methods for inducing an immune response against a tumor cell comprise contacting the tumor cell with a multispecific binding molecule described herein. In some embodiments, the methods for treatment comprise administering a therapeutically effective amount of the multispecific binder molecule to a subject in need thereof. In some embodiments, the multispecific binder molecule engages effector immune cells. In some embodiments, administering the multispecific binder molecule brings effector immune cells in proximity to tumor cells. In some embodiments, administering the multispecific binder molecule induces an immune response in the subject directed against tumor cells. In some embodiments, the multispecific binder molecule gains access to the BM microenvironment. In some embodiments, the multispecific binder molecule simultaneously binds an immune effector cell and a tumor cell. In some embodiments, the multispecific binder molecule simultaneously binds a T cell and a tumor cell. In some embodiments, the multispecific binder molecule simultaneously binds an NK cell and a tumor cell. In some embodiments, the multispecific binder molecule simultaneously binds an NKT cell and a tumor cell. In some embodiments, the simultaneous binding creates an immunological synapse. In some embodiments, a specific T cell response directed against MM tumor cells is elicited following the administering. In some embodiments, the administering reduces proliferation of MM plasma cells in the subject. In some embodiments, the administering enhances immune cell cytotoxicity directed against MM plasma cells in the subject.

In some aspects, a method for treating a cancer in a subject in need thereof described herein comprises administering a therapeutically effective amount of the multispecific binding molecule described herein to the subject. In some embodiments, the cancer is a hematological cancer. In some embodiments, the cancer is myeloma. In some embodiments, the cancer is multiple myeloma. In some embodiments, the therapeutically effective amount provides at least a PR, a VGPR, a CR, or a sCR and reduces a risk of an adverse event in the subject. In some embodiments, the adverse event that has a reduced risk is infection, neutropenia, nervous system disorder, alanine amino-transferase increased, anemia, aspartate amino-transferase increased, hyperglycemia, hyponatremia, a musculo-skeletal and connective tissue disorder, acute renal failure, or thrombocytopenia, or any combination thereof. In some embodiments, the method comprises co-administration with a first-line, second-line, or third-line treatment. In some embodiments, the first-line, second-line, or third-line treatment comprises administering a proteasome inhibitor to the subject. In some embodiments, the proteasome inhibitor comprises bortezomib, carfilzomib, or ixazomib. In some embodiments, the first-line, second-line, or third-line treatment comprises administering an immunomodulatory agent (IMiD) to the subject. In some embodiments, the IMiD comprises thalidomide, lenolidomide, or pomalidomide. In some embodiments, the first-line, second-line, or third-line treatment comprises administering an anti-CD38 monoclonal antibody to the subject. In some embodiments, the subject is determined to be a triple-class refractory MM patient refractory to proteasome inhibitors, IMiDs, and anti-CD38 monoclonal antibody therapies. In some embodiments, the method of treatment described herein comprises administering a multispecific binding molecule described herein as a monotherapy to a triple-class refractory MM subject. In some embodiments, the method of treatment described herein comprises administering a multispecific binding molecule described herein in combination with a separate therapeutic agent to a triple-class refractory MM subject. In some embodiments, the method of treatment described herein comprises administering a multispecific binding molecule described herein to a transplant ineligible subject. In some embodiments, the therapeutically effective amount reduces a risk of the subject developing cytokine release syndrome (CRS) following the administering. In some embodiments, the treatment results in a durable response in the subject. In some embodiments, the treatment improves progress-free survival (PFS) in the subject. In some embodiments, the treatment improves the depth of response in the subject. In some embodiments, the depth of response is measured in a CR rate or sCR rate. In some embodiments, the treatment increases minimal residual disease (MRD) negativity rate. In some embodiment, the treatment prolongs progression-free survival (PFS) and overall survival (OS) in the subject. In some embodiments, the treatment improves an MRD assessment. In some embodiments, the MRD assessment is performed by assaying a BM aspirate sample from the subject following a treatment. In some embodiments, the BM aspirate is assayed using multiparameter flow cytometry (MFC) to identify phenotypically aberrant clonal plasma cells. In some embodiments, the BM aspirate is assayed using qPCR to identify MM plasma cell-specific IGH gene rearrangements. In some embodiments, the BM aspirate is assayed using next-generation sequencing (NGS) to identify genomic alterations. In some embodiments, using NGS, DNA is amplified using primers designed for IGF-VDJH, IGF-DJH, or IGK and sequenced to determine the presence and quantity of clonal DNA sequence. In some embodiments, the BM aspirate is assayed using cytological analysis to identify chromosomal alterations. In some embodiments, the MRD assessment is performed by assaying a serum sample from the subject following a treatment. In some embodiments, the serum sample is assayed using LC MALDI-TOF or mass-fix mass spectrometry. In some embodiments, M-protein detection is determined by scanning the overall mass distribution of denatured intact immunoglobulin light chains. In some embodiments, the serum sample is assayed using clonotypic mass spectrometry. In some embodiments, the treatment increases overall survival (OS) in the subject. Overall survival (OS) is defined as the time from randomization to death. In some embodiments, the treatment increases progression free survival (PFS) in the subject. Progression free survival (PFS) is defined as the time from randomization until first evidence of disease progression or death. In some embodiments, the treatment reduces time to progression (TTP) in the subject. Time to progression (TTP) is defined as the time from randomization until first evidence of disease progression. In some embodiments, the treatment increases disease free survival (DFS) in the subject. Disease free survival (DFS) is defined as the time from randomization until evidence of disease recurrence. In some embodiments, the treatment increases duration of response (DOR) in the subject. (DOR): the length of time a tumor will respond to treatment without growing or metastasizing. In some embodiments, the treatment increase a length of event-free survival of the subject. Event-free survival (EFS) is defined as the time from randomization to an event which may include disease progression, discontinuation of the treatment for any reason, or death. In some embodiments, the treatment decreases time to next treatment (TTNT) in the subject. Time to next treatment (TTNT) is defined as the time from initiating treatment to initiating the next line of therapy. In some embodiments, the treatment increases duration of clinical benefit (DoCB) in the subject. Duration of clinical benefit (DoCB) is defined as the time from randomization to disease progression or death in patients who achieve complete response, partial response, or stable disease for 24 weeks or longer. In some embodiments, the treatment increases duration of response (DoR) in the subject. Duration of response (DoR) is defined as the time from randomization to disease progression or death in patients who achieve complete or partial response. In some embodiments, the treatment increases object response rate (ORR) in the subject. In some embodiments, the treatment improves disease control rate (DCR) in the subject. Disease control rate (DCR) describes the percentage of patients with advanced cancer whose therapeutic intervention has led to a complete response, partial response, or stable disease. In some embodiments, the treatment improves the clinical benefit rate in the subject. Clinical benefit rate (CBR) is defined as the percentage of advanced cancer patients who achieve complete response, partial response, or at least six months of stable disease as a result of therapy. In some embodiments, the treatment increases health-related quality of life (HRQOL) in the subject. Health-related quality of life (HRQOL) is an evaluation of a patient's quality of life with respect to health status over time. The goal of including HRQOL as a clinical endpoint is to complete the results quantitative endpoints such as OS. Quality of life is often used as a secondary clinical endpoint to compare treatments that have similar effects with differences in toxicity, but it can also be used as a co-primary endpoint with OS.

In some embodiments, the multispecific binder molecule is administered to a subject in need thereof to treat a hematological cancer. In some embodiments, the hematological cancer is a myeloma. In some embodiments, the hematological cancer is multiple myeloma (MM). In some embodiments, the MM is asymptomatic or smoldering MM. In some embodiments, the smoldering MM does not manifest in physical symptoms that affect the subject. In some embodiments, the MM is a hyperdiploid MM in which malignant cells have more chromosomes than normal. In some embodiments, the MM is a hypodiploid MM in which malignant cells have fewer chromosomes than normal. In some embodiments, the MM is a light chain myeloma. In some embodiments, the MM is a non-secretory myeloma. In some embodiments, the MM comprises a solitary plasmacytoma. In some embodiments, the MM comprises an extramedullary plasmacytoma. In some embodiments, the MM has progressed from MGUS. In some embodiments, the MM is an immunoglobulin G (IgG) myeloma. In some embodiments, the MM is an immunoglobulin A (IgA) myeloma. In some embodiments, the MM is an immunoglobulin M (IgM) myeloma. In some embodiments, the MM is an immunoglobulin D (IgD) myeloma. In some embodiments, the MM is an immunoglobulin E (IgE) myeloma. In some embodiments, the MM is an active myeloma.

In some embodiments, the subject in need of a treatment, wherein the treatment comprises administering the multispecific binder molecule, has multiple myeloma. In some embodiments, the subject has been diagnosed with active MM and has not begun an initial MM treatment. In some embodiments, the subject has been diagnosed with active MM and, after completing a period of treatment, has maintained a period of remission. In some embodiments, the subject has received one or more treatments for MM. In some embodiments, the subject has received two or more treatments for MM. In some embodiments, the subject has received three or more treatments for MM. In some embodiments, the subject has received four or more treatments for MM. In some embodiments, the subject has relapsed with MM symptoms one or more times following a treatment for MM. In some embodiments, the subject has relapsed with MM symptoms two or more times following a treatment for MM. In some embodiments, the subject has relapsed with MM symptoms three or more times following a treatment for MM. In some embodiments, the subject has relapsed with MM symptoms four or more times following a treatment for MM. In some embodiments, the subject has MM that is refractory to a previously effective treatment. In some embodiments, the refractory myeloma is non-responsive to a therapy or progresses within 60 days of the last line of therapy administered. In some embodiments, the subject has relapsed refractory multiple myeloma (RRMM).

In some embodiments, the administering an effective amount of the multispecific binder molecule increases the overall survival (OS) rate of the subject. In some embodiments, the multispecific binder molecule is administered systemically or locally. In some embodiments, the multispecific binder molecule administered systemically is administered by intravenous injection. In some embodiments, the multispecific binder molecule is administered systemically by infusion. In some embodiments, the multispecific binder molecule is administered systemically by drip infusion. In some embodiments, the multispecific binder molecule is administered systemically by pump infusion.

In some embodiments, the immune response induced against a tumor cell leads to targeted depletion of a plurality of tumor cells. In some embodiments, cytotoxic immune effector cells are activated following the induction of the immune response. In some embodiments, cytotoxic immune effector cells are able to penetrate the immunoprotected BM microenvironment. In some embodiments, cytotoxic immune effector cells are able to penetrate the immunoprotected BM microenvironment and target malignant plasma cells for lysis. In some embodiments, cytotoxic immune effector cells are able to reduce tumor burden in the subject following the administering. In some embodiments, cytotoxic immune effector cells are able to produce a partial response (PR) in the subject following the administering. In some embodiments, cytotoxic immune effector cells are able to produce a very good partial response (VGPR) in the subject following the administering. In some embodiments, cytotoxic immune effector cells are able to produce a complete remission (CR) in the subject following the administering. In some embodiments, cytotoxic immune effector cells are able to produce a stringent complete remission (sCR) in the subject following the administering.

In some embodiments, the administering improves one or more MM symptoms of the subject. In some embodiments, the one or more MM symptoms comprise pain, bone pain, nausea, constipation, frequent urination, loss of appetite, mental fogginess or confusion, fatigue, frequent infections, weight loss, weakness in extremities, leg numbness, excessive thirst, blood clots, nosebleed, bleeding gums, bruising, cloudy vision, hyperviscosity of blood, low platelet count, weakened bones, bone fractures, shortness of breath, dizziness, anemia, kidney damage, kidney dysfunction, or amyloidosis, or any combination thereof. In some embodiments, the administering significantly lowers a concentration of M protein in the blood of the subject. In some embodiments, the administering significantly lowers a concentration of M protein in the urine of the subject. In some embodiments, the administering significantly lowers a serum concentration of M protein in the subject to below about 90, 85, 80, 75, 70, 65, 60, 55, 50, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 g/L. In some embodiments, the administering improves anemia in the subject. In some embodiments, the administering improves anemia in the subject to a level about at least about 10 g/dL of a level of hemoglobin. In some embodiments, the administering improves hypercalcemia in the subject. In some embodiments, the administering improves hypercalcemia in the subject to a corrected calcium level of below about 2.7 mmol/L. In some embodiments, the administering improves kidney dysfunction in the subject. In some embodiments, the administering improves an involved/uninvolved light chain ratio in the subject to below 100. In some embodiments, the administering improves lytic bone lesions identified through X-ray, PET, MRI, or CT scanning. In some embodiments, the administering improves compression fractures. In some embodiments, the administering significantly decreases a number of plasma cells in a bone marrow sample of the treated subject. In some embodiments, the administering significantly decreases a number of plasma cells in a bone marrow sample of the treated subject so that the percentage of plasma cells versus total cells in the BM sample is less than about 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, or 5%. In some embodiments, the administering decreases a measurement of B2 microglobulin in the subject to less than about 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, or 2.5 mg/L. In some embodiments, the administering decreases a level of serum albumin to below about 3.5 g/dL. In some embodiments, the administering significantly decreases a serum level of LDH in the subject.

### Pharmaceutically Acceptable Excipients, Carriers, And Diluents

Compositions comprising the modified antibodies and multispecific binder molecules of the current disclosure are included in a pharmaceutical composition comprising one or more pharmaceutically acceptable excipients, carriers, and diluents. In some embodiments, the antibodies of the current disclosure are administered suspended in a sterile solution. In some embodiments, the antibodies of the current disclosure are administered suspended in an isotonic solution. In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate. In certain aspects, the solution comprises about 0.9% NaCl. In certain aspects, the solution comprises about 5.0% dextrose. In certain aspects, the solution further comprises one or more of: buffers, for example, acetate, citrate, histidine, succinate, phosphate, bicarbonate and Tris(hydroxymethyl)aminomethane; surfactants, for example, polysorbate 80 (Tween 80), polysorbate 20 (Tween 20); polyol/disaccharide/polysaccharides, for example, glucose, dextrose, mannose, mannitol, sorbitol, sucrose, and dextran 40; amino acids, for example, glycine or arginine; antioxidants, for example, ascorbic acid, methionine; or chelating agents, for example, EDTA or EGTA.

In certain aspects, the modified antibodies and multispecific binder molecules of the current disclosure are shipped and/or stored lyophilized and can then be reconstituted before administration. In certain aspects, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than about 7.5. In certain aspects, the pH can be between 4.5 and 7.5, 4.5 and 7.0, 4.5 and 6.5, 4.5 and 6.0, or 5.5 or 5.0.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this specification, the terms “multispecific binder molecule” and “multispecific binding molecule” are used interchangeably.

Throughout this specification, the terms “tetraspecific binder molecule” and “tetraspecific binding molecule” are used interchangeably.

Throughout this specification, the terms “T cell” and “T-cell” are used interchangeably.

Throughout this specification, the terms “T cells” and “T-cells” are used interchangeably.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “antibody” herein is used in the broadest sense and includes monoclonal antibodies, including intact antibodies and functional (antigen-binding) antibody fragments thereof, including fragment antigen-binding (Fab) fragments, F(ab′)2 fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region. An antibody includes, but is not limited to, full-length and native antibodies, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)2, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. A monoclonal antibody can comprise a human IgG1 constant region or a human IgG4 constant region.

The terms “complementarity determining region,” and “CDR,” which are synonymous with “hypervariable region” or “HVR,” are known in the art and refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). CDR-H1, CDR-H2, CDR-H3 are also referred to as HCDR1, HCDR2, and HCDR3 respectively. CDR-L1, CDR-L2, CDR-L3 are also referred to as LCDR1, LCDR2, and LCDR3 respectively. “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309 (3): 657-70, (“Aho” numbering scheme); and Whitelegg N R and Rees A R, “WAM: an improved algorithm for modelling antibodies on the WEB,” Protein Eng. 2000 December; 13 (12): 819-24 (“AbM” numbering scheme. In certain aspects, the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three CDRs (See e.g., Kindt et al. Kuby Immunology, 6th ed., W.H. Freeman and Co., page 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (See e.g., Portolano et al., J. Immunol. 150:880-887 (1993); Clarkson et al., Nature 352:624-628 (1991)).

Among the provided antibodies are antigen-binding fragments thereof. An “antigen-binding fragment thereof” derived from an antibody can refer to a molecule other than an intact antibody that comprises a portion of an intact antibody that binds the antigen to which the intact antibody binds. Examples of antigen-binding fragments thereof derived from an antibody include, but are not limited to, Fv, Fab, Fab′, Fab′-SH, F(ab′) 2; diabodies; linear antibodies; and single-chain antibody molecules (e.g. scFv or sFv). In particular aspects, the antibodies are single-chain antibody fragments comprising a variable heavy chain region and/or a variable light chain region, such as scFvs. Antigen-binding fragments thereof derived from an antibody can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., polypeptide linkers, and/or those that are not produced by enzyme digestion of a naturally-occurring intact antibody. The strength, or affinity of immunological binding interactions of antibodies and/or antigen-binding fragments thereof can be expressed in terms of the dissociation constant (K_{d}) of a specific interaction, wherein a smaller Kd represents a greater affinity for the antibody or antigen-binding fragment thereof to an antigen. Immunological binding properties of selected polypeptides described herein can be quantified using methods well known in the art. One such method involves measuring the rates of antigen-binding site/antigen complex formation and dissociation, wherein those rates depend on the concentrations of the complex partners, the affinity of the interaction, and various geometric parameters that equally influence the rate in both directions. Thus, both the “on rate constant” (Kon) and the “off rate constant” (K_{off}) may be determined by calculation of the concentrations and the actual rates of association and dissociation.

A “binding moiety” refers to a portion of a molecule, peptide, polypeptide, antibody, or antibody fragment that mediates specific binding to a recited target or antigen or epitope. By way of example, the binding moiety of an antibody may comprise a heavy-chain/light-chain variable region pair or one or more complementarity determining regions (CDRs).

A “target” as referred to herein refers to the portion of a molecule that participates with a binding moiety of a molecule, peptide, polypeptide, antibody, or antibody fragment. A target can comprise an amino acid sequence and/or a carbohydrate, lipid, or other chemical entity. An “antigen” is a target comprising a portion that is able to be bound by an adaptive immune molecule such as an antibody or antibody fragment, B-cell receptor, or T-cell receptor.

An “epitope” as described herein refers to the one or more contact regions of an antibody. The contact region of an antibody consists of a discreet number of amino acids contacted by amino acid residues of the antibody (generally CDR residues) and adjacent residues contiguous with the contact residues. For example, the contact region may consist of a continuous stretch of a target protein that is between 5 to 20 amino acids, 5 to 15 amino acids, or 5 to 10 amino acids. An antibody may bind more than one contact region that are separated by 10, 20, 30, 40, 50, 75, or 100 amino acids or more as a result of protein folding. Epitopes may be determined using X-ray crystallography, hydrogen-deuterium exchange mass-spec, alanine spanning mutagenesis, competition with excess synthetic peptides as determined by immunoblot, ELISA, surface plasmon radiance, flow cytometry or any other suitable protein binding assay.

A “humanized” antibody is an antibody in which all or substantially all CDR amino acid residues are derived from non-human CDRs and all or substantially all FR amino acid residues are derived from human FRs. A humanized antibody optionally can include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

Among the provided antibodies are camelid antibodies. A camelid antibody may be derived from a camel, a llama, an alpaca, or a related species. A camelid antibody may be constructed in the format of an antibody from a camel, a llama, an alpaca, or a related species. A camelid antibody may be may a chimeric antibody containing one or more elements derived from an antibody from a camel, a llama, an alpaca, or a related species and one or more elements derived from an antibody of a different species.

“ADCC” or “antibody dependent cell-mediated cytotoxicity” as used herein, refers to the cell-mediated reaction wherein nonspecific cytotoxic cells that express FcγRs recognize bound antibody on a target cell and subsequently cause lysis of the target cell. ADCC can be correlated with binding to FcγRIIIa wherein increased binding to FcγRIIIa leads to an increase in ADCC activity. “ADCP” or antibody dependent cell-mediated phagocytosis, as used herein, can refer to the cell-mediated reaction wherein nonspecific cytotoxic cells that express FcγRs recognize bound antibody on a target cell and subsequently cause phagocytosis of the target cell.

The terms “polypeptide” and “protein” are used interchangeably and refers to a polymer of amino acid residues, and are not limited to a minimum length. Polypeptides, including the provided antibodies and antibody chains and other peptides, e.g., linkers and binding peptides, can include amino acid residues including natural and/or non-natural amino acid residues. The terms also include post-expression modifications of the polypeptide, for example, glycosylation, sialylation, acetylation, phosphorylation, and the like. In some embodiments, the polypeptides can contain modifications with respect to a native or natural sequence, as long as the protein maintains the desired activity. These modifications can be deliberate, as through site-directed mutagenesis, or can be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Amino acid sequence variants of the modified antibodies provided herein can be contemplated and conceived. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody amino acid sequence variants of an antibody can be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. Antibody variants having one or more amino acid substitutions can be provided. Sites of interest for mutagenesis by substitution include the CDRs and FRs. Amino acid substitutions can be introduced into an antibody of interest and the products screened for a desired activity, e.g., retained/improved antigen binding, decreased immunogenicity, or improved ADCC or CDC.

The modified antibodies described herein can be encoded by a nucleic acid. A nucleic acid is a type of polynucleotide comprising two or more nucleotide bases. In certain aspects, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors, and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

The nucleic acids encoding the modified antibodies described herein can be used to infect, transfect, transform, or otherwise render a suitable cell transgenic for the nucleic acid, thus enabling the production of antibodies for commercial or therapeutic uses. Standard cell lines and methods for the production of antibodies from a large-scale cell culture are known in the art. See e.g., Li et al., “Cell culture processes for monoclonal antibody production.” Mabs. 2010 September-Oct; 2 (5): 466-477. In certain aspects, the cell is a Eukaryotic cell. In certain aspects, the Eukaryotic cell is a mammalian cell. In certain aspects, the mammalian cell is a cell line useful for producing antibodies is a Chines Hamster Ovary cell (CHO) cell, an NS0 murine myeloma cell, or a PER.C6® cell. In certain aspects, the nucleic acid encoding the antibody is integrated into a genomic locus of a cell useful for producing antibodies. In certain aspects, described herein is a method of making an antibody comprising culturing a cell comprising a nucleic acid encoding an antibody under conditions in vitro sufficient to allow production and secretion of said antibody.

The section headings used herein are for organizational purposes only and are not to be construed as limiting the subject matter described.

### EXAMPLES

The following examples are included for illustrative purposes only and are not intended to limit the scope of the invention.

### Example 1: In Vitro Selective Lysis of MM Cells and Regulation of T Cell Activation and Proliferation Using Multispecific Binder Molecule

The ability of a multispecific binder molecule to elicit a selective cytotoxic effect on human myeloma cancer cell lines is assayed. The ability of a multispecific binder molecule to elicit T cell activation, cytokine release from T cells, and T cell proliferation is assayed. The multispecific binder molecule is capable of inducing formation of a cytolytic synapse and thus directing specific lysis of target positive tumor cells by T-cells. Activated T-cells can then induce a cascade of cytotoxic events, including release of perforins and granzymes, which compromise the cell membrane of targeted cells. This process leads to the activation of caspases, fragmentation of DNA, morphological changes, and ultimately apoptosis of target cells. Additionally, the cytotoxic effects of T-cells are magnified by the release of cytokines that further stimulate T-cells to kill proximal target cells serially and proliferate. T-cell dependent cellular cytotoxicity, T cell activation, T cell cytokine release, and T cell proliferation are assayed according to the methods described in the following reference which is hereby incorporated by reference regarding methods of assaying target cells following T cell activation and T cell assays described therein (Nazarian A A et al. Characterization of bispecific T-cell Engager (BiTE) antibodies with a high-capacity T-cell dependent cellular cytotoxicity (TDCC) assay. J Biomol Screen. 2015 April; 20 (4): 519-27).

Human myeloma cancer cell lines are cultured according to recommended culturing conditions for a given human myeloma cancer cell line. Cell lines tested are i) NCI-H929 [H929] cells (CRL-3580™, ATCC), ii) OPM-2, as described in: Katagiri S, et al. Two distinct human myeloma cell lines originating from one patient with myeloma. Int J Cancer. 1985 Aug. 15; 36 (2): 241-6), or iii) MM.1S (CRL-2974™, ATCC), iii) MOLP-2 (DSMZ Product Number ACC 607), or any combination thereof. The MOLP-2 cell line is a human cell line established from the peripheral blood of a 55 year-old man with IgD lambda type multiple myeloma. The MOLP-2 multiple myeloma cell line is known to endogenously expresses FcRH5 at a low level comparable with that of MM patients. MOLP-2 multiple myeloma cells are described in: Li J, et al. Membrane-Proximal Epitope Facilitates Efficient T Cell Synapse Formation by Anti-FcRH5/CD3 and Is a Requirement for Myeloma Cell Killing. Cancer Cell. 2017 Mar. 13; 31 (3): 383-395, which is hereby incorporated by reference relating to the experimental assays relating to culturing of MOLP-2 cells, in vitro cytotoxicity assays, T cell activation assays, T cell proliferation assays, RNA expression, protein quantitation, and gene expression analysis. Each cell line tested is assayed for expression of tumor cells markers including GPRC5D, FcRH5, CD38, SLAMF7, and BCMA by flow cytometry and/or quantitative RT-PCR. Cell viability is monitored with a Vi-CELL automated cell counter. These cells lines tested are labeled as ‘target cells’. To generate a luminescent gene expression marker to be used for a quantitative T-cell dependent cellular cytotoxicity (TDCC) assay, stable pools of firefly luciferase-expressing cells are produced. Target cells are transduced with a reporter to constitutively express firefly luciferase. A constitutively active luciferase reporter is cloned into a lentiviral vector under control of the huEF1α promoter. Targets cells in culture are transduced with lentivirus and selected with 5-10 μg/mL of the selectable marker blasticidin to generate stable pools constitutively expressing the luciferase reporter. Luciferase expression is confirmed with the Steady-Glo Luciferase Assay System (Promega, Fitchburg, WI).

To obtain human pan T-cells, normal peripheral blood is collected via leukapheresis from normal healthy human donors. Normal peripheral blood pan T-cells are isolated by negative selection from mononuclear cells using indirect immunomagnetic methods and characterized by flow cytometry for viability and activation markers. Freshly isolated primary human T-cells or high viability thawed primary human T-cells are evaluated in the following described T-cell dependent cellular cytotoxicity (TDCC) assay.

Stable luciferase-expressing target cells having been characterized for their expression of tumor cells markers, are co-cultured with non-activated human pan T-cells at an effector-to-target (E-T) ratio of 10:1 in the presence of the multispecific binder molecule. Two-fold or three-fold dilution series for multispecific binder molecule is performed in the TDCC assay media. TDCC assays are performed in the following media: RPMI Medium with non-essential amino acids, 10 mM HEPES, 50 μM 2-β-mercaptoethanol, 1 mM sodium pyruvate, 100 U/mL penicillin, 100 μg/mL streptomycin, and 5% heat-inactivated fetal bovine serum. 2500 cells per well of target cells (20 μL per well) and 25,000 cells per well of T-cells (25 μL/well) are plated sequentially into 384-well, low-flange, white, flat-bottom, polystyrene tissue culture-treated microplates. Dilutions of the multispecific binder molecule and positive and negative controls are added (5 μL/well) and mixed three times to achieve consistency within each well (total volume, 50 μL/well). Plates are incubated for 48 hr at 37° C., 5% CO₂ before cell viability quantification.

To determine target cells' viability if the cells do not stably express luciferase, ATP quantification is performed with the CellTiter-Glo Luminescent Cell Viability Assay System (Promega). Cell supernatant is aspirated and washed at least twice with phosphate buffered saline (PBS) (50 μL/well), aspirated again, and followed by addition of PBS (20 μL/well). Per Promega's protocol, reconstituted CellTiter-Glo reagent is added (20 μL/well), and assay plates are incubated for 30 min at ambient temperature. Luminescence is quantified with an En Vision multilabel reader (Perkin Elmer, Waltham, MA) with an ultrasensitive luminescence detector.

To determine luciferase-based cell viability, target cells' viability is assayed via quantification of constitutively expressed firefly luciferase and is performed with the Steady-Glo Luciferase Assay System (Promega). Per Promega's protocol, reconstituted Steady-Glo reagent was added (25 μL per well), and assay plates were incubated for 30 min at room temperature. Luminescence was quantified with an En Vision multilabel reader (Perkin Elmer) with an ultrasensitive luminescence detector.

For analysis of bioactivity, percent (%) specific cytotoxicity from relative luminescence units (RLUs) is calculate as: % specific cytotoxicity=[1−(RLU live target cells+multispecific antibody)/(mean RLU live target cells−multispecific antibody)]×100.

Results are reported as the mean EC₅₀ inflection point (IP) value±standard error of the mean. Results of percent specific cytotoxicity are plotted on the y-axis and log concentration of the multispecific binder molecule used for each data point is plotted on the x-axis to generate a dose-response curve. As luminescence quantitation has linear correlation with target cell number, cell numbers per well are quantitated and luminescence signal is detected with Steady-Glo. The R² correlation coefficient between cell number and relative luminescence units (RLU) is determined and plotted. The TDCC assay 384-well plate layout includes 4 to 8 rows of replicates per plate and/or with duplicates on separate plates for each of the positive and negative control for the multispecific binder molecule.

T-cell activation is assessed by staining with anti-CD4, anti-CD8, anti-CD25, and anti-CD69 antibodies (Becton Dickinson, Franklin Lakes, NJ, USA). T-cell proliferation is assessed following labeling of T-cells with Carboxyfluorescein diacetate succinimidyl ester (CFDA-SE) (Becton Dickinson). Cells are sorted and analyzed for labeling by flow cytometry. Cytokine concentrations are analyzed from the supernatants of cytotoxicity assays by utilizing the V-PLEX Proinflammatory Panel1 (human) kit (Mesoscale Discovery, Rockville, MA, USA) according to instructions provided by the manufacturer.

Selective lysis effects of the multispecific binder molecule on primary myeloma plasma cells are quantified in comparison to the other test groups. Extent and profile of released cytokines from T cells is determined for the various test groups. T cell proliferation is quantified for the various test groups. The effectiveness of a multispecific binder molecule in mediating T-cell dependent cellular cytotoxicity if myeloma cells in determined in this example. More than one multispecific binder molecule is assayed to compare efficacies of various multispecific binder molecules under the standardized conditions described in this example.

### Example 2: In Vivo Test of Multispecific Binder Molecule for the Treatment of Multiple Myeloma

A xenograft mouse model of multiple myeloma is established. NCI-H929 cells (a human cell line of B lymphocytes originating from a malignant effusion in a female plasmacytoma myeloma patient, CRL-3580™, ATCC) are prepared in culture. The NCI-H929 cells (~5×10⁶) are injected subcutaneously into the left dorsal flank of sublethally irradiated (using a dosage of ~2 Gy) female NOD/SCID mice. On Day 8 following the NCI-H929 cells injection, animals receive a single intravenous dose of anti-asiola GM1 (WAKO, Neuss, Germany) antibody to deplete host NK cells. On Day 9, human T cells expanded in vitro using a T cell isolation, activation/expansion kit (Miltenyi Biotec) are injected into the peritoneal cavity (2×10⁷ cells) of test animals. On Day 12, animals are randomized based on tumor size and treatment with the multispecific binder molecule administered via injection is initiated on Day 13. A negative control group receives vehicle injections. Tumor growth is monitored and recorded daily by caliper measurements on treatment and control animals. The study is terminated on Day 30 and treatment and control group animals are necropsied to evaluate treatment effectiveness to control overall tumor burden.

### Example 3: In Vivo Test in MM Cells of Tetraspecific Binder Molecule for the Treatment of Multiple Myeloma

A xenograft mouse model of multiple myeloma is established. As anti-CD3 antibodies specific for human CD3 do not cross-react with mouse CD3, a mouse model with a reconstituted human immune system (“humanized mice”) is used to demonstrate in vivo anti-myeloma activity of the tetraspecific binder molecule. Sublethally irradiated (using a dosage of ~2 Gy) female NOD/scid gamma (NSG) mice (Strain #: 005557 from Jackson Laboratory) are transplanted with CD34+ selected human hematopoietic stem cells to generate humanized huNSG mice. These huNSG mice are obtained from Jackson Laboratory. MOLP-2 cells (a human cell line established from the peripheral blood of a 55 year-old man with IgD lambda type multiple myeloma) are prepared in culture. At 20 weeks post CD34+ cell transplantation, the MOLP-2 cells (between 2×10⁶ and 5×10⁶) in Hank's balanced salt solution/Matrigel are injected subcutaneously into the left dorsal flank of the female huNSG mice. MOLP-2 (DSMZ Product Number ACC 607) multiple myeloma cells express human FcRH5 endogenously and can be used as a xenograft tumor model. MOLP-2 cells express additional tumor-associated antigens that serve as binding targets for the tetraspecific binder molecule. For instance, MOLP-2 cells have been confirmed as expressing BCMA (Goldstein R L, et al., AMG 701 induces cytotoxicity of multiple myeloma cells and depletes plasma cells in cynomolgus monkeys. Blood Adv. 2020 Sep. 8; 4 (17): 4180-4194). Optionally, following the MOLP-2 cells injection but prior to the initiation of treatment, animals receive a single intravenous dose of anti-asiola GM1 (WAKO, Neuss, Germany) antibody to deplete host NK cells. When tumor volumes reach a volume range of 100-250 mm³, animals are randomized into two groups and the first treatments are administered at that time (Day 0). Group A receive treatment by administration of a tetraspecific binder molecule according to a selected configuration of Table 10 described herein. The tetraspecific binder molecule has specific binding affinity for MM tumor-associated antigens BCMA, FcRH5, and GPRCD5, as well as the T cell binding moiety CD3. Group B receive administration of an isotype control antibody. All treatments are administered once a week by intravenous (IV) tail vein injection. Clinical observations are performed daily to monitor the health of the animals. Tumor growth is monitored and recorded daily by caliper measurements on treatment animals (Group A) and control animals (Group B) and animal body weights are recorded at least twice a week. The study is terminated on Day 30 and treatment and control group animals are necropsied to evaluate treatment effectiveness to control overall tumor burden.

### Example 4: Treatment of Relapsed and Refractory Multiple Myeloma in Subjects Using the Multispecific Binder Molecule

Treatment of patients having relapsed and refractory multiple myeloma (RRMM) is undertaken to gain insight if targeting MM cells with a T cell engager having at least three binding moieties to MM tumor associated antigens can effectively target MM cells that may have lost expression of one or more antigens following previous treatment. Assessment of pretreatment BCMA expression, SLAMF7 expression, FcRH4 expression, CD38 expression, and GPRC5D expression on CD138+ bone marrow plasma cells derived from RRMM patients is conducted. Tumor-associated BCMA expression is assessed in formalin-fixed paraffin-embedded decalcified bone marrow biopsies (BMBs) by immunohistochemistry (IHC) using a monoclonal antibody directed against an intracellular BCMA epitope. SLAMF7, FcRH5, CD38, and GPRC5D protein expression levels are similarly assayed using monoclonal antibodies directed against each respective protein in the BMBs. Bone marrow mononuclear cells that were isolated from fresh bone marrow aspirates are analyzed by flow cytometry and tumor surface BCMA levels are quantified. Soluble BCMA (sBCMA), representing secreted protein from BCMA-expressing cells, is assessed in blood samples derived from RRMM subjects. sBCMA is evaluated by immunoassay (Luminex, catalog no. LXSAHM-01) in serum isolated from peripheral blood. Soluble FcRH5 (sFcRH5), representing secreted protein from FcRH5-expressing cells, is assessed in blood samples derived from RRMM subjects. Minimal residual disease (MRD) prior to the start of treatment in this example is evaluated for each subject by next-generation sequencing (NGS)-based approaches. Tumor responses are assessed using International Myeloma Working group criteria. Subjects receiving treatment are scored and grouped according to partial response (PR), very good partial response (VGPR), complete response (CR), and stringent complete response (sCR). Primary outcome measures include identifying the responsiveness of patients to treatment are referenced in Table 22. The proportion of patients with complete response, sub-complete response, very good partial response, partial response, no change, progressive disease, and relapse will be calculated.

TABLE 22
Response Criteria

| Response | IMWG criteria |
|---|---|
| sCR | CR as defined below plus normal free light chains ratio and absence of clonal |
| sub-complete | cells in BM by immunohistochemistry or immunofluorescence |
| response | |
| CR (complete | Negative immunofixation on serum and urine. Disappearance of any soft tissue |
| response) | plasmacytomas and <5% plasma cells in BM. |
| Very good | Serum and urine M-protein detectable by immunofixation but not on |
| partial | electrophoresis or >about 90% reduction in serum M-protein plus urine M- |
| response | protein level <100 mg/24 hr |
| (VGPR) | |
| Partial | >50% reduction of serum M-protein and reduction in 24 hours urinary M- |
| response | protein by >90% or to <200 mg/24 hr |
| (PR) | If serum and urine M-protein are unmeasurable, a >50% decrease in the |
| | difference between involved and uninvolved free light chains levels is required |
| | in place of the M-protein criteria. |
| | If serum and urine M-protein are not measurable, and serum free light chains |
| | assay is also unmeasurable, >50% reduction in plasma cells is required in |
| | place of M-protein, provided baseline bone marrow plasma cell percentage |
| | was >30%. |
| | In addition to the above criteria, if present at baseline, a >50% reduction in |
| | the size of soft tissue plasmacytomas is also required. |
| No | Not meeting criteria for CR, VGPR, PR, or progressive disease |
| change/Stable | |
| disease | |
| Progressive | Increase of >25% from lowest response value in any one or more of the |
| disease | following: |
| | Serum M-component and/or (the absolute increase must be >0.5 g/dL) |
| | Urine M-component and/or (the absolute increase must be >200 mg/24 h) |
| | Only in patients without measurable serum and urine M-protein levels; |
| | the difference between involved and uninvolved FLC levels. The |
| | absolute increase must be >10 mg/dL. |
| | Bone marrow plasma cell percentage; the absolute percentage must be >10% |
| | Identified development of new bone lesions or soft tissue |
| | plasmacytomas or definite increase in the size of existing bone lesions |
| | or soft tissue plasmacytomas. |
| | Identified development of hypercalcemia (corrected serum calcium >11.5 |
| | mg/dL or 2.65 mmol/L) that can be attributed solely to the plasma |
| | cell proliferative disorder. |
| Relapse | Clinical relapse requires one or more of: |
| | Direct indicators of increasing disease and/or end organ dysfunction |
| | (CRAB features). |
| | Development of new soft tissue plasmacytomas or bone lesions |
| | Definite increase in the size of existing plasmacytomas or bone lesions. |
| | A definite increase is defined as a 50% (and at least 1 cm) increase as |
| | measured serially by the sum of the products of the cross-diameters of |
| | the measurable lesion |
| | Hypercalcemia (>11.5 mg/dL) [2.65 mmol/L] |
| | Decrease in hemoglobin of >2 g/dL [1.25 mmol/L] |
| | Rise in serum creatinine by 2 mg/dL or more [177 mmol/L or more] |
| Relapse from | Any one or more of the following: |
| CR | Reappearance of serum or urine M-protein by immunofixation or |
| | electrophoresis |
| | Development of >5% plasma cells in the BM |
| | Appearance of any other sign of progression (e.g., new plasmacytoma, |
| | lytic bone lesion, or hypercalcemia) |

Dosing and administration schedule of the multispecific binder molecule to be used to assess effectiveness at reducing tumor burden is determined following IND-enabling studies assessing pharmacology, pharmacokinetics, and toxicology. sBCMA is evaluated at the time of screening, baseline (day 1), and until disease progression. All other cytokines and soluble factors are evaluated at the time of screening, baseline, days 1-6, and days 7, 9, 11, 14, and 21. Baseline and post-infusion levels of 24 immune-related soluble factors (Days 1-28) (GM-CSF, Granzyme A, Granzyme B, IFN-γ, IL-10, IL-13, IL-2, IL-4, IL-5, IL-6, MIP-1α, MIP-1β, sCD137, sFas, sFasL, TNFα, Ang-1, Ang-2, IL-15, IL-18, IL-2Rα, IL-7, IL-8, RANKL) in plasma or serum are evaluated by immunoassay in the peripheral blood using commercially available Luminex assays (Ampersand Biosciences, NY, USA). Clinical markers of inflammation, C-reactive protein, and ferritin are measured locally at each clinical study center. MRD status is evaluated in bone marrow aspirate (BMA) by next-generation sequencing at baseline and post-infusion at fixed time points until progression (e.g., baseline, month 1 (M1), month 2 (M2), month 3 (M3), month 6 (M6), month 9 (M9), month 12 (M12), month 18 (M18), and month 24 (M24)) agnostic of response. Standard markers of myeloma response (e.g., M-protein and FLC) are also measured.

While preferred aspects of the present invention have been shown and described herein, it will be obvious to those skilled in the art that such aspects are provided by way of example only. Numerous variations, changes, and substitutions will now occur to those skilled in the art without departing from the invention. It should be understood that various alternatives to the aspects of the invention described herein may be employed in practicing the invention. It is intended that the following claims define the scope of the invention and that methods and structures within the scope of these claims and their equivalents be covered thereby.

### ASPECTS

Aspect 1. A multispecific binder molecule, comprising three distinct tumor antigen binding moieties (a-c) comprising: a) a TNFRSF17 (BCMA) binding moiety, b) a CD38 binding moiety, and c) a FcRH5 binding moiety; and d) an immune cell engager.
Aspect 2. A multispecific binder molecule, comprising three distinct tumor antigen binding moieties (a-c) comprising: a) a TNFRSF17 (BCMA) binding moiety, b) a SLAMF7 binding moiety, and c) a FcRH5 binding moiety; and d) an immune cell engager.
Aspect 3. A multispecific binder molecule, comprising three distinct tumor antigen binding moieties (a-c) comprising: a) a GPRC5D binding moiety, b) a CD38 binding moiety, and c) a FcRH5 binding moiety; and d) an immune cell engager.
Aspect 4. A multispecific binder molecule, comprising three distinct tumor antigen binding moieties (a-c) comprising: a) a GPRC5D binding moiety, b) a SLAMF7 binding moiety, and c) a FcRH5 binding moiety; and d) an immune cell engager.
Aspect 5. A multispecific binder molecule, comprising three distinct tumor antigen binding moieties (a-c) comprising: a) a CD38 binding moiety, b) a SLAMF7 binding moiety, and c) a FcRH5 binding moiety; and d) an immune cell engager.
Aspect 6. The multispecific binder molecule of any one of aspects 1-5, wherein the immune cell engager comprises a T cell binding moiety, an NK cell binding moiety, or an NKT cell binding moiety.
Aspect 7. The multispecific binder molecule of any one of aspects 1-6, wherein the immune cell engager comprises a T cell binding moiety.
Aspect 8. The multispecific binder molecule of any one of aspect 1-7, wherein the T cell binding moiety comprises a CD3 binding moiety.
Aspect 9. The multispecific binder molecule of any one of aspects 1-8, wherein at least one of the three distinct tumor antigen binding moieties is an antibody or antigen binding fragment thereof.
Aspect 10. The multispecific binder molecule of aspect 9, wherein the antibody or antigen binding fragment thereof is a human antibody, a humanized antibody, or antigen binding fragment thereof.
Aspect 11. The multispecific binder molecule of aspect 9 or 10, wherein the antibody or antigen binding fragment thereof comprises a chimeric antibody, a monoclonal antibody, a monovalent Fab′, a divalent Fab2, a single-chain variable fragment (scFv), a diabody, a minibody, a nanobody, a single-domain antibody (sdAb), a camelid antibody, or antigen binding fragment thereof.
Aspect 12. The multispecific binder molecule of any one of aspects 1-11, comprising a first immunoglobulin chain constant region and a second immunoglobulin chain constant region.
Aspect 13. The multispecific binder molecule of aspect 12, wherein the TNFRSF17 (BCMA) binding moiety and the CD38 binding moiety are linked to the first immunoglobulin chain constant region, and wherein the CD3 binding moiety and the FcRH5 binding moiety are linked to the second immunoglobulin chain constant region.
Aspect 14. The multispecific binder molecule of aspect 12, wherein the TNFRSF17 (BCMA) binding moiety and the CD3 binding moiety are linked to the first immunoglobulin chain constant region, and wherein the CD38 binding moiety and the FcRH5 binding moiety are linked to the second immunoglobulin chain constant region.
Aspect 15. The multispecific binder molecule of aspect 12, wherein the TNFRSF17 (BCMA) binding moiety and the FcRH5 binding moiety are linked to the first immunoglobulin chain constant region, and wherein the CD38 binding moiety and the CD3 binding moiety are linked to the second immunoglobulin chain constant region.
Aspect 16. The multispecific binder molecule of aspect 12, wherein the TNFRSF17 (BCMA) binding moiety and the FcRH5 binding moiety are linked to the first immunoglobulin chain constant region, and wherein the SLAMF7 binding moiety and the CD3 binding moiety are linked to the second immunoglobulin chain constant region.
Aspect 17. The multispecific binder molecule of aspect 12, wherein the TNFRSF17 (BCMA) binding moiety and the SLAMF7 binding moiety are linked to the first immunoglobulin chain constant region, and wherein the FcRH5 binding moiety and the CD3 binding moiety are linked to the second immunoglobulin chain constant region.
Aspect 18. The multispecific binder molecule of aspect 12, wherein the TNFRSF17 (BCMA) binding moiety and the CD3 binding moiety are linked to the first immunoglobulin chain constant region, and wherein the FcRH5 binding moiety and the SLAMF7 binding moiety are linked to the second immunoglobulin chain constant region.
Aspect 19. The multispecific binder molecule of aspect 12, wherein the GPRC5D binding moiety and the CD38 binding moiety are linked to the first immunoglobulin chain constant region, and wherein the FcRH5 binding moiety and the CD3 binding moiety are linked to the second immunoglobulin chain constant region.
Aspect 20. The multispecific binder molecule of aspect 12, wherein the GPRC5D binding moiety and the CD3 binding moiety are linked to the first immunoglobulin chain constant region, and wherein the FcRH5 binding moiety and the CD38 binding moiety are linked to the second immunoglobulin chain constant region.
Aspect 21. The multispecific binder molecule of aspect 12, wherein the GPRC5D binding moiety and the FcRH5 binding moiety are linked to the first immunoglobulin chain constant region, and wherein the CD38 binding moiety and the CD3 binding moiety are linked to the second immunoglobulin chain constant region.
Aspect 22. The multispecific binder molecule of aspect 12, wherein the GPRC5D binding moiety and the FcRH5 binding moiety are linked to the first immunoglobulin chain constant region, and wherein the SLAMF7 binding moiety and the CD3 binding moiety are linked to the second immunoglobulin chain constant region.
Aspect 23. The multispecific binder molecule of aspect 12, wherein the GPRC5D binding moiety and the SLAMF7 binding moiety are linked to the first immunoglobulin chain constant region, and wherein the FcRH5 binding moiety and the CD3 binding moiety are linked to the second immunoglobulin chain constant region.
Aspect 24. The multispecific binder molecule of aspect 12, wherein the GPRC5D binding moiety and the CD3 binding moiety are linked to the first immunoglobulin chain constant region, and wherein the FcRH5 binding moiety and the SLAMF7 binding moiety are linked to the second immunoglobulin chain constant region.
Aspect 25. The multispecific binder molecule of aspect 12, wherein the CD38 binding moiety and the SLAMF7 binding moiety are linked to the first immunoglobulin chain constant region, and wherein the FcRH5 binding moiety and the CD3 binding moiety are linked to the second immunoglobulin chain constant region.
Aspect 26. The multispecific binder molecule of aspect 12, wherein the CD38 binding moiety and the FcRH5 binding moiety are linked to the first immunoglobulin chain constant region, and wherein the SLAMF7 binding moiety and the CD3 binding moiety are linked to the second immunoglobulin chain constant region.
Aspect 27. The multispecific binder molecule of aspect 12, wherein the CD38 binding moiety and the CD3 binding moiety are linked to the first immunoglobulin chain constant region, and wherein the FcRH5 binding moiety and the SLAMF7 binding moiety are linked to the second immunoglobulin chain constant region.
Aspect 28. The multispecific binder molecule of any one of aspects 1 or 6-15, wherein the a) TNFRSF17 (BCMA) binding moiety, b) CD38 binding moiety, c) FcRH5 binding moiety, and d) CD3 binding moiety, comprise an antibody or antigen binding fragment thereof, wherein the antibody or antigen binding fragment thereof comprises Fab′, scFv, IgG-scFv, nanobody, mini-antibody, minibody, scFv-CH3 KiH, or scFv-KiH.
Aspect 29. The multispecific binder molecule of any one of aspects 2, 6-12, or 16-18, wherein the a) TNFRSF17 (BCMA) binding moiety, b) SLAMF7 binding moiety, c) FcRH5 binding moiety, and d) CD3 binding moiety, comprise an antibody or antigen binding fragment thereof, wherein the antibody or antigen binding fragment thereof comprises Fab′, scFv, IgG-scFv, nanobody, mini-antibody, minibody, scFv-CH3 KiH, or scFv-KiH.
Aspect 30. The multispecific binder molecule of any one of aspects 3, 6-12, or 19-21, wherein the a) GPRC5D binding moiety, b) CD38 binding moiety, c) FcRH5 binding moiety, and d) CD3 binding moiety, comprise an antibody or antigen binding fragment thereof, wherein the antibody or antigen binding fragment thereof comprises Fab′, scFv, IgG-scFv, nanobody, mini-antibody, minibody, scFv-CH3 KiH, or scFv-KiH.
Aspect 31. The multispecific binder molecule of any one of aspects 4, 6-12, or 22-24, wherein the a) GPRC5D binding moiety, b) SLAMF7 binding moiety, c) FcRH5 binding moiety, and d) CD3 binding moiety, comprise an antibody or antigen binding fragment thereof, wherein the antibody or antigen binding fragment thereof comprises Fab′, scFv, IgG-scFv, nanobody, mini-antibody, minibody, scFv-CH3 KiH, or scFv-KiH.
Aspect 32. The multispecific binder molecule of any one of aspects 5-12, or 25-27, wherein the a) CD38 binding moiety, b) SLAMF7 binding moiety, c) FcRH5 binding moiety, and d) CD3 binding moiety, comprise an antibody or antigen binding fragment thereof, wherein the antibody or antigen binding fragment thereof comprises Fab′, scFv, IgG-scFv, nanobody, mini-antibody, minibody, scFv-CH3 KiH, or scFv-KiH.
Aspect 33. The multispecific binder molecule of any one of aspects 1-32, wherein the first or second immunoglobulin chain constant region comprises a heavy chain constant region of IgG1, IgG2, and IgG4, or a variant thereof.
Aspect 34. The multispecific binder molecule of any one of aspects 1-33, wherein the first or second immunoglobulin chain constant regions form a IgG1 heavy chain or a variant thereof.
Aspect 35. The multispecific binder molecule of aspect 34, comprising the TNFRSF17 (BCMA) binding moiety, wherein the TNFRSF17 (BCMA) binding moiety is coupled to the N-terminus of the IgG1 heavy chain or a variant thereof.
Aspect 36. The multispecific binder molecule of aspect 35, comprising the TNFRSF17 (BCMA) binding moiety, wherein the TNFRSF17 (BCMA) binding moiety is coupled to the C-terminus of the IgG1 heavy chain or a variant thereof.
Aspect 37. The multispecific binder molecule of aspect 35, comprising the GPRC5D binding moiety, wherein the GPRC5D binding moiety is coupled to the N-terminus of the IgG1 heavy chain or a variant thereof.
Aspect 38. The multispecific binder molecule of aspect 35, comprising the GPRC5D binding moiety, wherein the GPRC5D binding moiety is coupled to the C-terminus of the IgG1 heavy chain or a variant thereof.
Aspect 39. The multispecific binder molecule of aspect 35, comprising the CD38 binding moiety, wherein the CD38 binding moiety is coupled to the N-terminus of the IgG1 heavy chain or a variant thereof.
Aspect 40. The multispecific binder molecule of aspect 35, comprising the CD38 binding moiety, wherein the CD38 binding moiety is coupled to the C-terminus of the IgG1 heavy chain or a variant thereof.
Aspect 41. The multispecific binder molecule of aspect 35, comprising the SLAMF7 binding moiety, wherein the SLAMF7 binding moiety is coupled to the N-terminus of the IgG1 heavy chain or a variant thereof.
Aspect 42. The multispecific binder molecule of aspect 35, comprising the SLAMF7 binding moiety, wherein the SLAMF7 binding moiety is coupled to the C-terminus of the IgG1 heavy chain or a variant thereof.
Aspect 43. The multispecific binder molecule of any one of aspects 34-42, wherein the FcRH5 binding moiety is coupled to the N-terminus of the IgG1 heavy chain or a variant thereof.
Aspect 44. The multispecific binder molecule of any one of aspects 34-42, wherein the FcRH5 binding moiety is coupled to the C-terminus of the IgG1 heavy chain or a variant thereof.
Aspect 45. The multispecific binder molecule of any one of aspects 34-44, wherein the CD3 binding moiety is coupled to the N-terminus of the IgG1 heavy chain or a variant thereof.
Aspect 46. The multispecific binder molecule of any one of aspects 34-44, wherein the CD3 binding moiety is coupled to the C-terminus of the IgG1 heavy chain or a variant thereof.
Aspect 47. The multispecific binder molecule of any one of aspect 33-46, wherein the IgG1 heavy chain comprises a mutation that reduces FcγR binding.
Aspect 48. The multispecific binder molecule of aspect 47, wherein the mutation comprises N297A mutation.
Aspect 49. The multispecific binder molecule of any one of aspects 1-48, wherein at least one of the three distinct tumor antigen binding moieties is coupled to an immunoglobulin kappa light chain domain.
Aspect 50. The multispecific binder molecule of any one of aspects 1-48, wherein at least one of the three distinct tumor antigen binding moieties is coupled to an immunoglobulin lambda light chain domain.
Aspect 51. The multispecific binder molecule of any one of aspects 1-50, wherein one of the three distinct tumor antigen binding moieties is coupled to an immunoglobulin lambda light chain domain and another of the three distinct tumor antigen binding moieties is coupled to an immunoglobulin kappa light chain domain.
Aspect 52. The multispecific binder molecule of any one of aspects 1-51, comprising the BCMA binding moiety, wherein the BCMA binding moiety is derived from AMG 701, Alnuctamab, Belantamab mafodotin, Elranatamab, Linvoseltamab, Teclistamab, ABBV-383, HPN217, ISB 2001, GSK287916, MEDI2228, TNB-383B, REGN545, or AMG 420.
Aspect 53. The multispecific binder molecule of any one of aspects 1-52, comprising the BCMA binding moiety, wherein the BCMA binding moiety is derived from Teclistamab.
Aspect 54. The multispecific binder molecule of any one of aspects 1-53, comprising the GPRC5D binding moiety, wherein the GPRC5D binding moiety is derived from Talquetamab or RG6234.
Aspect 55. The multispecific binder molecule of any one of aspects 1-54, comprising the GPRC5D binding moiety, wherein the GPRC5D binding moiety is derived from Talquetamab.
Aspect 56. The multispecific binder molecule of any one of aspects 53-83, wherein the FcRH5 binding moiety is derived from Cevostamab or DFRF4539A.
Aspect 57. The multispecific binder molecule of any one of aspects 1-56, comprising the CD38 binding moiety, wherein the CD38 binding moiety is derived from Daratumumab, Isatuximab, I S B 2001, or MOR202.
Aspect 58. The multispecific binder molecule of any one of aspects 1-56, comprising the CD38 binding moiety, wherein the CD38 binding moiety is derived from Daratumumab.
Aspect 59. The multispecific binder molecule of any one of aspects 1-58, comprising the SLAMF7 binding moiety, wherein SLAMF7 binding moiety is derived from ABBV-838, or Elotuzumab.
Aspect 60. The multispecific binder molecule of any one of aspects 1-59, comprising the SLAMF7 binding moiety, wherein SLAMF7 binding moiety is derived from Elotuzumab.
Aspect 61. The multispecific binder molecule of any one of aspects 1-60, wherein the FcRH5 binding moiety is derived from Cevostamab.
Aspect 62. The multispecific binder molecule of any one of aspects 1-61, wherein the CD3 binding moiety is derived from AMG 701, Alnuctamab, Elranatamab, Linvoseltamab, Teclistamab, ABBV-383, HPN217, XmAb969, ISB1347, Talquetamab, RG6234, Cevostamab, SAR442257, F182112, or ISB 2001.
Aspect 63. The multispecific binder molecule of any one of aspects 1-62, wherein at least one of the three distinct tumor antigen binding moieties comprises a nucleic acid aptamer, a peptide aptamer, a designed ankyrin repeat protein (DARPin), an affimers, an inhibitor cysteine knot (knottin), an avimer, a monobody, an anticalin, a fynomer, or an affibody.
Aspect 64. The multispecific binder molecule of any one of aspects 1-63, wherein the immune cell engager comprises a nucleic acid aptamer, a peptide aptamer, a designed ankyrin repeat protein (DARPin), an affimers, an inhibitor cysteine knot (knottin), an avimer, a monobody, an anticalin, a fynomer, or an affibody.
Aspect 65. A method for inducing an immune response against a tumor cell, comprising contacting the tumor cell with the multispecific binder molecule of any one of aspects 1-64.
Aspect 66. The method of aspect 65, wherein the contacting induces proximity between the tumor cell and one or more immune effector cells.
Aspect 67. The method of aspect 65 or 66, wherein the contacting supports formation of a cytolytic immunological synapse between the tumor cell and one or more immune effector cells.
Aspect 68. The method of any aspect 66 or 67, wherein the one or more immune effector cells comprise one or more activated T cells.
Aspect 69. The method of aspect 68, wherein the one or more activated T cells release primary cytokines that induce the activation of other immune cell types wherein the other immune cell types comprise macrophages, dendritic cells, monocytes, or any combination thereof.
Aspect 70. The method of aspect 69, wherein the other immune cell types produce elevated levels of secondary cytokines, wherein the secondary cytokines comprise IL6, IL10, IL5, or any combination thereof.
Aspect 71. The method of any one of aspects 66-70, wherein the one or more immune effector cells induce apoptosis in a target tumor cell.
Aspect 72. The method of any one of aspects 66-71, wherein the one or more immune effector cells induce pyroptosis in a target tumor cell.
Aspect 73. The method of any one of aspects 66-72, wherein the one or more immune effector cells release one or more cytotoxic proteases of the granzyme family following the contacting.
Aspect 74. The method of aspect 73, wherein the one or more cytotoxic proteases of the granzyme family comprise Granzyme B (GrB).
Aspect 75. The method of any one of aspects 66-74, wherein the one or more immune effector cells release one or more lytic proteins following the contacting.
Aspect 76. The method of aspect75, wherein the one or more lytic proteins comprise perforin.
Aspect 77. The method of any one of aspects 66-76, wherein the contacting engages a T cell receptor (TCR) on one or more T cells.
Aspect 78. The method of aspect 77, wherein the one or more T cells comprise naïve T cells, helper T cells, cytotoxic T cells, or any combination thereof.
Aspect 79. The method of aspect 77 or 78, wherein the one or more T cells receive a co-stimulatory signal resulting in T cell proliferation, differentiation, and survival.
Aspect 80. The method of any one of aspects 77-79, wherein the contacting induces co-stimulation independent polyclonal T-cell activation.
Aspect 81. The method of any one of aspects 65-80, wherein the tumor cell is a myeloma tumor cell.
Aspect 82. The method of any one of aspects 65-81, wherein the tumor cell is a multiple myeloma tumor cell.
Aspect 83. A method for treating a cancer in a subject in need thereof, comprising administering a therapeutically effective amount of the multispecific binder molecule of any one of aspects 1-64 to the subject.
Aspect 84. The method of aspect 83, wherein the cancer is a hematological cancer.
Aspect 85. The method of aspect 83, wherein the cancer is myeloma.
Aspect 86. The method of aspect 83, wherein the cancer is multiple myeloma.
Aspect 87. A multispecific binder molecule, comprising a first binding moiety to a first tumor associated antigen, a second binding moiety to a second tumor associated antigen, a third binding moiety to a third tumor associated antigen, and an immune cell engager, wherein the first, second, and third binding moieties are selected from a group consisting of a GPRC5D binding moiety, a TNFRSF17 (BCMA) binding moiety, a CD38 binding moiety, and a SLAMF7 (CD319) binding moiety.
Aspect 88. The multispecific binder molecule of aspect 87, wherein the immune cell engager comprises a T cell binding moiety, an NK cell binding moiety, or an NKT cell binding moiety.
Aspect 89. The multispecific binder molecule of aspect 87, wherein the immune cell engager comprises a T cell binding moiety.
Aspect 90. The multispecific binder molecule of aspect 88 or 89, wherein the T cell binding moiety comprises a CD3 binding moiety.
Aspect 91. The multispecific binder molecule of any one of aspects 87-90, wherein the first, second, and third binding moieties comprise a GPRC5D binding moiety, a TNFRSF17 (BCMA) binding moiety, and a CD38 binding moiety.
Aspect 92. The multispecific binder molecule of any one of aspects 87-90, wherein the first, second, and third binding moieties comprise a GPRC5D binding moiety, a TNFRSF17 (BCMA) binding moiety, and v) a SLAMF7 (CD319) binding moiety.
Aspect 93. The multispecific binder molecule of any one of aspects 87-90, wherein the first, second, and third binding moieties comprise a GPRC5D binding moiety, a CD38 binding moiety, and v) a SLAMF7 (CD319) binding moiety.
Aspect 94. The multispecific binder molecule of any one of aspects 87-90, wherein the first, second, and third binding moieties comprise: a TNFRSF17 (BCMA) binding moiety, a CD38 binding moiety, and v) a SLAMF7 (CD319) binding moiety.
Aspect 95. The multispecific binder molecule of any one of aspects 87-94, comprising i) the CD3 binding moiety, ii) the GPRC5D binding moiety, and iii) the TNFRSF17 (BCMA) binding moiety.
Aspect 96. The multispecific binder molecule of any one of aspects 87-94, comprising i) the CD3 binding moiety, ii) the GPRC5D binding moiety, iii) the TNFRSF17 (BCMA) binding moiety, and iv) the CD38 binding moiety.
Aspect 97. The multispecific binder molecule of any one of aspects 87-94, comprising i) the CD3 binding moiety, ii) the GPRC5D binding moiety, iii) the TNFRSF17 (BCMA) binding moiety, and v) the SLAMF7 (CD319) binding moiety.
Aspect 98. The multispecific binder molecule of any one of aspects 87-94, comprising i) the CD3 binding moiety, ii) the GPRC5D binding moiety, iv) the CD38 binding moiety, and v) the SLAMF7 (CD319) binding moiety.
Aspect 99. The multispecific binder molecule of any one of aspects 87-94, comprising i) the CD3 binding moiety, iii) the TNFRSF17 (BCMA) binding moiety, iv) the CD38 binding moiety, and v) the SLAMF7 (CD319) binding moiety.
Aspect 100. The multispecific binder molecule of any one of aspects 87-99, wherein at least one of the at least three binding moieties is an antibody or antigen binding fragment thereof.
Aspect 101. The multispecific binder molecule of aspect 100, wherein the antibody or antigen binding fragment thereof is a human antibody, a humanized antibody, or antigen binding fragment thereof.
Aspect 102. The multispecific binder molecule of aspect 100 or 101, wherein the antibody or antigen binding fragment thereof comprises a chimeric antibody, a monoclonal antibody, a monovalent Fab′, a divalent Fab2, a single-chain variable fragment (scFv), a diabody, a minibody, a nanobody, a single-domain antibody (sdAb), a camelid antibody, or antigen binding fragment thereof.
Aspect 103. The multispecific binder molecule of any one of aspects 87-102, further comprising a first immunoglobulin chain constant region and a second immunoglobulin chain constant region.
Aspect 104. The multispecific binder molecule of aspect 103, wherein the TNFRSF17 (BCMA) binding moiety and the GPRC5D binding moiety are linked to the first immunoglobulin chain constant region and CD3 binding moiety and optionally the one of the CD38 binding moiety or the SLAMF7 (CD319) binding moiety are linked to the second immunoglobulin chain constant region.
Aspect 105. The multispecific binder molecule of aspect 103, wherein the TNFRSF17 (BCMA) binding moiety and the CD3 binding moiety are linked to the first immunoglobulin chain constant region and the GPRC5D binding moiety and optionally one of the CD38 binding moiety or the SLAMF7 (CD319) binding moiety are linked to the second immunoglobulin chain constant region.
Aspect 106. The multispecific binder molecule of aspect 103, wherein the CD3 binding moiety and the GPRC5D binding moiety are linked to the first immunoglobulin chain constant region and the TNFRSF17 (BCMA) binding moiety and optionally one of the CD38 binding moiety or the SLAMF7 (CD319) binding moiety are linked to the second immunoglobulin chain constant region.
Aspect 107. The multispecific binder molecule of aspect 103, wherein the CD3 binding moiety and the CD38 binding moiety are linked to the first immunoglobulin chain constant region and the SLAMF7 (CD319) binding moiety and GPRC5D binding moiety are linked to the second immunoglobulin chain constant region.
Aspect 108. The multispecific binder molecule of aspect 103, wherein the CD3 binding moiety and the CD38 binding moiety are linked to the first immunoglobulin chain constant region and the TNFRSF17 (BCMA) binding moiety and SLAMF7 (CD319) binding moiety are linked to the second immunoglobulin chain constant region.
Aspect 109. The multispecific binder molecule of any one of aspects 87-108, wherein the i) a CD3 binding moiety, ii) a GPRC5D binding moiety, iii) a TNFRSF17 (BCMA) binding moiety, iv) a CD38 binding moiety, and v) a SLAMF7 (CD319) binding moiety comprises an antibody or antigen binding fragment thereof, wherein the antibody or antigen binding fragment thereof comprises Fab′, scFv, IgG-scFv, nanobody, mini-antibody, minibody, scFv-CH3 KiH, or scFv-KiH.
Aspect 110. The multispecific binder molecule of any one of aspects 87-109, wherein the first or second immunoglobulin chain constant region comprises a heavy chain constant region of IgG1, IgG2, and IgG4, or a variant thereof.
Aspect 111. The multispecific binder molecule of any one of aspects 87-110, wherein the first or second immunoglobulin chain constant regions form a IgG1 heavy chain or a variant thereof.
Aspect 112. The multispecific binder molecule of aspect 111, wherein the TNFRSF17 (BCMA) binding moiety is coupled to the C-terminus of the IgG1 heavy chain or a variant thereof.
Aspect 113. The multispecific binder molecule of aspect 111 or 112, wherein the CD38 binding moiety is coupled to the C-terminus of the IgG1 heavy chain or a variant thereof.
Aspect 114. The multispecific binder molecule any one of aspects 111-113, wherein the GPRC5D binding moiety is coupled to the C-terminus of the IgG1 heavy chain or a variant thereof.
Aspect 115. The multispecific binder molecule of any one of aspects 111-114, wherein the CD3 binding moiety is coupled to the C-terminus of the IgG1 heavy chain or a variant thereof.
Aspect 116. The multispecific binder molecule of any one of aspects 111-115, wherein the SLAMF7 (CD319) binding moiety is coupled to the C-terminus of the IgG1 heavy chain or a variant thereof.
Aspect 117. The multispecific binder molecule of any one of aspect 111-116, wherein the IgG1 heavy chain comprises a mutation that reduces FcγR binding.
Aspect 118. The multispecific binder molecule of aspect 117, wherein the mutation comprises N297A mutation.
Aspect 119. The multispecific binder molecule of any one of aspects 87-118, wherein the at least one of the at least three binding moieties is coupled to an immunoglobulin kappa light chain domain.
Aspect 120. The multispecific binder molecule of any one of aspects 87-119, wherein the at least one of the at least three binding moieties is coupled to an immunoglobulin lambda light chain domain.
Aspect 121. The multispecific binder molecule of any one of aspects 87-120, wherein one of the at least three binding moieties is coupled to an immunoglobulin lambda light chain domain and another of the at least three binding moieties is coupled to an immunoglobulin kappa light chain domain.
Aspect 122. The multispecific binder molecule of any one of aspects 87-121, wherein the CD38 binding moiety is derived from Daratumumab, Isatuximab, ISB 2001, or MOR202.
Aspect 123. The multispecific binder molecule of any one of aspects 87-122, wherein the CD38 binding moiety is derived from Daratumumab.
Aspect 124. The multispecific binder molecule of any one of aspects 87-123, wherein the SLAMF7 (CD319) binding moiety is derived from ABBV-838, or Elotuzumab.
Aspect 125. The multispecific binder molecule of any one of aspects 87-124, wherein the SLAMF7 (CD319) binding moiety is derived from Elotuzumab.
Aspect 126. The multispecific binder molecule of any one of aspects 87-125, wherein the TNFRSF17 (BCMA) binding moiety is derived from AMG 701, Alnuctamab, Elranatamab, Linvoseltamab, Teclistamab, ABBV-383, HPN217, ISB 2001, GSK287916, MEDI2228, or AMG 420.
Aspect 127. The multispecific binder molecule of any one of aspects 87-126, wherein the TNFRSF17 (BCMA) binding moiety is derived from Teclistamab.
Aspect 128. The multispecific binder molecule of any one of aspects 87-127, wherein the CD3 binding moiety is derived from AMG 701, Alnuctamab, Elranatamab, Linvoseltamab, Teclistamab, ABBV-383, HPN217, XmAb969, ISB1347, Talquetamab, RG6234, Cevostamab, SAR442257, or ISB 2001.
Aspect 129. The multispecific binder molecule of any one of aspects 87-128, wherein the GPRC5D binding moiety is derived from Talquetamab or RG6234.
Aspect 130. The multispecific binder molecule of any one of aspects 87-129, wherein the GPRC5D binding moiety is derived from Talquetamab.
Aspect 131. The multispecific binder molecule of any one of aspects 87-100, wherein at least one of the at least three binding moieties is a nucleic acid aptamer, a peptide aptamer, a designed ankyrin repeat protein (DARPin), an affimers, an inhibitor cysteine knot (knottin), an avimer, a monobody, an anticalin, a fynomer, or an affibody.
Aspect 132. A method for inducing immune response against a tumor cell, comprising contacting the tumor cell with the multispecific binder molecule of any one of aspects 87-131.
Aspect 133. The method of aspect 132, wherein the tumor cell is a myeloma tumor cell.
Aspect 134. The method of aspect 132, wherein the tumor cell is a multiple myeloma tumor cell.
Aspect 135. A method for treating a cancer in a subject in need thereof, comprising administering a therapeutically effective amount of the multispecific binder molecule of any one of aspects 87-131.
Aspect 136. The method of aspect 135, wherein the cancer is a hematological cancer.
Aspect 137. The method of aspect 135 or 136, wherein the cancer is myeloma.
Aspect 138. The method of aspect 135 or 136, wherein the cancer is multiple myeloma.
Aspect 139. A multispecific binder molecule, comprising three distinct tumor antigen binding moieties (a-c) comprising: a) a TNFRSF17 (BCMA) binding moiety, b) a GPRC5D binding moiety, and c) a FcRH5 binding moiety; and d) an immune cell engager.
Aspect 140. The multispecific binder molecule of aspect 139, wherein the immune cell engager comprises a T cell binding moiety, an NK cell binding moiety, or an NKT cell binding moiety.
Aspect 141. The multispecific binder molecule of aspect 139 or 140, wherein the immune cell engager comprises a T cell binding moiety.
Aspect 142. The multispecific binder molecule of aspect 140 or 141, wherein the T cell binding moiety comprises a CD3 binding moiety.
Aspect 143. The multispecific binder molecule of any one of aspects 139-142, wherein at least one of the three distinct tumor antigen binding moieties is an antibody or antigen binding fragment thereof.
Aspect 144. The multispecific binder molecule of aspect 143, wherein the antibody or antigen binding fragment thereof is a human antibody, a humanized antibody, or antigen binding fragment thereof.
Aspect 145. The multispecific binder molecule of aspect 143 or 144, wherein the antibody or antigen binding fragment thereof comprises a chimeric antibody, a monoclonal antibody, a monovalent Fab′, a divalent Fab2, a single-chain variable fragment (scFv), a diabody, a minibody, a nanobody, a single-domain antibody (sdAb), a camelid antibody, or antigen binding fragment thereof.
Aspect 146. The multispecific binder molecule of any one of aspects 139-145, comprising a first immunoglobulin chain constant region and a second immunoglobulin chain constant region.
Aspect 147. The multispecific binder molecule of aspect 146, wherein the TNFRSF17 (BCMA) binding moiety and the GPRC5D binding moiety are linked to the first immunoglobulin chain constant region, and wherein the CD3 binding moiety and the FcRH5 binding moiety are linked to the second immunoglobulin chain constant region.
Aspect 148. The multispecific binder molecule of aspect 146, wherein the TNFRSF17 (BCMA) binding moiety and the CD3 binding moiety are linked to the first immunoglobulin chain constant region, and wherein the GPRC5D binding moiety and the FcRH5 binding moiety are linked to the second immunoglobulin chain constant region.
Aspect 149. The multispecific binder molecule of aspect 146, wherein the TNFRSF17 (BCMA) binding moiety and the FcRH5 binding moiety are linked to the first immunoglobulin chain constant region, and wherein the GPRC5D binding moiety and the CD3 binding moiety are linked to the second immunoglobulin chain constant region.
Aspect 150. The multispecific binder molecule of any one of aspects 139-149, wherein the a) TNFRSF17 (BCMA) binding moiety, b) GPRC5D binding moiety, c) FcRH5 binding moiety, and d) CD3 binding moiety, comprise an antibody or antigen binding fragment thereof, wherein the antibody or antigen binding fragment thereof comprises Fab′, scFv, IgG-scFv, nanobody, mini-antibody, minibody, scFv-CH3 KiH, or scFv-KiH.
Aspect 151. The multispecific binder molecule of any one of aspects 139-150, wherein the first or second immunoglobulin chain constant region comprises a heavy chain constant region of IgG1, IgG2, and IgG4, or a variant thereof.
Aspect 152. The multispecific binder molecule of any one of aspects 139-151, wherein the first or second immunoglobulin chain constant regions form a IgG1 heavy chain or a variant thereof.
Aspect 153. The multispecific binder molecule of aspect 152, wherein the TNFRSF17 (BCMA) binding moiety is coupled to the N-terminus of the IgG1 heavy chain or a variant thereof.
Aspect 154. The multispecific binder molecule of aspect 152, wherein the TNFRSF17 (BCMA) binding moiety is coupled to the C-terminus of the IgG1 heavy chain or a variant thereof.
Aspect 155. The multispecific binder molecule of any one of aspects 152-154, wherein the GPRC5D binding moiety is coupled to the N-terminus of the IgG1 heavy chain or a variant thereof.
Aspect 156. The multispecific binder molecule of any one of aspects 152-154, wherein the GPRC5D binding moiety is coupled to the C-terminus of the IgG1 heavy chain or a variant thereof.
Aspect 157. The multispecific binder molecule of any one of aspects 152-156, wherein the FcRH5 binding moiety is coupled to the N-terminus of the IgG1 heavy chain or a variant thereof.
Aspect 158. The multispecific binder molecule of any one of aspects 152-156, wherein the FcRH5 binding moiety is coupled to the C-terminus of the IgG1 heavy chain or a variant thereof.
Aspect 159. The multispecific binder molecule of any one of aspects 152-158, wherein the CD3 binding moiety is coupled to the N-terminus of the IgG1 heavy chain or a variant thereof.
Aspect 160. The multispecific binder molecule of any one of aspects 152-158, wherein the CD3 binding moiety is coupled to the C-terminus of the IgG1 heavy chain or a variant thereof.
Aspect 161. The multispecific binder molecule of any one of aspect 151-160, wherein the IgG1 heavy chain comprises a mutation that reduces FcγR binding.
Aspect 162. The multispecific binder molecule of aspect 161, wherein the mutation comprises N297A mutation.
Aspect 163. The multispecific binder molecule of any one of aspects 139-162, wherein at least one of the three distinct tumor antigen binding moieties is coupled to an immunoglobulin kappa light chain domain.
Aspect 164. The multispecific binder molecule of any one of aspects 139-163, wherein at least one of the three distinct tumor antigen binding moieties is coupled to an immunoglobulin lambda light chain domain.
Aspect 165. The multispecific binder molecule of any one of aspects 139-164, wherein one of the three distinct tumor antigen binding moieties is coupled to an immunoglobulin lambda light chain domain and another of the three distinct tumor antigen binding moieties is coupled to an immunoglobulin kappa light chain domain.
Aspect 166. The multispecific binder molecule of any one of aspects 139-165, wherein the TNFRSF17 (BCMA) binding moiety is derived from AMG 701, Alnuctamab, Belantamab mafodotin, Elranatamab, Linvoseltamab, Teclistamab, ABBV-383, HPN217, ISB 2001, GSK287916, MEDI2228, TNB-383B, REGN545, or AMG 420.
Aspect 167. The multispecific binder molecule of any one of aspects 139-166, wherein the TNFRSF17 (BCMA) binding moiety is derived from Teclistamab.
Aspect 168. The multispecific binder molecule of any one of aspects 139-167, wherein the GPRC5D binding moiety is derived from Talquetamab or RG6234.
Aspect 169. The multispecific binder molecule of any one of aspects 139-168, wherein the GPRC5D binding moiety is derived from Talquetamab.
Aspect 170. The multispecific binder molecule of any one of aspects 139-169, wherein the FcRH5 binding moiety is derived from Cevostamab or DFRF4539A.
Aspect 171. The multispecific binder molecule of any one of aspects 139-170, wherein the FcRH5 binding moiety is derived from Cevostamab.
Aspect 172. The multispecific binder molecule of any one of aspects 139-171, wherein the CD3 binding moiety is derived from AMG 701, Alnuctamab, Elranatamab, Linvoseltamab, Teclistamab, ABBV-383, HPN217, XmAb969, ISB1347, Talquetamab, RG6234, Cevostamab, SAR442257, F182112, or ISB 2001.
Aspect 173. The multispecific binder molecule of any one of aspects 139-172, wherein at least one of the three distinct tumor antigen binding moieties comprises a nucleic acid aptamer, a peptide aptamer, a designed ankyrin repeat protein (DARPin), an affimers, an inhibitor cysteine knot (knottin), an avimer, a monobody, an anticalin, a fynomer, or an affibody.
Aspect 174. The multispecific binder molecule of any one of aspects 139-173, wherein the immune cell engager comprises a nucleic acid aptamer, a peptide aptamer, a designed ankyrin repeat protein (DARPin), an affimers, an inhibitor cysteine knot (knottin), an avimer, a monobody, an anticalin, a fynomer, or an affibody.
Aspect 175. A method for inducing an immune response against a tumor cell, comprising contacting the tumor cell with the multispecific binder molecule of any one of aspects 139-174.
Aspect 176. The method of aspect 175, wherein the contacting induces proximity between the tumor cell and one or more immune effector cells.
Aspect 177. The method of aspect 175 or 176, wherein the contacting supports formation of a cytolytic immunological synapse between the tumor cell and one or more immune effector cells.
Aspect 178. The method of any aspect 176 or 177, wherein the one or more immune effector cells comprise one or more activated T cells.
Aspect 179. The method of aspect 178, wherein the one or more activated T cells release primary cytokines that induce the activation of other immune cell types wherein the other immune cell types comprise macrophages, dendritic cells, monocytes, NK cells, or any combination thereof.
Aspect 180. The method of aspect 179, wherein the other immune cell types produce elevated levels of secondary cytokines, wherein the secondary cytokines comprise IL6, IL10, IL5, or any combination thereof.
Aspect 181. The method of any one of aspects 175-180, wherein the one or more immune effector cells induce apoptosis in a target tumor cell.
Aspect 182. The method of any one of aspects 175-181, wherein the one or more immune effector cells induce pyroptosis in a target tumor cell.
Aspect 183. The method of any one of aspects 175-182, wherein the one or more immune effector cells release one or more cytotoxic proteases of the granzyme family following the contacting.
Aspect 184. The method of aspect 183, wherein the one or more cytotoxic proteases of the granzyme family comprise Granzyme B (GrB).
Aspect 185. The method of any one of aspects 175-184, wherein the one or more immune effector cells release one or more lytic proteins following the contacting.
Aspect 186. The method of aspect 185, wherein the one or more lytic proteins comprise perforin.
Aspect 187. The method of any one of aspects 175-186, wherein the contacting engages a T cell receptor (TCR) on one or more T cells.
Aspect 188. The method of aspect 187, wherein the one or more T cells comprise naïve T cells, helper T cells, cytotoxic T cells, or any combination thereof.
Aspect 189. The method of aspect 187 or 188, wherein the one or more T cells receive a co-stimulatory signal resulting in T cell proliferation, differentiation, and survival.
Aspect 190. The method of aspect 187 or 188, wherein the contacting induces co-stimulation independent polyclonal T-cell activation.
Aspect 191. The method of any one of aspects 175-190, wherein the tumor cell is a myeloma tumor cell.
Aspect 192. The method of any one of aspects 175-191, wherein the tumor cell is a multiple myeloma tumor cell.
Aspect 193. A method for treating a cancer in a subject in need thereof, comprising administering a therapeutically effective amount of the multispecific binder molecule of any one of aspects 139-174 to the subject.
Aspect 194. The method of aspect 193, wherein the cancer is a hematological cancer.
Aspect 195. The method of aspect 193 or 194, wherein the cancer is myeloma.
Aspect 196. The method of any one of aspects 193-195, wherein the cancer is multiple myeloma.
Aspect 197. The method of any one of aspects 193-196, wherein the therapeutically effective amount reduces a risk of the subject developing cytokine release syndrome (CRS) following the administering.

1. A multispecific binder molecule, comprising three distinct tumor antigen binding moieties (a-c) comprising: a) a TNFRSF17 (BCMA) binding moiety, b) a GPRC5D binding moiety, and c) a FcRH5 binding moiety; and d) an immune cell engager.

2. The multispecific binder molecule of claim 1, wherein the immune cell engager comprises a T cell binding moiety, a natural killer (NK) cell binding moiety, or a NKT cell binding moiety.

3. The multispecific binder molecule of claim 1, wherein the immune cell engager comprises a T cell binding moiety, wherein the T cell binding moiety comprises a CD3 binding moiety.

4. (canceled)

5. The multispecific binder molecule of claim 3, wherein at least one of the three distinct tumor antigen binding moieties is an antibody or antigen binding fragment thereof, wherein the antibody or antigen binding fragment thereof comprises a chimeric antibody, a monoclonal antibody, a human antibody, a humanized antibody, a monovalent Fab′, a divalent Fab2, a single-chain variable fragment (scFv), a diabody, a minibody, a single-domain antibody (sdAb), or a camelid antibody.

6. (canceled)

7. (canceled)

8. The multispecific binder molecule of claim 5, wherein the multispecific binder molecule comprises a first immunoglobulin chain constant region and a second immunoglobulin chain constant region.

9. The multispecific binder molecule of claim 8, wherein the TNFRSF17 (BCMA) binding moiety and the GPRC5D binding moiety are linked to the first immunoglobulin chain constant region, and wherein the CD3 binding moiety and the FcRH5 binding moiety are linked to the second immunoglobulin chain constant region.

10. The multispecific binder molecule of claim 8, wherein the TNFRSF17 (BCMA) binding moiety and the CD3 binding moiety are linked to the first immunoglobulin chain constant region, and wherein the GPRC5D binding moiety and the FcRH5 binding moiety are linked to the second immunoglobulin chain constant region.

11. The multispecific binder molecule of claim 8, wherein the TNFRSF17 (BCMA) binding moiety and the FcRH5 binding moiety are linked to the first immunoglobulin chain constant region, and wherein the GPRC5D binding moiety and the CD3 binding moiety are linked to the second immunoglobulin chain constant region.

12. The multispecific binder molecule of claim 5, wherein the a) TNFRSF17 (BCMA) binding moiety, b) GPRC5D binding moiety, c) FcRH5 binding moiety, and d) CD3 binding moiety, comprise an antibody or antigen binding fragment thereof, wherein the antibody or antigen binding fragment thereof comprises a Fab′, scFv, IgG-scFv, sdAb, mini-antibody, minibody, scFv-CH3 knob-in-hole (KiH), or scFv-KiH.

13. (canceled)

14. The multispecific binder molecule of claim 8, wherein the first or the second immunoglobulin chain constant regions form an IgG1 heavy chain or a variant thereof.

15. The multispecific binder molecule of claim 14, wherein the TNFRSF17 (BCMA) binding moiety is coupled to an N-terminus of the IgG1 heavy chain or the variant thereof or to a C-terminus of the IgG1 heavy chain or the variant thereof.

16. (canceled)

17. The multispecific binder molecule of claim 14, wherein the GPRC5D binding moiety is coupled to an N-terminus of the IgG1 heavy chain or the variant thereof or to a C-terminus of the IgG1 heavy chain or the variant thereof.

18. (canceled)

19. The multispecific binder molecule of claim 14, wherein the FcRH5 binding moiety is coupled to an N-terminus of the IgG1 heavy chain or the variant thereof or to a C-terminus of the IgG1 heavy chain or the variant thereof.

20. (canceled)

21. The multispecific binder molecule of claim 14, wherein the CD3 binding moiety is coupled to an N-terminus of the IgG1 heavy chain or the variant thereof or to a C-terminus of the IgG1 heavy chain or the variant thereof.

22. (canceled)

23. The multispecific binder molecule of claim 14, wherein the IgG1 heavy chain comprises a mutation that reduces FcγR binding.

24. The multispecific binder molecule of claim 23, wherein the mutation comprises a N297A mutation.

25. (canceled)

26. (canceled)

27. The multispecific binder molecule of claim 14, wherein one of the three distinct tumor antigen binding moieties is coupled to an immunoglobulin lambda light chain domain and another of the three distinct tumor antigen binding moieties is coupled to an immunoglobulin kappa light chain domain.

28. The multispecific binder molecule of claim 3, wherein (a) the TNFRSF17 (BCMA) binding moiety is derived from Teclistamab, AMG 701, Alnuctamab, Belantamab mafodotin, Elranatamab, Linvoseltamab, ABBV-383, HPN217, ISB 2001, GSK287916, MEDI2228, TNB-383B, REGN545, or AMG 420; (b) the GPRC5D binding moiety is derived from Talquetamab or RG6234; (c) the FcRH5 binding moiety is derived from Cevostamab or DFRF4539A; or (d) the CD3 binding moiety is derived from AMG 701, Alnuctamab, Elranatamab, Linvoseltamab, Teclistamab, ABBV-383, HPN217, XmAb969, ISB1347, Talquetamab, RG6234, Cevostamab, SAR442257, F182112, or ISB 2001.

29-31. (canceled)

32. A method for inducing an immune response against a tumor cell, comprising contacting the tumor cell with the multispecific binder molecule of claim 1.

33-48. (canceled)

49. A method for treating a cancer in a subject in need thereof, comprising administering to the subject a therapeutically effective amount of the multispecific binder molecule of claim 1 to the subject.

50-52. (canceled)


SEQUENCE LISTING (461 sequences)

SEQ ID NO: 1 - protein, 120 aa, synthetic construct
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 2 - protein, 119 aa, synthetic construct
EVKLLESGGGLVQPGGSLKLSCAASGFDFSRYWMSWVRQAPGKGLEWIGEINPDSSTINYTPSLKDKFIISRDNAKNTLYLQMSKVRSEDTALYYCARPDGNYWYFDVWGAGTTVTVSS

SEQ ID NO: 3 - protein, 121 aa, synthetic construct
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSS

SEQ ID NO: 4 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 5 - protein, 116 aa, synthetic construct
EVQLLESGGGLVQPGGSLRLSCAASGFTFSDNAMGWVRQAPGKGLEWVSAISGPGSSTYYADSVKGRFTISRDNSKNTLYLQMNSLRAEDTAVYYCAKVLGWFDYWGQGTLVTVSS

SEQ ID NO: 6 - protein, 125 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 7 - protein, 125 aa, synthetic construct
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSS

SEQ ID NO: 8 - protein, 125 aa, synthetic construct
EVQLVESGGGLVQPGGSLRLSCAASGFTFSNFWMTWVRQAPGKGLEWVANMNQDGSEKYYVDSVKGRFTISRDNAKSSLYLQMNSLRAEDTAVYYCARDREYCISTSCYDDFDYWGQGTLVTVSS

SEQ ID NO: 9 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSS

SEQ ID NO: 10 - protein, 120 aa, synthetic construct
EVQLVESGPGLVKPSETLSLTCTVSGFSLTRFGVHWVRQPPGKGLEWLGVIWRGGSTDYNAAFVSRLTISKDNSKNQVSLKLSSVTAADTAVYYCSNHYYGSSDYALDNWGQGTLVTVSS

SEQ ID NO: 11 - protein, 119 aa, synthetic construct
EVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSS

SEQ ID NO: 12 - protein, 109 aa, synthetic construct
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQ

SEQ ID NO: 13 - protein, 107 aa, synthetic construct
DIVMTQSHKFMSTSVGDRVSITCKASQDVGIAVAWYQQKPGQSPKLLIYWASTRHTGVPDRFTGSGSGTDFTLTISNVQSEDLADYFCQQYSSYPYTFGGGTKLEIK

SEQ ID NO: 14 - protein, 107 aa, synthetic construct
DIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKVEIK

SEQ ID NO: 15 - protein, 109 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 16 - protein, 109 aa, synthetic construct
EIVLTQSPGTLSLSPGERATLSCRASQSVSDEYLSWYQQKPGQAPRLLIHSASTRATGIPDRFSGSGSGTDFTLAISRLEPEDFAVYYCQQYGYPPDFTFGQGTKVEIK

SEQ ID NO: 17 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 18 - protein, 109 aa, synthetic construct
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVL

SEQ ID NO: 19 - protein, 120 aa, synthetic construct; identical to SEQ ID NO: 10

SEQ ID NO: 20 - protein, 119 aa, synthetic construct; identical to SEQ ID NO: 11

SEQ ID NO: 21 - protein, 492 aa, synthetic construct
  VARIANT 15..40: note = Residue may be deleted
XXXXXXXXXXXXXXXXXXXXXXXXXXXXXXXXXXXXXXXXEVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 490 aa, synthetic construct
  VARIANT 15..40: note = Residue may be deleted
XXXXXXXXXXXXXXXXXXXXXXXXXXXXXXXXXXXXXXXXQVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 486 aa, synthetic construct
  VARIANT 15..40: note = Residue may be deleted
XXXXXXXXXXXXXXXXXXXXXXXXXXXXXXXXXXXXXXXXEVQLVESGGGLVQPGGSLRLSCAASGFTFSDYYMAWVRQAPGKGLEWVASINYDGSSTYYVDSVKGRFTISRDNAKNSLYLQMNSLRAEDTAVYYCARDRGYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 489 aa, synthetic construct
  VARIANT 15..40: note = Residue may be deleted
XXXXXXXXXXXXXXXXXXXXXXXXXXXXXXXXXXXXXXXXEVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 487 aa, synthetic construct
  VARIANT 15..40: note = Residue may be deleted
XXXXXXXXXXXXXXXXXXXXXXXXXXXXXXXXXXXXXXXXEVQLVESGGGLVQPGGSLRLSCAASGFTFSDYYMTWVRQAPGKGLEWVAFIRNRARGYTSDHNPSVKGRFTISRDNAKNSLYLQMNSLRAEDTAVYYCARDRPSYYVLDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCRVRCPRCPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSRLTVDKSRWQQGNVFSCSVMHEALHNHYTQKSLSLSPGK

SEQ ID NO: 26 - protein, 481 aa, synthetic construct
  VARIANT 15..40: note = Residue may be deleted
XXXXXXXXXXXXXXXXXXXXXXXXXXXXXXXXXXXXXXXXEVQLLESGGGLVQPGGSLRLSCAASGFTFSSYPMSWVRQAPGKGLEWVSAIGGSGGSLPYADIVKGRFTISRDNSKNTLYLQMNSLRAEDTAVYYCARYWPMDIWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCEVECPECPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCEVKGFYPSDIAVEWESNGQPENNYKTTPPMLDSDGSFFLYSKLTVDKSRWQQGNVFSCSVMHEALHNHYTQKSLSLSPGK

SEQ ID NO: 27 - protein, 489 aa, synthetic construct
  VARIANT 15..40: note = Residue may be deleted
XXXXXXXXXXXXXXXXXXXXXXXXXXXXXXXXXXXXXXXXEVQLVESGGGLVQPGGSLRLSCAASGFTFSNFWMTWVRQAPGKGLEWVANMNQDGSEKYYVDSVKGRFTISRDNAKSSLYLQMNSLRAEDTAVYYCARDREYCISTSCYDDFDYWGQGTLTVSSASTKGPSVFPLAPCSRSTSESTAALGCLVKDYFPEPVTVSWNSGALTSGVHTFPALQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 490 aa, synthetic construct
  VARIANT 15..40: note = Residue may be deleted
XXXXXXXXXXXXXXXXXXXXXXXXXXXXXXXXXXXXXXXXEVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 29 - protein, 488 aa, synthetic construct
  VARIANT 15..40: note = Residue may be deleted
XXXXXXXXXXXXXXXXXXXXXXXXXXXXXXXXXXXXXXXXQLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 492 aa, synthetic construct
  VARIANT 15..40: note = Residue may be deleted
XXXXXXXXXXXXXXXXXXXXXXXXXXXXXXXXXXXXXXXXEVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 31 - protein, 512 aa, synthetic construct
  VARIANT 15..40: note = Residue may be deleted
XXXXXXXXXXXXXXXXXXXXXXXXXXXXXXXXXXXXXXXXEVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSGGGGSEVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 32 - protein, 490 aa, synthetic construct
  VARIANT 15..40: note = Residue may be deleted
XXXXXXXXXXXXXXXXXXXXXXXXXXXXXXXXXXXXXXXXEVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 490 aa, synthetic construct
  VARIANT 15..40: note = Residue may be deleted
XXXXXXXXXXXXXXXXXXXXXXXXXXXXXXXXXXXXXXXXEVQLVESGPGLVKPSETLSLTCTVSGFSLTRFGVHWVRQPPGKGLEWLGVIWRGGSTDYNAAFVSRLTISKDNSKNQVSLKLSSVTAADTAVYYCSNHYYGSSDYALDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 489 aa, synthetic construct
  VARIANT 15..40: note = Residue may be deleted
XXXXXXXXXXXXXXXXXXXXXXXXXXXXXXXXXXXXXXXXEVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 35 - protein, 254 aa, synthetic construct
  VARIANT 15..40: note = Residue may be deleted
XXXXXXXXXXXXXXXXXXXXXXXXXXXXXXXXXXXXXXXXEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 254 aa, synthetic construct
  VARIANT 15..40: note = Residue may be deleted
XXXXXXXXXXXXXXXXXXXXXXXXXXXXXXXXXXXXXXXXDIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 260 aa, synthetic construct
  VARIANT 15..40: note = Residue may be deleted
XXXXXXXXXXXXXXXXXXXXXXXXXXXXXXXXXXXXXXXXDVVMTQTPLSLSVTPGQPASISCRSSQSLVHSNGNTYLHWYLQKPGQSPQLLIYKVSNRFSGVPDRFSGSGSGTDFTLKISRVEAEDVGVYFCSQSTHVPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 254 aa, synthetic construct
  VARIANT 15..40: note = Residue may be deleted
XXXXXXXXXXXXXXXXXXXXXXXXXXXXXXXXXXXXXXXXDIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 259 aa, synthetic construct
  VARIANT 15..40: note = Residue may be deleted
XXXXXXXXXXXXXXXXXXXXXXXXXXXXXXXXXXXXXXXXDIVMTQSPDSLAVSLGERATINCKSSQSLFNVRSRKNYLAWYQQKPGQPPKLLISWASTRESGVPDRFSGSGSGTDFTLTISSLQAEDVAVYYCKQSYDLFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 255 aa, synthetic construct
  VARIANT 15..40: note = Residue may be deleted
XXXXXXXXXXXXXXXXXXXXXXXXXXXXXXXXXXXXXXXXEIVLTQSPGTLSLSPGERATLSCRASQSVSSSYLAWYQQKPGQAPRLLMYDASIRATGIPDRFSGSGSGTDFTLTISRLEPEDFAVYYCQQYQSWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 255 aa, synthetic construct
  VARIANT 15..40: note = Residue may be deleted
XXXXXXXXXXXXXXXXXXXXXXXXXXXXXXXXXXXXXXXXDIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 254 aa, synthetic construct
  VARIANT 15..40: note = Residue may be deleted
XXXXXXXXXXXXXXXXXXXXXXXXXXXXXXXXXXXXXXXXSYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 43 - protein, 255 aa, synthetic construct
  VARIANT 15..40: note = Residue may be deleted
XXXXXXXXXXXXXXXXXXXXXXXXXXXXXXXXXXXXXXXXQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 44 - protein, 254 aa, synthetic construct
  VARIANT 15..40: note = Residue may be deleted
XXXXXXXXXXXXXXXXXXXXXXXXXXXXXXXXXXXXXXXXEIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 254 aa, synthetic construct
  VARIANT 15..40: note = Residue may be deleted
XXXXXXXXXXXXXXXXXXXXXXXXXXXXXXXXXXXXXXXXDIQMTQSPSSLSASVGDRVTITCKASQDVRNLVVWFQQKPGKAPKLLIYSGSYRYSGVPSRFSGSGSGTDFTLTISSLQPEDFATYYCQQHYSPPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 259 aa, synthetic construct
  VARIANT 15..40: note = Residue may be deleted
XXXXXXXXXXXXXXXXXXXXXXXXXXXXXXXXXXXXXXXXDIVMTQSPDSLAVSLGERATINCKSSQSLLNSRTRKNYLAWYQQKPGQSPKLLIYWTSTRKSGVPDRFSGSGSGTDFTLTISSLQAEDVAVYYCKQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 986 aa, synthetic construct
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 507 aa, synthetic construct
EVQLVESGGGLVQPGRSLTLSCAASTNIFSISPYGWYRQAPGKQRELVAAIHGTSTLYADSVKGRFTISRDNAKNSIYLQMNSLRPEDTALYYCNKVPWGDYHPGNVYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 49 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 51 - protein, 330 aa, Homo sapiens [sequence omitted]

SEQ ID NO: 52 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 57 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 110 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAK

SEQ ID NO: 61 - protein, 110 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAK

SEQ ID NO: 62 - protein, 107 aa, synthetic construct
GQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 107 aa, synthetic construct
GQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 64 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 66 - protein, 227 aa, Homo sapiens
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 330 aa, synthetic construct
ASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 69 - protein, 107 aa, Homo sapiens [sequence omitted]

SEQ ID NO: 70 - protein, 24 aa, synthetic construct
MDPKGSLSWRILLFLSLAFELSYG

SEQ ID NO: 71 - protein, 20 aa, synthetic construct
METDTLLLWVLLLWVPGSTG

SEQ ID NO: 72 - protein, 19 aa, synthetic construct
MGWSLILLFLVAVATRVHS

SEQ ID NO: 73 - protein, 20 aa, synthetic construct
MRVPAQLLGLLLLWLPGARC

SEQ ID NO: 74 - protein, 19 aa, synthetic construct
MKLWLNWVFLLTLLHGIQC

SEQ ID NO: 75 - protein, 22 aa, synthetic construct
MDFQVQIFSFLLISASVIVSRG

SEQ ID NO: 76 - protein, 30 aa, synthetic construct
  VARIANT 6..30: note = GGGGS repeats may be deleted
GGGGSGGGGSGGGGSGGGGSGGGGSGGGGS

SEQ ID NO: 77 - protein, 15 aa, synthetic construct
GGGGSGGGGSGGGGS

SEQ ID NO: 78 - protein, 20 aa, synthetic construct
GGGGSGGGGSGGGGSGGGGS

SEQ ID NO: 79 - protein, 6 aa, synthetic construct
GSGSGS

SEQ ID NO: 80 - protein, 11 aa, synthetic construct
GGSGGSGGSGG

SEQ ID NO: 81 - protein, 16 aa, synthetic construct
GGGGSLVPRGSGGGGS

SEQ ID NO: 82 - protein, 11 aa, synthetic construct
GGSGGHMGSGG

SEQ ID NO: 83 - protein, 5 aa, synthetic construct
GGSGG

SEQ ID NO: 84 - protein, 5 aa, synthetic construct
GGSSG

SEQ ID NO: 85 - protein, 12 aa, synthetic construct
GSGSGSGSGGSG

SEQ ID NO: 86 - protein, 20 aa, synthetic construct
  VARIANT 3..20: note = GS repeats may be deleted
GSGSGSGSGSGSGSGSGSGS

SEQ ID NO: 87 - protein, 30 aa, synthetic construct; identical to SEQ ID NO: 76
  VARIANT 6..30: note = GGGGS repeats may be deleted

SEQ ID NO: 88 - protein, 8 aa, synthetic construct
GFTFNSFA

SEQ ID NO: 89 - protein, 5 aa, synthetic construct
SFAMS

SEQ ID NO: 90 - protein, 10 aa, synthetic construct
GFTFNSFAMS

SEQ ID NO: 91 - protein, 7 aa, synthetic construct
GFTFNSF

SEQ ID NO: 92 - protein, 6 aa, synthetic construct
NSFAMS

SEQ ID NO: 93 - protein, 8 aa, synthetic construct
GYTFTDYW

SEQ ID NO: 94 - protein, 5 aa, synthetic construct
DYWMQ

SEQ ID NO: 95 - protein, 10 aa, synthetic construct
GYTFTDYWMQ

SEQ ID NO: 96 - protein, 7 aa, synthetic construct
GYTFTDY

SEQ ID NO: 97 - protein, 6 aa, synthetic construct
TDYWMQ

SEQ ID NO: 98 - protein, 8 aa, synthetic construct
GFTFSSYY

SEQ ID NO: 99 - protein, 5 aa, synthetic construct
SYYMN

SEQ ID NO: 100 - protein, 10 aa, synthetic construct
GFTFSSYYMN

SEQ ID NO: 101 - protein, 7 aa, synthetic construct
GFTFSSY

SEQ ID NO: 102 - protein, 6 aa, synthetic construct
SSYYMN

SEQ ID NO: 103 - protein, 8 aa, synthetic construct
GFDFSRYW

SEQ ID NO: 104 - protein, 5 aa, synthetic construct
RYWMS

SEQ ID NO: 105 - protein, 10 aa, synthetic construct
GFDFSRYWMS

SEQ ID NO: 106 - protein, 7 aa, synthetic construct
GFDFSRY

SEQ ID NO: 107 - protein, 6 aa, synthetic construct
SRYWMS

SEQ ID NO: 108 - protein, 8 aa, synthetic construct
GFTFSDYY

SEQ ID NO: 109 - protein, 5 aa, synthetic construct
DYYMA

SEQ ID NO: 110 - protein, 10 aa, synthetic construct
GFTFSDYYMA

SEQ ID NO: 111 - protein, 7 aa, synthetic construct
GFTFSDY

SEQ ID NO: 112 - protein, 6 aa, synthetic construct
SDYYMA

SEQ ID NO: 113 - protein, 8 aa, synthetic construct
GYTFTNHI

SEQ ID NO: 114 - protein, 5 aa, synthetic construct
NHIIH

SEQ ID NO: 115 - protein, 10 aa, synthetic construct
GYTFTNHIIH

SEQ ID NO: 116 - protein, 7 aa, synthetic construct
GYTFTNH

SEQ ID NO: 117 - protein, 6 aa, synthetic construct
TNHIIH

SEQ ID NO: 118 - protein, 8 aa, synthetic construct
GFTFNKYA

SEQ ID NO: 119 - protein, 5 aa, synthetic construct
KYAMN

SEQ ID NO: 120 - protein, 10 aa, synthetic construct
GFTFNKYAMN

SEQ ID NO: 121 - protein, 7 aa, synthetic construct
GFTFNKY

SEQ ID NO: 122 - protein, 6 aa, synthetic construct
NKYAMN

SEQ ID NO: 123 - protein, 8 aa, synthetic construct
GFTFSDNA

SEQ ID NO: 124 - protein, 5 aa, synthetic construct
DNAMG

SEQ ID NO: 125 - protein, 10 aa, synthetic construct
GFTFSDNAMG

SEQ ID NO: 126 - protein, 7 aa, synthetic construct
GFTFSDN

SEQ ID NO: 127 - protein, 6 aa, synthetic construct
SDNAMG

SEQ ID NO: 128 - protein, 8 aa, synthetic construct
GFTFSTYA

SEQ ID NO: 129 - protein, 5 aa, synthetic construct
TYAMN

SEQ ID NO: 130 - protein, 10 aa, synthetic construct
GFTFSTYAMN

SEQ ID NO: 131 - protein, 7 aa, synthetic construct
GFTFSTY

SEQ ID NO: 132 - protein, 6 aa, synthetic construct
STYAMN

SEQ ID NO: 133 - protein, 5 aa, synthetic construct
DYYMT

SEQ ID NO: 134 - protein, 10 aa, synthetic construct
GFTFSDYYMT

SEQ ID NO: 135 - protein, 6 aa, synthetic construct
SDYYMT

SEQ ID NO: 136 - protein, 8 aa, synthetic construct
GFTFSSYP

SEQ ID NO: 137 - protein, 5 aa, synthetic construct
SYPMS

SEQ ID NO: 138 - protein, 10 aa, synthetic construct
GFTFSSYPMS

SEQ ID NO: 139 - protein, 6 aa, synthetic construct
SSYPMS

SEQ ID NO: 140 - protein, 8 aa, synthetic construct
GFTFSNFW

SEQ ID NO: 141 - protein, 5 aa, synthetic construct
NFWMT

SEQ ID NO: 142 - protein, 10 aa, synthetic construct
GFTFSNFWMT

SEQ ID NO: 143 - protein, 7 aa, synthetic construct
GFTFSNF

SEQ ID NO: 144 - protein, 6 aa, synthetic construct
SNFWMT

SEQ ID NO: 145 - protein, 8 aa, synthetic construct
GFTFDDYS

SEQ ID NO: 146 - protein, 5 aa, synthetic construct
DYSMH

SEQ ID NO: 147 - protein, 10 aa, synthetic construct
GFTFDDYSMH

SEQ ID NO: 148 - protein, 7 aa, synthetic construct
GFTFDDY

SEQ ID NO: 149 - protein, 6 aa, synthetic construct
DDYSMH

SEQ ID NO: 150 - protein, 8 aa, synthetic construct
GFTFNTYA

SEQ ID NO: 151 - protein, 10 aa, synthetic construct
GFTFNTYAMN

SEQ ID NO: 152 - protein, 7 aa, synthetic construct
GFTFNTY

SEQ ID NO: 153 - protein, 6 aa, synthetic construct
NTYAMN

SEQ ID NO: 154 - protein, 10 aa, synthetic construct
GGSISSGSYF

SEQ ID NO: 155 - protein, 7 aa, synthetic construct
SGSYFWG

SEQ ID NO: 156 - protein, 12 aa, synthetic construct
GGSISSGSYFWG

SEQ ID NO: 157 - protein, 9 aa, synthetic construct
GGSISSGSY

SEQ ID NO: 158 - protein, 8 aa, synthetic construct
SSGSYFWG

SEQ ID NO: 159 - protein, 8 aa, synthetic construct
GFTVSSYG

SEQ ID NO: 160 - protein, 5 aa, synthetic construct
SYGMS

SEQ ID NO: 161 - protein, 10 aa, synthetic construct
GFTVSSYGMS

SEQ ID NO: 162 - protein, 7 aa, synthetic construct
GFTVSSY

SEQ ID NO: 163 - protein, 6 aa, synthetic construct
SSYGMS

SEQ ID NO: 164 - protein, 8 aa, synthetic construct
GFTFDDYA

SEQ ID NO: 165 - protein, 5 aa, synthetic construct
DYAMH

SEQ ID NO: 166 - protein, 10 aa, synthetic construct
GFTFDDYAMH

SEQ ID NO: 167 - protein, 6 aa, synthetic construct
DDYAMH

SEQ ID NO: 168 - protein, 8 aa, synthetic construct
TNIFSISP

SEQ ID NO: 169 - protein, 5 aa, synthetic construct
ISPYG

SEQ ID NO: 170 - protein, 10 aa, synthetic construct
TNIFSISPYG

SEQ ID NO: 171 - protein, 7 aa, synthetic construct
TNIFSIS

SEQ ID NO: 172 - protein, 6 aa, synthetic construct
SISPYG

SEQ ID NO: 173 - protein, 5 aa, synthetic construct
KYAIN

SEQ ID NO: 174 - protein, 10 aa, synthetic construct
GFTFNKYAIN

SEQ ID NO: 175 - protein, 6 aa, synthetic construct
NKYAIN

SEQ ID NO: 176 - protein, 8 aa, synthetic construct
GFSLTRFG

SEQ ID NO: 177 - protein, 5 aa, synthetic construct
RFGVH

SEQ ID NO: 178 - protein, 10 aa, synthetic construct
GFSLTRFGVH

SEQ ID NO: 179 - protein, 7 aa, synthetic construct
GFSLTRF

SEQ ID NO: 180 - protein, 6 aa, synthetic construct
TRFGVH

SEQ ID NO: 181 - protein, 8 aa, synthetic construct
GFTFTSYY

SEQ ID NO: 182 - protein, 5 aa, synthetic construct
SYYIH

SEQ ID NO: 183 - protein, 10 aa, synthetic construct
GFTFTSYYIH

SEQ ID NO: 184 - protein, 7 aa, synthetic construct
GFTFTSY

SEQ ID NO: 185 - protein, 6 aa, synthetic construct
TSYYIH

SEQ ID NO: 186 - protein, 8 aa, synthetic construct
ISGSGGGT

SEQ ID NO: 187 - protein, 17 aa, synthetic construct
AISGSGGGTYYADSVKG

SEQ ID NO: 188 - protein, 10 aa, synthetic construct
AISGSGGGTY

SEQ ID NO: 189 - protein, 6 aa, synthetic construct
SGSGGG

SEQ ID NO: 190 - protein, 13 aa, synthetic construct
WVSAISGSGGGTY

SEQ ID NO: 191 - protein, 8 aa, synthetic construct
IYPGDGDT

SEQ ID NO: 192 - protein, 17 aa, synthetic construct
TIYPGDGDTGYAQKFQG

SEQ ID NO: 193 - protein, 10 aa, synthetic construct
TIYPGDGDTG

SEQ ID NO: 194 - protein, 6 aa, synthetic construct
YPGDGD

SEQ ID NO: 195 - protein, 13 aa, synthetic construct
WIGTIYPGDGDTG

SEQ ID NO: 196 - protein, 8 aa, synthetic construct
ISGDPSNT

SEQ ID NO: 197 - protein, 17 aa, synthetic construct
GISGDPSNTYYADSVKG

SEQ ID NO: 198 - protein, 10 aa, synthetic construct
GISGDPSNTY

SEQ ID NO: 199 - protein, 6 aa, synthetic construct
SGDPSN

SEQ ID NO: 200 - protein, 13 aa, synthetic construct
WVSGISGDPSNTY

SEQ ID NO: 201 - protein, 8 aa, synthetic construct
INPDSSTI

SEQ ID NO: 202 - protein, 17 aa, synthetic construct
EINPDSSTINYTPSLKD

SEQ ID NO: 203 - protein, 10 aa, synthetic construct
EINPDSSTIN

SEQ ID NO: 204 - protein, 6 aa, synthetic construct
NPDSST

SEQ ID NO: 205 - protein, 13 aa, synthetic construct
WIGEINPDSSTIN

SEQ ID NO: 206 - protein, 8 aa, synthetic construct
INYDGSST

SEQ ID NO: 207 - protein, 17 aa, synthetic construct
SINYDGSSTYYVDSVKG

SEQ ID NO: 208 - protein, 10 aa, synthetic construct
SINYDGSSTY

SEQ ID NO: 209 - protein, 6 aa, synthetic construct
NYDGSS

SEQ ID NO: 210 - protein, 13 aa, synthetic construct
WVASINYDGSSTY

SEQ ID NO: 211 - protein, 17 aa, synthetic construct
EINPDSSTINYAPSLKD

SEQ ID NO: 212 - protein, 8 aa, synthetic construct
INPYPGYH

SEQ ID NO: 213 - protein, 17 aa, synthetic construct
YINPYPGYHAYNEKFQG

SEQ ID NO: 214 - protein, 10 aa, synthetic construct
YINPYPGYHA

SEQ ID NO: 215 - protein, 6 aa, synthetic construct
NPYPGY

SEQ ID NO: 216 - protein, 13 aa, synthetic construct
WMGYINPYPGYHA

SEQ ID NO: 217 - protein, 10 aa, synthetic construct
IRSKYNNYAT

SEQ ID NO: 218 - protein, 19 aa, synthetic construct
RIRSKYNNYATYYADSVKD

SEQ ID NO: 219 - protein, 12 aa, synthetic construct
RIRSKYNNYATY

SEQ ID NO: 220 - protein, 8 aa, synthetic construct
RSKYNNYA

SEQ ID NO: 221 - protein, 15 aa, synthetic construct
WVARIRSKYNNYATY

SEQ ID NO: 222 - protein, 8 aa, synthetic construct
ISGPGSST

SEQ ID NO: 223 - protein, 17 aa, synthetic construct
AISGPGSSTYYADSVKG

SEQ ID NO: 224 - protein, 10 aa, synthetic construct
AISGPGSSTY

SEQ ID NO: 225 - protein, 6 aa, synthetic construct
SGPGSS

SEQ ID NO: 226 - protein, 13 aa, synthetic construct
WVSAISGPGSSTY

SEQ ID NO: 227 - protein, 19 aa, synthetic construct
RIRSKYNNYATYYADSVKG

SEQ ID NO: 228 - protein, 15 aa, synthetic construct
WVSRIRSKYNNYATY

SEQ ID NO: 229 - protein, 10 aa, synthetic construct
IRNRARGYTS

SEQ ID NO: 230 - protein, 19 aa, synthetic construct
FIRNRARGYTSDHNPSVKG

SEQ ID NO: 231 - protein, 12 aa, synthetic construct
FIRNRARGYTSD

SEQ ID NO: 232 - protein, 8 aa, synthetic construct
RNRARGYT

SEQ ID NO: 233 - protein, 15 aa, synthetic construct
WVAFIRNRARGYTSD

SEQ ID NO: 234 - protein, 8 aa, synthetic construct
IGGSGGSL

SEQ ID NO: 235 - protein, 17 aa, synthetic construct
AIGGSGGSLPYADIVKG

SEQ ID NO: 236 - protein, 10 aa, synthetic construct
AIGGSGGSLP

SEQ ID NO: 237 - protein, 6 aa, synthetic construct
GGSGGS

SEQ ID NO: 238 - protein, 13 aa, synthetic construct
WVSAIGGSGGSLP

SEQ ID NO: 239 - protein, 8 aa, synthetic construct
MNQDGSEK

SEQ ID NO: 240 - protein, 17 aa, synthetic construct
NMNQDGSEKYYVDSVKG

SEQ ID NO: 241 - protein, 10 aa, synthetic construct
NMNQDGSEKY

SEQ ID NO: 242 - protein, 6 aa, synthetic construct
NQDGSE

SEQ ID NO: 243 - protein, 13 aa, synthetic construct
WVANMNQDGSEKY

SEQ ID NO: 244 - protein, 8 aa, synthetic construct
ISWNSGSK

SEQ ID NO: 245 - protein, 17 aa, synthetic construct
GISWNSGSKGYADSVKG

SEQ ID NO: 246 - protein, 10 aa, synthetic construct
GISWNSGSKG

SEQ ID NO: 247 - protein, 6 aa, synthetic construct
SWNSGS

SEQ ID NO: 248 - protein, 13 aa, synthetic construct
WVSGISWNSGSKG

SEQ ID NO: 249 - protein, 7 aa, synthetic construct
IYYSGIT

SEQ ID NO: 250 - protein, 16 aa, synthetic construct
SIYYSGITYYNPSLKS

SEQ ID NO: 251 - protein, 9 aa, synthetic construct
SIYYSGITY

SEQ ID NO: 252 - protein, 5 aa, synthetic construct
YYSGI

SEQ ID NO: 253 - protein, 12 aa, synthetic construct
WIGSIYYSGITY

SEQ ID NO: 254 - protein, 19 aa, synthetic construct
RIRSKYNNYATYYAASVKG

SEQ ID NO: 255 - protein, 8 aa, synthetic construct
IRGSDGST

SEQ ID NO: 256 - protein, 17 aa, synthetic construct
GIRGSDGSTYYADSVKG

SEQ ID NO: 257 - protein, 10 aa, synthetic construct
GIRGSDGSTY

SEQ ID NO: 258 - protein, 6 aa, synthetic construct
RGSDGS

SEQ ID NO: 259 - protein, 13 aa, synthetic construct
WVSGIRGSDGSTY

SEQ ID NO: 260 - protein, 8 aa, synthetic construct
ISWNSGSI

SEQ ID NO: 261 - protein, 17 aa, synthetic construct
GISWNSGSIGYADSVKG

SEQ ID NO: 262 - protein, 10 aa, synthetic construct
GISWNSGSIG

SEQ ID NO: 263 - protein, 13 aa, synthetic construct
WVSGISWNSGSIG

SEQ ID NO: 264 - protein, 6 aa, synthetic construct
IHGTST

SEQ ID NO: 265 - protein, 15 aa, synthetic construct
AIHGTSTLYADSVKG

SEQ ID NO: 266 - protein, 8 aa, synthetic construct
AIHGTSTL

SEQ ID NO: 267 - protein, 4 aa, synthetic construct
HGTS

SEQ ID NO: 268 - protein, 11 aa, synthetic construct
LVAAIHGTSTL

SEQ ID NO: 269 - protein, 19 aa, synthetic construct
RIRSKYNNYATYYADQVKD

SEQ ID NO: 270 - protein, 7 aa, synthetic construct
IWRGGST

SEQ ID NO: 271 - protein, 16 aa, synthetic construct
VIWRGGSTDYNAAFVS

SEQ ID NO: 272 - protein, 9 aa, synthetic construct
VIWRGGSTD

SEQ ID NO: 273 - protein, 5 aa, synthetic construct
WRGGS

SEQ ID NO: 274 - protein, 12 aa, synthetic construct
WLGVIWRGGSTD

SEQ ID NO: 275 - protein, 8 aa, synthetic construct
IYPENDNT

SEQ ID NO: 276 - protein, 17 aa, synthetic construct
WIYPENDNTKYNEKFKD

SEQ ID NO: 277 - protein, 10 aa, synthetic construct
WIYPENDNTK

SEQ ID NO: 278 - protein, 6 aa, synthetic construct
YPENDN

SEQ ID NO: 279 - protein, 13 aa, synthetic construct
WIGWIYPENDNTK

SEQ ID NO: 280 - protein, 15 aa, synthetic construct
AKDKILWFGEPVFDY

SEQ ID NO: 281 - protein, 13 aa, synthetic construct
DKILWFGEPVFDY

SEQ ID NO: 282 - protein, 14 aa, synthetic construct
AKDKILWFGEPVFD

SEQ ID NO: 283 - protein, 13 aa, synthetic construct
ARGDYYGSNSLDY

SEQ ID NO: 284 - protein, 11 aa, synthetic construct
GDYYGSNSLDY

SEQ ID NO: 285 - protein, 12 aa, synthetic construct
ARGDYYGSNSLD

SEQ ID NO: 286 - protein, 13 aa, synthetic construct
ARDLPLVYTGFAY

SEQ ID NO: 287 - protein, 11 aa, synthetic construct
DLPLVYTGFAY

SEQ ID NO: 288 - protein, 12 aa, synthetic construct
ARDLPLVYTGFA

SEQ ID NO: 289 - protein, 12 aa, synthetic construct
ARPDGNYWYFDV

SEQ ID NO: 290 - protein, 10 aa, synthetic construct
PDGNYWYFDV

SEQ ID NO: 291 - protein, 11 aa, synthetic construct
ARPDGNYWYFD

SEQ ID NO: 292 - protein, 10 aa, synthetic construct
ARDRGYYFDY

SEQ ID NO: 293 - protein, 8 aa, synthetic construct
DRGYYFDY

SEQ ID NO: 294 - protein, 9 aa, synthetic construct
ARDRGYYFD

SEQ ID NO: 295 - protein, 14 aa, synthetic construct
ARDGYYRDTDVLDY

SEQ ID NO: 296 - protein, 12 aa, synthetic construct
DGYYRDTDVLDY

SEQ ID NO: 297 - protein, 13 aa, synthetic construct
ARDGYYRDTDVLD

SEQ ID NO: 298 - protein, 16 aa, synthetic construct
VRHGNFGNSYISYWAY

SEQ ID NO: 299 - protein, 14 aa, synthetic construct
HGNFGNSYISYWAY

SEQ ID NO: 300 - protein, 15 aa, synthetic construct
VRHGNFGNSYISYWA

SEQ ID NO: 301 - protein, 9 aa, synthetic construct
AKVLGWFDY

SEQ ID NO: 302 - protein, 7 aa, synthetic construct
VLGWFDY

SEQ ID NO: 303 - protein, 8 aa, synthetic construct
AKVLGWFD

SEQ ID NO: 304 - protein, 16 aa, synthetic construct
VRHGNFGNSYVSWFAY

SEQ ID NO: 305 - protein, 14 aa, synthetic construct
HGNFGNSYVSWFAY

SEQ ID NO: 306 - protein, 15 aa, synthetic construct
VRHGNFGNSYVSWFA

SEQ ID NO: 307 - protein, 12 aa, synthetic construct
ARDRPSYYVLDY

SEQ ID NO: 308 - protein, 10 aa, synthetic construct
DRPSYYVLDY

SEQ ID NO: 309 - protein, 11 aa, synthetic construct
ARDRPSYYVLD

SEQ ID NO: 310 - protein, 8 aa, synthetic construct
ARYWPMDI

SEQ ID NO: 311 - protein, 6 aa, synthetic construct
YWPMDI

SEQ ID NO: 312 - protein, 7 aa, synthetic construct
ARYWPMD

SEQ ID NO: 313 - protein, 18 aa, synthetic construct
ARDREYCISTSCYDDFDY

SEQ ID NO: 314 - protein, 16 aa, synthetic construct
DREYCISTSCYDDFDY

SEQ ID NO: 315 - protein, 17 aa, synthetic construct
ARDREYCISTSCYDDFD

SEQ ID NO: 316 - protein, 17 aa, synthetic construct
AKYGSGYGKFYHYGLDV

SEQ ID NO: 317 - protein, 15 aa, synthetic construct
YGSGYGKFYHYGLDV

SEQ ID NO: 318 - protein, 16 aa, synthetic construct
AKYGSGYGKFYHYGLD

SEQ ID NO: 319 - protein, 13 aa, synthetic construct
ARHDGAVAGLFDY

SEQ ID NO: 320 - protein, 11 aa, synthetic construct
HDGAVAGLFDY

SEQ ID NO: 321 - protein, 12 aa, synthetic construct
ARHDGAVAGLFD

SEQ ID NO: 322 - protein, 16 aa, synthetic construct
ARHGNFGNSYVSWFAY

SEQ ID NO: 323 - protein, 15 aa, synthetic construct
ARHGNFGNSYVSWFA

SEQ ID NO: 324 - protein, 12 aa, synthetic construct
AKQGENDGPFDH

SEQ ID NO: 325 - protein, 10 aa, synthetic construct
QGENDGPFDH

SEQ ID NO: 326 - protein, 11 aa, synthetic construct
AKQGENDGPFD

SEQ ID NO: 327 - protein, 16 aa, synthetic construct
AKDSRGYGDYRLGGAY

SEQ ID NO: 328 - protein, 14 aa, synthetic construct
DSRGYGDYRLGGAY

SEQ ID NO: 329 - protein, 15 aa, synthetic construct
AKDSRGYGDYRLGGA

SEQ ID NO: 330 - protein, 14 aa, synthetic construct
NKVPWGDYHPGNVY

SEQ ID NO: 331 - protein, 12 aa, synthetic construct
VPWGDYHPGNVY

SEQ ID NO: 332 - protein, 13 aa, synthetic construct
NKVPWGDYHPGNV

SEQ ID NO: 333 - protein, 16 aa, synthetic construct
VRHANFGNSYISYWAY

SEQ ID NO: 334 - protein, 14 aa, synthetic construct
HANFGNSYISYWAY

SEQ ID NO: 335 - protein, 15 aa, synthetic construct
VRHANFGNSYISYWA

SEQ ID NO: 336 - protein, 17 aa, synthetic construct
AKYGSGYGKFYYYGMDV

SEQ ID NO: 337 - protein, 15 aa, synthetic construct
YGSGYGKFYYYGMDV

SEQ ID NO: 338 - protein, 16 aa, synthetic construct
AKYGSGYGKFYYYGMD

SEQ ID NO: 339 - protein, 14 aa, synthetic construct
SNHYYGSSDYALDN

SEQ ID NO: 340 - protein, 12 aa, synthetic construct
HYYGSSDYALDN

SEQ ID NO: 341 - protein, 13 aa, synthetic construct
SNHYYGSSDYALD

SEQ ID NO: 342 - protein, 12 aa, synthetic construct
ARDGYSRYYFDY

SEQ ID NO: 343 - protein, 10 aa, synthetic construct
DGYSRYYFDY

SEQ ID NO: 344 - protein, 11 aa, synthetic construct
ARDGYSRYYFD

SEQ ID NO: 345 - protein, 6 aa, synthetic construct
QSVSSY

SEQ ID NO: 346 - protein, 11 aa, synthetic construct
RASQSVSSYLA

SEQ ID NO: 347 - protein, 7 aa, synthetic construct
SSYLAWY

SEQ ID NO: 348 - protein, 6 aa, synthetic construct
QDVSTV

SEQ ID NO: 349 - protein, 11 aa, synthetic construct
KASQDVSTVVA

SEQ ID NO: 350 - protein, 7 aa, synthetic construct
STVVAWY

SEQ ID NO: 351 - protein, 6 aa, synthetic construct
NLRHYY

SEQ ID NO: 352 - protein, 11 aa, synthetic construct
SGDNLRHYYVY

SEQ ID NO: 353 - protein, 7 aa, synthetic construct
HYYVYWY

SEQ ID NO: 354 - protein, 6 aa, synthetic construct
QDVGIA

SEQ ID NO: 355 - protein, 11 aa, synthetic construct
KASQDVGIAVA

SEQ ID NO: 356 - protein, 7 aa, synthetic construct
GIAVAWY

SEQ ID NO: 357 - protein, 11 aa, synthetic construct
QSLVHSNGNTY

SEQ ID NO: 358 - protein, 16 aa, synthetic construct
RSSQSLVHSNGNTYLH

SEQ ID NO: 359 - protein, 12 aa, synthetic construct
VHSNGNTYLHWY

SEQ ID NO: 360 - protein, 6 aa, synthetic construct
QDISNY

SEQ ID NO: 361 - protein, 11 aa, synthetic construct
QASQDISNYLN

SEQ ID NO: 362 - protein, 7 aa, synthetic construct
SNYLNWY

SEQ ID NO: 363 - protein, 9 aa, synthetic construct
TGAVTSGNY

SEQ ID NO: 364 - protein, 14 aa, synthetic construct
GSSTGAVTSGNYPN

SEQ ID NO: 365 - protein, 10 aa, synthetic construct
VTSGNYPNWV

SEQ ID NO: 366 - protein, 7 aa, synthetic construct
QSVSDEY

SEQ ID NO: 367 - protein, 12 aa, synthetic construct
RASQSVSDEYLS

SEQ ID NO: 368 - protein, 8 aa, synthetic construct
SDEYLSWY

SEQ ID NO: 369 - protein, 9 aa, synthetic construct
TGAVTTSNY

SEQ ID NO: 370 - protein, 14 aa, synthetic construct
GSSTGAVTTSNYAN

SEQ ID NO: 371 - protein, 10 aa, synthetic construct
VTTSNYANWV

SEQ ID NO: 372 - protein, 12 aa, synthetic construct
QSLFNVRSRKNY

SEQ ID NO: 373 - protein, 17 aa, synthetic construct
KSSQSLFNVRSRKNYLA

SEQ ID NO: 374 - protein, 13 aa, synthetic construct
FNVRSRKNYLAWY

SEQ ID NO: 375 - protein, 7 aa, synthetic construct
QSVSSSY

SEQ ID NO: 376 - protein, 12 aa, synthetic construct
RASQSVSSSYLA

SEQ ID NO: 377 - protein, 8 aa, synthetic construct
SSSYLAWY

SEQ ID NO: 378 - protein, 6 aa, synthetic construct
QSISSY

SEQ ID NO: 379 - protein, 11 aa, synthetic construct
RASQSISSYLN

SEQ ID NO: 380 - protein, 7 aa, synthetic construct
SSYLNWY

SEQ ID NO: 381 - protein, 14 aa, synthetic construct
RSSTGAVTTSNYAN

SEQ ID NO: 382 - protein, 6 aa, synthetic construct
NIGSKS

SEQ ID NO: 383 - protein, 11 aa, synthetic construct
GGNNIGSKSVH

SEQ ID NO: 384 - protein, 7 aa, synthetic construct
SKSVHWY

SEQ ID NO: 385 - protein, 6 aa, synthetic construct
QSVSSN

SEQ ID NO: 386 - protein, 11 aa, synthetic construct
RASQSVSSNLA

SEQ ID NO: 387 - protein, 7 aa, synthetic construct
SSNLAWY

SEQ ID NO: 388 - protein, 14 aa, synthetic construct
ASSTGAVTSGNYPN

SEQ ID NO: 389 - protein, 6 aa, synthetic construct
QDVRNL

SEQ ID NO: 390 - protein, 11 aa, synthetic construct
KASQDVRNLVV

SEQ ID NO: 391 - protein, 7 aa, synthetic construct
RNLVVWF

SEQ ID NO: 392 - protein, 12 aa, synthetic construct
QSLLNSRTRKNY

SEQ ID NO: 393 - protein, 17 aa, synthetic construct
KSSQSLLNSRTRKNYLA

SEQ ID NO: 394 - protein, 13 aa, synthetic construct
LNSRTRKNYLAWY

SEQ ID NO: 395 - protein, 7 aa, synthetic construct
DASNRAT

SEQ ID NO: 396 - protein, 10 aa, synthetic construct
LLIYDASNRA

SEQ ID NO: 397 - protein, 7 aa, synthetic construct
SASYRYI

SEQ ID NO: 398 - protein, 10 aa, synthetic construct
RLIYSASYRY

SEQ ID NO: 399 - protein, 7 aa, synthetic construct
GDSKRPS

SEQ ID NO: 400 - protein, 10 aa, synthetic construct
LVIYGDSKRP

SEQ ID NO: 401 - protein, 7 aa, synthetic construct
WASTRHT

SEQ ID NO: 402 - protein, 10 aa, synthetic construct
LLIYWASTRH

SEQ ID NO: 403 - protein, 7 aa, synthetic construct
KVSNRFS

SEQ ID NO: 404 - protein, 10 aa, synthetic construct
LLIYKVSNRF

SEQ ID NO: 405 - protein, 7 aa, synthetic construct
YTSRLHT

SEQ ID NO: 406 - protein, 10 aa, synthetic construct
LLIYYTSRLH

SEQ ID NO: 407 - protein, 7 aa, synthetic construct
GTKFLAP

SEQ ID NO: 408 - protein, 10 aa, synthetic construct
GLIGGTKFLA

SEQ ID NO: 409 - protein, 7 aa, synthetic construct
SASTRAT

SEQ ID NO: 410 - protein, 10 aa, synthetic construct
LLIHSASTRA

SEQ ID NO: 411 - protein, 7 aa, synthetic construct
GTNKRAP

SEQ ID NO: 412 - protein, 10 aa, synthetic construct
GLIGGTNKRA

SEQ ID NO: 413 - protein, 7 aa, synthetic construct
WASTRES

SEQ ID NO: 414 - protein, 10 aa, synthetic construct
LLISWASTRE

SEQ ID NO: 415 - protein, 7 aa, synthetic construct
DASIRAT

SEQ ID NO: 416 - protein, 10 aa, synthetic construct
LLMYDASIRA

SEQ ID NO: 417 - protein, 7 aa, synthetic construct
AASSLQS

SEQ ID NO: 418 - protein, 10 aa, synthetic construct
LLIYAASSLQ

SEQ ID NO: 419 - protein, 7 aa, synthetic construct
DDSDRPS

SEQ ID NO: 420 - protein, 10 aa, synthetic construct
VVVYDDSDRP

SEQ ID NO: 421 - protein, 7 aa, synthetic construct
GASTRAT

SEQ ID NO: 422 - protein, 10 aa, synthetic construct
LLIYGASTRA

SEQ ID NO: 423 - protein, 4 aa, synthetic construct
GTKF

SEQ ID NO: 424 - protein, 7 aa, synthetic construct
GTKFLVP

SEQ ID NO: 425 - protein, 10 aa, synthetic construct
GLIGGTKFLV

SEQ ID NO: 426 - protein, 7 aa, synthetic construct
SGSYRYS

SEQ ID NO: 427 - protein, 10 aa, synthetic construct
LLIYSGSYRY

SEQ ID NO: 428 - protein, 7 aa, synthetic construct
WTSTRKS

SEQ ID NO: 429 - protein, 10 aa, synthetic construct
LLIYWTSTRK

SEQ ID NO: 430 - protein, 9 aa, synthetic construct
QQRSNWPPT

SEQ ID NO: 431 - protein, 8 aa, synthetic construct
QQRSNWPP

SEQ ID NO: 432 - protein, 9 aa, synthetic construct
QQHYSPPYT

SEQ ID NO: 433 - protein, 8 aa, synthetic construct
QQHYSPPY

SEQ ID NO: 434 - protein, 10 aa, synthetic construct
QTYTGGASLV

SEQ ID NO: 435 - protein, 9 aa, synthetic construct
QTYTGGASL

SEQ ID NO: 436 - protein, 9 aa, synthetic construct
QQYSSYPYT

SEQ ID NO: 437 - protein, 8 aa, synthetic construct
QQYSSYPY

SEQ ID NO: 438 - protein, 10 aa, synthetic construct
SQSTHVPPFT

SEQ ID NO: 439 - protein, 9 aa, synthetic construct
SQSTHVPPF

SEQ ID NO: 440 - protein, 9 aa, synthetic construct
QQGNTLPWT

SEQ ID NO: 441 - protein, 8 aa, synthetic construct
QQGNTLPW

SEQ ID NO: 442 - protein, 9 aa, synthetic construct
VLWYSNRWV

SEQ ID NO: 443 - protein, 8 aa, synthetic construct
VLWYSNRW

SEQ ID NO: 444 - protein, 10 aa, synthetic construct
QQYGYPPDFT

SEQ ID NO: 445 - protein, 9 aa, synthetic construct
QQYGYPPDF

SEQ ID NO: 446 - protein, 9 aa, synthetic construct
ALWYSNLWV

SEQ ID NO: 447 - protein, 8 aa, synthetic construct
ALWYSNLW

SEQ ID NO: 448 - protein, 8 aa, synthetic construct
KQSYDLFT

SEQ ID NO: 449 - protein, 7 aa, synthetic construct
KQSYDLF

SEQ ID NO: 450 - protein, 9 aa, synthetic construct
QQYQSWPLT

SEQ ID NO: 451 - protein, 8 aa, synthetic construct
QQYQSWPL

SEQ ID NO: 452 - protein, 10 aa, synthetic construct
QQSYSTPPIT

SEQ ID NO: 453 - protein, 9 aa, synthetic construct
QQSYSTPPI

SEQ ID NO: 454 - protein, 11 aa, synthetic construct
QVWDSSSDHVV

SEQ ID NO: 455 - protein, 10 aa, synthetic construct
QVWDSSSDHV

SEQ ID NO: 456 - protein, 9 aa, synthetic construct
QQYNNWPWT

SEQ ID NO: 457 - protein, 8 aa, synthetic construct
QQYNNWPW

SEQ ID NO: 458 - protein, 9 aa, synthetic construct
TLWYSNRWV

SEQ ID NO: 459 - protein, 8 aa, synthetic construct
TLWYSNRW

SEQ ID NO: 460 - protein, 8 aa, synthetic construct
KQSFILRT

SEQ ID NO: 461 - protein, 7 aa, synthetic construct
KQSFILR